(19) United States
(12) Patent Application Publication
(10) Pub. No.: US 2022/0290247 A1
(43) Pub. Date: Sep. 15, 2022

(54) COMPOSITIONS AND METHODS FOR DIAGNOSIS AND TREATMENT OF BLADDER CANCER

(71) Applicant: The Regents of the University of California, Oakland, CA (US)

(72) Inventors: Lawrence FONG, San Francisco, CA (US); Chun Jimmie YE, San Francisco, CA (US); David Y. OH, San Francisco, CA (US)

(21) Appl. No.: 17/633,922

(22) PCT Filed: Aug. 6, 2020
(86) PCT No.: PCT/US2020/045263

§ 371 Date: Feb. 8, 2022

Related U.S. Application Data
(60) Provisional application No. 62/885,120, filed on Aug. 9, 2019.

(51) Int. Cl. C12Q 1/6886; A61P 35/00; C12N 5/0783; C07K 16/28; C12Q 1/6806; C12Q 1/6813

(52) U.S. Cl. CPC C12Q 1/6886; A61P 35/00; C12N 5/0637; C07K 16/2827; C12Q 1/6806; C12N 5/0638; C12Q 1/6813; C12Q 2600/106; C12Q 2600/158

(57) ABSTRACT

The present disclosure relates generally to, inter alia, therapeutic and diagnostic methods and compositions for treatment of bladder cancer. In particular, the disclosure relates to defining pre-treatment gene signatures that are predictive of response to anti-PD-L1 therapy and to the use of such gene signatures as biomarkers to identify individuals having or suspected of having bladder cancer who are most likely to respond to an anti-PD-L1 therapy. In some embodiments, various methods for the treatment of bladder cancer in individuals identified by the diagnostic methods disclosed herein are also provided.

### CROSS-REFERENCE TO RELATED APPLICATIONS

This application claims the benefit of priority to U.S. Provisional Patent Application Ser. No. 62/885,120, filed on Aug. 9, 2019. The disclosure of the above-referenced application is herein expressly incorporated by reference it its entirety, including any drawings.

### STATEMENT REGARDING FEDERALLY SPONSORED R&D

This invention was made with government support under grants no. K08 A1139375, R01 CA194511, and U01 CA233100 awarded by The National Institutes of Health. The government has certain rights in the invention.

### FIELD

Disclosed herein, inter alfa, are therapeutic and diagnostic methods and compositions for treatment of bladder cancer. In particular, the disclosure relates to defining pre-treatment gene signatures that are predictive of response to anti-Programmed Death Ligand 1 (PD-L1) therapy and to the use of such gene signatures as biomarkers to identify individuals having or suspected of having bladder cancer who are most likely to respond to an anti-PD-L1 therapy.

### BACKGROUND

Cancers, or malignant tumors, metastasize and grow rapidly in an uncontrolled manner, making timely detection and treatment extremely difficult. Therefore, cancer remains one of the most deadly threats to human health. In the U.S., cancer is the second leading cause of death after heart disease, accounting for approximately 1 in 4 deaths. Solid tumors are responsible for most of those deaths, and bladder cancer is among the most common malignancies worldwide. In particular, metastatic urothelial bladder cancer is associated with poor outcomes and represents a major unmet medical need with few effective therapies to date.

Recent clinical studies have indicated that although bladder cancer responds to immunotherapies, rates of clinical response are generally low. The contribution of T cells other than cytotoxic CDS⁺ to tumor rejection is unknown. For example, bladder cancer can be responsive to immunotherapies such as anti-PD-1 and anti-PD-L1 checkpoint inhibitors, which are believed to relieve inhibition of cytotoxic CDS⁺ T cells resulting in tumor cell killing. However, although immunotherapies such as anti-PD-1 and anti-PD-L1 checkpoint inhibitors have shown some promise in treating bladder cancer, the overall response rates have remained low. In addition, while cytotoxic CD8⁺ T cells are thought to mediate tumor rejection, the contribution of other tumor-resident T cells, which may possess heterogeneity in their antigenic repertoire and function, is unknown.

Given the importance of immune checkpoint pathways in regulating an immune response, the need exists for developing additional therapeutic and diagnostic approaches, including gene expression analysis and immunotherapies, for more effectively treating and diagnosing cancer such as bladder cancer.

### SUMMARY

The present disclosure relates generally to, inter alfa, therapeutic and diagnostic methods and compositions for treatment of bladder cancer, and particularly relates to defining pre-treatment gene signatures that are predictive of responsiveness to anti-PD-L1 therapy and to the use of such gene signatures as biomarkers to identify individuals as predicted to have an increased responsiveness to the anti-PD-L1 immunotherapy, e.g., individuals who are most likely to respond to an anti-PD-L1 therapy. In some particular embodiments, the disclosure further provides therapeutic methods for the treatment of bladder cancer in individuals identified by the diagnostic methods disclosed herein. The gene signatures disclosed herein not been previously described, and may have advantages over existing signatures in that they may outperform the ability of existing signatures to predict response to, or prognosticate longer survival with, anti-PD-L1 therapy of bladder cancer. As describes in greater detail below, some embodiments of the disclosure provide novel single-gene signatures and composite gene signatures that are associated with specific types of tumor-infiltrating T cells in human bladder tumors. For example, the data presented herein demonstrated that these gene signatures are associated with subsequent response to and/or longer survival with cancer immunotherapies, particularly anti-PD-L1 antibodies, in metastatic bladder cancer based on expression analysis in a pre-treatment tumor biopsy. In some embodiments, the disclosure provides compositions and methods for selecting individuals having bladder cancer to be subjected to a therapeutic treatment including a PD-L1 antagonist. In some particular embodiments, the disclosure also provides kits and systems useful for predicting responsiveness of a bladder cancer to an anti-PD-L1 therapy. In particular, various kits and systems of using a gene expression platform to derive gene signature biomarkers of anti-cancer response to a PD-L1 therapy and to test patient samples for predictive gene signature biomarkers are disclosed.

In one aspect, provided herein are methods for predicting responsiveness of an individual having or suspected of having bladder cancer to a therapy including an antagonist of Programmed Death Ligand 1 (PD-L1). The methods include (a) profiling expression levels of a panel of genes associated with T-cell specialization and/or T-cell exhaustion expressed in a T cell population from a biological sample obtained from the individual to generate a cell composition profile of the T cell population; (b) determining the presence of a gene signature biomarker in the T cell population based at least in part upon the measured expression levels and the generated cell composition profile, wherein said gene signature biomarker includes one or more genes whose expression is specifically upregulated in proliferating and/or non-proliferating cytotoxic CD4+ T cells while remains unchanged in CD8+ T cells; and (c) identifying the individual as predicted to have an increased responsiveness to the anti-PD-L1 therapy if the gene signature is present in the biological sample.

In another aspect, provided herein are methods for selecting an individual having bladder cancer to be subjected to a therapy including a PD-L1 antagonist, the method includes (a) profiling expression levels of a panel of genes associated with T-cell specialization and/or T-cell exhaustion expressed in a T cell population from a biological sample obtained from an individual to generate a cell composition profile of the T cell population; (b) determining the presence of a gene signature biomarker in the T cell population based at least in part upon the measured expression level and the generated cell composition profile, wherein said gene signature biomarker includes one or more genes whose expression is specifically upregulated in proliferating and/or non-proliferating cytotoxic CD4+ T cells while remains unchanged in CD8+ T cells; and (c) selecting the individual who is determined to have the gene signature present in the biological sample as an individual to be subjected to a therapy including a PD-L1 antagonist.

In another aspect, provided herein are methods for treating an individual having bladder cancer, the methods include: (a) profiling expression levels of a panel of genes associated with T-cell specialization and/or T-cell exhaustion expressed in a T cell population from a biological sample obtained from said individual to generate a cell composition profile of the T cell population; (b) determining the presence of a gene signature biomarker in the T cell population based at least in part upon the measured expression levels and the generated cell composition profile, wherein said gene signature biomarker includes one or more genes whose expression is specifically upregulated in proliferating and/or non-proliferating cytotoxic CD4+ T cells while remains unchanged in CD8+ T cells; (c) selecting a therapy including a PD-Ll antagonist; and (d) administering a therapeutically effective amount of the selected therapy to said individual.

Non-limiting exemplary embodiments of the methods according to the present disclosure include one or more of the following features. In some embodiments, the cell composition profile includes relative proportions of the following T cell subpopulations: tumor-reactive ENTPD1+CD8+ T cells, naïve CD8+ T cells, HSP+CD8+ T cells, mucosal-associated invariant CD8+ T cells, FGFBP2+CD8+ T cells, XCL1+XCL2+CD8+ T cells, central memory CD8+ T cells, effector memory CD8+ T cells, exhausted CD8+ T cells, proliferating CD8+ T cells, regulatory CD4+ T cells, central memory CD4+ T cells, exhausted CD4+ T cells, proliferating cytotoxic CD4+ T cells, and non-proliferating cytotoxic CD4+ T cells. In some embodiments the gene signature biomarker includes one or more of the following parameters: (i) one or more genes identified in Table 2 or Table 7 as upregulated in proliferating CD8⁺ T cells; (ii) one or more genes identified in Table 3 or Table 10 as upregulated in proliferating CD4⁺ T cells; (iii) one or more genes identified in Table 4 or Table 8 as upregulated in regulatory CD4⁺ T cells; (iv) one or more genes or identified in Table 9 as upregulated in cytotoxic CD4+ T cells; and (v) one of more genes identified in Table 5 as upregulated in proliferative cytotoxic CD4⁺ T cells. In some embodiments, the gene signature biomarker includes at least 2, at least 3, at least 5, at least 10, at least 20, at least 30, at least 40, at least 50 genes. In some embodiments, the gene signature biomarker includes one or more of ABCB1, ACTB, ABCB1, ATP5E, CARD16, CXCL13, GPR18, GZMB, HIST1H4C, IGLL5, IL2RA, IL32, KIAA0101, KIF15, MIR4435-1HG, MYL6, PEG10, SLAMF7, STMN1, TIGIT, TMSB10, TUBA1B, TUBB, GZMK, HLA-DR, PDCD1, TIM3, KLRG1, and combinations of any thereof. In some embodiments, the gene signature biomarker includes one or more of ABCB1, ACTB, APBA2, GPR18, HIST1H4C, IGLL5, IL2RA, IL32, MIR4435-1HG, MYL6, SLAMF7, STMN1, TMSB10, TUBB, and combinations of any thereof In some embodiments, the gene signature biomarker includes one or more of GZMK, HLA-DR, PDCD1, TIM3, KLRG1, and combinations of any thereof.

In some embodiments, the biological sample includes bladder cancer cells. In some embodiments, the biological sample includes peripheral blood. In some embodiments, the bladder cancer is selected from the group consisting of squamous cell carcinoma, non-squamous cell carcinoma, adenocarcinoma, and small cell carcinoma. In some embodiments, the bladder cancer is selected from the group consisting of metastatic bladder cancer, non-metastatic bladder cancer, early-stage bladder cancer, non-invasive bladder cancer, muscle-invasive bladder cancer (MIBC), non-muscle-invasive bladder cancer (NMIBC), primary bladder cancer, advanced bladder cancer, locally advanced bladder cancer, bladder cancer in remission, progressive bladder cancer, and recurrent bladder cancer. In some embodiments, the bladder cancer is metastatic bladder cancer.

In some embodiments, the PD-L1 antagonist includes an anti-PD-L1 antibody. In some embodiments, the anti-PD-L1 antibody includes one or more of atezolizumab (MPDL3280A), BMS-936559 (MDX-1105), durvalumab (MEDI4736), avelumab (MSB0010718C), YW243.55.570, and combinations of any thereof. In some embodiments, the anti-PD-L1 antibody includes atezolizumab. In some embodiments, the PD-L1 antagonist includes an anti-PD1 antibody. In some embodiments, the anti-PD1-antibody includes pembrolizumab, nivolumab, cemiplimab, pidilizumab, lambrolizumab, MEDI-0680, PDR001, REGN2810, and combinations of any thereof In some embodiments, the anti-PD1 antibody comprises pembrolizumab. In some embodiments, the gene signature biomarker includes one or more genes whose expression is upregulated in proliferating CD4+ T cells and/or upregulated in non-proliferating CD4+ T cells while remains substantially unchanged in CD8+ T cells. In some embodiments, the gene signature biomarker includes one or more genes selected from the group consisting of ABCB1, APBA2, SLAMF7, GPR18, PEG10, and combinations of any thereof. In some embodiments, the gene signature biomarker includes one or more genes selected from the group consisting of GZMK, GZMB, HLA-DR, PDCD1, TIM3, and combinations of any thereof. In some embodiments, the gene signature biomarker includes a gene combination selected from the group consisting of: (a) expression of CD4, GZMB, and HLA-DR; (b) expression of CD4, GZMK and HLA-DR; and (c) expression of CD4, GZMK, PDCD1, and TIM3. In some embodiments, the gene signature biomarker further includes undetectable expression of FOXP3 and CCR7.

In some embodiments, the gene signature biomarker includes one or more genes selected from the group consisting of GZMB, GZMK, HLA-DR, PDCD1, Ki67, TIM3, and combinations of any thereof. In some embodiments, the gene signature biomarker includes a gene combination selected from the group consisting of: (a) expression of CD8, GZMB, and TIM3: (b) expression of CD8, GZMB, PDCD1, and TIM3; (c) expression of CD8, GZMK, and TIM3; (d) expression of CD8, GZMK, PDCD1, and TIM3; (e) expression of CD8, GZMK, and HLA-DR; (f) expression of CD8, GZMK, and Ki67; and (g) expression of CD8, GZMK, HLA-DR, and Ki67. In some embodiments, the gene signature biomarker further includes undetectable expression of CCR7.

In some embodiments, the profiling expression levels of a panel of genes associated with T-cell specialization and/or T-cell exhaustion includes a nucleic acid-based analytical assay selected from the group consisting of single-cell RNA sequencing, T-cell receptor (TCR) sequencing, single sample gene set enrichment analysis, northern blotting, fluorescent in-situ hybridization (FISH), polymerase chain reaction (PCR), real-time PCR, reverse transcription polymerase chain reaction (RT-PCR), quantitative reverse transcription PCR (qRT-PCR), serial analysis of gene expression (SAGE), microarray, tiling arrays. In some embodiments, the nucleic acid-based analytical assay includes single-cell RNA sequencing. In some embodiments, the profiling expression levels of a panel of genes associated with T-cell specialization and/or T-cell exhaustion includes a protein expression-based analytical assay selected from the group consisting of ELISA, immunohistochemistry, western blotting, mass spectrometry, flow cytometry, protein-microarray, immunofluorescence, multiplex detection assay, and combinations of any thereof. In some embodiments, the protein-expression-based analytical assay includes flow cytometry.

In some embodiments, the disclosed methods further include treating the bladder cancer by administering to the individual a first therapy including therapeutically effective amount of the PD-L1 antagonist. In some embodiments, the methods of the disclosure further include (a) selecting a PD-L1 antagonist appropriate for a therapy of the bladder cancer in the individual based on whether the gene signature biomarker is present in the individual; and (b) administering a first therapy including a therapeutically effective amount of the selected PD-Ll antagonist to the individual.

In some embodiments, the methods of the disclosure include administering to the individual the first therapy in combination with a second therapy. In some embodiments, the second therapy is selected from the group consisting of chemotherapy, radiation therapy, immunotherapy, immunoradiotherapy, hormonal therapy, toxin therapy, and surgery. In some embodiment, the second therapy is an anti-PD-1 therapy. In some embodiments, the second therapy is an anti-transforming growth factor β (TGF-β) therapy. In some embodiments, the first therapy and the second therapy are administered concomitantly. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy.

In another aspect, provided herein are various kits for use in predicting responsiveness of a bladder cancer to an anti-PD-L1 therapy and/or in treating a bladder cancer in an individual. The kits include (a) one or more detection reagents capable of detecting and/or profiling expression levels of a panel of genes associated with T-cell specialization and/or T-cell exhaustion expressed in a T cell population to generate a cell composition profile of the T cell population, and (b) instructions for use in predicting responsiveness of a bladder cancer to an anti-PD-L1 therapy and/or in treating a bladder cancer in an individual. In some embodiments, the kits include (a) one or more detection agents capable of detecting one or more of the following parameters in a biological sample from a subject: (i) one or more genes identified in Table 2 or Table 7 as upregulated in proliferating CD8⁺ T cells; (ii) one or more genes identified in Table 3 or Table 10 as upregulated in proliferating CD4+ T cells; (iii) one or more genes identified in Table 4 or Table 8 as upregulated in regulatory CD4⁺ T cells; (iv) one or more genes identified in Table 9 as upregulated in cytotoxic CD4+ T cells and (v) one of more genes identified in Table 5 as upregulated in proliferative cytotoxic CD4⁺ T cells; and (b) instructions for use in predicting responsiveness of a bladder cancer to an anti-PD-L1 therapy and/or in treating a bladder cancer in an individual. In some embodiments, the disclosed kits further include an antagonist of PD-L1 and optionally an antagonist of PD-1 or a combination thereof.

Also provided, in another aspect, are various system including (a) at least one processor; and (b) at least one memory including program code which when executed by the one memory provides operations for performing a method as disclosed herein. In some embodiments, the operations include (a) acquiring knowledge of the presence of a gene signature biomarker in a biological sample from an individual; and (b) providing, via a user interface, a prognosis for the subject based at least in part on the acquired knowledge.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIGS. 1A-1B show an overview of the experimental approach and relative abundance of CD4⁺ and CD8⁺ T cells in bladder tumors. FIG. 1A: Schematic of processing for paired tumor, adjacent non-malignant tissue, and blood from either anti-PD-Ll-treated, or untreated/chemotherapy-treated, cystectomy patients. FACS-sorted CD4⁺ or CD8⁺ T cells were subjected to droplet-based single-cell RNA sequencing (dscRNAseq) with paired T cell receptor (TCR) sequencing as described in the text. FIG. 1B: Parallel flow cytometry data from the same single-cell digest used for dscRNAseq from 4 anti-PD-Ll-treated tumors, showing the percentage of CD4⁺ or CD8⁺ T cells from total CD3⁺ cells.

FIGS. 2A-2D summarize the results of experiments showing that intratumoral CD8⁺ T cells in bladder tumors include known populations of MATT, effector, central memory, and proliferating T cells. FIG. 2A: tSNE plots of 11,794 single sorted CD3⁺ CD8⁺ T cells obtained from bladder tumors and adjacent non-malignant tissue from 7 patients. Phenotypic clusters (left) and compartment of origin (tumor and non-malignant are blue and red, right) are shown. FIG. 2B: Relative intensity of expression of select genes superimposed upon the tSNE projections shown in FIG. 2A. FIG. 2C: Heatmap showing all single cells (columns) grouped by the unbiased clusters shown in FIG. 2A and by tissue of origin (colors at top of heatmap), with relative expression of the top 5 ranked differentially expressed genes for each cluster compared to the CCR7⁺ tCD8-c0 cluster and conserved across tumor and non-malignant compartments based on meta testing (genes as rows, ordered by fold change, all P_{adj} values <0.05) displayed. Select marker genes are identified that are differentially expressed between subpopulations based on pairwise population testing. FIG. 2A: The abundance of cells in individual populations is shown as a percentage of total CD8⁺ cells within either tumor or non-malignant compartments across all patients (orange=tumor, N=7 samples; blue=non-malignant, N=3 samples). *, P <0.05, **, P <0.01 and FDR <0.1 by unpaired two-tailed T test assuming unequal variances with Benjamini-Hochberg correction for multiple testing.

FIG. 3 depicts tSNE plots showing cluster representation for CD4⁺ and CD8⁺ TIL from individual patients.

FIGS. 4A-4C pictorially summarize the results of experiments illustrating that CD4⁺ T cells in bladder tumors are composed of canonical and novel functional populations.

FIG. 4A: t-Distributed Stochastic Neighbor Embedding (tSNE) plots of 21,932 single sorted CD3⁺ CD4⁺ T cells obtained from bladder tumors and adjacent non-malignant tissue from 7 patients. At left, each distinct phenotypic cluster identified using graph-based k-nearest neighbor (KNN) methods (Seurat) is identified with a distinct color. At right, the sample of origin of the same cells (tumor or non-malignant) is indicated by appropriate colors (blue or red, respectively). Annotation of each unbiased cluster was performed by manual inspection of highest-ranked differentially expressed genes for each cluster, and also using reference signature-based correlation methods (SingleR) as described in the text. FIG. 4B: Relative intensity of expression of select genes superimposed upon the tSNE projections shown in FIG. 4A. FIG. 4C: Heatmap showing all single cells (columns) grouped by the unbiased clusters shown in (A) and by tissue of origin, patient, and treatment (colors at top of heatmap), with relative expression of the top 5 ranked differentially expressed genes for each cluster compared to the CCR7⁺ tCD4-cl cluster (genes as rows, ordered by fold change, all P_{adj} values <0.05) displayed. Select marker genes are labeled at right that are differentially expressed between subpopulations based on pairwise population testing. Cluster names and annotation of cell type are shown for each cluster.

FIG. 5 depicts the abundance of cells in individual populations as—determined by manual gating of flow cytometry data is shown as a percentage of total CD4⁺ cells within tumor.

FIG. 6 depicts gating strategy for flow cytometric analysis of populations in CD4⁺ and CD8⁺ T cells from RNAseq. (A) CD4⁺ and CD8⁺ populations were gated out of CD3⁺ CD45+ single live cells. CD4⁺ cells were further gated as FoxP3⁻ and FoxP3⁺ . Treg cells are gated as FOXP3⁺ CD25⁺ cells. FOXP3⁻ CD4⁺ and CD8⁺ cells were gated into central memory (CM, CCR7⁺ CD45RA⁻), and effector memory plus effector (EM+E, CCR7⁻ CD45RA⁻ and CCR7⁻ CD45RA⁺ respectively). Boolean gating of EM+E was used to obtain GZMK⁺ GZA4B⁺ , GZMK+ GZMB⁻, GZMK⁻ GZMB⁺ and HLADR⁺ Ki67⁺ populations for further marker analysis. Plots are shown here to demonstrate the presence of these populations. (B) Representative gate for each marker shown here for CD4⁺ are used for Boolean gating for the populations described above as well as for analyzing the expression of each marker in the following populations: CM, TregCD25¹′, TregCD25^{hi}, EM+E GZMK⁺ GZ1V1B⁺ , EM+E_GZMK⁺ GZMB⁻, EM+E_GZMK⁻ GZMB⁺ , EM+E_CXCL13 and EM+E_HLADR⁺ Ki67⁺ .

FIGS. 7A-7I summarize the results of experiments illustrating that regulatory CD4⁺ T cell populations include heterogeneous populations, which are enriched and clonally expanded in bladder tumors. FIG. 7A: Violin plots of select marker genes that are differentially expressed between regulatory subpopulations (regulatory T cell populations labeled in red). FIG. 7B: For regulatory populations (red) and other CD4+ T cell populations (black), the abundance of cells in individual populations by scRNAseq is shown as a percentage of total CD4⁺ cells within either tumor or non-malignant compartments across all patients (orange=tumor, N=7 samples; blue=non-malignant, N=6 samples). *, P <0.05, **, P <0.01 and FDR <0.1 by unpaired two-tailed T test assuming unequal variances with Benjamini-Hochberg correction for multiple testing. FIG. 7C: Representative flow cytometry plot from CD4⁺ FOXP3⁺ tumor-infiltrating lymphocytes from an unrelated bladder tumor showing gating strategy for CD25″g, CD25¹° ′ and CD25 (upper left), and histograms of TNFRSF18 staining from each CD25 gate (upper right). MFI of TNFRSF18 and 9/0 TNFRSF18+from parent gate are shown for CD25 gates across samples (N=7 tumors, mean +s.e.m.). *, P <0.05 by Wilcoxon paired T test. FIG. 7D: percentage of cells in CD25¹′, CD25, and other manually gated populations by flow cytometry are shown from total CD4⁺ T cells (N=7 tumors). FIG. 7E: The percentage of unique paired TRA and TRB CDR3 nucleotide sequences that are expressed by one cell (blue), shared by two cells (green), or shared by three or more cells (red) is indicated for CD4⁺ T cells from individual tumor (darker shades) and non-malignant tissues (lighter shades) from anti-PD-L1-treated (“PD-L1”), untreated, and chemotherapy-treated (“chemo”) patients. Triplicate control samples from a single healthy donor's CD4⁺ T cells sorted from peripheral blood and processed for scRNAseq and TCR in identical fashion in separate sequencing runs is shown (“healthy 1-3”), as well as reference publicly available data from peripheral blood CD4⁺ from a healthy donor. FIG. 7F: Lorenz curves showing the cumulative frequency distributions for unique CD4⁺ T cells and unique CD4⁺ T cell clonotypes for tumor, non-malignant tissues, and healthy donor blood. FIG. 7G: Gini coefficients for CD4⁺ T cell clonotypes from tumor, non-malignant tissues, and healthy donor blood, calculated from the Lorenz curves in (F); P=0.005 by Wilcoxon for tumor versus non-malignant tissues. For (F) and (G): N=7 tumor samples; 6 non-malignant samples, 4 healthy donor samples (3 triplicates from one healthy donor, 1 data set from 10× Genomics). FIG. 7H: Gini coefficients for regulatory (red) and other (black) CD4⁺ T cell populations within tumor and non-malignant compartments across all samples (*, P <0.05, **, P <0.01 and FDR <0.1 by Wilcoxon test with Benjamini-Hochberg correction for multiple testing). N=7 tumor samples; 6 non-malignant samples. FIG. 7I: Left, Single cells expressing the top 10 most expanded clonotypes found in the combined regulatory populations (tCD4-c0, tCD4-c5, tCD4-c6) are shown in red in the same tSNE space as FIG. 4A. The regions composed of regulatory T cells are outlined and superimposed upon the tSNE projection color-coded by expanded clones (left) or color-coded by tumor or non-malignant tissue origin (right).

FIG. 8 depicts volcano plots showing nominal P values versus log2(FC) for differential testing of genes between tumor and non-malignant compartments for regulatory T cell populations (tCD4-c0, tCD4-c5, tCD4-c6) and cytotoxic CD4+populations (tCD4-c4, tCD4-c7, tCD4-c9, tCD4-c10). Genes whose expression is significantly different between compartments with P <0.05 and llog₂(FC) >1.41 are shown in red.

FIG. 9A: Gini coefficients for tissue-infiltrating CD4⁺ in individual populations, separated by treatment type. FIGS. 9B-9D: aired TRA/TRB clonotype sharing between cells, Lorenz curves, and Gini coefficients for CD8⁺ clonotype data as in FIGS. 9G-9I. FIGS. 9E-9F: Gini coefficients for CD8⁺ T cells in individual populations, separated by tumor versus non-malignant tissue (FIG. 9E) and treatment type (FIG. 9F).

FIGS. 10A-10K summarize the results of experiments illustrating that bladder tumors possess multiple cytotoxic CD4⁺ T cell populations, which are clonally expanded in bladder tumors and can lyse autologous tumor cells. FIG. 10A: Violin plots of select marker genes that are differentially expressed between cytotoxic and regulatory subpopulations. Cytotoxic populations are shown in purple, regulatory populations in red. FIG. 10B: At left, Representative flow cytometry plot of GZMB and GZMK expression within the combination of CCRT CD4⁺ FOXP3⁻ populations (e.g., effector memory CCRT CD45RA⁻ and effector CCR7⁻ CD45RA⁺ ) from tumor-infiltrating lymphocytes obtained from an unrelated bladder tumor. Middle and right, representative flow cytometry plots of GZMB and GZMK (versus CD3) expression in total CD4⁺ T cells, with superimposed manual gate used for Boolean analysis. FIG. 10C: percentage of cells expressing GZMB, GZMK, or both by flow cytometry are shown from total CD4⁺ T cells (N=7 tumors, mean +s.e.m.). FIG. 10D: Abundance of cells within each cytotoxic population by scRNAseq as a percentage of total CD4⁺ T cells within tumor or non-malignant compartments across all patients (orange=tumor, N=7 samples; blue=non-malignant, N=6 samples). FIG. 10E: The ratio of abundances of all regulatory T cell populations (tCD4-c0 +tCD4-c5 +tCD4-c6) to all cytotoxic CD4⁺ populations (tCD4-c4 + tCD4-c7 +tCD4-c9 +tCD4-c10) across all tumor and non-malignant samples is shown. For FIG. 10D and 10E: *, P <0.05, **, P <0.01 and FDR <0.1 by unpaired two-tailed T test assuming unequal variances with Benjamini-Hochberg correction for multiple testing. FIG. 10F: Gini coefficients for each of the cytotoxic CD4⁺ populations within tumor and non-malignant compartments across all samples (*, P <0.05, **, P <0.01 and FDR <0.1 by Wilcoxon test with Benjamini-Hochberg correction for multiple testing). N=7 tumor samples, 6 non-malignant samples. FIG. 10G: Specific timepoints from a time-lapse microscopy experiment are shown where sorted CD4⁺ TIL (with regulatory T cells excluded) from a localized bladder tumor were isolated, cultured ex vivo (see Methods), and re-incubated with autologous tumor cells at an effector:target ratio of approximately 30:1 at timepoint 0. Timepoints involving recognition of tumor by TILs (as evidenced by cluster formation), and killing (with increase in uptake of red cell death indicator) are displayed at the indicated times. (H-K) Analysis of the increase in the number of dead cells over time from the same killing assay for CD4⁺ TIL at 30:1 effector:target ratio (FIG. 10H), CD8⁺ TIL at 30:1 effector:target ratio (FIG. 10I), CD4⁺ TIL at 30:1 effector:target ratio with MHC-II blockade (FIG. 10J), or CD8⁺ TIL at 30:1 effector:target ratio with MHC-I blockade (FIG. 10K), are shown. Control traces from the same wells but restricting analysis to free TIL, or from separate wells with tumor only, are included. All traces were normalized to the number of dead cells per mm² at timepoint 0. The observation of autologous tumor killing within hours by CD4⁺ and CD8⁺ TIL above the background level of spontaneous death of TIL from the same wells is representative of 2 independent experiments involving distinct aliquots from the same patient.

FIG. 11 depicts unbiased clustering of CD4⁺ T cells from tumor and adjacent non-malignant tissue from a single patient (anti-PD-L1 C), performed jointly without canonical correlation analysis alignment. Left panel: tSNE plot showing individual cells coded by cluster or by tissue of origin. Right panel: heatmap showing top 5 differentially expressed marker genes for each unbiased cluster.

FIG. 12 depicts annotations of single CD4⁺ T cells from tumor and adjacent non-malignant tissue using SingleR. “Other cell types” that were assigned include: adipocytes (12 cells), class-switched memory B cells (11 cells), common lymphoid progenitor (78 cells), dendritic cells (3 cells), epithelial cells (5 cells), erythrocytes (4 cells), fibroblasts (2 cells), granulocyte-macrophage progenitor (1 cell), hematopoietic stem cell (2 cells), keratinocytes (2 cells), M1 macrophages (1 cell), memory B cells (1 cell), megakaryocyte-erythroid progenitor (8 cells), naive B cells (1 cell), NK cells (32 cells), plasma cells (8 cells).

FIG. 13 depicts correlation matrix of all CD4⁺ and CD8⁺ populations from tissue (combined tumor and non-malignant tissues) based on expression of shared genes. Populations were arranged based on hierarchical clustering.

FIG. 14 summarize the results of experiments performed to demonstrate an association of single gene expression (log2(counts per million +1) expression, broken down into tertiles of expression) as well as single-sample gene set scores with overall survival (left column), binary response to therapy (middle column), or binary response to therapy subdivided by immune subtype (immune desert, immune excluded, or inflamed; right column) in the IMvigor 210 metastatic bladder cancer data set, testing the single genes MKI67, CXCL1 3 and GNLY, and 50-gene signatures from the proliferating tCD4-c11, proliferating tCD8-c9, and regulatory tCD4-c0 scRNAseq signatures. Association with overall survival was performed by Kaplan-Meier analysis of correlation between gene set scores and binary response to anti-PD-Ll therapy was done using two-tailed Wilcoxon.

FIGS. 15A-15I summarize the results of experiments illustrating that anti-PD-Ll therapy shifts T cell proliferation towards cytotoxic CD4⁺ T cells, which predict clinical response to anti-PD-Ll. FIG. 15A: Violin plots of select marker genes that are differentially expressed between proliferating (tCD4-c11), regulatory (tCD4-c0, tCD4-c5, tCD4-c6) and cytotoxic (tCD4-c4, tCD4-c7, tCD4-c9, tCD4-c10) T cell subpopulations. The proliferating population is shown in green, while regulatory populations are red and cytotoxic populations are purple. FIG. 15B: Heatmap showing expression of select proliferating, regulatory, and cytotoxic marker genes (rows) for individual single cells (columns) within the proliferating tCD4-c11 cluster. Both genes and samples were hierarchically clustered. Loge-transformed expression of each gene was row scaled. FIG. 15C: Abundance of cells within the proliferating tCD4-c11 population as a percentage of total CD4⁺ T cells within tumor or non-malignant compartments across all patients (orange=tumor, N=7 samples; blue=non-malignant, N=6 samples). *, P <0.05, **, P <0.01 by unpaired two-tailed T test assuming unequal variances with Benjamini-Hochberg correction for multiple testing. FIG. 15D: Gini coefficients for the proliferating tCD4-c11 population within tumor and non-malignant compartments across all samples (*, P <0.05, **, P <0.01 and FDR <0.1 by Wilcoxon test with Benj amini-Hochberg correction for multiple testing). N=7 tumor samples, 6 non-malignant samples. FIG. 15E: Pseudotime trajectories of anti-PD-L1-treated tumors (N=4) or untreated tumors (N=2). Cells with expanded TCRs from the proliferating (tCD4-c11, green), regulatory (tCD4-c0, tCD4-c5, tCD4-c6, shades of red), and cytotoxic (tCD4-c4, tCD4-c7, tCD4-c9, tCD4-c10, shades of purple) populations were used for this analysis. Specific branches in anti-PD-Ll-treated samples (left) corresponding to proliferating cytotoxic cells (branch 1), non-proliferative cytotoxic cells (branch 2), proliferating regulatory cells (branch 3), and non-proliferative regulatory cells (branch 5) are labeled. Also labeled are branch points that discriminate proliferating and non-proliferative cytotoxic CD4⁺ T cells (branch point 1), and proliferating and non-proliferative regulatory T cells (branch point 2). For comparison, branches corresponding to proliferative cytotoxic CD4⁺ and regulatory T cells in untreated samples are shown at right. FIG. 15F: Single cells expressing the top 10 most expanded clonotypes found in the proliferating CD4⁺ T cell population (tCD4-c11) are shown in red in the same tSNE space as FIG. 1A, for anti-PD-Ll-treated samples (left) and untreated samples (right). The regions composed of proliferating, regulatory, and cytotoxic T cells are outlined and superimposed upon the tSNE projection for visualization. FIG. 15G: Heatmap summarizing the degree of significant sharing (P value for observed sharing <0.05) of the same unique paired TRA and TRB clonotype CDR3 nucleotide sequences between 2 phenotypic populations within tumor. Sharing between populations that is only seen in anti-PD-Ll-treated tumors (red), untreated tumors (green), or both groups (yellow) is indicated. FIG. 1511: Association of single-sample gene set scores with response to therapy in the IMvigor 210 data set, testing 50-gene signatures from the proliferating tCD4-c11 scRNAseq signature, as well as the best scRNAseq signature associated with proliferative cytotoxic CD4⁺ T cells (branch point 1, cluster 5) and proliferating regulatory T cells (branch point 2, cluster 7) from pseudotime analysis in FIG. 15E. Analysis of correlation between gene set scores and binary response to anti-PD-L1 therapy was done using two-tailed Wilcoxon.

FIG. 16. Top panel depicts pseudotime trajectory from anti-PD-Ll-treated samples as in FIG. 15E, with specific branches colored by branch state. Bottom, results of hierarchical clustering of all differentially expressed genes between branches for branch point 1 (cytotoxic cells, left), and branch point 2 (regulatory cells, right). Specific branches are color-coded to match the trajectories at top. Specific clusters are also color-coded and labeled and reflect arbitrary clustering of genes based on co-regulation in specific branches.

FIGS. 17A-17C graphically summarize the results of experiments illustrating that bladder cancer contains canonical CD8+ T cell states. FIG. 17A depicts uniform manifold approximation and projection (UMAP) plots of 10,762 single sorted CD3⁺ CD8⁺ T cells obtained from bladder tumors and adjacent nonmalignant tissue (N=7 patients). Phenotypic clusters are represented in distinct colors. FIG. 17B: Relative intensity of expression of select genes superimposed on the UMAP projections in FIG. 17A. FIG. 17C: Violin plots showing the relative expression of select differentially expressed genes (columns) for each cluster shown in FIG. 17A (rows) (all Padj <0.05). FIG. 17D: The frequency of cells expressing MAIT-associated TRAV1-2/TRAJ33+TCRs within each defined CD8+phenotypic cluster. FIG. 17E: The frequency of cells in individual clusters shown as a proportion of total CD8+cells within tumor or non-malignant compartments across all patients (orange, tumor; blue, non-malignant). For each cluster, a box and whisker plot is shown with the median, interquartile range (IQR, a box with lower and upper bounds representing 25th and 75th percentiles, respectively), and 1.5 times the IQR (whiskers). Outlier points are shown if more than 1.5 times the IQR beyond the lower and upper quartiles. Statistical testing was done using an exact permutation test. FIG. 17F: Density plots showing distribution of cells in tumor or non-malignant samples.

FIGS. 18A-18E graphically summarize the results of experiments illustrating that CD4+ T cells in bladder tumors are composed of multiple distinct functional states. FIG. 18A depicts UMAP plots of 19,842 single sorted CD3+CD4+ T cells obtained from bladder tumors and adjacent non-malignant tissue (N=7 patients). Each distinct phenotypic cluster identified using Leiden clustering is identified with a distinct color. Annotation of each unbiased cluster was performed by manual inspection of the highest-ranked differentially expressed genes for each cluster and using reference signature-based correlation methods (SingleR) as described in the text. FIG. 18B: Relative intensity of expression of select genes superimposed on the UMAP projections shown in FIG. 18A. FIG. 18C: Violin plot showing relative expression of select differentially expressed genes (columns) for each cluster shown in (A) (rows) (all Padj <0.05). FIG. 18D: Density plots showing distribution of cells in tumor or non-malignant samples. FIG. 18E: The frequency of cells in individual CD4+ T cell states defined by scRNA-seq clustering is shown as a proportion of total CD4+cells within either tumor or nonmalignant compartments across all patients (orange, tumor; blue, non-malignant). A box and whisker plot is shown with formatting as in FIG. 17E. *p <0.05, **p <0.01 by exact permutation test.

FIGS. 19A-19D graphically summarize the results of experiments performed to demonstrate that regulatory CD4+ T cells are heterogeneous, enriched, and clonally expanded in bladder tumors. FIG. 19A: Heatmap showing the expression of select regulatory T cell marker genes (rows) for individual single cells (columns) within the CD4IL2RAHI and CD4m7RLo clusters compared with the CD4cm cluster. Cells were grouped based on their annotations by tissue (tumor or non-malignant), treatment, and patient. Log2-transformed expression of each gene was row scaled. FIG. 19B: Flowcytometry staining of CD4+FOXP3+TILs from a bladder tumor, showing the gating strategy for CD25″g, CD25¹⁰w, ₐₙd CD25^{h1} (top left), and histograms of

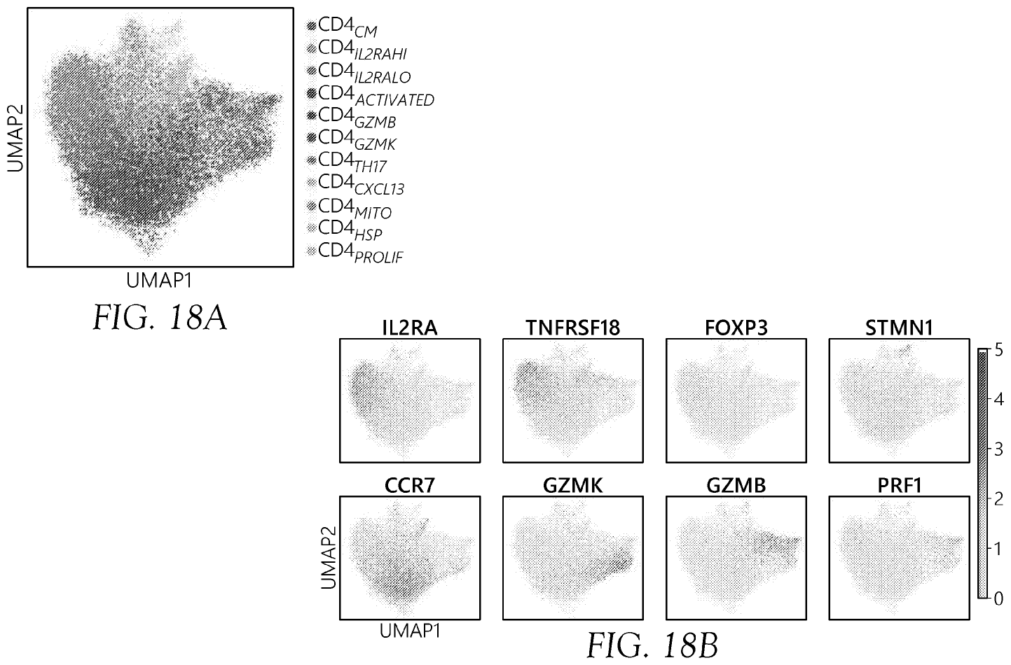

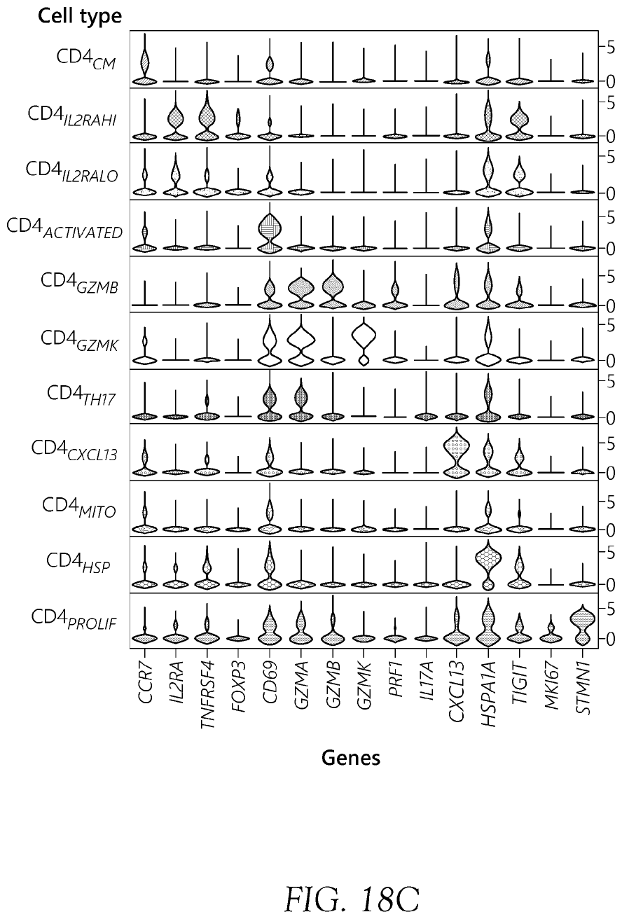

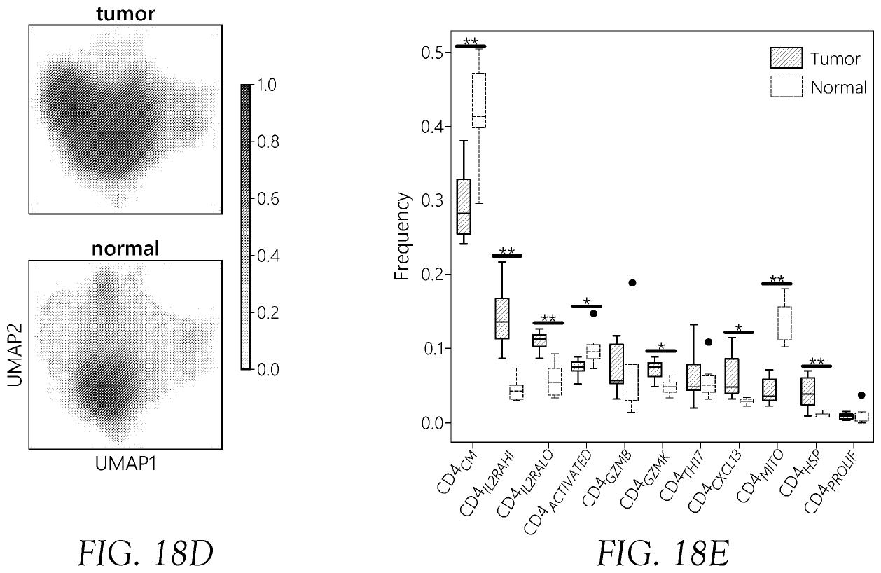

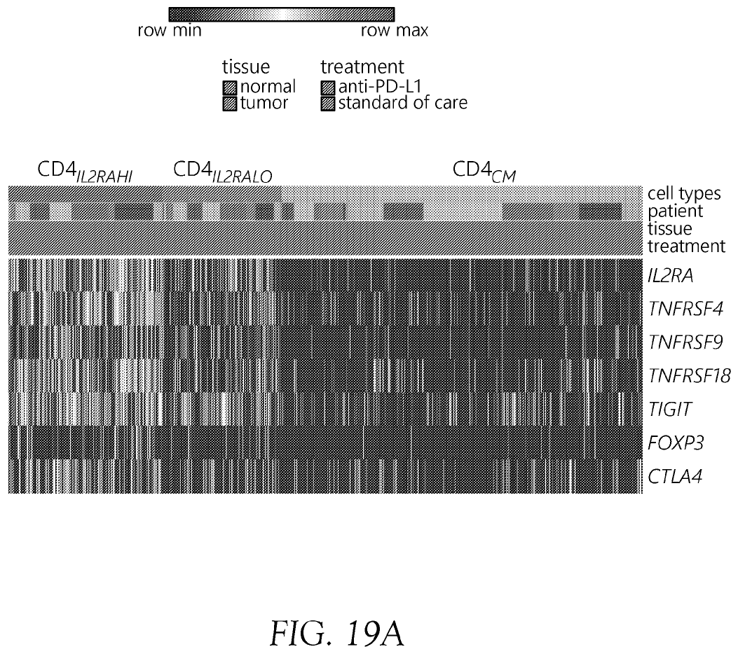

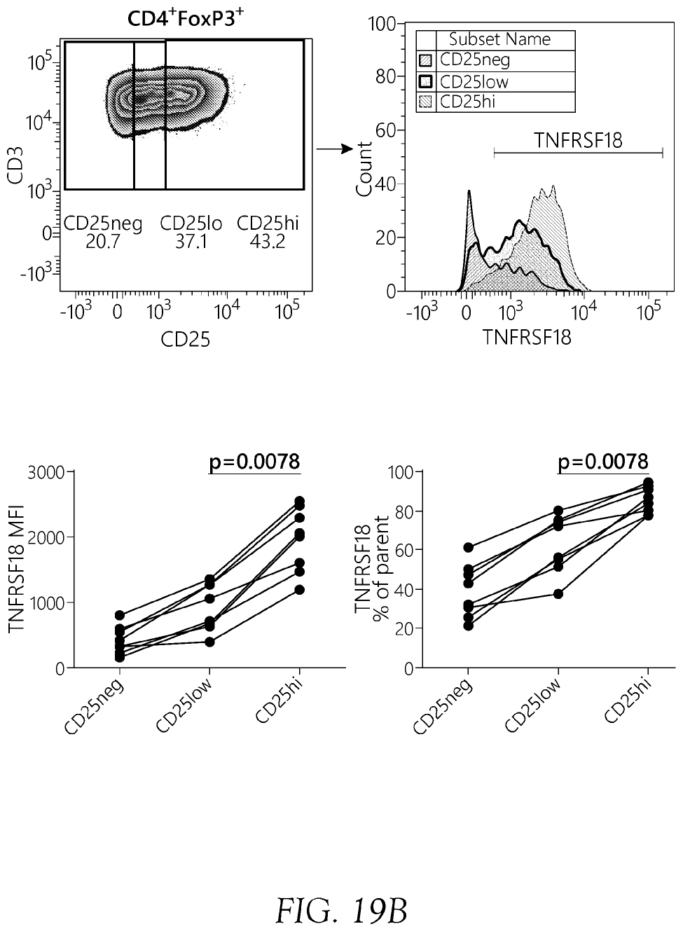

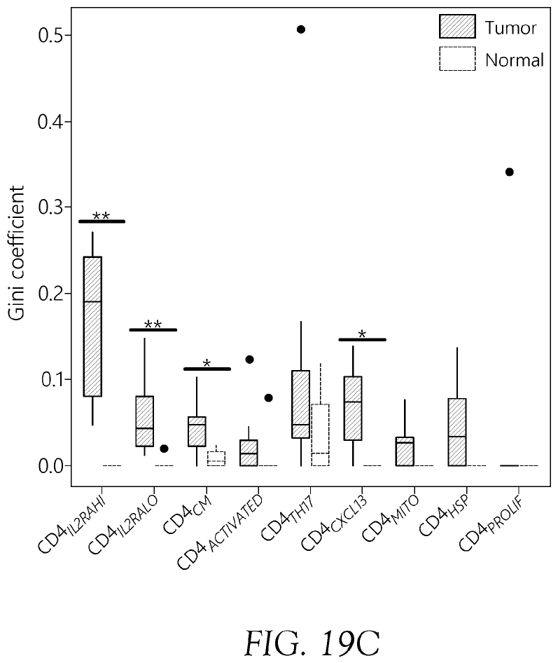

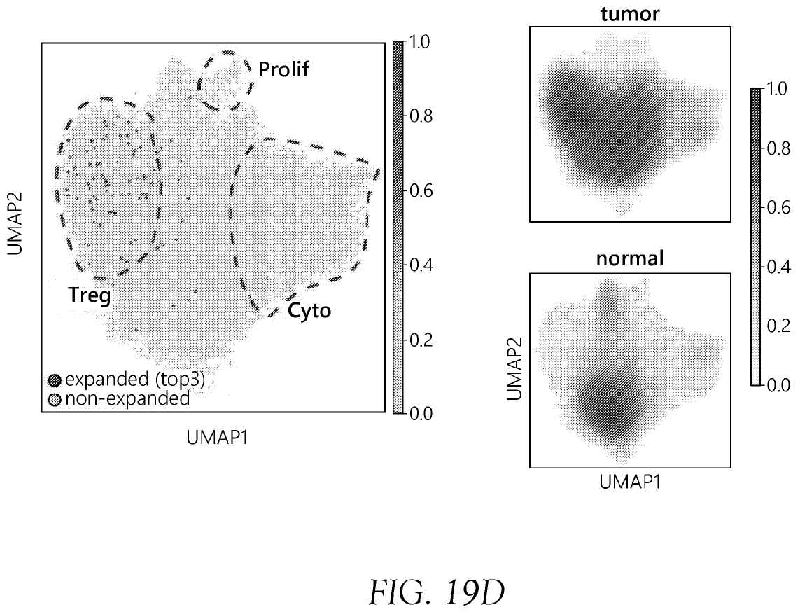

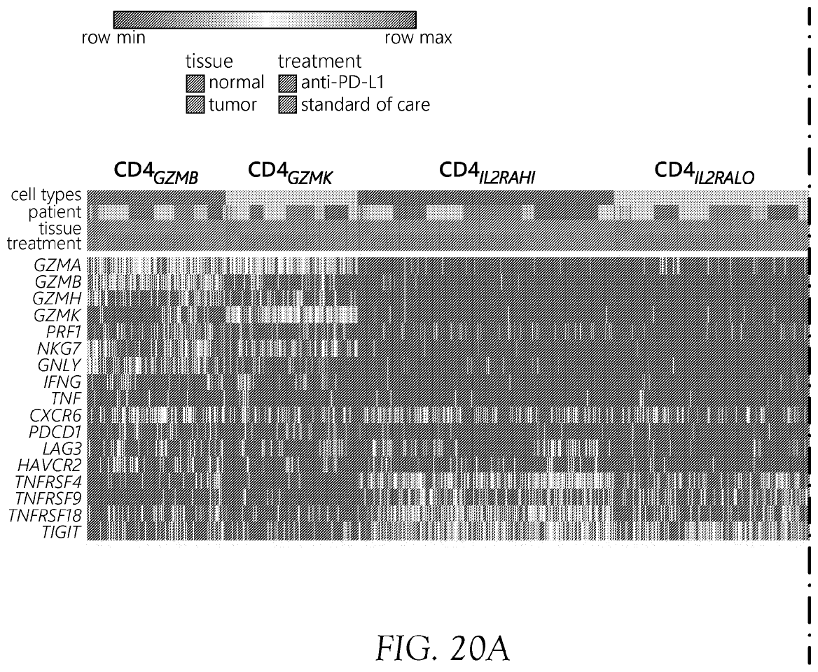

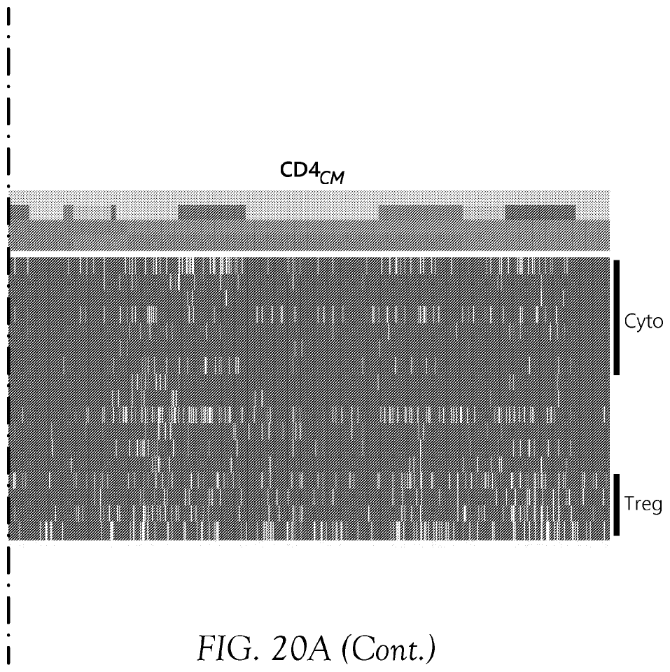

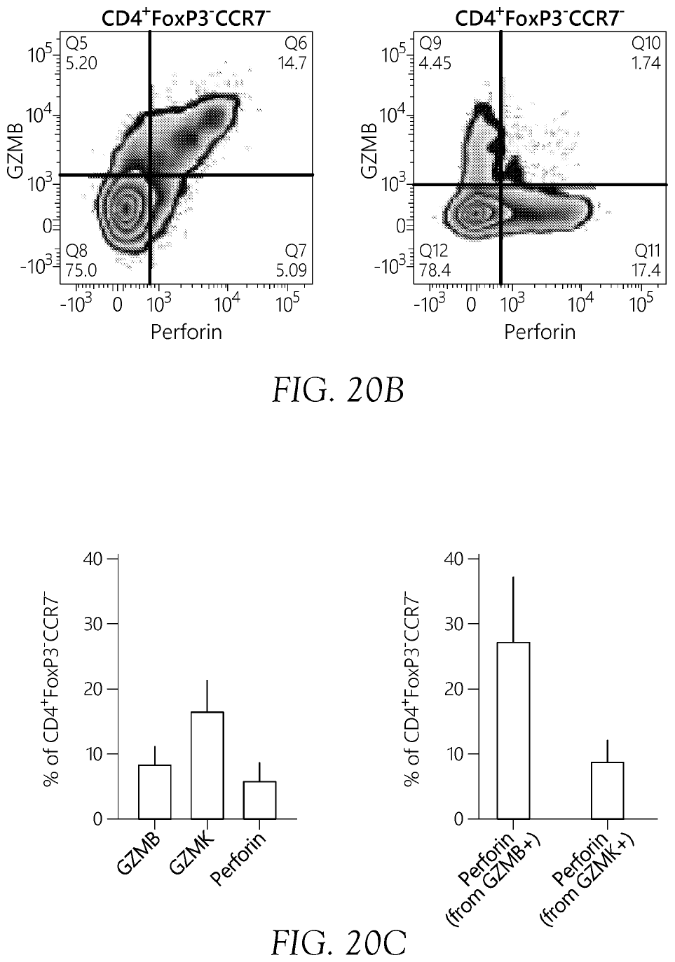

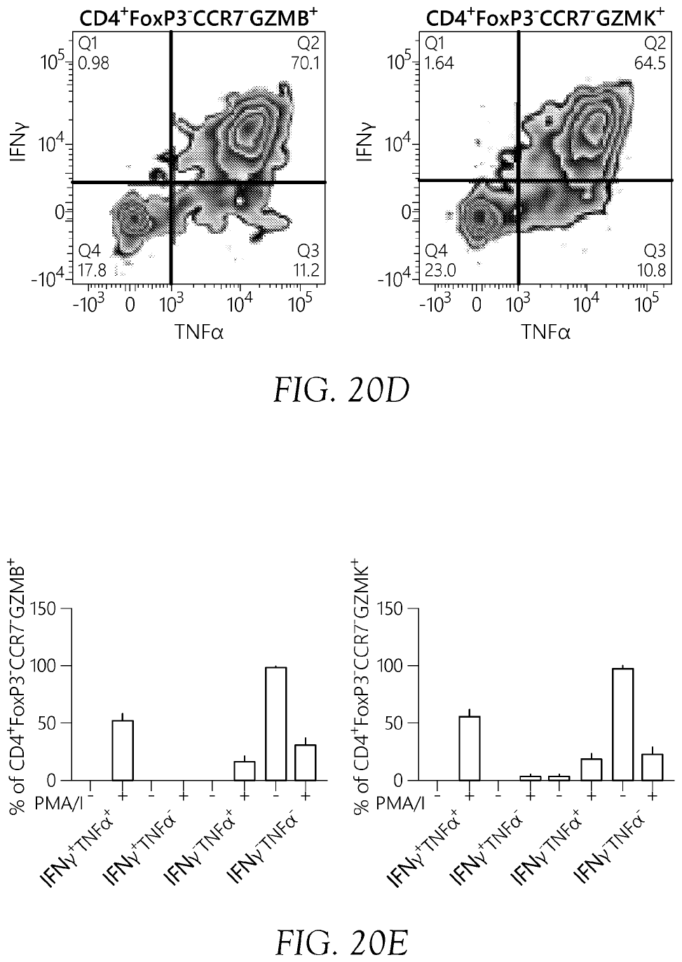

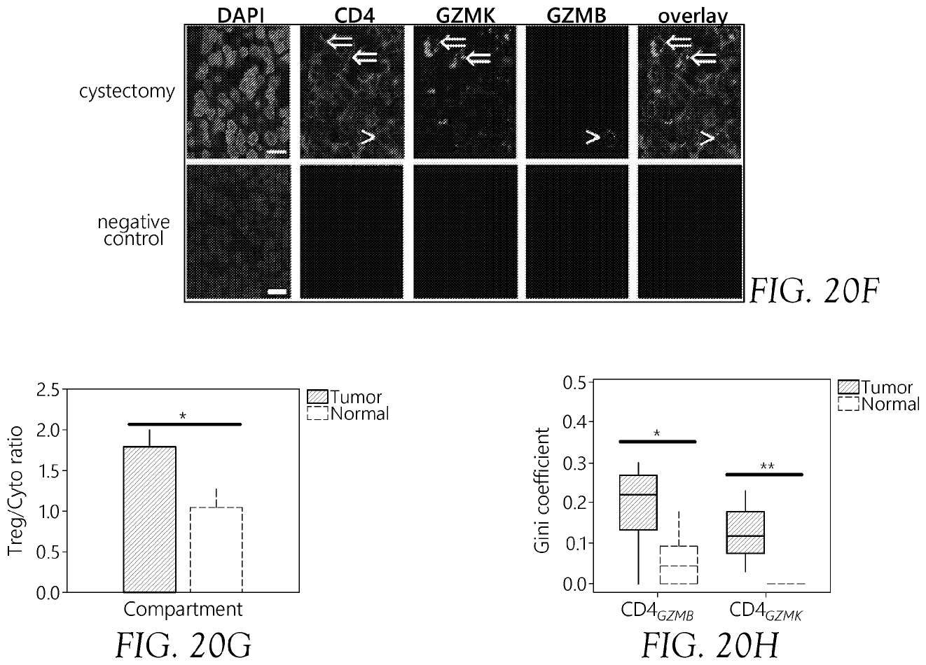

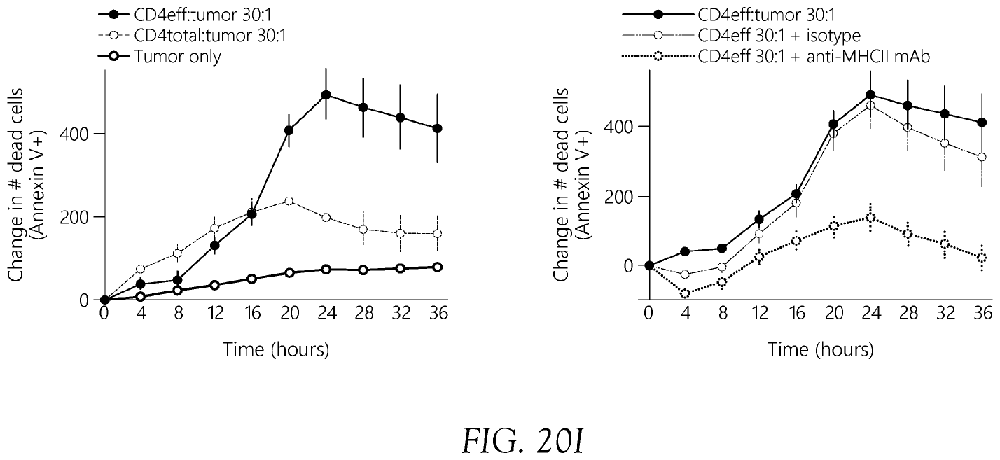

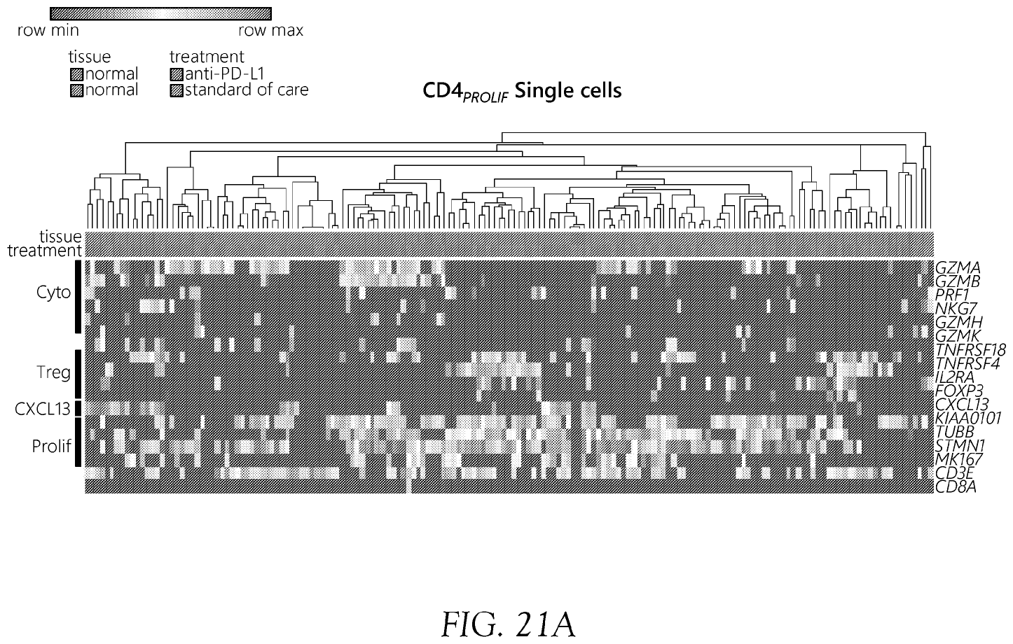

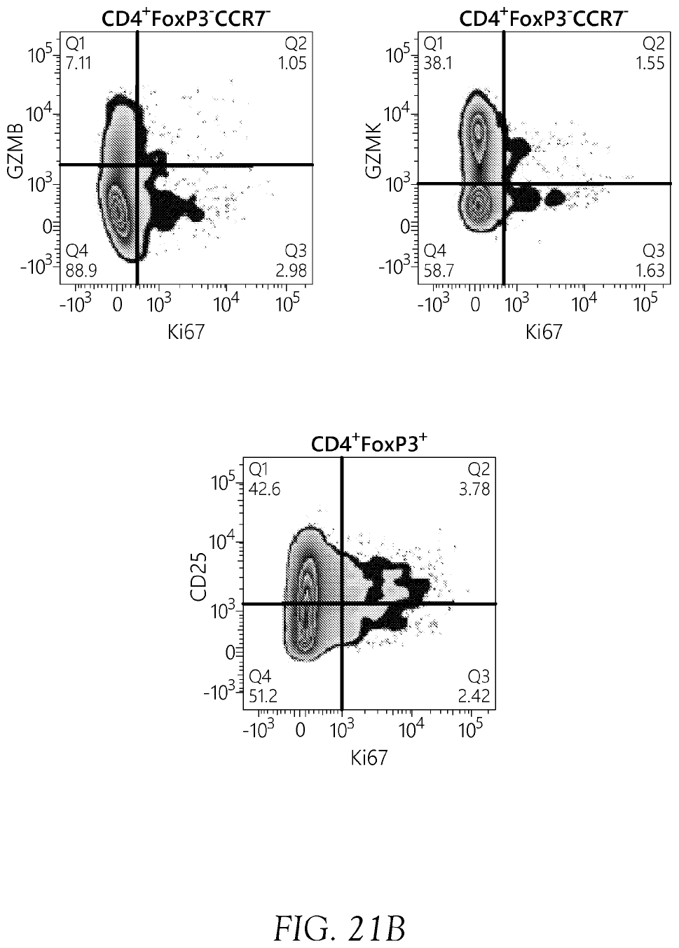

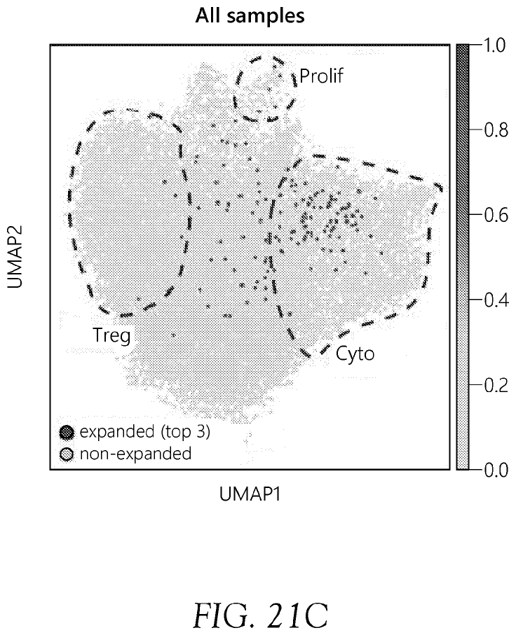

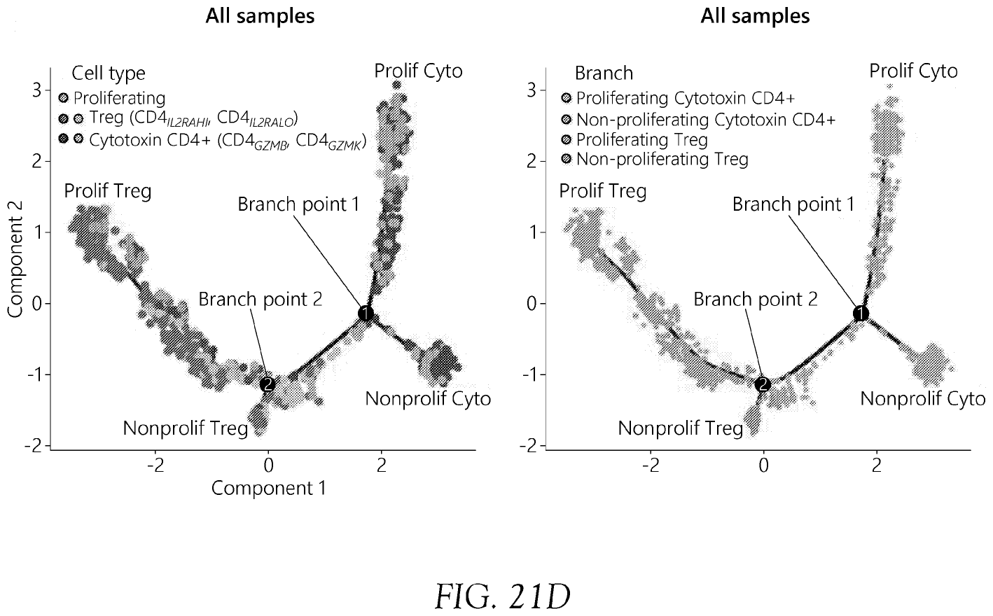

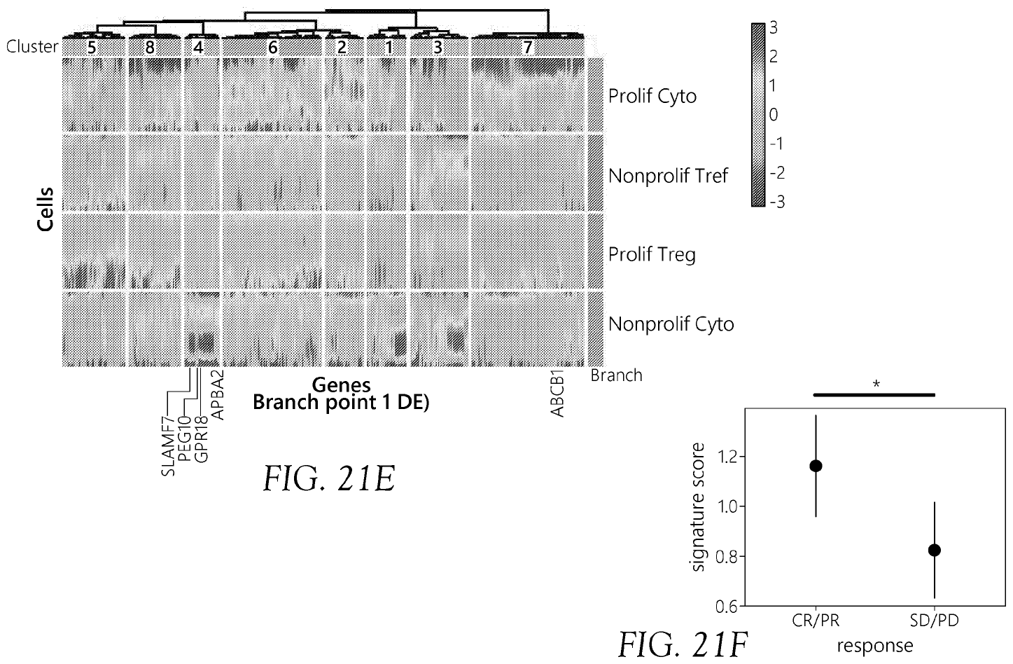

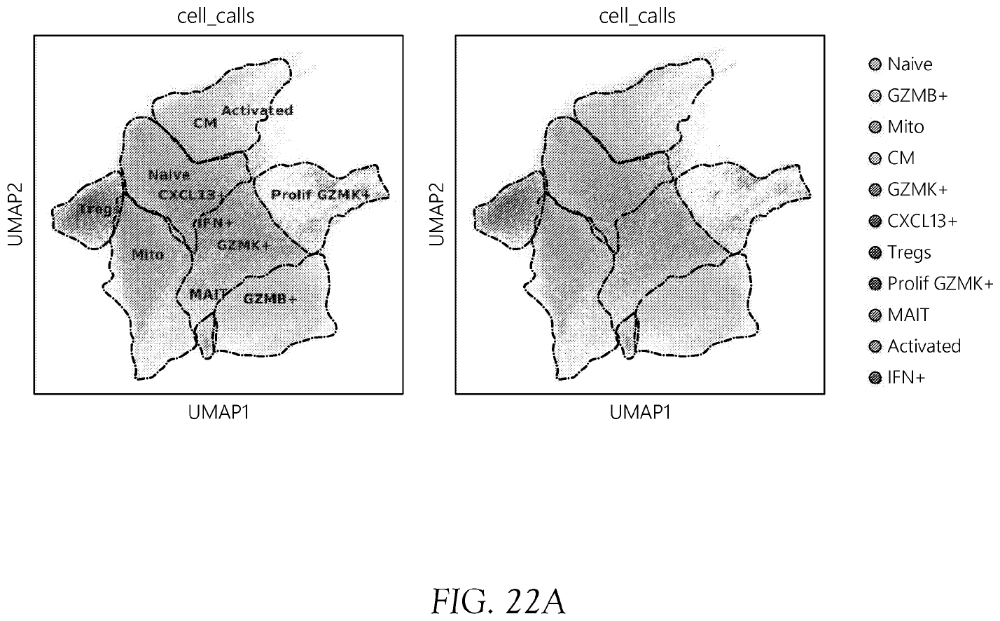

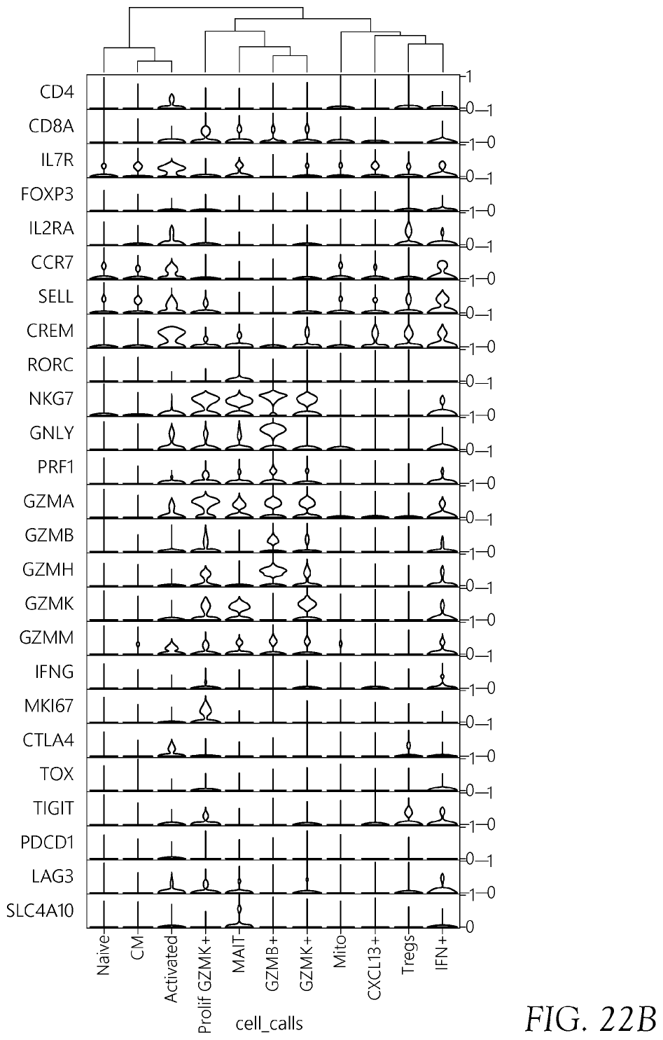

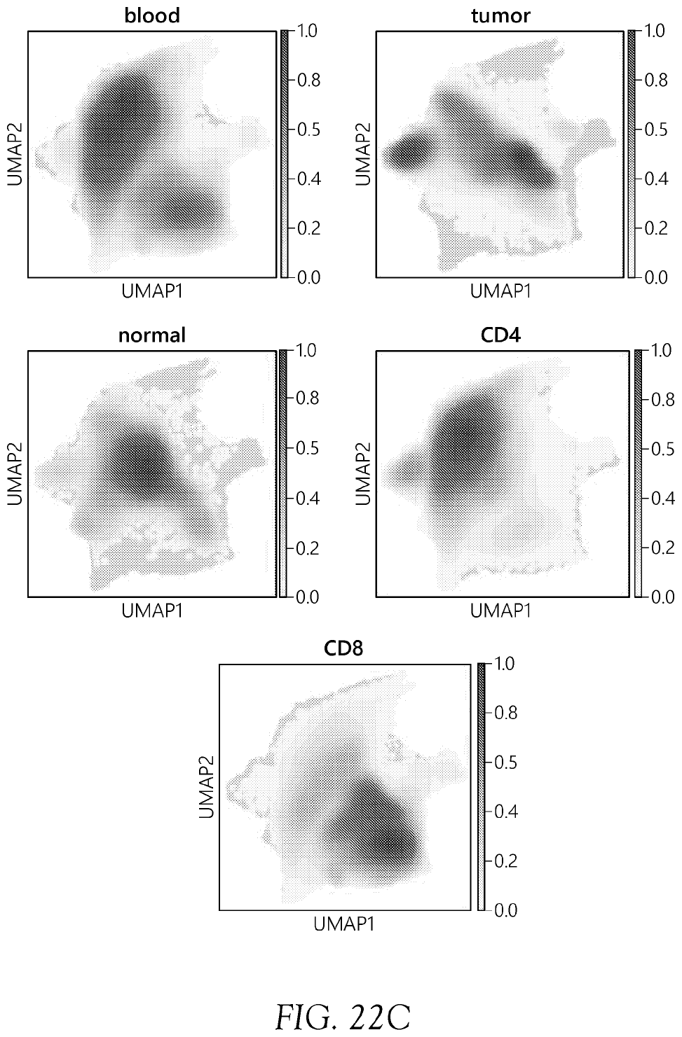

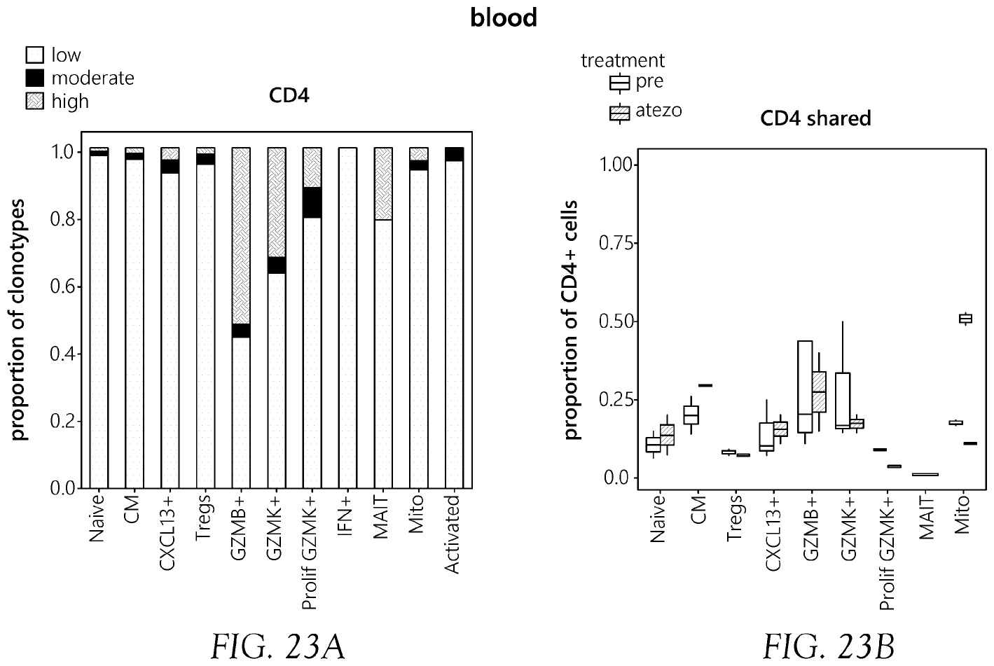

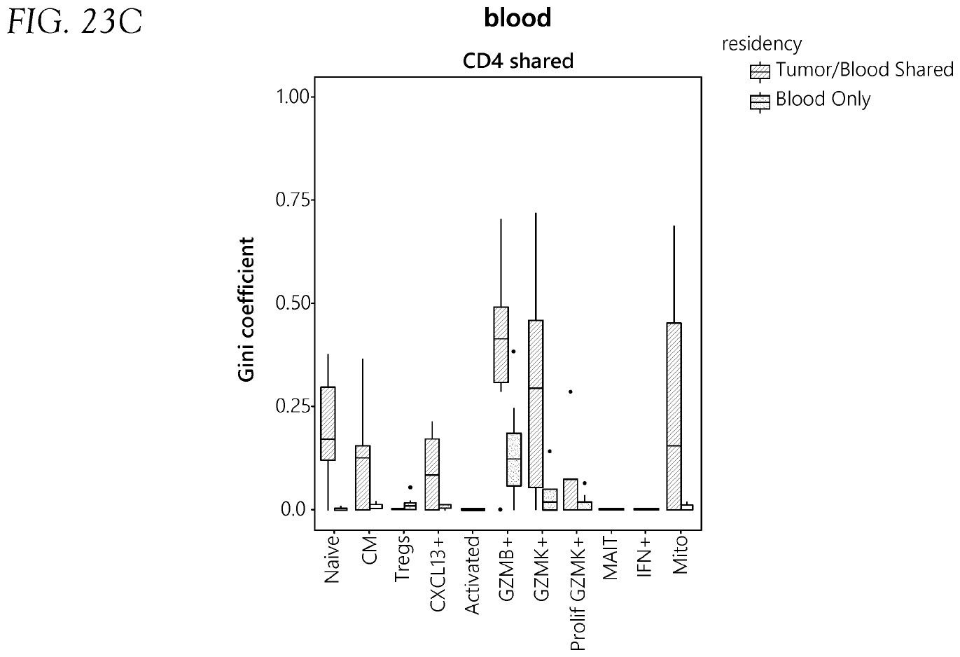

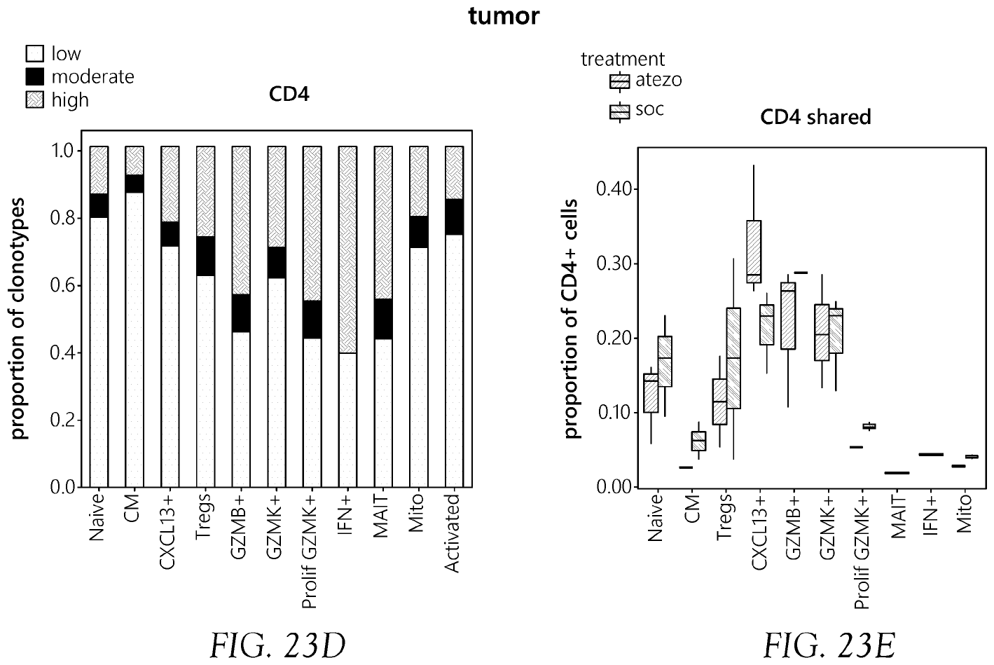

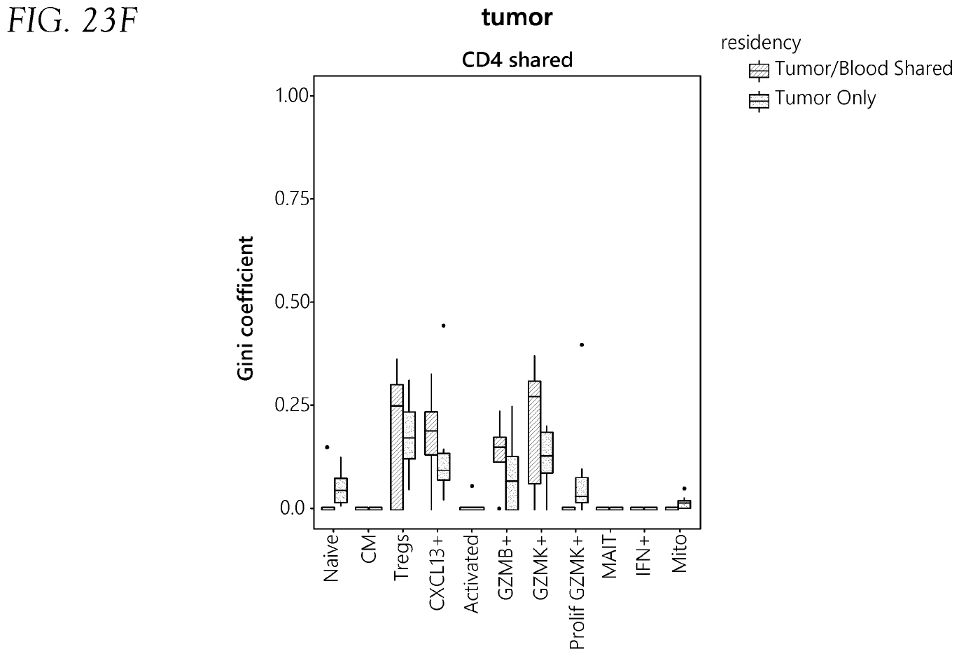

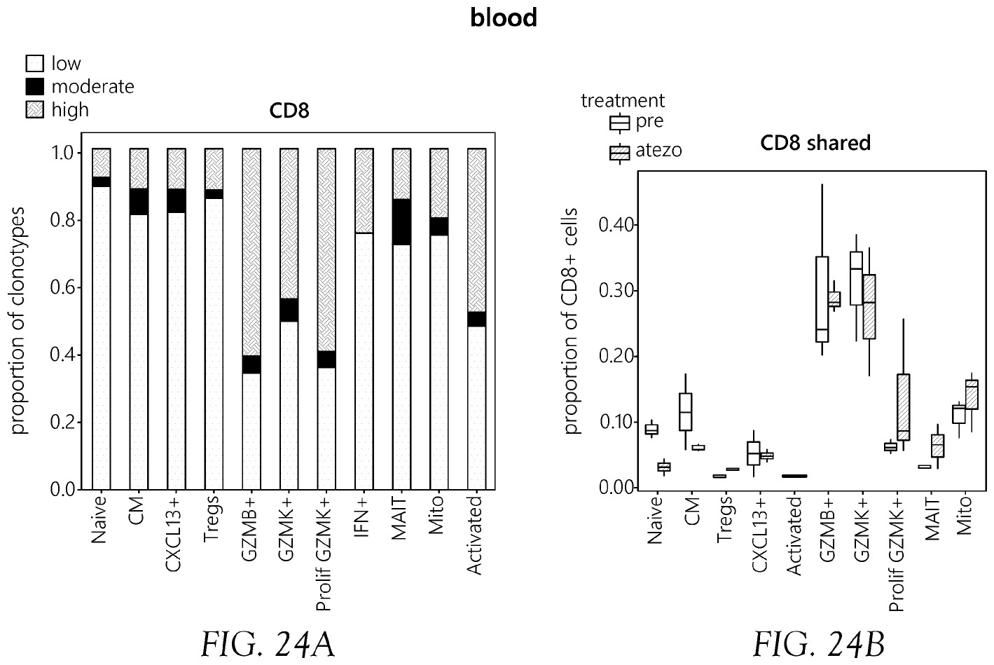

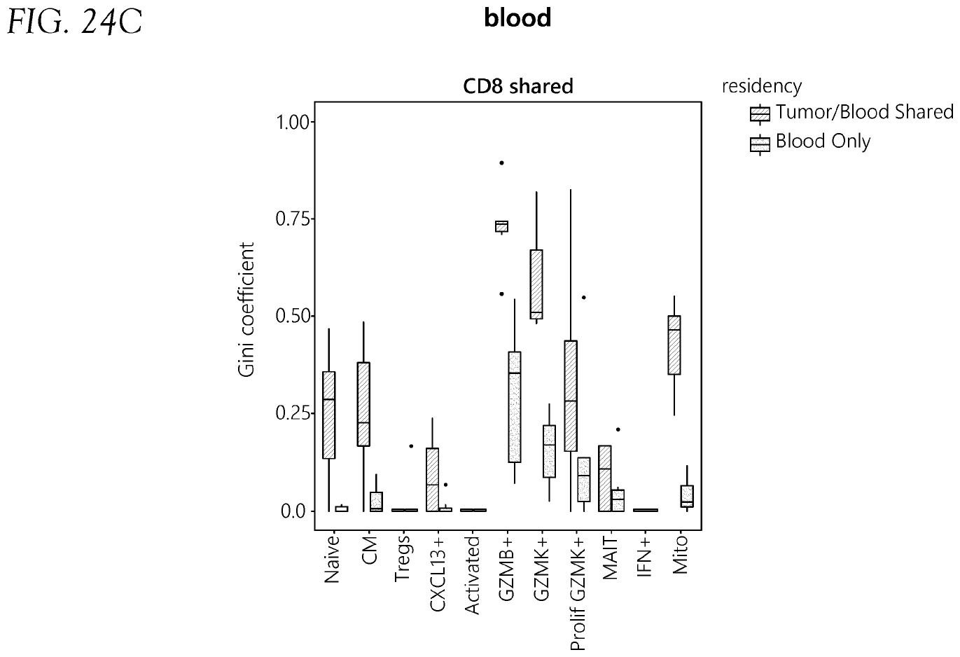

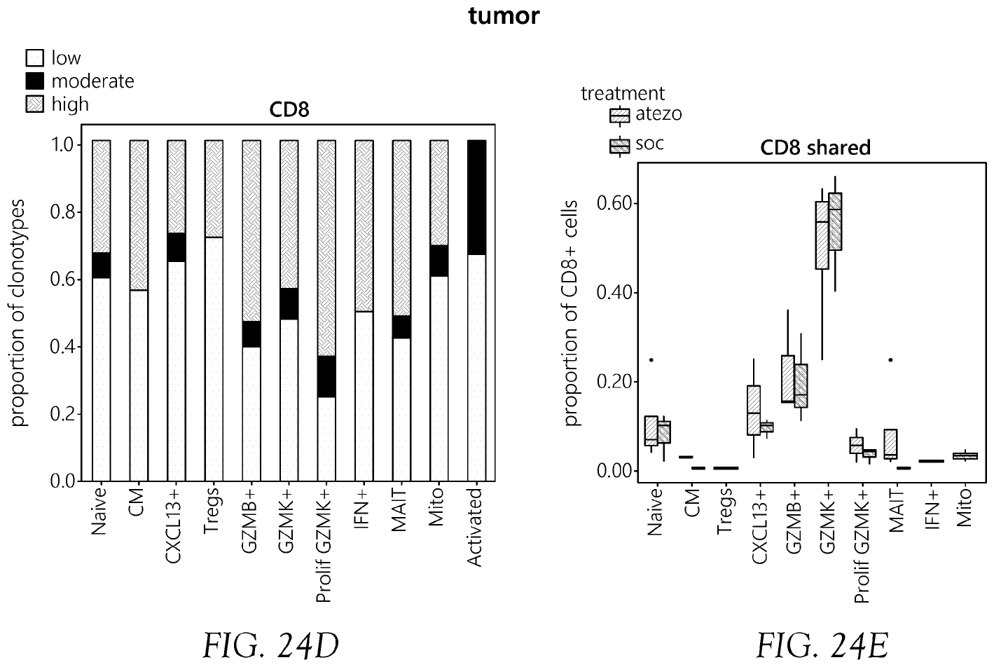

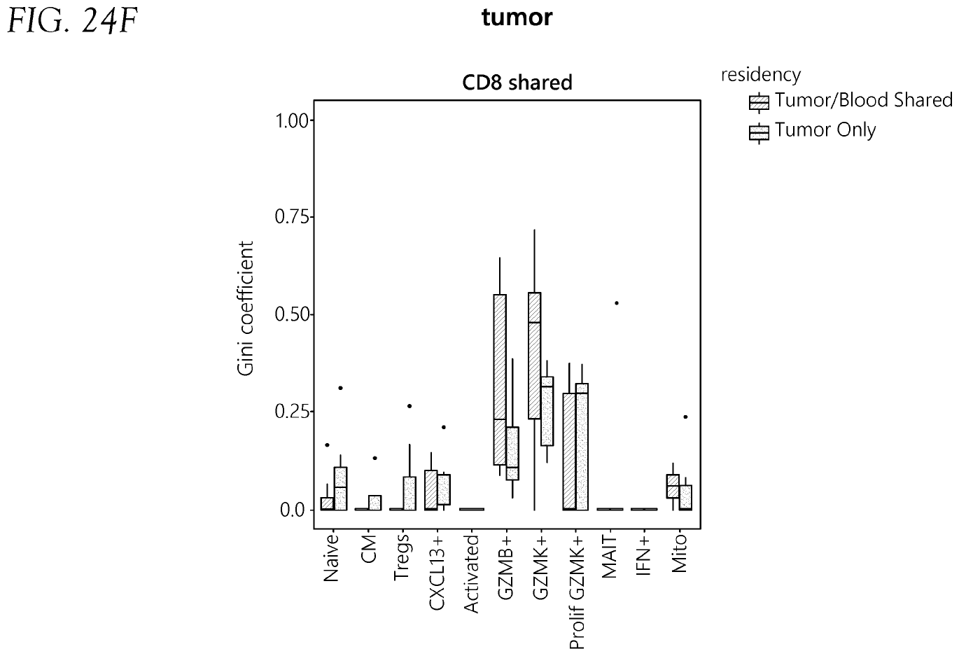

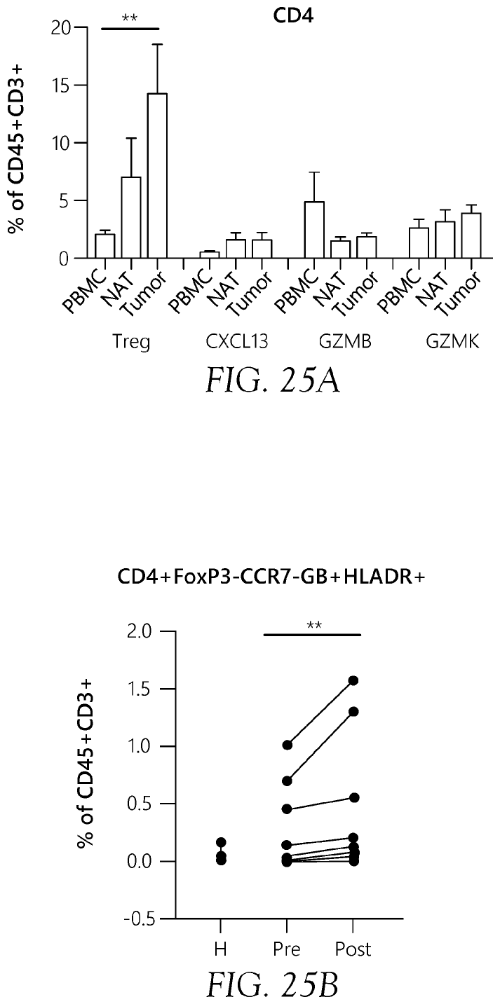

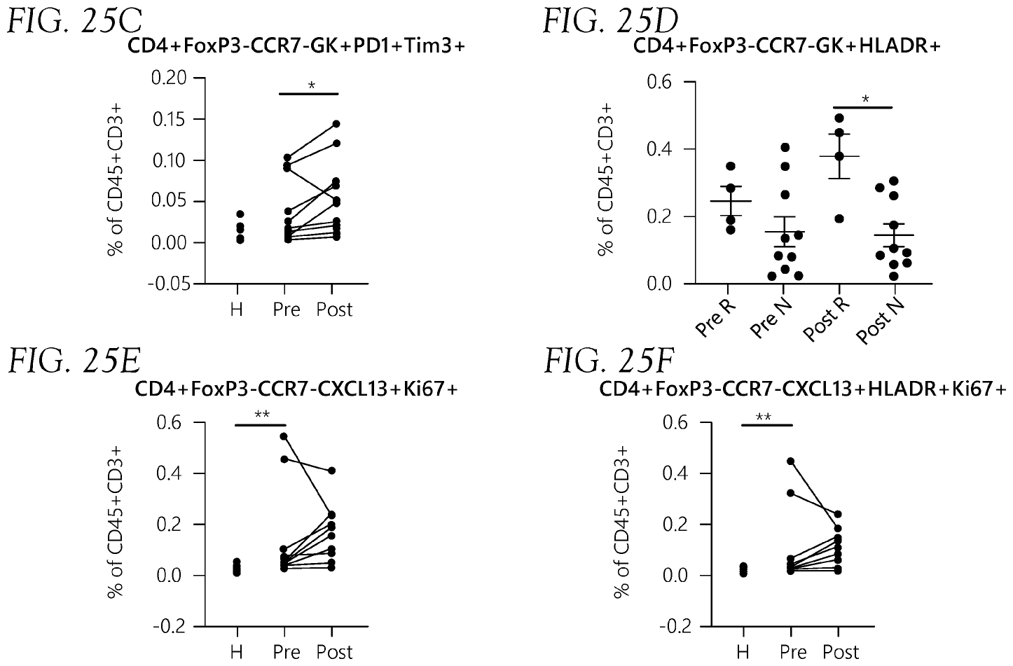

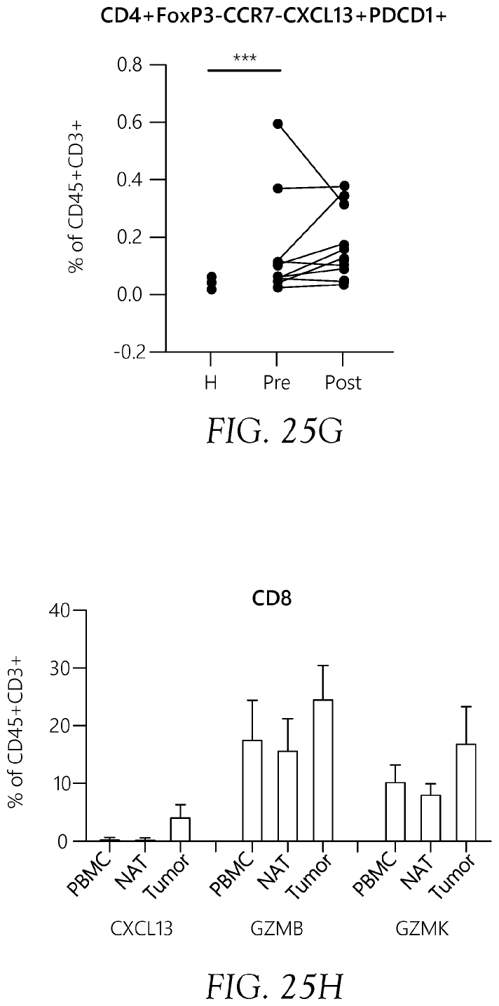

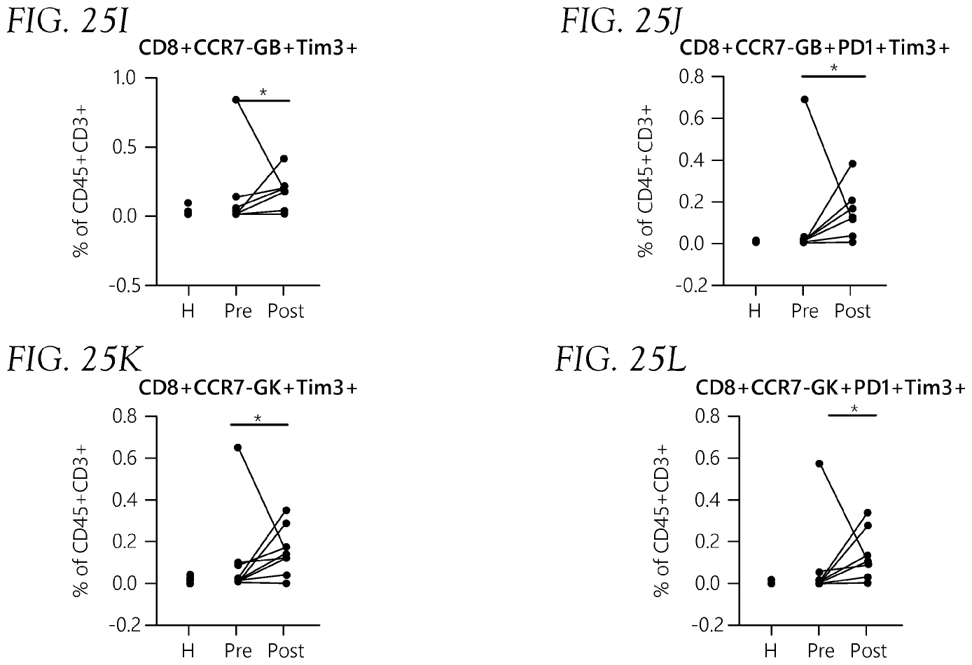

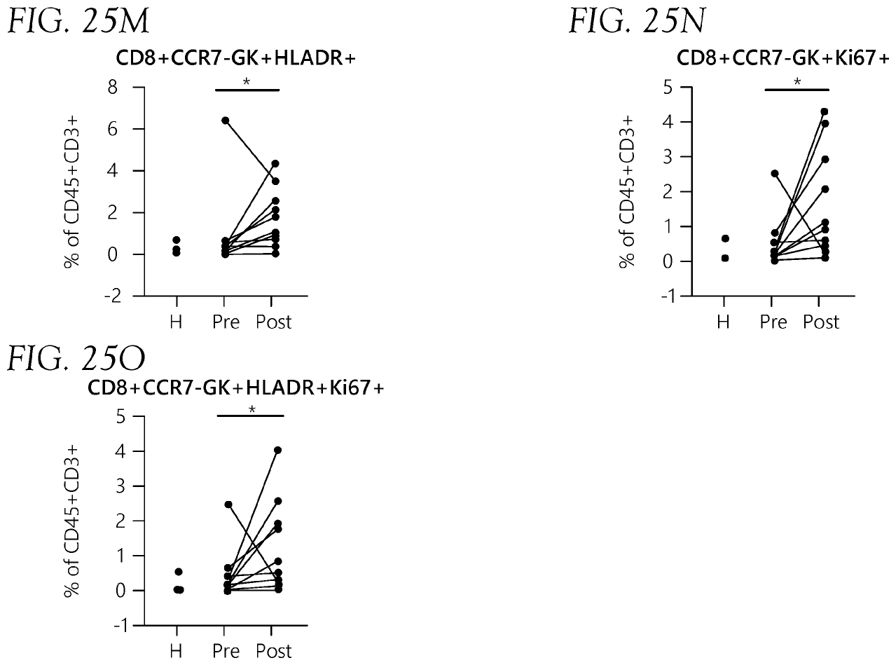

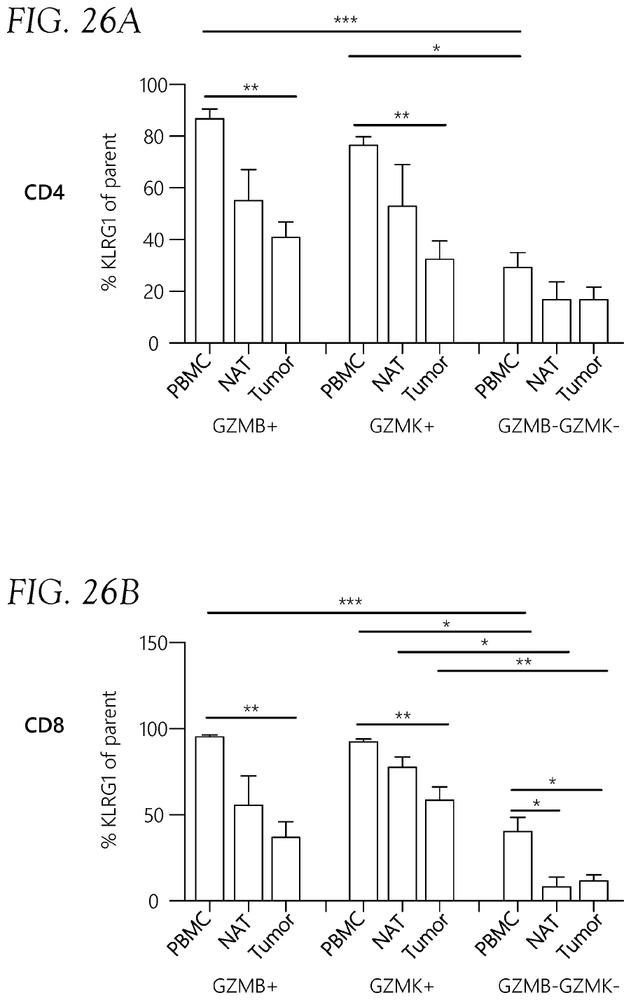

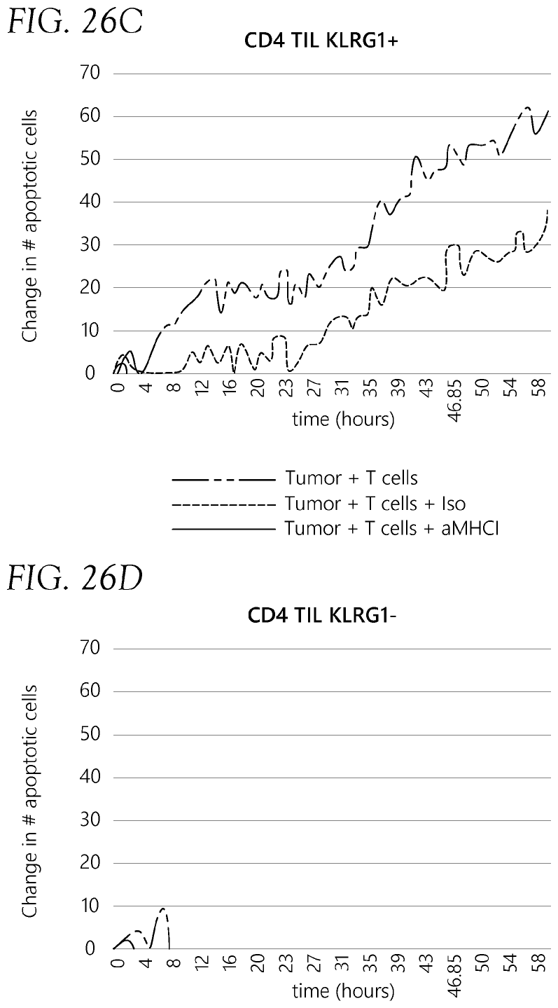

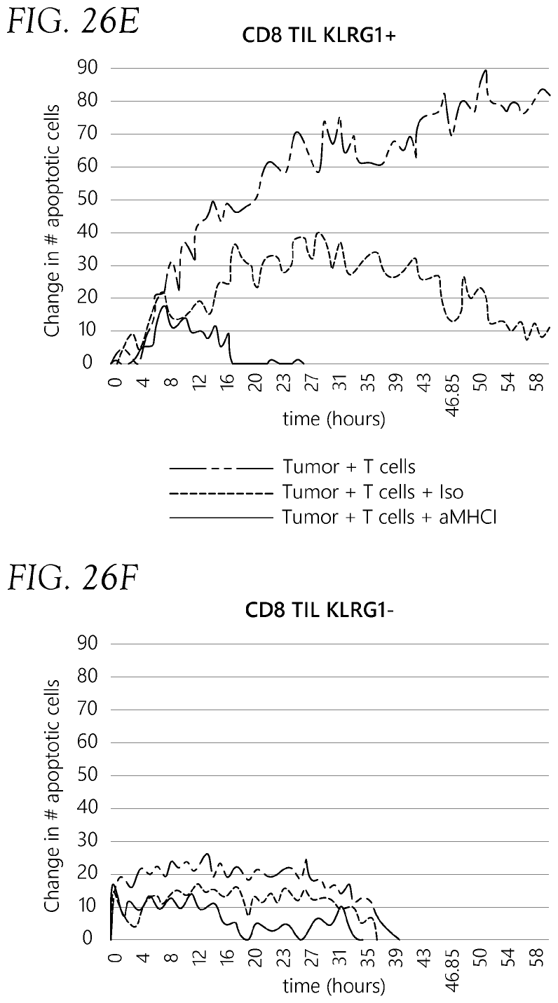

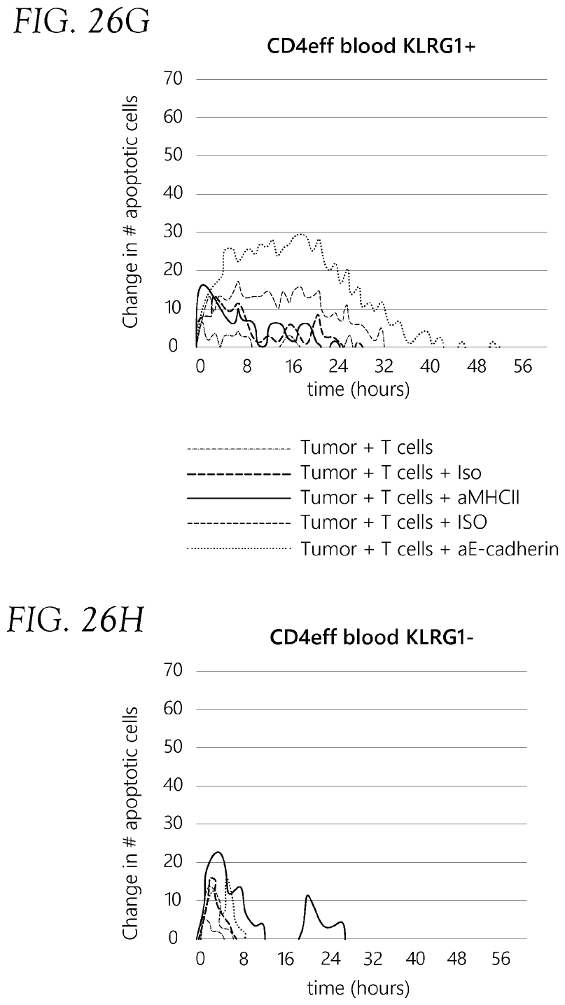

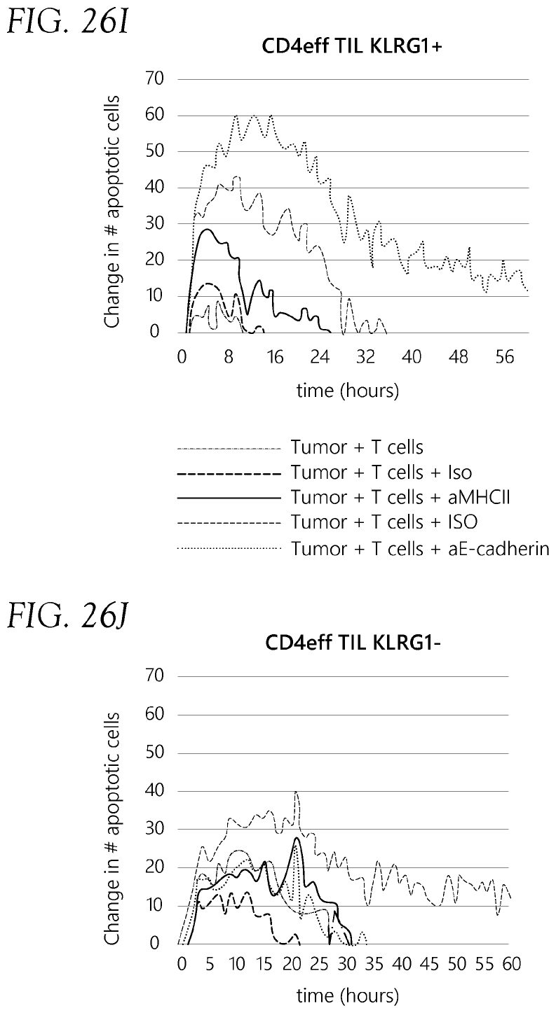

TNFRSF18 staining from each CD25 gate (top right). Mean fluorescence intensity of TNFRSF18 and percent TNFRSF18+from the parental gate are shown for CD25 gates across samples (N=7 tumors, mean ±SEM). *p <0.05 by Wilcoxon paired t test. FIG. 19C: Gini coefficients for regulatory populations (CD4ILRA2HI and CD4IL2RALO, red labels at far left) and other CD4+ T cell populations within tumor and non-malignant compartments across all samples. For each cluster, a box and whisker plot is shown with the median, IQR (box), and 1.5 times the IQR (whiskers), with outliers exceeding 1.5 times the IQR beyond lower and upper quartiles. *p <0.05, **p <0.01 by exact permutation test. N=7 tumor samples and 6 non-malignant samples. FIG. 19D: Left panel: single cells expressing the top 3 most expanded clonotypes found in the combined regulatory populations (CD4ThRA2Ht and CD4m2RAL0) are shown in red in the same UMAP space as in FIG. 18A. The regions composed of regulatory, cytotoxic, and proliferating T cells are outlined and superimposed on the UMAP projection. Right panel: density plots for total CD4+ T cell distribution within tumor and non-malignant compartments are reproduced from FIG. 18D for ease of visual comparison.

FIGS. 20A-20I graphically summarize the results of experiments performed to demonstrate that multiple cytotoxic CD4+ T cell states are enriched and clonally expanded in bladder tumors and possess lytic capacity against tumors. FIG. 20A: Heatmap showing the expression of select cytotoxic or regulatory T cell marker genes (rows) for individual single cells (columns) within the cytotoxic CD4_{G}ZMB and CD4_{GZMK} clusters compared with regulatory (CD4m2RAHI and CD4m2RLo) and CD4cm clusters. Cells were grouped based on their annotations by tissue (tumor or non-malignant), treatment, and patient. Log2-transformed expression of each gene was row scaled. FIG. 20B: Flow cytometry staining of GZMB, perforin, or GZMK in CCRT CD4+FOXP3′ T cells. FIG. 20C: Percentage of cells expressing GZMB, GZMK, or perforin from CCRT CD4+FOXP3⁻ T cells by flow cytometry (left) and the percentage of cells co-expressing perforin within GZMB+or GZMK+CCRI CD4+FOXP3′ T cells (right) (N=7 tumors, mean +SEM). FIG. 20D: Representative flow cytometry staining of IFNγand TNF-αexpression in GZMB+or GZMK+CCM⁻ CD4+FOXP3⁻ T cells stimulated with PMA and ionomycin.

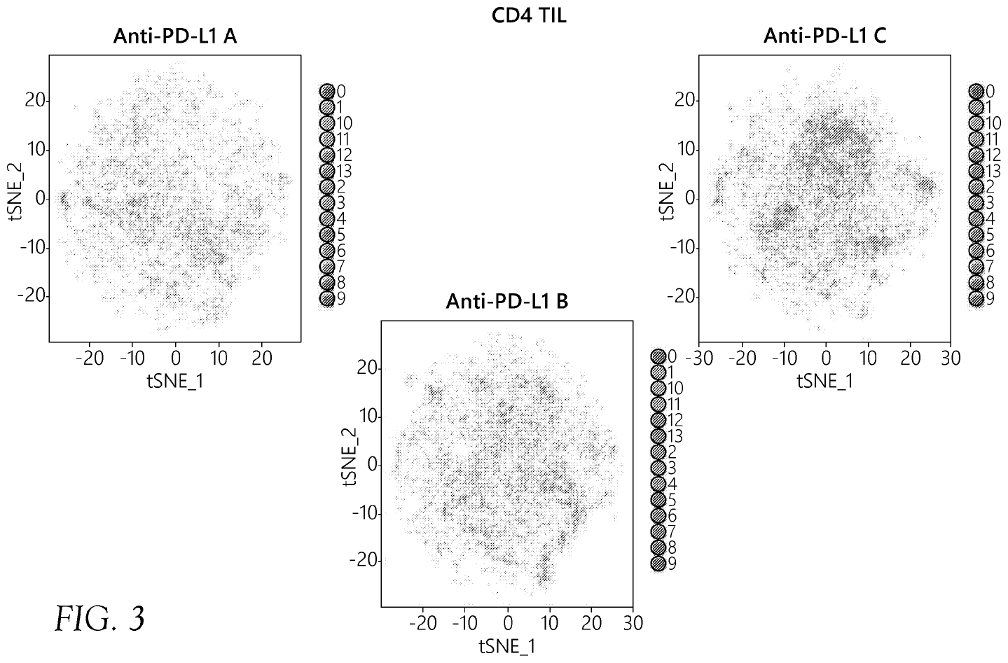

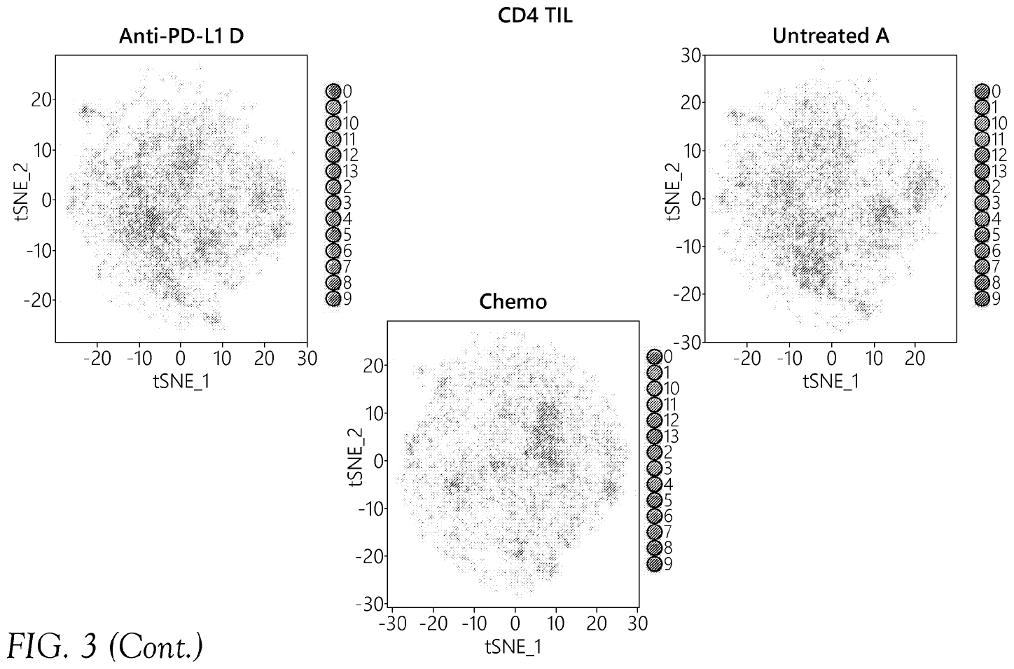

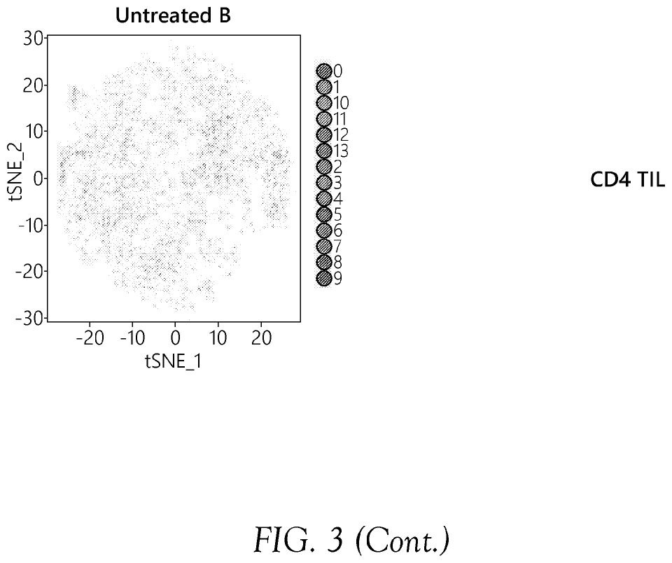

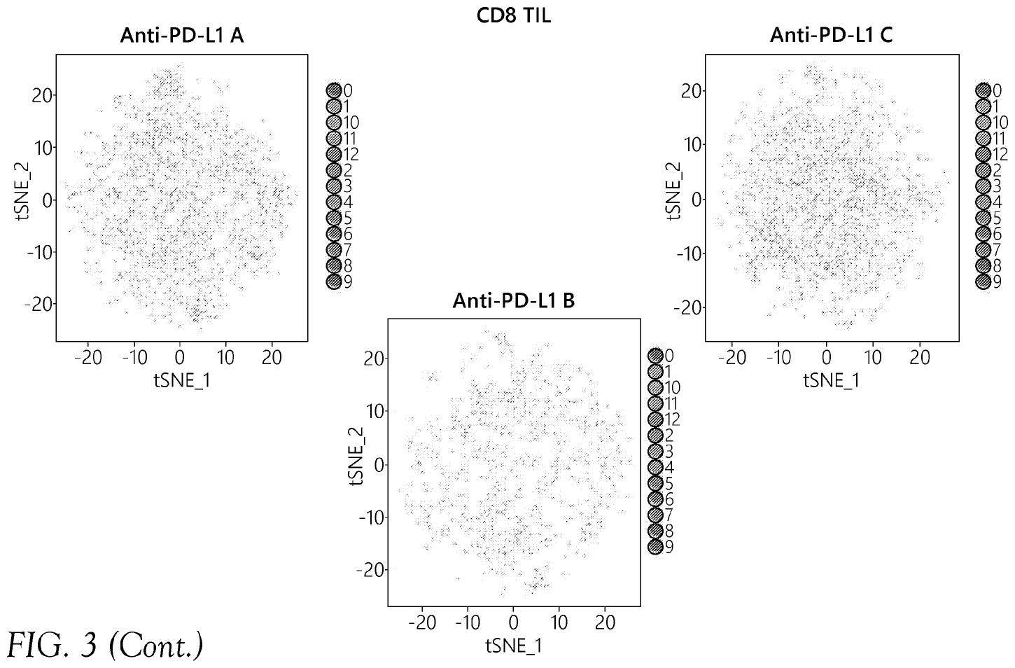

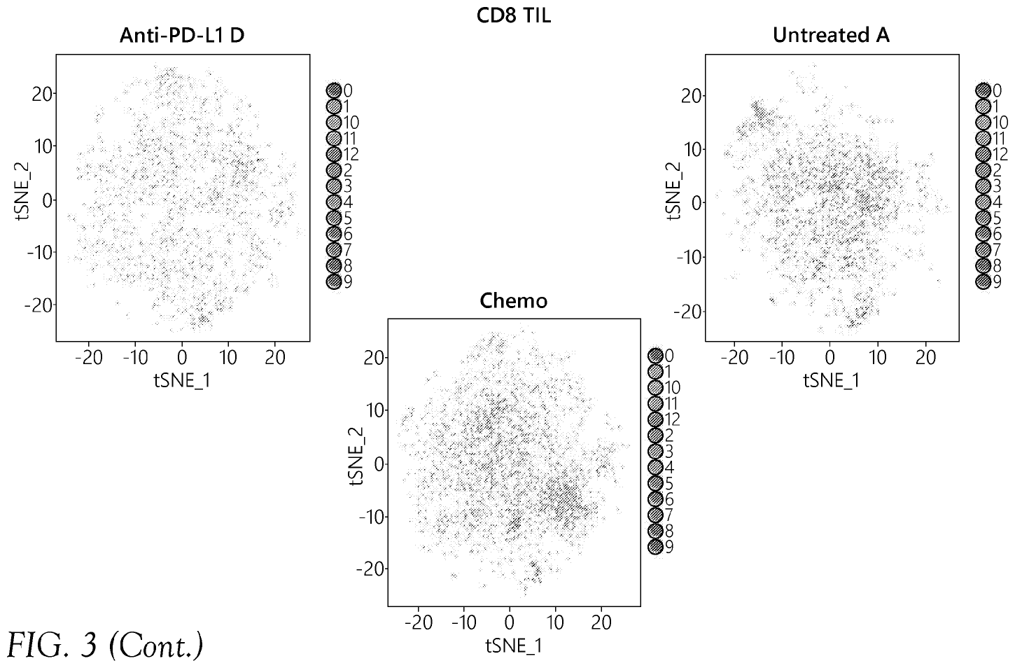

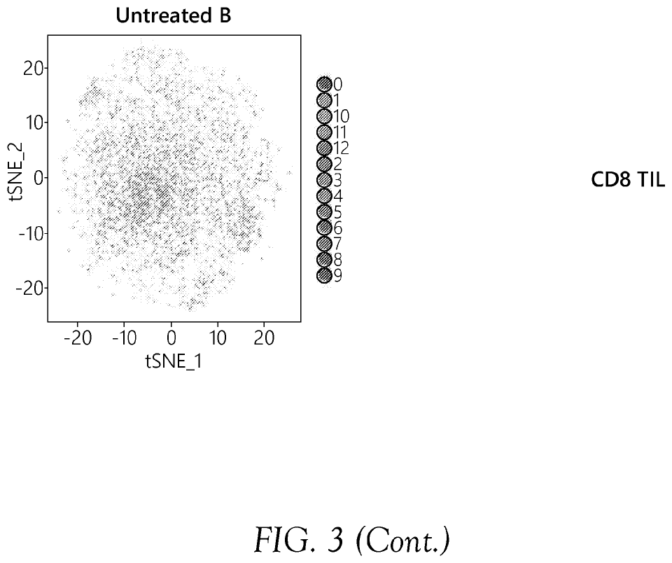

FIG. 20E: Percentages of cells expressing IFNγ, TNF-α, or both from GZMB+or GZMK+CCR7′ CD4+FOXP3′ T cells with and without stimulation (N=11 tumors, mean +SEM). FIG. 20F: Multiplex immunofluorescent staining of DAPI (blue), CD4 (immunohistochemistry, red), GZMK (RNAscope probe, green), and GZMB (RNAscope probe, white) and overlay without DAPI from a cystectomy tumor region from a patient with parallel scRNA-seq and TCR-seq data (anti-PD-L1 C, top row) and from a corresponding tumor field with negative control staining (bottom row). CD4+cells that co-express GZMK (arrows) or GZMB (arrowhead) are indicated. Scale bar, 10 mm. FIG. 20G: The ratio of abundances of all regulatory T cell populations (CD4ALRAm and CD4i2RALo) to all cytotoxic CD4+populations (CD4_{G}ZMB and CD4_{G}ZMK) across all tumor and non-malignant samples (mean +SEM shown; *p <0.05 by unpaired t test, assuming unequal variance). FIG. 201I: Gini coefficients for each of the cytotoxic CD4+populations within tumor and non-malignant compartments across all samples (box and whisker plot is shown with formatting as in FIG. 3C; *p <0.05, **p <0.01, exact permutation test, N=7 tumor samples and 6 non-malignant samples). FIG. 201: Left panel: quantitation of Annexin V+apoptotic cells over time from a time-lapse cytotoxicity experiment with tumor cells cultured alone or with bulk CD4+ TILs (CD4ₜₒₜₐi) or CD4+ TILs depleted of regulatory T cells (CD4_{eff}) at a 30:1 effector:target ratio. Right panel: CD4_{eff}-TILs and tumor cells (30:1 effector:target ratio) were co-cultured with a pan-anti-MHC class II antibody or isotype control. All traces were from the same culture and cytotoxicity assay from the same patient. All traces show relative change in cell death from time point 0. Cytotoxicity with CD4_{d}f is representative of independent experiments from 4 different patients. Mean +SEM from multiple technical replicates for each experiment is shown.

FIGS. 21A-21F graphically summarize the results of experiments performed to demonstrate that proliferating CD4+ T cells contain regulatory and cytotoxic cell states. FIG. 21A: Heatmap showing expression of select cytotoxic, regulatory, and proliferating marker genes (rows) for individual single cells (columns) within the CD4PROLW cluster. Samples were hierarchically clustered. Log2-transformed expression of each gene was row scaled. FIG. 21B: Representative flow cytometry staining from a bladder tumor showing expression of CD25, GB/1B, GZMK, and Ki67. FIG. 21C: Single cells expressing the top 3 most expanded clonotypes found in the CD4ₚRour T cell population are shown in red in the same UMAP space as in FIG. 18A. The regions composed of proliferating, regulatory, and cytotoxic T cells are outlined and superimposed on the UMAP projection for visualization. FIG. 21D: Left panel: pseudotime trajectories derived from all tumors (N=7 samples) and non-malignant samples (N=6 samples). Cells with expanded TCRs from the proliferating (CD4_{PROLIF}, green), regulatory (CD4m2RAHI and CD4IL2RALO, shades of red), and cytotoxic (CD4_{GZMB} and CD4_{G}ZMK, shades of purple) states were used for this analysis. Specific branches corresponding to proliferating cytotoxic cells (top right), non-proliferating cytotoxic cells (bottom right), proliferating regulatory cells (top left), and non-proliferating regulatory cells (bottom left) are labeled. Right panel: branches are color-coded according to the above proliferating or non-proliferating identities. Also labeled are branch points that discriminate proliferating and non-proliferating cytotoxic CD4+ T cells (branch point 1) and proliferating and non-proliferating regulatory T cells (branch point 2). FIG. 21E: Heatmap showing all differentially expressed genes (columns) between branches for branch point 1 across cells in the pseudotime analysis (rows). Cells are grouped by their proliferating or non-proliferating branch assignments, color-coded at the right of the heatmap and corresponding to colors in FIG. 21D. Genes are grouped by color-coded clusters (1-8) shown at the top of the plot, which result from hierarchical clustering based on co-regulation in specific branches. FIG. 21F: Cytotoxic CD4+ T cell gene signature scores were plotted in clinical responders (complete response or partial response) versus non-responders (stable disease or progressive disease) from baseline metastatic biopsies from bladder cancer patients with inflamed tumors on the IMvigor210 clinical trial (N=62 tumors). The signature score was obtained from the IMvigor210 bulk RNA-seq dataset for the cytotoxic CD4+ T cell-specific genes derived from non-proliferating (cluster 4) and proliferating (cluster 7) cytotoxic CD4+clusters from the pseudotime analysis shown below the heatmap in FIG. 21E. Median ±SEM is shown; *p=0.037 by two-tailed t test.

FIGS. 22A-22C graphically summarize canonical T cell populations in the blood and tumor of bladder cancer patients. FIG. 22A is a UMAP plot showing the results of clustering matched blood, tumor, and normal adjacent tissue together from 7 patients with localized bladder cancer. Colors indicate discrete clusters which are also outlined. FIG. 22B is a violin plot showing select genes overexpressed in each cluster in FIG. 22A. FIG. 22C shows density plots showing the overall representation of cells in distinct compartments (blood, tumor, normal) and CD4+and CD8+sorted populations.

FIGS. 23A-23F is a graphical summary of TCR repertoire analysis of CD4+ T cells in matched blood and tumor. FIGS. 23A-C pertain to CD4+ T cells isolated from blood. FIG. 23A shows the proportion of unique TCR clonotypes that are shared by >2 cells (“high”), 2 cells (“moderate”) or only 1 cell (“low”), across the phenotypic CD4+clusters shown on the x axis. FIG. 23B shows, for CD4+ T cells that share an exact TCR clonotype with a CD4+cell from tumor, the relative proportion of these cells by phenotypic cluster for pre-treatment blood samples (“pre”, green) and post-atezolizumab treatment blood samples (“atezo”, red). FIG. 23C shows the Gini coefficient for CD4+ T cells that share an exact TCR clonotype with a CD4+cell from tumor (“tumor/blood shared”, red), or for CD4+ T cells whose TCR clonotype is only found in blood (“blood only”, blue). FIGS. 23D-F are as FIGS. 23A-C but for CD4+ T cells isolated from tumor.

FIGS. 24A-24F is a graphical summary of TCR repertoire analysis of CD8+ T cells in matched blood and tumor. All panels are similar to FIG. 23, and pertain to CD8+ T cells isolated from blood (FIGS. 24A-C) or from tumor (FIGS. 24D-F).

FIGS. 25A-25O demonstrate the presence of specific cytotoxic CD4+and CD8+ T cells in the blood of atezolizumab-treated patients that correlate with response to atezolizumab therapy. The blood samples used here were analyzed by flow cytometry, and include paired pre- and post-treatment PBMCs from the blood of 14 bladder cancer patients treated with atezolizumab on this clinical trial, including 4 patients who had responses (pathologic downstaging of their tumor at the time of surgical cystectomy compared to initial diagnostic biopsy), and 10 patients who did not have responses. These included the 4 patients for whom scRNAseq/TCRseq data were obtained. Staining was also performed on PBMCs from 8 healthy individuals for comparison. FIG. 25A shows the percentage of total CD45+CD3+T cells from PBMC, tumor, and normal adjacent tissue (NAT) that are regulatory, CXCL13+, GZMB+, or GZMK+CD4+ T cells. Additional CD4+populations that show significant increases (* p <0.05) with atezolizumab treatment (CD4+FOXP3- CCR7- GZMB+HLA-DR+, CD4+FOXP3-CCR7- GZMK+PD-1+Tim3+cytotoxic T cells, FIG. 25B-25C), with response to atezolizumab in post-treatment PBMC samples (CD4+GZMK+HLA-DR+cytotoxic T cells, FIG. 25D), and with the cancer state relative to healthy controls (various CD4+CXCL13+T cells, FIG. 25E-25G) are shown. FIG. 2511 shows the percentage of total CD45+CD3+T cells from PBMC, tumor, and normal adjacent tissue (NAT) that are CXCL13+, GZMB+, or GZMK+CD8+ T cells. Additional CD8+populations that show significant increases (* p <0.05) with atezolizumab treatment (exhausted CD8+CCR7- GZMB+and GZMK+T cells that are Tim3+ or PD-1+Tim3+, FIG. 25I-25L; also activated CD8+CCR7- GZMK+T cells that express HLA-DR, Ki67, or both (FIGS. 25M-25O) are shown.

FIGS. 26A-26J demonstrates that KLRG1 identifies, and enriches for, cytotoxic CD4+and CD8+ T cells with autologous tumor killing activity. FIGS. 26A-26B show the proportion of GZMB+, GZMK+, and GZMB-GZMK- T cells that express KLRG1 in PBMC, tumor, and NAT, for CD4+ T cells (FIG. 26A) and CD8+ T cells (FIG. 26B). FIGS. 26C-26F show that in expanded tumor-infiltrating CD4+(FIGS. 26C-26D) and CD8+(FIGS. 26E-26F) T cells, the populations sorted for KLRG1 expression (FIGS. 26C and 26E) have enhanced killing activity compared to KLRG1- sorted populations (FIGS. 26D and 26F) when co-cultured with autologous tumor, and that this killing is blocked by an antibody to MHC FIGS. 26G-26J show that in blood (FIGS. 26G-26H) and tumor (FIGS. 26I-26J) from a distinct patient as in FIGS. 26D-26F, the populations sorted for KLRG1 (FIGS. 26G and 26I) have enhanced killing activity compared to KLRG1- sorted populations (FIGS. 26H and 26J), and that this activity is enhanced by co-incubation with an antibody against E-cadherin.

### DETAILED DESCRIPTION OF THE DISCLOSURE

The present disclosure relates generally to, inter alfa, therapeutic and diagnostic methods and compositions for treatment of bladder cancer, and particularly relates to defining pre-treatment gene signatures that are predictive of response to anti-PD-L1 therapy and to the use of such gene signatures as biomarkers to identify individuals having, or suspected of having, or at risk of having, a bladder cancer who are most likely to respond to an anti-PD-L1 therapy. The experimental results presented herein have identified single gene signatures and composite gene signatures from single-cell RNA sequencing data that are associated with specific types of tumor-infiltrating T cells in human bladder tumors. These genes or gene signatures are associated with subsequent response to, and/or longer survival with, cancer immunotherapies (specifically, anti-PD-L1 antibodies) in metastatic bladder cancer based on expression in a pre-treatment tumor biopsy.

Immunotherapies have changed the landscape of cancer treatment by producing durable and long-lasting responses through triggering of anti-tumor cell-mediated immunity. In particular, checkpoint inhibitors (CPI) targeting immune inhibitory molecules CTLA-4 and PD-1 in T lymphocytes have been approved based on responses and improved overall survival in multiple malignancies, particularly those with high mutational burden (Martincorena and Campbell, 2015; Cancer Genome Atlas Research Network, 2008). However, even in the most responsive malignancies, CPIs as monotherapies are efficacious in only —20% of patients (Hargadon et al., 2018). This could be partly due to the heterogeneity of tumor-infiltrating T lymphocytes (TILs) and their differential ability to confer a therapeutic benefit upon treatment.

Currently, cytotoxic CD8⁺ T cells are the main focus of efforts to understand how immunotherapy elicits anti-tumor immunity. In melanoma, expression and chromatin state signatures of cytotoxicity and exhaustion (Tirosh et al., 2016; Philip et al., 2017; Ayers et al., 2017; Herbst et al., 2014) and the presence of CD8⁺ T cells at the tumor invasive margin pre-treatment (Tumeh et al., 2014) are significantly correlated with subsequent responses to PD-1-directed therapy. However, in metastatic transitional cell carcinoma (TCC) of the bladder, where response rates to PD-1 blockade are —15-20% in platinum chemotherapy-refractory patients and >20% in frontline platinum-ineligible patients, predictive biomarkers of response are unclear, including PD-L1 expression (Koshkin and Grivas, 2018). Recently, a detailed interrogation of the pre-treatment tumor microenvironment in TCC found that a higher score of CD8⁺ gene signature and tumor mutational burden, and conversely a lower score of transforming growth factor-beta (TGF-I3) gene signature particularly in immune excluded tumors, were associated with response to the anti-PD-L1 agent atezolizumab (Mariathasan et al., 2018). However, the importance of heterogeneous subsets of TILs in TCC beyond canonical cytotoxic and exhausted phenotypes in responses to PD-1 blockade remains unexplored. Detailed characterization of the T lymphocytes in the tumor is needed for precisely mapping the cells responsible for tumor recognition and control and defining predictive markers of response to CPI in bladder cancer.

As described in greater detail below, various experiments were performed to interrogate the tumor microenvironment of patients with localized muscle-invasive bladder TCC, who either received or did not receive neoadjuvant anti-PD-L1 immunotherapy (atezolizumab, Roche/Genentech) prior to surgical resection. Droplet single-cell RNA-sequencing (dscRNA-seq) and paired TCR sequencing of >30,000 CD4⁺ and CD8⁺ T cells from paired tumor and adjacent non-malignant tissues reveals heterogeneity in known CD4⁺ populations such as regulatory T cells, which are also enriched and clonally expanded in tumor (see, e.g., Examples 2-3). In addition, several novel populations of cytotoxic CD4⁺ expressing cytolytic effector proteins are clonally expanded in tumor indicative of tumor specificity, which is validated by direct autologous tumor killing by these cytotoxic CD4⁺ effectors ex vivo. Proliferating CD4⁺ T cells are also seen in tumor and are composed of cells with both regulatory and cytotoxic phenotypes; while regulatory cells are more closely associated with the proliferative state in untreated bladder tumors based on transcriptional and clonotypic data, this balance is shifted by anti-PD-L1 therapy to favor proliferative cytotoxic CD4⁺ T cells and away from proliferative regulatory cells. Finally, as illustrated in Example 8, in an orthogonal RNAseq data set of 168 metastatic bladder cancer patients treated with anti-PD-L1, the proliferating T cell signature, and a signature of proliferative cytotoxic CD4⁺ T cells, are predictive of response to PD-1 blockade, while a signature of proliferative regulatory cells is not predictive. Taken together, the findings described in the present disclosure highlight the importance of CD4⁺ T cell heterogeneity and the relative balance between activation of novel cytotoxic CD4⁺ effectors and inhibitory regulatory cells for response to PD-1 blockade in bladder cancer.

In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

### Definitions

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B.”

“Acquire” or “acquiring” as the terms are used herein, refer to obtaining possession of a physical entity, or a value, e.g., a numerical value, by “directly acquiring” or “indirectly acquiring” the physical entity or value. “Directly acquiring” means performing a process (e.g., performing a genetic, synthetic, or analytical method or technique) to obtain the physical entity or value. “Indirectly acquiring” refers to receiving the physical entity or value from another party or source (e.g., a third party laboratory that directly acquired the physical entity or value).

The terms “administration” and “administering”, as used herein, refer to the delivery of a bioactive composition or formulation by an administration route including, but not limited to, oral, intravenous, intra-arterial, intramuscular, intraperitoneal, subcutaneous, intramuscular, and topical administration, or combinations thereof. The term includes, but is not limited to, administering by a medical professional and self-administering.

“Cancer” refers to the presence of cells possessing several characteristics typical of cancer-causing cells, such as uncontrolled proliferation, immortality, metastatic potential, rapid growth and proliferation rate, and certain characteristic morphological features. Some types of cancer cells can aggregate into a mass, such as a tumor, but some cancer cells can exist alone within a subject. A tumor can be a solid tumor, a non-solid tumor, a soft tissue tumor, or a metastatic lesion. As used herein, the term “cancer” also encompasses other types of non-tumor cancers. Non-limiting examples include blood cancers or hematological malignancies, such as leukemia, lymphoma, and myeloma. Cancer can include premalignant, as well as malignant cancers.

As used herein, and unless otherwise specified, a “therapeutically effective amount” of an agent is an amount sufficient to provide a therapeutic benefit in the treatment or management of the cancer, or to delay or minimize one or more symptoms associated with the cancer. A therapeutically effective amount of a compound means an amount of therapeutic agent, alone or in combination with other therapeutic agents, which provides a therapeutic benefit in the treatment or management of the cancer. The term “therapeutically effective amount” can encompass an amount that improves overall therapy, reduces or avoids symptoms or causes of the cancer, or enhances the therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Likely to” or “increased likelihood,” as used herein, refers to an increased probability that an item, object, thing or individual will occur. Thus, in one example, an individual that is likely to respond to treatment with an antagonist of PD-L1, alone or in combination with another therapy (e.g., PD-1 therapy), has an increased probability of responding to treatment with the inhibitor alone or in combination, relative to a reference individual or group of individuals. “Unlikely to” refers to a decreased probability that an event, item, object, thing or individual will occur with respect to a reference. Thus, an individual that is unlikely to respond to treatment with an antagonist of PD-L1, alone or in combination with another therapy (e.g., PD-1 therapy), has a decreased probability of responding to treatment with a kinase inhibitor, alone or in combination, relative to a reference individual or group of individuals.

The term “Programmed Death 1” or “PD-1” include isoforms, mammalian, e.g., human PD-1, species homologs of human PD-1, and analogs comprising at least one common epitope with PD-1. The amino acid sequence of PD-1, e.g., human PD-1, is known in the art, e.g., Shinohara T et al. (1994) Genomics 23(3):704-6; Finger L R, et al. Gene (1997) 197(1-2):177-87.

The term or “PD-Ligand 1” or “PD-L1” include isoforms, mammalian, e.g., human PD-1, species homologs of human PD-L1, and analogs comprising at least one common epitope with PD-Ll. The amino acid sequence of PD-L1, e.g., human PD-L1, is known in the art.

As used herein, a “subject” or an “individual” includes animals, such as human (e.g., human subjects) and non-human animals. In some embodiments, a “subject” or “individual” is a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, and non-mammals, such as non-human primates, e.g., sheep, dogs, cows, chickens, amphibians, reptiles, etc.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about”, as used herein, has its ordinary meaning of approximately. The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value

As will be understood by one having ordinary skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is understood that aspects and embodiments of the disclosure described herein include “comprising,” “consisting,” and “consisting essentially of” aspects and embodiments. As used herein, “comprising” is synonymous with “including,” “containing,” or “characterized by,” and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or step.

Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

Headings, e.g., (a), (b), (i) etc., are presented merely for ease of reading the specification and claims. The use of headings in the specification or claims does not require the steps or elements be performed in alphabetical or numerical order or the order in which they are presented.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub- combination was individually and explicitly disclosed herein.

Current efforts to dissect the mechanism of tumor immune surveillance and enhance efficacy of cancer immunotherapies have primarily focused on conventional cytotoxic CD8⁺ T cell-mediated response. However, given the known functional diversity of CD4⁺ T cell effector responses, and emerging data that CD4⁻ T cell recognition may be important for anti-tumor responses for instance in the context of a neoantigen vaccine (Ott et al, 2017; Sahin et al., 2017), the role of specific CD4⁺ populations in enhancing or suppressing immune responses in the tumor microenvironment, and how these are modulated by systemic therapies including immunotherapy, remain unknown.

In the present disclosure, unbiased massively parallel genotypic and phenotypic profiling of the T cell compartment in localized bladder tumors and the adjacent non-malignant compartment, including those treated with anti-PD-L1 immunotherapy, were employed as a tool to finely dissect heterogeneity in both known and novel CD4⁺ subsets. From these experiments, specific subpopulations with functional relevance for response to immunotherapy and clinical outcomes were identified.

As described below in, e.g., Examples 2 and 10, the experimental data presented herein identified distinct states of regulatory T cells, some of which differ based on level of expression of IL2RA and immune checkpoints such as TNFRSF18 which was then confirmed at the protein level. Notably, it was observed that one of the regulatory states that expresses higher levels of IL2RA/TNFSF 18 (tCD4-c5) is more closely linked to the proliferative state in untreated tumors based on both transcriptional and clonotypic information. Without being bound to any particular theory, since a gene signature from checkpoint-high regulatory T cells was found to be associated with worse outcome in non-small cell lung cancer (Guo et al, 2018), this finding suggests that the basal state in untreated bladder tumors favors activation of specific regulatory cells linked to more potent immunosuppression and adverse outcomes.

Additional experimental data presented herein further identified heterogenous populations of cytotoxic CD4⁺ T cells, which differed in their expression of canonical cytolytic effector molecules (granyzmes, perforin) as well as other granule-associated proteins (granulysin, NKG7) which may have roles in target cell killing (see, e.g., Examples 4 and 12). It was subsequently demonstrated that these are distinct populations based on both scRNAseq and flow cytometric validation. The annotation using SingleR indicated that novel effector populations such as cytotoxic CD4⁺ T cells found in the tumor microenvironment may not yet be annotated, and based on “best-fit” comparisons to external reference data and transcriptional correlation within internal data, these cells are in fact most similar to conventional cytotoxic CD8⁺ T cells. While cytotoxic CD4⁺ T cells have been described in non-small cell lung and hepatocellular carcinoma (Zheng et al., 2017a; Guo et al., 2018), have been shown in the circulation to mediate antigen-specific killing following ipilimumab treatment in metastatic melanoma (Kitano et al., 2013), and also are found in an infectious context where they represent a clonally expanded dengue virus-specific effector subset (Patil et al, 2018), the extent of their heterogeneity in other solid tumors (including bladder cancer), and whether these cells are modulated by systemic immunotherapy have remained unclear prior to the work discussed herein. As described in the Examples, it was found that most cytotoxic CD4⁺ subsets in bladder tumors are clonally expanded, suggesting recognition and expansion in response to cognate bladder tumor antigens. Their functional importance is indicated by their ability to kill autologous tumor when expanded ex vivo in the absence of autologous regulatory T cells that may inhibit their activity. Without being bound to any particular theory, the mechanism by which these cells kill target tumor cells involves contact-dependent mechanisms based on inhibition of killing by anti-MHC II antibodies, although other mechanisms may also contribute. Remarkably, cytotoxic CD4⁺ T cells were observed to generally lack surface expression of many immune checkpoints currently being tested with therapeutic antibodies in pre-clinical and clinical testing, suggesting that this effector population may have distinct requirements for activation.

Importantly, while proliferative ON⁻ are heterogeneous and likely include both activated regulatory and cytotoxic CIA⁺ T cells, the data presented herein identified an increased relationship between cytotoxic CD4⁺ T cells and the proliferative state after anti-PD-L1 therapy based on both transcriptional and clonotypic information. Based on pseudotime analysis, it was found that a signature of proliferative cytotoxic CD4⁺ T cells, but not of regulatory CD4⁺ T cells, is predictive of response to anti-PD-L1 therapy in 168 patients with metastatic bladder cancer. While the presence of this signature does not necessarily demonstrate quantitative enrichment of these cell types, the component genes of this signature are largely specific to proliferative cytotoxic CD4⁺ T cells and not to heterogeneous proliferating CD4⁺ or cytotoxic CD4⁺ T cells based on the gene signatures described herein. This finding highlights how anti-PD-L1 therapy may alter the immune microenvironment to favor activation of novel cytotoxic CD4⁺ effectors, particularly in patients with some degree of pre-existing cytotoxic CD4⁺ T cell activation as in the pre-treatment bladder tumor biopsies in this metastatic bladder cancer dataset. The importance of the relative balance between regulatory and effector T cell populations is well-known for conventional effectors, as the regulatory CD4^{+.}cytotoxic CD8⁺ ratio has been associated with improved survival or response to therapy in several cancers including bladder (Preston et al., 2013; Sato et al., 2005; Baras et al., 2016; Takada et al., 2018). The results described herein identify the biological importance of another axis involving the relative balance of regulatory T cells and these cytotoxic CD4⁺ effectors, which needs to be directly examined and would not be captured based solely on assessment of cytolytic effector proteins such as granzymes/perforin which are shared between cytotoxic CD4⁺ and CD8⁻ T cells. The finding described herein also suggests that the interplay between cytotoxic and regulatory populations can also be altered in additional ways for therapeutic benefit, as ex vivo expansion of cytotoxic CD4⁺ T cells in the absence of autologous regulatory cells resulted in their ability to kill autologous tumor cells.

Hence, this work illustrates an important foundation for efforts to enhance bladder tumor immunotherapy. The experimental results presented herein identified novel cytotoxic CD4⁺ effectors whose distinct expression of cytolytic molecules and other marker genes will lead to further efforts to isolate and enhance activity of specific cytotoxic subsets, as well as to discover the bladder tumor antigens they are recognizing. At the same time, this work points to specific regulatory T cell populations which may be more suppressive in bladder cancer and therefore represent ideal targets for parallel approaches to inhibit their activity. In particular, the experimental data presented herein identified a proliferating CD4⁺ signature which predicts response to anti-PD-L1 therapy, which will be of broader utility in orthogonal patient cohorts but also point to the importance of understanding the underlying balance of effector and suppressive T cell activation in determining response to PD-1 blockade.

The gene signatures described herein could be applied to pre-treatment tumor biopsies before starting anti-PD-L1 antibodies to determine the likelihood of responding to or surviving longer with this therapy. The signature could be obtained using a variety of commercially available platforms for RNA expression from archival tumor material, including

Nanostring platform (targeted RNA quantitation), Tempus platform (whole-exome sequencing), and Illumina platform (whole-exome sequencing). The signature itself has not been previously described, and may outperform the ability of existing signatures to predict response, or prognosticate longer survival, with anti-PD-L1 therapy in bladder cancer.

### Programmed Death Ligand 1 (PD-L1)

Programmed Death Ligand 1 (PD-L1), which is also known as cluster of differentiation 274 (CD274) or B7 homolog 1 (B7-H1), is a 40 kDa type 1 transmembrane protein. PD-L1 binds to its receptor, PD-1, found on activated T cells, B cells, and myeloid cells, to modulate activation or inhibition. Both PD-L1 and PD-L2 are B7 homologs that bind to PD-1, but do not bind to CD28 or CTLA-4. Binding of PD-L1 with its receptor PD-1 on T cells delivers a signal that inhibits TCR-mediated activation of IL-2 production and T cell proliferation. It has been reported that the mechanism involves inhibition of ZAP70 phosphorylation and its association with CD3. PD-1 signaling attenuates PKC-6 activation loop phosphorylation resulting from TCR signaling, necessary for the activation of transcription factors NF-xB and AP-1, and for production of IL-2. PD-L1 also binds to the costimulatory molecule CD80 (B7-1), but not CD86 (B7-2).

Expression of PD-L1 on the cell surface has been shown to be upregulated through IFN-y stimulation. PD-L1 expression has been found in many cancers, including human lung, ovarian and colon carcinoma and various myelomas, and is often associated with poor prognosis. PD-L1 has been suggested to play a role in tumor immunity by increasing apoptosis of antigen-specific T-cell clones. It has also been suggested that PD-L1 might be involved in intestinal mucosal inflammation and inhibition of PD-L1 suppresses wasting disease associated with colitis.

Non-limiting examples of mAbs that bind to human PD-L1, and useful in any of the various aspects and embodiments of the compositions and methods disclosed herein include those described in WO2013/019906, WO2010/077634 A1 and U.S. Pat. No. 8,383,796. Specific anti-human PD-L1 mAbs useful as the PD-1 antagonist in the various aspects and embodiments of the compositions and methods disclosed herein include MPDL3280A (atezolizumab), BMS-936559, MEDI4736, MSB0010718C (avelumab).

### Methods of the Disclosure

As described in greater detail below, one aspect of the present disclosure relates to methods for predicting responsiveness of an individual having, or suspected of having, or at risk of having, a bladder cancer to a treatment including an antagonist of Programmed Death Ligand 1 (PD-L1). The method includes (a) profiling expression levels of a panel of genes associated with T-cell specialization and/or T-cell exhaustion expressed in a T cell population from a biological sample obtained from an individual to generate a cell composition profile of the T cell population; (c) determining the presence of a gene signature biomarker in the tumor sample based at least in part upon the measured expression levels, wherein said gene signature biomarker includes one or more genes whose expression is specifically upregulated in proliferating and/or non-proliferating cytotoxic CD4+ T cells while remains unchanged in CD8+ T cells; and (d) identifying the individual as predicted to have an increased responsiveness to the anti-PD-L1 therapy if the gene signature is present in the tumor sample.

In some embodiments, provided herein are methods for selecting an individual having bladder cancer to be subjected to a therapy including a PD-L1 antagonist, the method includes: (a) profiling expression levels of a panel of genes associated with T-cell specialization and/or T-cell exhaustion expressed in a T cell population from a biological sample obtained from an individual to generate a cell composition profile of the T cell population; (b) determining the presence of a gene signature biomarker in the tumor sample based at least in part upon the measured expression levels, wherein said gene signature biomarker includes one or more genes whose expression is specifically upregulated in proliferating and/or non-proliferating cytotoxic CD4+ T cells while remains unchanged in CD8+ T cells; and (c) selecting the individual who is determined to have the gene signature present in the biological sample as an individual to be subjected to a therapy including a PD-L1 antagonist.

In some embodiments, provided herein are methods for treating an individual having bladder cancer, the methods include: (a) profiling expression levels of a panel of genes associated with T-cell specialization and/or T-cell exhaustion expressed in a T cell population from a biological sample obtained from said individual to generate a cell composition profile of the T cell population; (b) determining the presence of a gene signature biomarker in the T cell population based at least in part upon the measured expression levels and the generated cell composition profile, wherein said gene signature biomarker includes one or more genes whose expression is specifically upregulated in proliferating and/or non-proliferating cytotoxic CD4+ T cells while remains unchanged in CD8+ T cells; (c) selecting a therapy including a PD-L1 antagonist; and (d) administering a therapeutically effective amount of the selected therapy to said individual.

The term “biological sample” as used herein refers to materials obtained from or derived from an individual, a subject, or a patient. A biological sample includes sections of tissues, such as biopsy (e.g., tumor biopsy) and autopsy samples, resected tissues (e.g., resected tumors), and frozen sections taken for histological purposes. Such samples include bodily fluids such as blood and blood fractions or products (e.g., serum, plasma, platelets, red blood cells, circulating tumor cells, and the like), lymph, sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. As the proliferative cytolytic CD4⁺ T cell population was found to be specific to the bladder tumor environment, it is contemplated that the biological sample may be obtained from an individual with a bladder cancer tumor. Thus, in some embodiments, the biologcal sample includes at least one bladder cancer cell. In some embodiments, the at least one bladder cancer cell may be obtained via resection. In some embodiments, the at least one bladder cancer cell may be obtained via tumor biopsy.The term “tumor biopsy” refers to tumor tissue sample taken by appropriate means,such as via fine needle biopsy, core needle biopsy, excisional or incisional biopsy, endoscopic biopsy, laparscopic biopsy, thorascopic mediastrinoscopic biopsy, laparotomy, thoracotomy, skin biopsy, and sentinel lymph node mapping and biopsy. Any suitable method for obtaining a tissue sample of a tumor may be used in conjunction with the methods as provided herein.

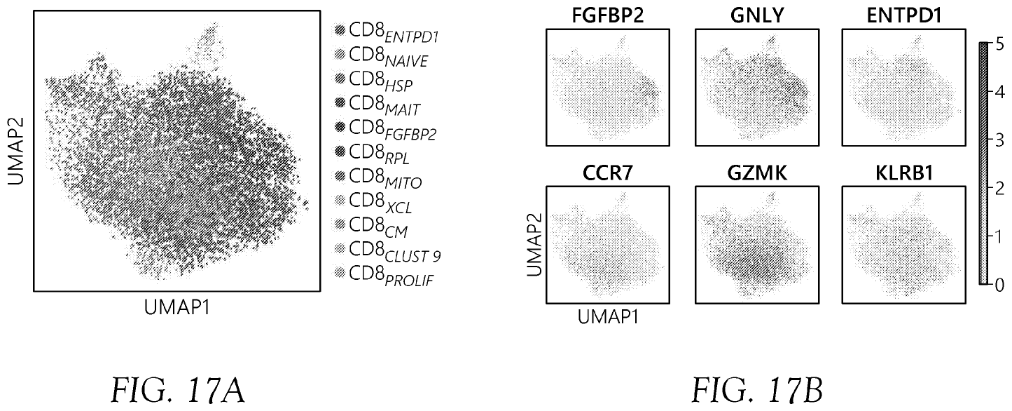

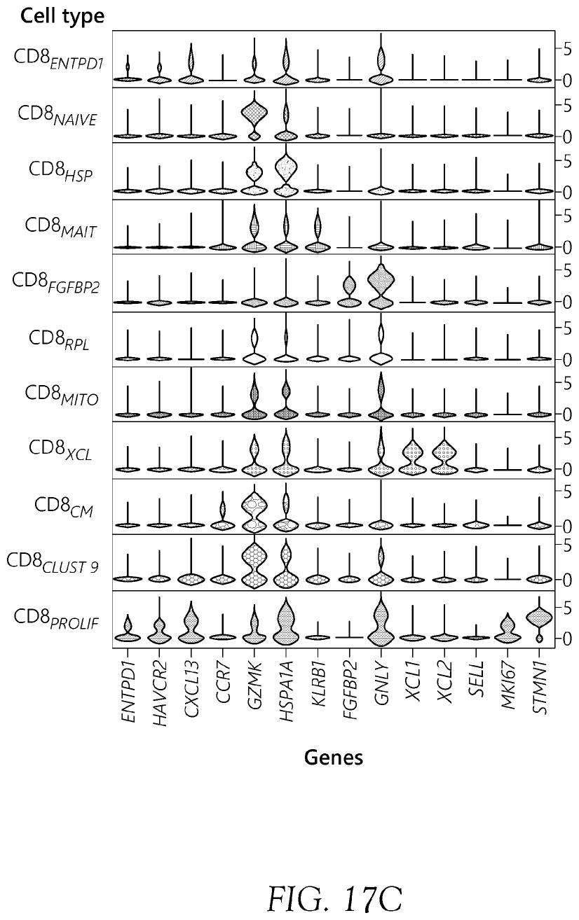

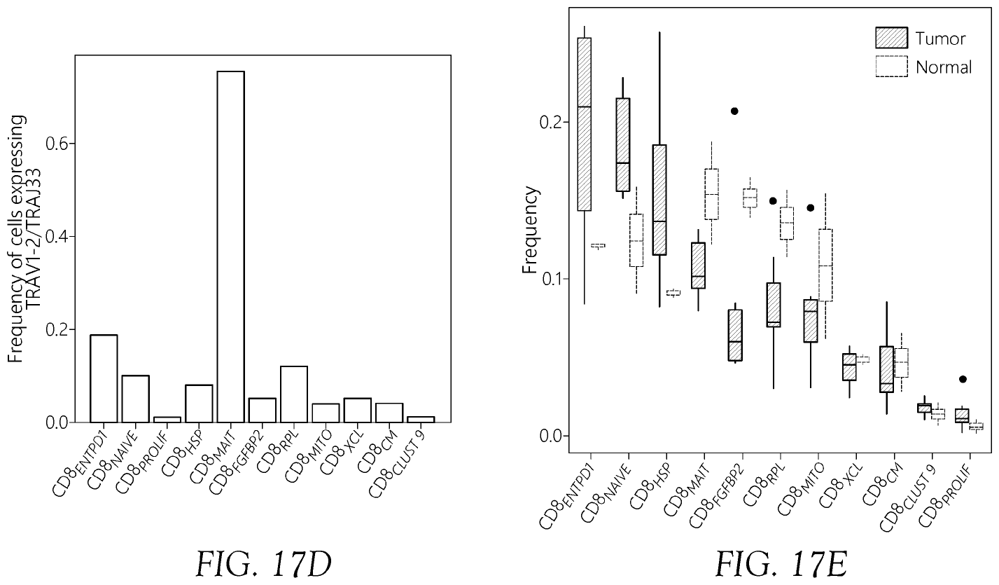

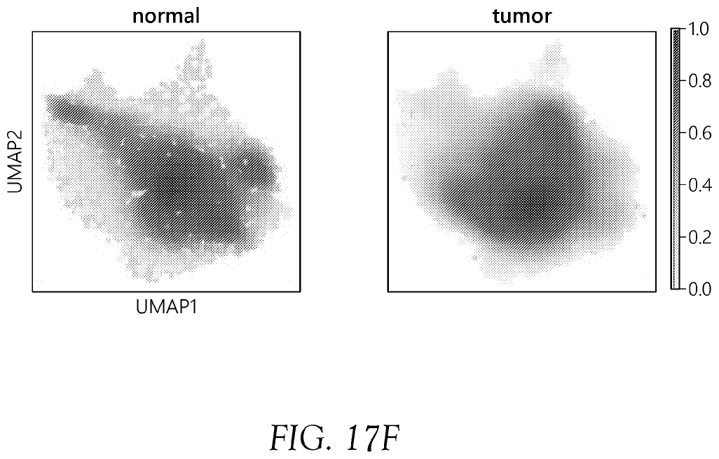

Non-limiting exemplary embodiments of the methods according to the present disclosure include one or more of the following features. In some embodiments, the cell composition profile includes relative proportions of the following T cell subpopulations: tumor-reactive ENTPD1+CD8+ T cells, naïve CD8+ T cells, HSP+CD8+ T cells, mucosal-associated invariant CD8+ T cells, FGFBP2+CD8+ T cells, XCL1+XCL2+CD8+ T cells, central memory CD8+ T cells, effector memory CD8+ T cells, exhausted CD8+ T cells, proliferating CD8+ T cells, regulatory CD4+ T cells, central memory CD4+ T cells, exhausted CD4+ T cells, proliferating cytotoxic CD4+ T cells, and non-proliferating cytotoxic CD4+ T cells. In some embodiments, the cell composition profile includes relative proportions of the eleven (11) CD8+ T cell subpopulations described in FIG. 17A. In some embodiments, the cell composition profile includes relative proportions of the eleven (11) CD4+ T cell subpopulations described in FIG. 18A. In some embodiments, the gene signature biomarker includes one or more of the following parameters: (i) one or more genes identified in Table 2 or Table 7 as upregulated in proliferating CD8⁺ T cells; (ii) one or more genes identified in Table 3 or Table 10 as upregulated in proliferating CD4+ T cells; (iii) one or more genes identified in Table 4 or Table 8 as upregulated in regulatory CD4⁺ T cells; (iv) one or more genes identified in Table 9 as upregulated in cytotoxic CD4+ T cells; and (v) one or more genes identified in Table 5 as upregulated in proliferative cytotoxic CD4⁺ T cells.

Accordingly, in some embodiments, the gene signature biomarker includes one or more genes that are upregulated in proliferating CD8⁺ T cells such as IGLL5, STMN1, TUBB, CXCL13, GZMB, TUBA1B, KIAA0101, UBE2C, HIST1H4C, CCL3, MKI67, ACTB, TOP2A, HLA-DRA, RRM2, CENPF, GNLY, HMGB2, TYMS, CKS1B, SMC4, NUSAP1, S100A4, GAPDH, HMGB1, LGALS1, FKBP1A, HAVCR2, HIST1H1D, CORO1A, HMGN2, NUCKS1, ACTG1, RPA3, BIRC5, ANXAS, TK1, PFN1, CALM3, NUDT1, MT2A, RANBP1, UBE2T, ANAPC11, HLA-DRB1, HOPX, MAD2L1, DUT, PKM, and PCNA (see, e.g., Example 7 and Table 2). Additional suitable genes whose expression is upregulated in proliferating CD8⁺ T cells include UBE2C, SPC25, AURKB, DLGAP5, BIRC5, RRM2, CCNB2, APOBEC3B, CDCA8, GTSE1, ZWINT, TK1, RAD51AP1, KIAA0101, MKI67, STMN1, TYMS, CDC20, KIFC1, CCNA2, TOP2A, NUF2, ASPM, ORC6, CENPW, SGOL1, NCAPG, TPX2, CKAP2L, ASF1B, CKS1B, CDKN3, HIST1H2AJ, CDK1, UBE2T, HIST1H1B, CENPU, NUSAP1, CCNB1, GGH, TUBB, CENPF, MAD2L1, SMC2, PRC1, CLSPN, RNASEH2A, CENPE, MCMI, and FBX05 (see, e.g., Example 9 and Table 7).

In some embodiments, the gene signature biomarker includes one or more genes that are upregulated in proliferating CD4⁺ T cells such as STMN1, TUBB, HIST1H4C, TUBA1B, KIAA0101, HLA-DRA, HMGB2, GZMB, RRM2, LGALS1, TK1, TYMS, GNLY, MT2A, UBE2C, PFN1, GAPDH, ACTB, HLA-DRB1, PKM, CKS1B, DUT, NUSAP1, HMGB1, PCNA, RANBP1, CCL4, TOP2A, MKI67, CD74, ZWINT, PTTG1, TPI1, CENPF, H2AFZ, S100A4, EN01, ANXA5, COTL1, PPP1CA, BIRC5, CORO1A, ACTG1, MIR4435-1HG, CDK1, NUDT1, CALM3, ARPC1B, HIST1H1D, and HLA-DPA1 (see, e.g., Example 8 and Table 3). Additional suitable genes whose expression is upregulated in proliferating CD4+ T cells include RRM2, KIAA0101, UBE2C, TK1, TYMS, BIRC5, CCNB2, MKI67, GGH, RAD51AP1, CCNA2, ZWINT, ASF1B, TOP2A, CENPU, CENPW, STMN1, CLSPN, FBX05, CKS1B, MCMI, CDK1, CENPF, UBE2T, NUSAP1, DTYMK, SMC2, CDKN3, TMEM106C, FEN1, TUBB, MAD2L1, CENPK, NUDT1, MCM3, MCM5, RFC2, PCNA, TUBA1B, DUT, EZH2, HIST1H4C, DEK, SAE1, HMGB2, STRA13, NME1, HLA-DRA, DNAJC9, and CBX5 (see, e.g., Example 15 and Table 10).

In some embodiments, the gene signature biomarker includes one or more genes that are upregulated in regulatory CD4⁺ T cells such as IL2RA, IL32, MIR4435-1HG, TIGIT, CARD16, MAGEH1, PMAIP1, HLA-DRB1, LINC00152, CD74, CD27, HLA-DRA, SAT1, TNFRSF9, CTSC, DUSP4, AC002331.1, TNFRSF18, BATF, HLA-DPB1, TNFRSF4, CXCR6, AC017002.1, LAYN, HPGD, RTKN2, ICA1, LAIR2, HTATIP2, IL1R2, HLA-DPA1, CTLA4, GBP2, GLRX, CST7, S100A4, DNPH1, ACP5, SOX4, ENTPD1, HLA-DQA1, LTB, HLA-DMA, BTG3, HLA-DRB5, TBC1D4, PARK7, USP15, UCP2, and GBP5 (see, e.g., Example 8 and Table 4). Additional suitable genes whose expression is upregulated in regulatory CD4⁺ T cells include IL1R2, IL2RA, EBI3, AC145110.1, TNFRSF4, C14orf182, CADM1, LAIR2, TNFRSF18, FANK1, AC017002.1, LAYN, CUL9, MZB1, FOXP3, SOX4, ZBTB32, LAPTM4B, AC002331.1, TNFRSF9, NGFRAP1, IL32, CRADD, PTPLA, CARD16, MAGEH1, GCNT1, CD79B, CD27, EPHX2, SYNGR2, HLF, LTA, ACP5, PTP4A3, TIGIT, DNPH1, CTSC, HTATIP2, PKM, SAT1, BATF, OTUD5, ADAT2, OAST, CTLA4, GLRX, MIR4435-1HG, LTB, TBC1D4, FANK1, IL2RA, AC002331.1, RTKN2, TNFRSF9, RP11-1399P15.1, SAT1, PMAIP1, IL32, LAYN, HPGD, MAGEH1, TIGIT, MIR4435-1HG, FOXP3, CARD16, HTATIP2, TBC1D4, LTB, and LINC00152 (see, e.g., Example 10 and Table 8).

In some embodiments, the gene signature biomarker includes one or more genes that are upregulated in cytotoxic CD4⁺ T cells such as TMSB10, ACTB, MYL6, ATP5E, KIF'15, MYBL2, ACTG1, ARPC1B, EN01, UQCRB, DNA2, UQCR11.1, TPI1, YWHAB, STMN1, PKM, CDT1, DMC1, COX7C, KIAA0101, LDHB, C9orf16, NDUFA13, ZNF724P, TMEM258, EIF3H, NDUFA4, COX5B, TRAPPC1, PARK7, ECH1, CALM3, CHAF1B, UCK2, CDC6, GAPDH, PRDX5, FAM72B, ATP5A1, MKI67, HNRNPA1, ATP5J2, FKBP1A, PPP1R7, RPL23, SHMT1, PPM1G, DBNL, DPP7, and NOP10 (see, e.g., Example 12 and Table 9).

In some embodiments, the gene signature biomarker includes one or more genes that are upregulated in proliferative cytotoxic CD4⁺ T cells, which are selected from the group consisting of TMSB10, ACTB, MYL6, ATP5E, KIF15, MYBL2, ACTG1, ARPC1B, EN01, UQCRB, DNA2, UQCR11.1, TPI1, YWHAB, STMN1, PKM, CDT1, DMC1, COX7C, KIAA0101, LDHB, C9orf16, NDUFA13, ZNF724P, TMEM258, EIF3H, NDUFA4, COX5B, TRAPPC1, PARK7, ECH1, CALM3, CHAF1B, UCK2, CDC6, GAPDH, PRDX5, FAM72B, ATP5A1, MKI67, HNRNPA1, ATP5J2, FKBP1A, PPP1R7, RPL23, SHMT1, PPM1G, DBNL, DPP7, and NOP10 (see, e.g., Table 5).

In some embodiments, the gene signature biomarker includes at least 2 genes, such as, e.g., at least 2 genes, at least 5 genes, at least 10, at least 20, at least 30, at least 40, at least 50 genes. In some embodiments, the gene signature biomarker includes at least 2, 3, 4, 5, 6, 7, 8, 9, 10 genes. In some embodiments, the gene signature biomarker includes between about 2 to 50 genes, such as e.g., about 5 to 40 genes, about 10 to 30 genes, about 15 to 20 genes, about 20 to 50 genes, about 30 to 50 genes, about 5 to 50 genes, about 5 to 50 genes, or about 5 to 50 genes.

In some embodiments, the gene signature biomarker includes one or more of ABCB1, ACTB, APBA2, ATP5E, CARD16, CXCL13, GPR18, GZMB, HIST1H4C, IGLL5, IL2RA, IL32, KIAA0101, KIF'15, MIR4435-1HG, MYL6, PEG10, SLAMF7, STMN1, TIGIT, TMSB10, TUBA1B, TUBB, GZMK, HLA-DR, PDCD1, TIM3, KLRG1, and combinations of any thereof. In some embodiments, the gene signature biomarker includes one or more of ABCB1, ACTB, APBA2, GPR18, HIST1H4C, IGLL5, IL2RA, IL32, MIR4435-1HG, MYL6, SLAMF7, STMN1, TMSB10, TUBB, and combinations of any thereof. In some embodiments, the gene signature biomarker includes one or more of GZMK, HLA-DR, PDCD1, TIM3, KLRG1, and combinations of any thereof

In some embodiments, the biological sample includes bladder cancer cells obtained from the individual. In some embodiments, the biological sample includes peripheral blood obtained from the individual. In some embodiments, the bladder cancer is squamous cell carcinoma. In some embodiments, the bladder cancer is non-squamous cell carcinoma. In some embodiments, the bladder cancer is adenocarcinoma. In some embodiments, the bladder cancer is small cell carcinoma. In some embodiments, the bladder cancer is selected from the group consisting of early stage bladder cancer, metastatic bladder cancer, non-metastatic bladder cancer, early-stage bladder cancer, non-invasive bladder cancer, muscle-invasive bladder cancer (MIBC), non-muscle-invasive bladder cancer (NMIBC), primary bladder cancer, advanced bladder cancer, locally advanced bladder cancer, bladder cancer in remission, progressive bladder cancer, and recurrent bladder cancer. In some embodiments, the bladder cancer is localized resectable, localized unresectable, or unresectable. In some embodiments, the bladder cancer is a high grade, non-muscle-invasive cancer that has been refractory to standard intra-bladder infusion (intravesical) therapy. In some embodiments, the bladder cancer is metastatic bladder cancer.

The term “PD-L1 antagonist” as defined herein is any molecule or compound that partially or fully blocks, inhibits, or neutralizes a biological activity and/or function mediated by a PD-L1 polypeptide. In some embodiments, such PD-L1 antagonist binds to PD-Ll. In some embodiments, the PD-L1 antagonist is a polypeptide antagonist. In some embodiments, the PD-L1 antagonist is a small molecule antagonist. In some embodiments, the PD-L1 antagonist is a polynucleotide antagonist, such as an antisense molecule, a ribozyme, a double-stranded RNA molecule, a triple helix molecule, that hybridizes to a nucleic acid encoding the gene biomarker, or a transcription regulatory region that blocks or reduces mRNA expression of the gene biomarker. In some embodiments, the PD-L1 antagonist is an anti-PD-L1 antibody or an anti-PD-1 antibody. Non-limiting examples of anti-PD-1 antibodies suitable for the compositions and methods disclosed herein include pembrolizumab (Keytruda®, MK-3475), nivolumab, pidilizumab, lambrolizumab, MEDI-0680, PDR001, and REGN2810. Additional anti-PD-1 antibodies suitable for the compositions and methods disclosed herein include, but are not limited to those described in, e.g., U.S. Pat. Nos. 7,521,051, U.S. Pat. No. 8,008,449, U.S. Pat. No. 8,354,509, and PCT Pat. Pub. Nos. WO2009/114335, WO2015/026634, WO2008/156712, WO2015/026634, WO2003/099196, WO2009/101611, WO2010/027423, WO2010/027827, WO2010/027828, WO2008/156712, WO2008/15671, WO2013/173223, WO2015/026634, and WO2008/156712. In some embodiments, the anti-PD1 antibody includes pembrolizumab. In some embodiments, the PD-L1 antagonist is an anti-PD-L1 antibody. Non-limiting examples of anti-PD-1 antibodies suitable for the compositions and methods disclosed herein include atezolizumab (MPDL3280A), BMS-936559 (MDX-1105), durvalumab (MEDI4736), avelumab (MSB0010718C), YW243.55.570. Additional anti-PD-L1 antibodies suitable for the compositions and methods disclosed herein include, but are not limited to those described in, e.g., PCT Pat. Pub. Nos. WO2015026634, WO2013/019906, WO2010077634, WO2010077634, WO2007005874, WO2016007235, and U.S. Pat. No. 8,383,796. In some embodiments, the anti-PD-L1 antibody includes one or more of atezolizumab (MPDL3280A), BMS-936559 (MDX-1105), durvalumab (MEDI4736), avelumab (MSB0010718C), YW243.55.570, and combinations of any thereof. In some embodiments, the anti-PD-L1 antibody includes atezolizumab.

In some embodiments, the anti-PD-L1 antibody includes atezolizumab. In instances where the anti-PD-L1 antibody includes atezolizumab, the gene signature biomarker includes one or more genes whose expression is upregulated in proliferating CD4+ T cells and/or upregulated in non-proliferating CD4+ T cells while remains substantially unchanged in CD8+ T cells. In some embodiments, the gene signature biomarker includes one or more genes selected from the group consisting of ABCB1, APBA2, SLAMF7, GPR18, PEG10, and combinations of any thereof. In some embodiments, the gene signature biomarker includes one or more genes selected from the group consisting of GZMK, GZMB, HLA-DR, PDCD1, TIM3, and combinations of any thereof In some embodiments, the gene signature biomarker includes a gene combination selected from the group consisting of: (a) expression of CD4, GZMB, and HLA-DR; (b) expression of CD4, GZMK and HLA-DR; and (c) expression of CD4, GZMK, PDCD1, and TIM3. In some embodiments, the gene signature biomarker further includes undetectable expression of FOXP3 and CCR73.

In some embodiments, the gene signature biomarker includes one or more genes selected from the group consisting of GZMB, GZMK, HLA-DR, PDCD1, Ki67, TIM3, and combinations of any thereof In some embodiments, the gene signature biomarker comprises a gene combination selected from the group consisting of: (a) expression of CD8, GZMB, and TIM3: (b) expression of CD8, GZMB, PDCD1, and TIM3; (c) expression of CD8, GZMK, and TIM3; (d) expression of CD8, GZMK, PDCD1, and TIM3; (e) expression of CD8, GZMK, and HLA-DR; (f) expression of CD8, GZMK, and Ki67; and (g) expression of CD8, GZMK, HLA-DR, and Ki67. In some embodiments, the gene signature biomarker further includes undetectable expression of CCR7.

One skilled in the art will appreciate that the expression level of a gene generally refers to a determined level of gene expression. This may be a determined level of gene expression as an absolute value or compared to a reference gene (e.g. a housekeeping gene), to the average of two or more reference genes, or to a computed average expression value (e.g., in DNA chip analysis) or to another informative gene without the use of a reference sample. The expression level of a gene may be measured directly, e.g., by obtaining a signal wherein the signal strength is correlated to the amount of mRNA transcripts of that gene or it may be obtained indirectly at a protein level, e.g., by immunohistochemistry, flow cytometry, CISH, ELISA or RIA methods. The expression level may also be obtained by way of a competitive reaction to a reference sample. An expression value which is determined by measuring some physical parameter in an assay, e.g. fluorescence emission, may be assigned a numerical value which may be used for further processing of information.

In some embodiments, the profiling expression levels of a panel of genes associated with T-cell specialization and/or T-cell exhaustion includes one or more nucleic-acid-based analytical assays such as, for example, single-cell RNA sequencing, single sample gene set enrichment analysis, northern blotting, fluorescent in-situ hybridization (FISH), polymerase chain reaction (PCR), real-time PCR, reverse transcription polymerase chain reaction (RT-PCR), quantitative reverse transcription PCR (qRT-PCR), serial analysis of gene expression (SAGE), microarray, or tiling arrays. In some embodiments, the nucleic acid-based analytical assay includes single-cell RNA sequencing (see, e.g., Examples 5 and 20).

In some embodiments, the profiling expression levels of a panel of genes associated with T-cell specialization and/or T-cell exhaustion includes one or more protein expression-based analytical assays such as, for example, ELISA, CISH, RIA, immunohistochemistry, western blotting, mass spectrometry, flow cytometry, protein-microarray, immunofluorescence, or multiplex detection assay. In some embodiments, the protein expression-based analytical assay includes flow cytometry (see, e.g., Example 18).

Identifying accurate predictive biomarkers for an anti-PD-L1 therapy has several direct clinical applications. In bladder cancer, one could foresee that patients with high responsiveness level to an anti-PD-L1 therapy could receive, e.g., anti-PD-L monotherapy, whereas those with intermediate/low responsiveness levels could be treated, e.g., with the more active (but more toxic) combination antagonists. In addition, this approach could stratify patients between anti-PD-L1 and other active agents such as cytotoxic chemotherapy.

Accordingly, some embodiments of the disclosure provide methods for treating an individual having, suspected of having, or at risk of having, a cancer, e.g., a bladder cancer, by administering to the individual an effective amount of an agent (e.g., a therapeutic agent) that targets and/or inhibits the PD-Ll/PD-1 pathway. In some embodiments, the disclosed methods further include treating the bladder cancer by administering to the individual a therapeutically effective amount of a PD-L1 antagonist. In some embodiments, the methods of the disclosure further include (a) selecting a PD-L1 antagonist appropriate for the treatment of the bladder cancer in the individual based on whether the gene signature biomarker is present in the individual; and (b) administering a therapeutically effective amount of the selected PD-Ll antagonist to the individual. In some embodiments, the methods further include one or more of the following: (a) selecting the individual as predicted to have an increased responsiveness to a therapy with a PD-L1 antagonist if a gene signature biomarker as disclosed herein is detected in a biological sample from the individual; (b) selecting the patient as predicted to not have an increased responsiveness to a therapy with a PD-L1 antagonist if a gene signature biomarker as disclosed herein is not detected in the biological sample.

In some embodiments, the individual has a bladder cancer, or suspected of having or at risk of having a bladder cancer. The bladder cancer can be at any forms or stages of disease, e.g., any states described herein, including but are not limited to, squamous cell carcinoma, non-squamous cell carcinoma, adenocarcinoma, and small cell carcinoma. In some embodiments, the bladder cancer is selected from the group consisting of metastatic bladder cancer, non-metastatic bladder cancer, early-stage bladder cancer, non-invasive bladder cancer, non-muscle-invasive bladder cancer, primary bladder cancer, advanced bladder cancer, locally advanced bladder cancer, bladder cancer in remission, progressive bladder cancer, and recurrent bladder cancer. In some embodiments, the bladder cancer is metastatic bladder cancer. In some embodiments, the individual has, or suspected of having or at risk of having a bladder cancer, wherein the bladder cancer includes an expression alteration in e.g., one or more of the genes set forth in Tables 2-5, e.g., an overexpression or repression as described herein. In some embodiments, the bladder cancer comprises, or is identified as having, an expression alteration in one or more of the genes selected from ABCB1, ACTB, ABCB1, ATP5E, CARD16, CXCL13, GPR18, GZMB, HIST1H4C, IGLL5, IL2RA, IL32, KIAA0101, KIF15, MIR4435-1HG, MYL6, PEG10, SLAMF7, STMN1, TIGIT, TMSB10, TUBA1B, TUBB, GZMK, HLA-DR, PDCD1, TIM3, KLRG1, and combinations of any thereof In some embodiments, the gene signature biomarker includes one or more of ABCB1, ACTB, APBA2, GPR18, HIST1H4C, IGLL5, IL2RA, IL32, MIR4435-1HG, MYL6, SLAMF7, STMN1, TMSB10, TUBB, and combinations of any thereof. In some embodiments, the gene signature biomarker includes one or more of GZMK, HLA-DR, PDCD1, TIM3, KLRG1, and combinations of any thereof. In some embodiments, the subject is identified, or has been previously identified, as having a bladder cancer.

In some embodiments, the individual is a human, e.g., a human patient having a bladder cancer, e.g., a metastatic bladder cancer, as described herein.

In some embodiments, the individual is undergoing or has undergone treatment with a different (e.g., non-PD-1 and/or non-PD-L1) therapeutic agent or therapeutic regimen. In some embodiments, the different therapeutic agent or therapeutic regimen is a chemotherapy, a radiation therapy, an immunotherapy, an immunoradiotherapy, a hormonal therapy, an oncolytic virotherapy, a surgical procedure, or any combination thereof

In some embodiments, the individual is a bladder cancer patient who has participated in a clinical trial for an antagonist of PD-L1 and/or PD-1. In some embodiments, the individual is a bladder patient who has participated in a clinical trial for a different (e.g., non-PD-1 and/or non-PD-L1) therapeutic agent or therapeutic regimen.

In some embodiments, the individual is a human patient (e.g., a male or female of any age group), e.g., a pediatric patient (e.g., infant, child, adolescent); or adult patient (e.g., young adult, middle-aged adult or senior adult). In some embodiments, the individual is an adult individual (e.g., male or female adult individual) having, or at risk of having, a melanoma as described herein. In some embodiments, the individual is an individual of or above 10, 15, 20, 25, 30, 35, 40, 45, 50, 55, 60, 65, 70, 75, 80, 85, 90 years of age, or more. In some embodiments, an individual is an individual between 0-10 years of age, 10-20 years of age, 20-30 years of age, 30-40 years of age, 40-50 years of age, 50-60 years of age, 60-70 years of age, 70-80 years of age, or 80-90 years of age. In certain embodiments, the individual is an individual between 25 and 29 years of age. In other embodiments, the individual is an individual between 15 and 29 years of age. In some embodiments, the individual is female and is between 15 and 29 years of age. In certain embodiments, the individual is 65 years of age, or more. In some embodiments, the individual is 60 years of age, or older. In another embodiment, the individual is between 45 and 60 years of age. In yet some other embodiments, the individual is 45 years of age, or younger. In still some other embodiments, the individual is 30 years of age, or younger. In some embodiments, the individual is 45 years of age, or older, and is a male. In some other embodiments, the individual is 45 years of age, or younger, and is a female. In some embodiments, the individual has a family history of bladder cancer.

As discussed supra, the PD-L1 antagonist can be administered in combination with one or more additional therapies such as, for example, chemotherapeutics or anti-cancer agents or anti-cancer therapies. By “in combination with,” it is not intended to imply that the anti-PD-L1 therapy and the additional therapies must be administered at the same time and/or formulated for delivery together, although these methods of delivery are within the scope of the disclosure. The therapies can be administered concurrently with, prior to, or subsequent to, one or more other additional therapies or therapeutic agents. In general, each therapy or therapeutic agent will be administered at a dose and/or on a time schedule determined for that therapy or therapeutic agent. It will further be appreciated that therapies and therapeutic agents utilized in a combination can be administered together in a single composition or administered separately in different compositions. The particular combination to employ in a regimen will take into account compatibility of the first therapeutically active agent with the additional therapeutically active agent(s) and/or the desired therapeutic effect to be achieved.

In some embodiments, the one or more additional therapies, chemotherapeutics, anti-cancer agents, or anti-cancer therapies is selected from the group consisting of chemotherapy, radiotherapy, immunotherapy, hormonal therapy, toxin therapy, and surgery. “Chemotherapy” and “anti-cancer agent” are used interchangeably herein. Various classes of anti-cancer agents can be used. Non-limiting examples include: alkylating agents, antimetabolites, anthracyclines, plant alkaloids, topoisomerase inhibitors, podophyllotoxin, antibodies (e.g., monoclonal or polyclonal), tyrosine kinase inhibitors (e.g., imatinib mesylate (Gleevec® or Glivec®)), hormone treatments, soluble receptors and other antineoplastics.

Topoisomerase inhibitors are also another class of anti-cancer agents that can be used herein. Topoisomerases are essential enzymes that maintain the topology of DNA. Inhibition of type I or type II topoisomerases interferes with both transcription and replication of DNA by upsetting proper DNA supercoiling. Some type I topoisomerase inhibitors include camptothecins: irinotecan and topotecan. Examples of type II inhibitors include amsacrine, etoposide, etoposide phosphate, and teniposide. These are semisynthetic derivatives of epipodophyllotoxins, alkaloids naturally occurring in the root of American Mayapple (Podophyllum peltatum).

Antineoplastics include the immunosuppressant dactinomycin, doxorubicin, epirubicin, bleomycin, mechlorethamine, cyclophosphamide, chlorambucil, ifosfamide. The antineoplastic compounds generally work by chemically modifying a cell's DNA.

Alkylating agents can alkylate many nucleophilic functional groups under conditions present in cells. Cisplatin and carboplatin, and oxaliplatin are alkylating agents. They impair cell function by forming covalent bonds with the amino, carboxyl, sulfhydryl, and phosphate groups in biologically important molecules.

Vinca alkaloids bind to specific sites on tubulin, inhibiting the assembly of tubulin into microtubules (M phase of the cell cycle). The vinca alkaloids include: vincristine, vinblastine, vinorelbine, and vindesine.

Anti-metabolites resemble purines (azathioprine, mercaptopurine) or pyrimidine and prevent these substances from becoming incorporated in to DNA during the “S” phase of the cell cycle, stopping normal development and division. Anti-metabolites also affect RNA synthesis.

Plant alkaloids and terpenoids are obtained from plants and block cell division by preventing microtubule function. Since microtubules are vital for cell division, without them, cell division cannot occur. The main examples are vinca alkaloids and taxanes.

Podophyllotoxin is a plant-derived compound which has been reported to help with digestion as well as used to produce two other cytostatic drugs, etoposide and teniposide. They prevent the cell from entering the GI phase (the start of DNA replication) and the replication of DNA (the S phase).

Taxanes as a group includes paclitaxel and docetaxel. Paclitaxel is a natural product, originally known as Taxol and first derived from the bark of the Pacific Yew tree. Docetaxel is a semi-synthetic analogue of paclitaxel. Taxanes enhance stability of microtubules, preventing the separation of chromosomes during anaphase.

In some embodiments, the anti-cancer agents can be selected from remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, methotrexate, gefitinib (Iressa0), taxol, taxotere, fluorouracil, leucovorin, irinotecan, xeloda, CPT-11, interferon alpha, pegylated interferon alpha (e.g., PEG INTRON-A), capecitabine, cisplatin, thiotepa, fludarabine, carboplatin, liposomal daunorubicin, cytarabine, doxetaxol, pacilitaxel, vinblastine, IL-2, GM-C SF, dacarbazine, vinorelbine, zoledronic acid, palmitronate, biaxin, busulphan, prednisone, bortezomib (Velcade®), bisphosphonate, arsenic trioxide, vincristine, doxorubicin (Doxil®), paclitaxel, ganciclovir, adriamycin, estrainustine sodium phosphate (Emcyt®), sulindac, etoposide, and combinations of any thereof

In other embodiments, the anti-cancer agent can be selected from bortezomib, cyclophosphamide, dexamethasone, doxorubicin, interferon-alpha, lenalidomide, melphalan, pegylated interferon-alpha, prednisone, thalidomide, or vincristine.

In some embodiments, the methods of treatment as described herein further include an immunotherapy. In some embodiments, the immunotherapy includes administration of one or more checkpoint inhibitors. Accordingly, some embodiments of the methods of treatment described herein include further administration of a compound that inhibits one or more immune checkpoint molecules. In some embodiments, the one or more immune checkpoint molecules include one or more of CTLA4, A2AR, B7-H3, B7-H4, TIM3, and combinations of any thereof. In some embodiments, the compound that inhibits the one or more immune checkpoint molecules includes an antagonistic antibody.

In some aspects, the one or more anti-cancer therapy is radiation therapy. In some embodiments, the radiation therapy can include the administration of radiation to kill cancerous cells. Radiation interacts with molecules in the cell such as DNA to induce cell death. Radiation can also damage the cellular and nuclear membranes and other organelles. Depending on the radiation type, the mechanism of DNA damage may vary as does the relative biologic effectiveness. For example, heavy particles (e.g., protons, neutrons) damage DNA directly and have a greater relative biologic effectiveness. Electromagnetic radiation results in indirect ionization acting through short-lived, hydroxyl free radicals produced primarily by the ionization of cellular water. Clinical applications of radiation consist of external beam radiation (from an outside source) and brachytherapy (using a source of radiation implanted or inserted into the patient). External beam radiation consists of X-rays and/or gamma rays, while brachytherapy employs radioactive nuclei that decay and emit alpha particles, or beta particles along with a gamma ray. Radiation also contemplated herein includes, for example, the directed delivery of radioisotopes to cancer cells. Other forms of DNA damaging factors are also contemplated herein such as microwaves and UV irradiation.

Radiation may be given in a single dose or in a series of small doses in a dose-fractionated schedule. The amount of radiation contemplated herein ranges from about 1 to about 100 Gy, including, for example, about 5 to about 80, about 10 to about 50 Gy, or about 10 Gy. The total dose may be applied in a fractioned regime. For example, the regime may include fractionated individual doses of 2 Gy. Dosage ranges for radioisotopes vary widely, and depends on the half-life of the isotope and the strength and type of radiation emitted. When the radiation includes use of radioactive isotopes, the isotope may be conjugated to a targeting agent, such as a therapeutic antibody, which carries the radionucleotide to the target tissue (e.g., tumor tissue).

Surgery described herein includes resection in which all or part of a cancerous tissue is physically removed, exercised, and/or destroyed. Tumor resection refers to physical removal of at least part of a tumor. In addition to tumor resection, treatment by surgery includes laser surgery, cryosurgery, electrosurgery, and microscopically controlled surgery (Mohs surgery). Removal of precancers or normal tissues is also contemplated herein.

Accordingly, in some embodiments of the therapeutic methods disclosed herein, the first therapy comprising a PD-L1 antagonist is administered to the individual in combination with a second therapy such as an anti-cancer agent, a chemotherapeutic, or anti-cancer therapy. In some embodiments, the second anti-cancer therapy is selected from the group consisting of chemotherapy, radiotherapy, immunotherapy, hormonal therapy, toxin therapy, and surgery.

In some embodiments, the second therapy includes an anti-PD1 therapy. In some embodiments, the anti-PD1 therapy includes one or more PD-1 antagonists. The term “PD-1 antagonist” refers to any chemical compound or biological molecule that blocks binding of PD-L1 expressed on a cancer cell to PD-1 expressed on an immune cell (T cell, B cell or NKT cell) and optionally also blocks binding of PD-L2 expressed on a cancer cell to the immune-cell expressed PD-1. In some embodiments, where a human individual is being treated, the PD-1 antagonist blocks binding of human PD-L1 to human PD-1, and optionally blocks binding of both human PD-L1 and PD-L2 to human PD-1. PD-1 antagonists useful in the compositions and methods disclosed herein include PD-1 antibodies (e.g., monoclonal antibodies - mAb), or antigen binding fragment thereof, which specifically binds to PD-1 or PD-Ll. In some embodiments, the PD-1 antibodies suitable for the compositions and methods disclosed herein include those capable of specifically binding to human PD-1 or human PD-Ll.

Non-limiting examples of PD-1 antibodies suitable for an anti-PD1 therapy include pembrolizumab (Keytruda®, MK-3475), nivolumab, pidilizumab, lambrolizumab, MEDI-0680, PDR001, and REGN2810. Additional anti-PD-1 antibodies suitable for an anti-PD1 therapy include, but are not limited to those described in, e.g. ,U U.S. Pat. Nos. 7,521,051, U.S. Pat. No. 8,008,449, U.S. Pat. No. 8,354,509, and PCT Pat. Pub. Nos. WO2009/114335, WO2015/026634, WO2008/156712, WO2015/026634, WO2003/099196, WO2009/101611, WO2010/027423, WO2010/027827, WO2010/027828, WO2008/156712, WO2008/15671, WO2013/173223, WO2015/026634, and WO2008/156712. Examples of mAbs that bind to human PD-1, and useful in the various aspects and embodiments of the present disclosure, are described in U.S. Pat. Nos. 7,521,051; 8,008,449; and 8,354,509. Specific anti-human PD-1 mAbs useful as the PD-1 antagonist in various aspects and embodiments of the present invention include: pembrolizumab, a humanized IgG4 mAb with the structure described in WHO Drug Information, Vol. 27, No. 2, pages 161-162 (2013), nivolumab (BMS-936558), a human IgG4 mAb with the structure described in WHO Drug Information, Vol. 27, No. 1, pages 68-69 (2013); pidilizumab (CT-011, also known as hBAT or hBAT-1); and the humanized antibodies h409A1 1; h409A16 and h409A17, which are described in PCT Pub. No. WO2008/156712.

In some embodiments, the second therapy includes an anti-TGF-I3 therapy. In some embodiments, the anti-TGF-i3 therapy includes one or more TGF-I3 antagonists. In some embodiments, the one or more TGF-I3 antagonists are selected from the group consisting of an antibody directed against one or more isoforms of TGF-I3, a TGF-I3 receptor, an antibody directed against one or more TGF-I3 receptors, latency associated peptide, large latent TGF-P, a TGF-(3 inhibiting proteoglycan, somatostatin, mannose-6-phosphate, mannose-1 —phosphate, prolactin, insulin-like growth factor II, IP- 10, an Arg-Gly-Asp containing peptide, an antisense oligonucleotide, and a protein involved in TGF-I3 signaling. In some embodiments, the TGF-(3 inhibiting proteoglycan is selected from the group consisting of fetuin, decorin, biglycan, fibromodulin, lumican, and endoglin. In some embodiments, the protein involved in TGF-I3 signaling is selected from the group consisting of SMADs, MADs, Ski, and Sno.

In some embodiments, the first therapy and the second therapy are administered concomitantly. In some embodiments, the first therapy and the second therapy are administered sequentially. In some embodiments, the first therapeutic agent is administered before the second therapy. In some embodiments, the first therapy is administered before and/or after the second therapy. In some embodiments, the first therapy and the second therapy are administered in rotation. In some embodiments, the first therapy is administered at the same time as the second therapy. In some embodiments, the first therapy and the second therapy are administered together in a single formulation.

### Systems and Kits

In one aspect of the disclosure, provided herein are various kits for use in predicting responsiveness of a bladder cancer to an anti-PD-L1 therapy and/or in treating a bladder cancer in an individual. The kits include (a) one or more detection reagents, capable of detecting and/or profiling expression levels of a panel of genes associated with T-cell specialization and/or T-cell exhaustion expressed in a T cell population to generate a cell composition profile of the T cell population. In some embodiments, the kits include (a) one or more detection reagents, capable of detecting one or more of the following parameters in a biological sample from an individual having, or suspected of having cancer (e.g., a bladder cancer patient): (i) one or more genes identified in Table 2 or Table 7 as upregulated in proliferating CD8⁺ T cells; (ii) one or more genes identified in Table 3 or Table 10 as upregulated in proliferating CD4⁺ T cells; (iii) one or more genes identified in Table 4 or Table 8 as upregulated in regulatory CD4⁺ T cells; (iv) one or more genes identified in Table 9 as upregulated in cytotoxic CD4+ T cells; and (v) one of more genes identified in Table 5 as upregulated in proliferative cytotoxic CD4⁺ T cells; and b) instructions for use in predicting responsiveness of a bladder cancer to an anti-PD-L1 therapy and/or in treating a bladder cancer in an individual. In some embodiments, the disclosed kits further include an antagonist of PD-L1 and optionally an antagonist of PD-1 or a combination thereof.

In some embodiments, the kits of the disclosure further include one or more syringes (including pre-filled syringes) and/or catheters (including pre-filled syringes) used to administer any one of the provided PD-L1 antagonists and/or PD-1 antagonists to a subject in need thereof. In some embodiments, a kit can have one or more additional therapeutic agents that can be administered simultaneously or sequentially with the other kit components for a desired purpose, e.g., for treating a bladder cancer in a subject in need thereof.

For example, any of the above-described kits can further include one or more additional reagents, where such additional reagents can be selected from: dilution buffers; reconstitution solutions, wash buffers, control reagents, negative controls, and positive controls.

In some embodiments, the components of a kit can be in separate containers. In some other embodiments, the components of a kit can be combined in a single container

In another aspect, also provided herein are various systems including (a) at least one processor; and (b) at least one memory including program code which when executed by the one memory provides operations for performing a method as disclosed herein. In some embodiments, the operations include (a) acquiring knowledge of the presence of a gene signature biomarker in a biological sample from an individual; and (b) providing, via a user interface, a prognosis for the subject based at least in part on detected knowledge. In some embodiments, provided herein are systems for evaluating an individual having, or suspected of having, or at risk of having a cancer, e.g., a bladder cancer. The systems include at least one processor operatively connected to a memory, the at least one processor when executing is configured to (a) acquire knowledge of the presence of a gene signature biomarker in a biological sample from an individual; and (b) provide, via a user interface, a prognosis for the subject based at least in part on detected knowledge.

Additional embodiments are disclosed in further detail in the following examples, which are provided by way of illustration and are not in any way intended to limit the scope of this disclosure or the claims.

### EXAMPLES

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning. A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferre, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

As described in greater detail in the Examples below, single-cell RNA and paired T cell receptor (TCR) sequencing were performed on T cells from tumors and paired non-malignant tissue from patients with localized muscle-invasive bladder cancer. Patients treated with anti-PD-L1 before surgery were also assessed. It was observed that the composition and repertoire of CD8⁺ populations are not altered in tumors. However, ay+T cells were found to demonstrate several tumor-specific states. These included three distinct states of regulatory T cells that were enriched and clonally expanded in tumors. Experimental data presented herein also identified several populations of cytotoxic CD4⁺ , which were clonally expanded in tumor and could kill autologous tumor. In particular, experimental data presented herein identified a

WO 2021/030156 PCT/US2020/045263 heterogeneous proliferating CD4⁺ state comprised of regulatory and cytotoxic CD4⁺ populations. It was further observed that while untreated bladder tumors were enriched for regulatory cells in the proliferative state, anti-PD-L1 treatment biased cytotoxic populations towards the proliferative state. A gene signature of proliferative cytotoxic CD4⁺ in tumors could predict clinical response in 168 metastatic bladder cancer patients treated with anti-PD-Ll. Taken together, the experimental data disclosed herein reveals the importance of cytotoxic CD4⁺ effectors in response to PD-1 blockade.

### Example 1

### Canonical CD8+ T Cell Populations are not Enriched in the Bladder Tumor Microenvironment

This Example describes experiments performed to assess the T cell composition of the tumor environment. T cells from dissociated bladder tumors and adjacent uninvolved bladder tissues were profiled using single-cell RNA and T-cell receptor (TCR) sequencing (see, e.g., Table 1 below).

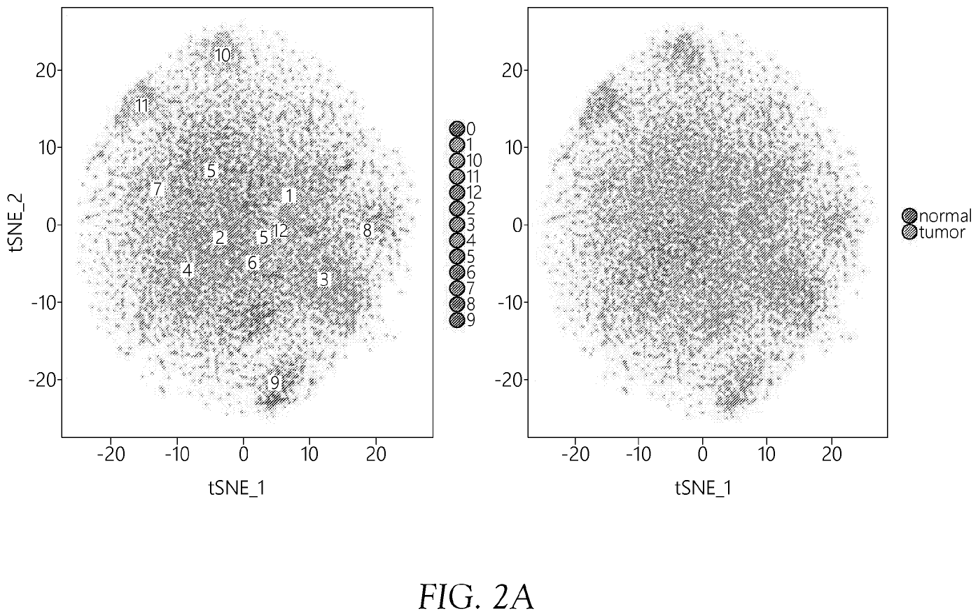

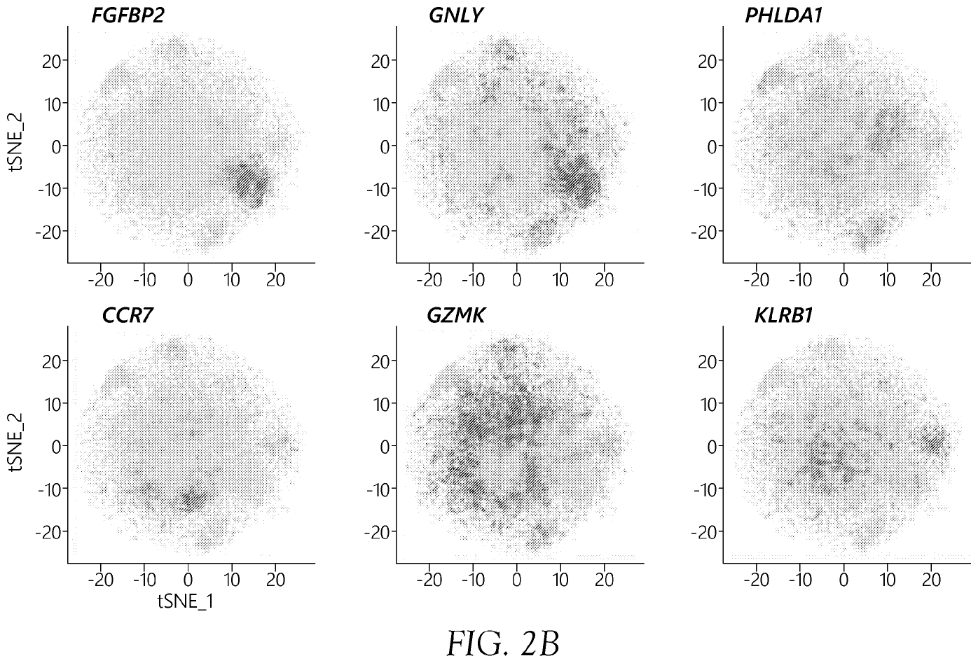

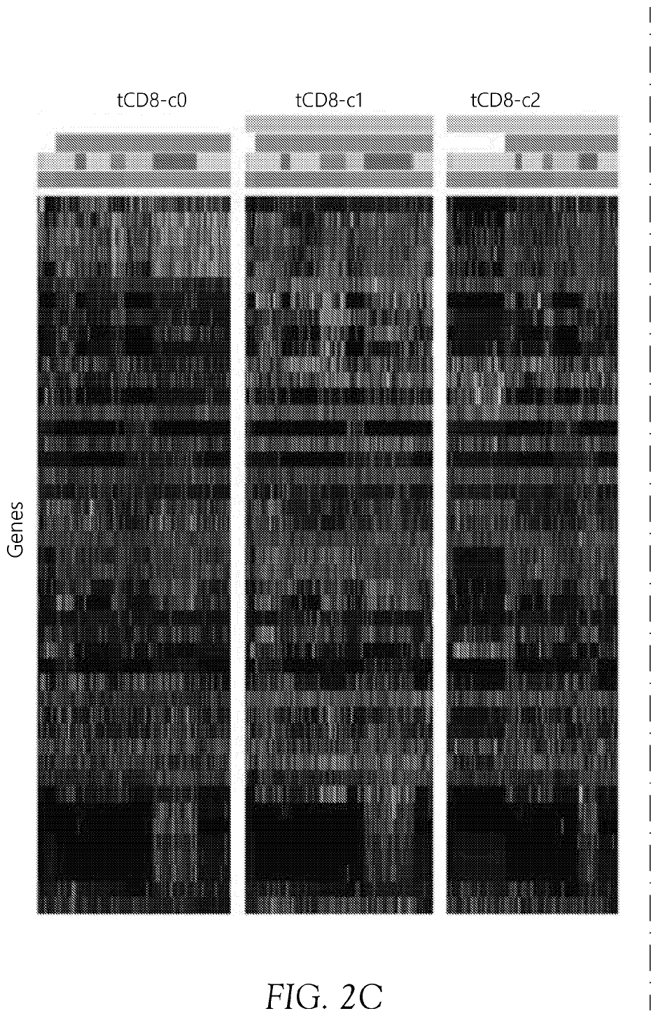

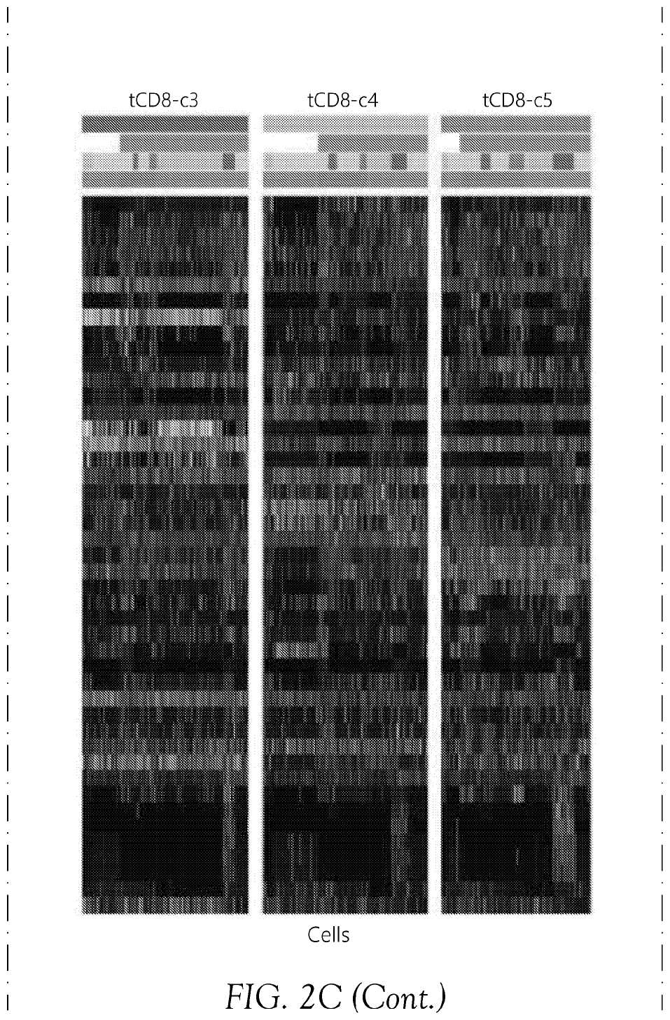

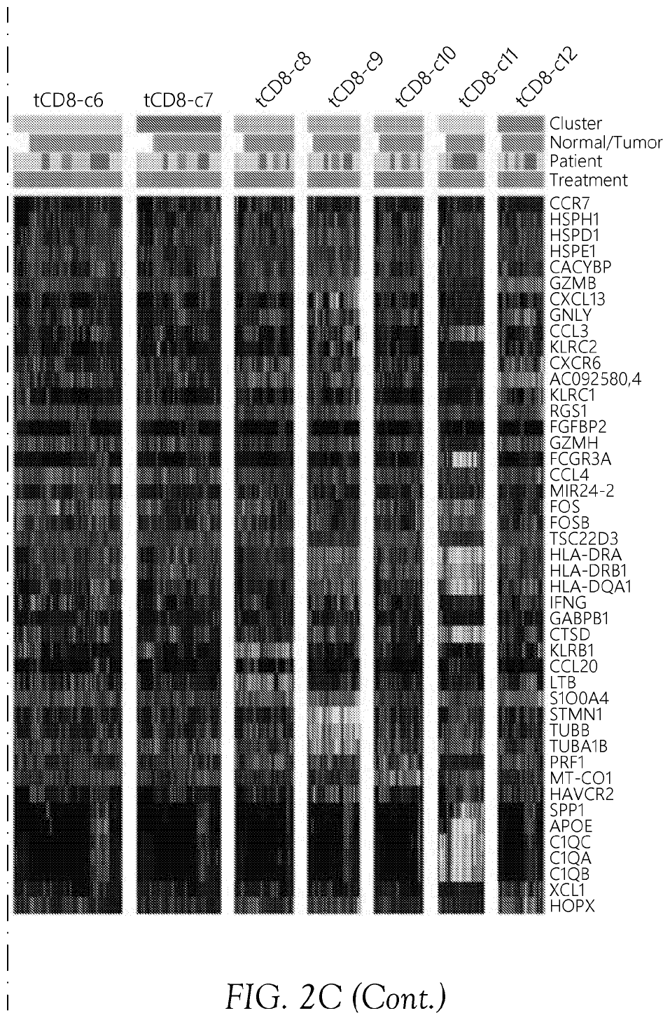

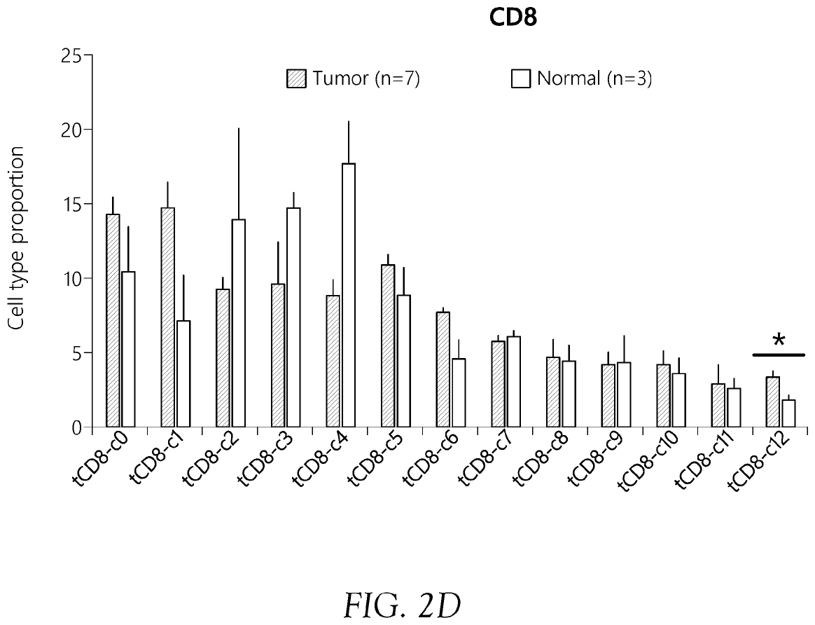

The 10× Genomics Chromium platform (Zheng et al., 2017b) was used to sequence 10,145 tumor- and 2,288 non-malignant-derived CD8⁻ T cells from 7 patients (Table 1). All samples were muscle-invasive bladder cancer (MIBC) from: 2 standard-of-care untreated patients (“untreated”), 1 chemotherapy-treated patient (gemcitabine +carboplatin, “chemo”), and 4 anti-PD-Ll-treated patients (“anti-PD-L1”) with detailed clinical annotations (Table 1). To assess the heterogeneity of T cells across samples while controlling for technical and biological artifacts, the analysis was restricted to highly variable genes and used canonical correlation analysis (CCA) to identify common sources of variation among samples and to project the data onto maximally correlated subspaces (Butler et al., 2018). Following CCA, the k-nearest neighbor graph on a 20-dimensional manifold of the data was calculated and used Louvain community detection (Blondel et al., 2008) to define clusters which were visualized using t-Stochastic Neighbor Embedding (tSNE) (van der Maaten and Hinton, 2008). Tumor- and non-malignant-derived CD8⁺ T cells form 13 clusters (denoted tCD8-c0 through -c12) that were populated by cells from each individual sample without noticeable patient-specific artifacts (FIG. 2A and FIG. 3).

TABLE 1
Characteristics of samples analyzed by scRNAseq for this study.

| | | | MIBC or | | Tumor at | Path | Path | # tumor | # normal | # tumor | # normal |
|---|---|---|---|---|---|---|---|---|---|---|---|
| Pt ID | Age | M/F | NMIBC | Neoadj tx | surgery? | T stage | N stage | CD4 | CD4 | CD8 | CD8 |
| Anti-PD-L1 A | 74 | M | MIBC | Atezo x 1 | Y (2.0 cm) | ypTa | ypN0 | 2396 | NA | 1301 | NA |
| Anti-PD-L1 B | 64 | M | MIBC | Atezo x 2 | Y (6.5 cm) | ypT4b | ypN2 | 2843 | 441 | 663 | NA |
| Anti-PD-L1 C | 68 | F | MIBC | Atezo x 2 | Y (6.8 cm) | ypT1 | ypN0 | 3694 | 672 | 1402 | NA |
| Anti-PD-L1 D | 71 | M | MIBC | Atezo x 2 | Y (1.5 cm) | ypT2b | ypN0 | 3721 | 347 | 1023 | NA |
| Chemo | 67 | F | MIBC | Chemo | Y (<0.1 cm) | ypTis | ypN0 | 1826 | 1075 | 1975 | 437 |
| Untreated A | 82 | M | MIBC | None | Y (3 cm) | pT3b | pN0 | 3378 | 135 | 1911 | NA |
| Untreated B | 76 | M | MIBC | None | Y (4 cm) | pT3b | pN0 | 1039 | 365 | 1425 | 1564 |
| Healthy | NA | NA | NA | NA | NA | NA | NA | NA | 13634 | NA | 7054 |
| | | | | | | | | | (blood) | | (blood) |

Each of 12 clusters were compared to a CCR7′ central memory population as reference (tCD8-c0) to focus on relative differences between clusters. This approach identified 724 genes that were differentially expressed in at least one cluster within the tumors (P_{adj} <0.05, llog2(FC)1>0.5). The identified populations include cells expressing HAVCR2 (TIM-3), LAG3, and ENTPD1 (tCD8-cl: log₂(FC) 0.85-1.1) previously described as tumor-reactive CD8⁺ T cells (Duhen et al., 2018); effector cells expressing FGFBP2 (tCD8-c3: log2(FC)=2.2) or activation markers such as AMC (tCD8-c5: log2(FC): 0.67-1.1) or CD69 and IFNG (tCD8-c6: 1og2(FC)=0.75-0.79); mucosal-associated invariant cells expressing KLRB1 (tCD8-c8: log₂(FC)=1.8) that frequently use the semi-invariant TCR alpha chains TRAV1-2/TRAJ33 in internal TCR data in agreement with published findings (Kurioka et al., 2016); and contaminating myeloid cells expressing CD 14/CD68/CST3 (tCD8-cl 1) (FIGS. 2B-2C). Similar populations were also identified in the tumor environment of hepatocellular carcinoma based on scRNA-seq (Zheng et al., 2017a). It was observed that the identified CD8⁺ populations did not display any significant differences in abundance between the tumor and non-malignant bladder (FIG. 2D).

### Example 2

### Regulatory CD4+ T Cells Include Heterogeneous Populations

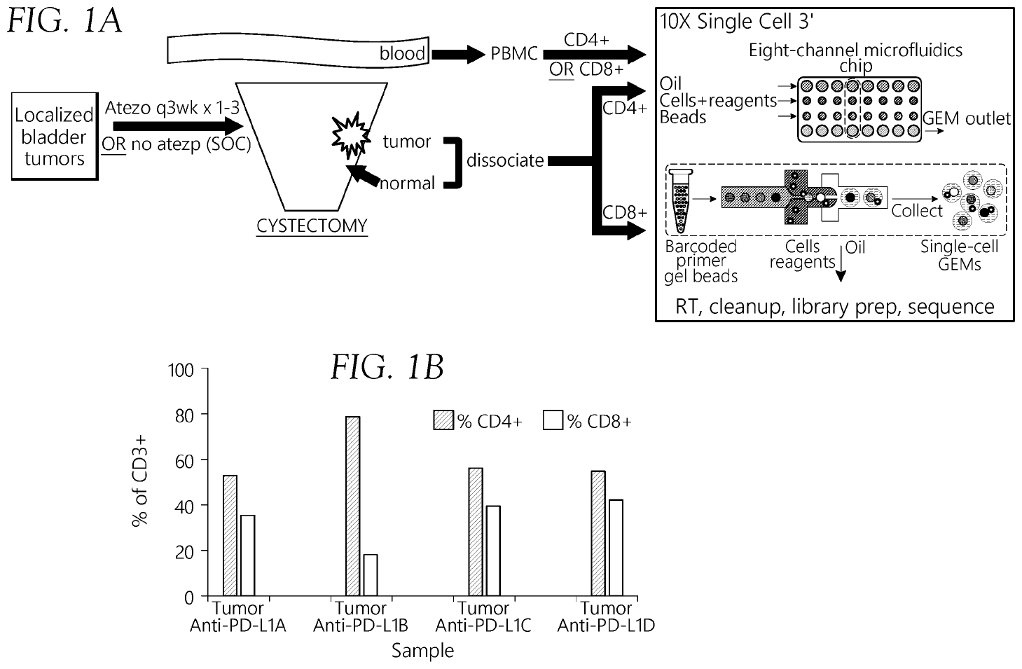

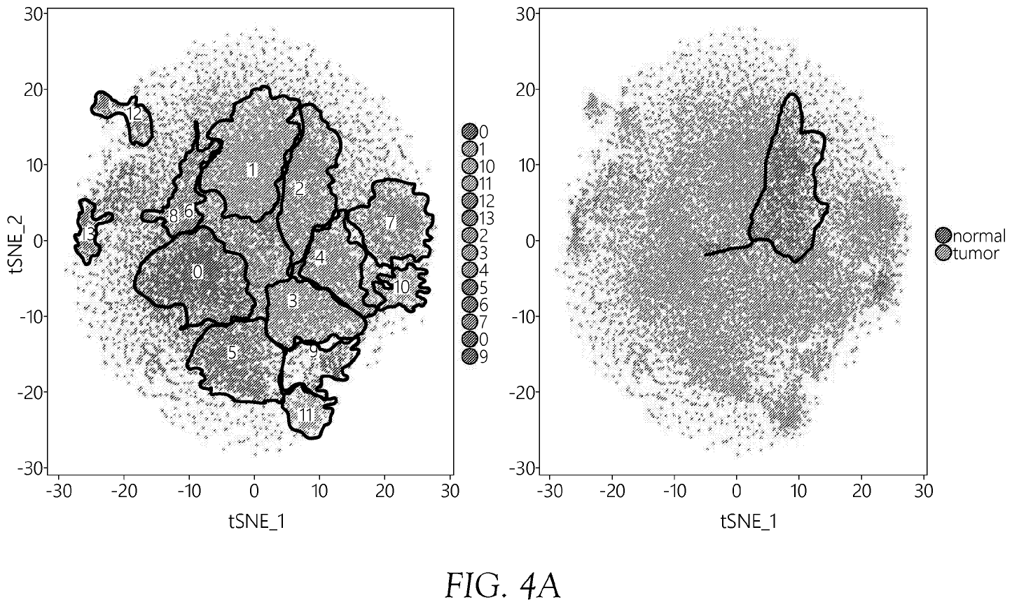

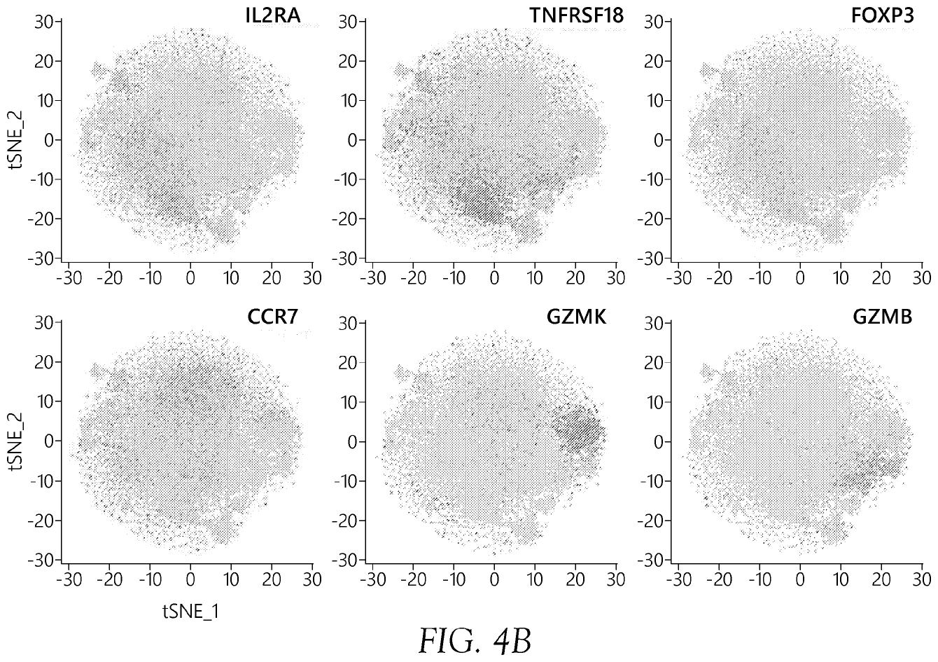

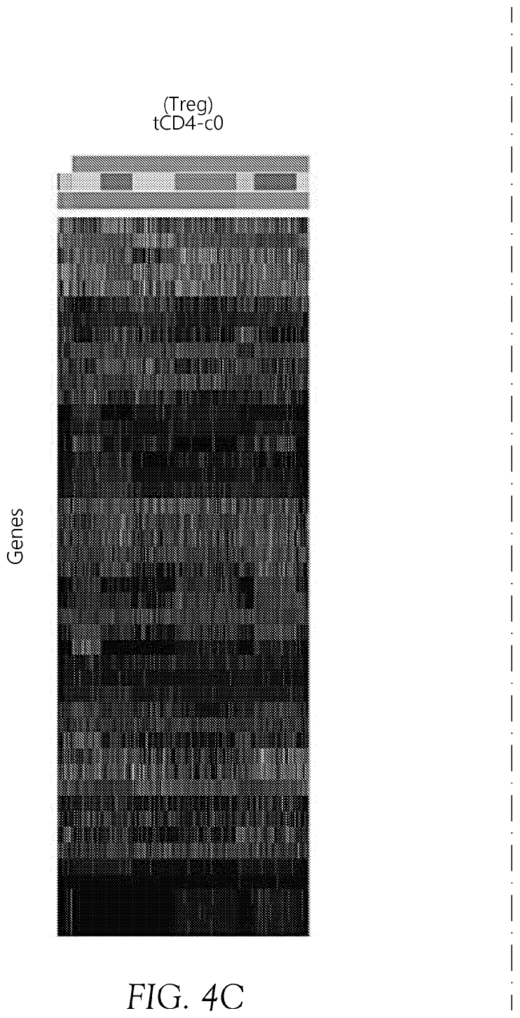

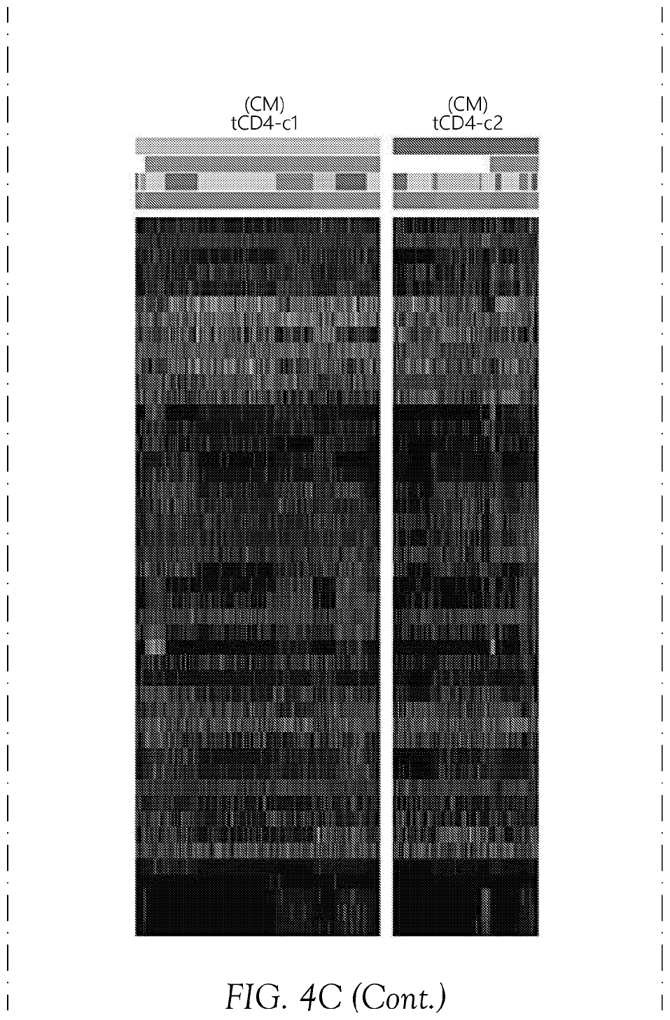

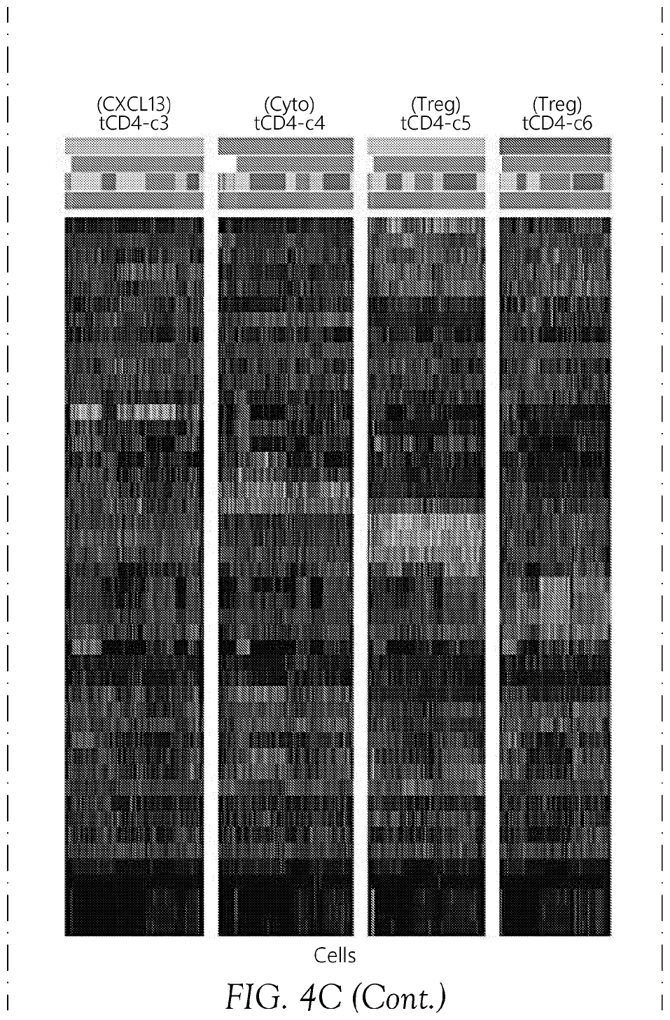

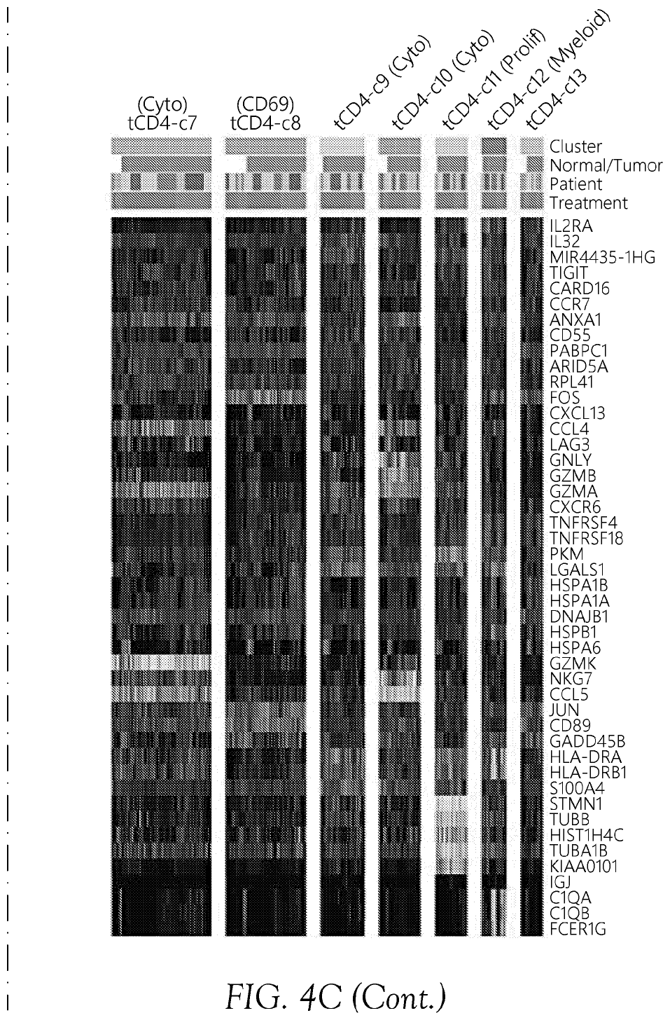

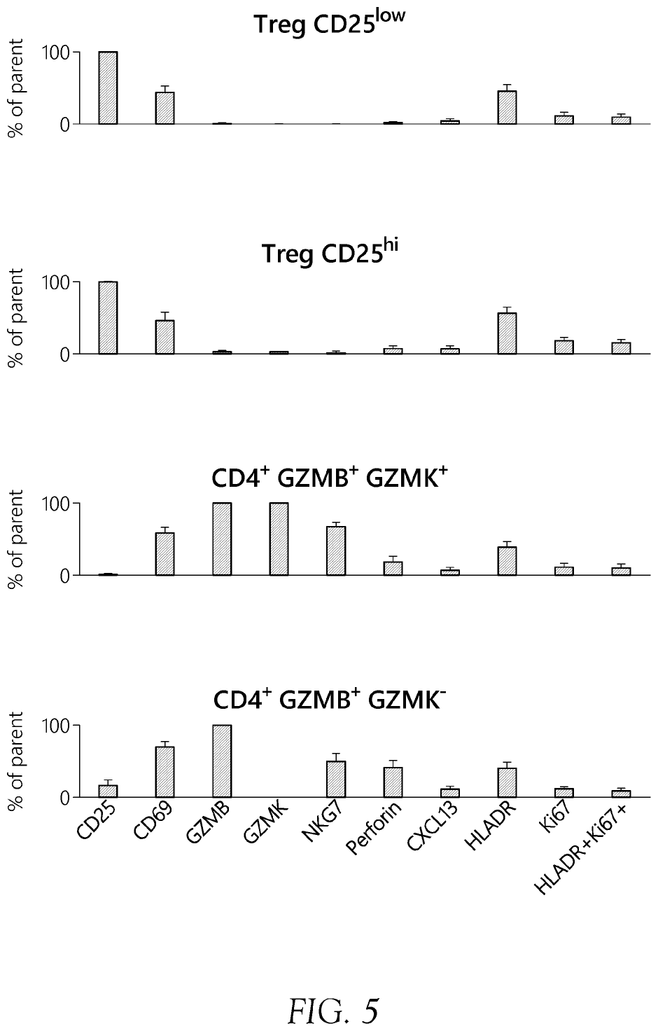

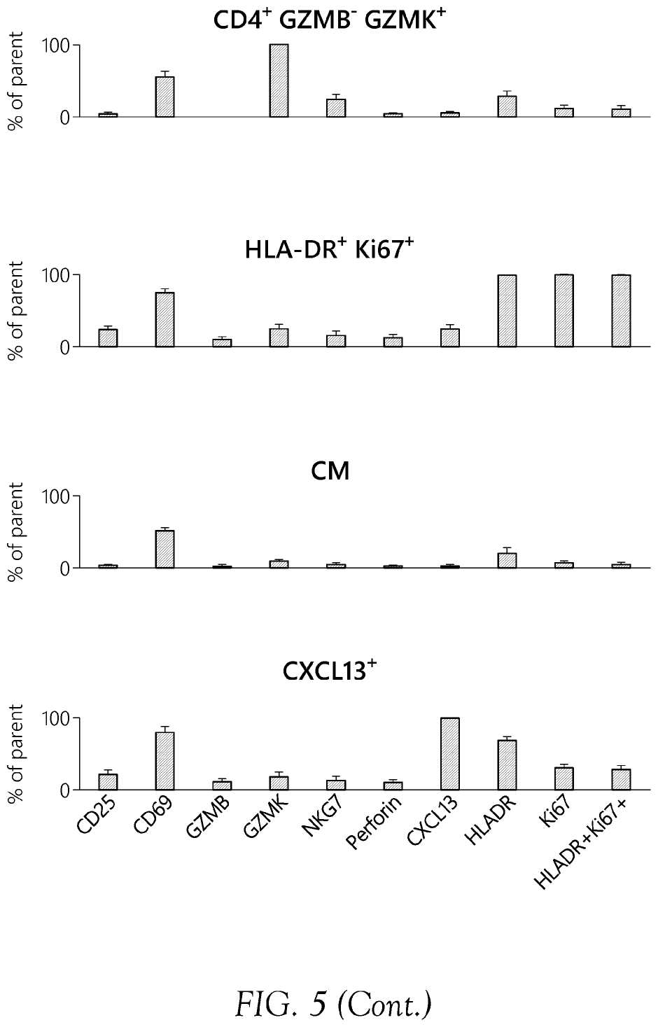

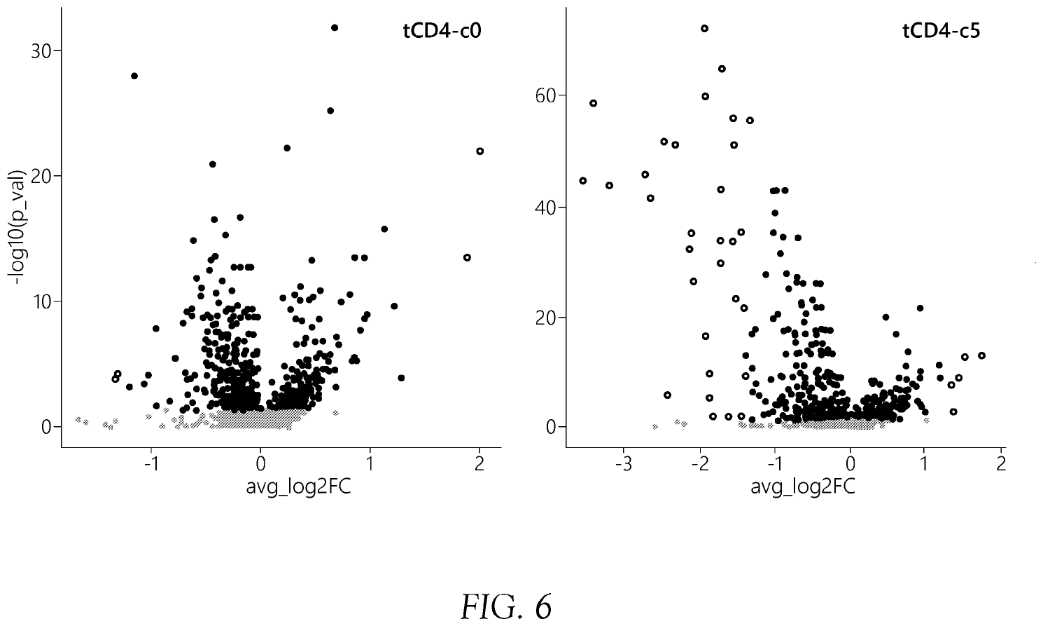

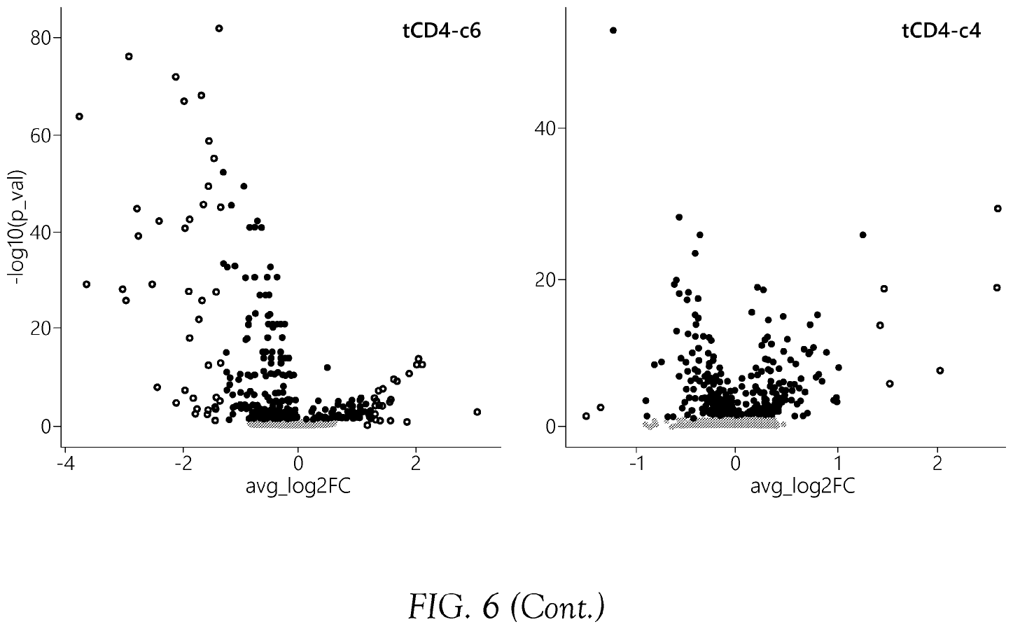

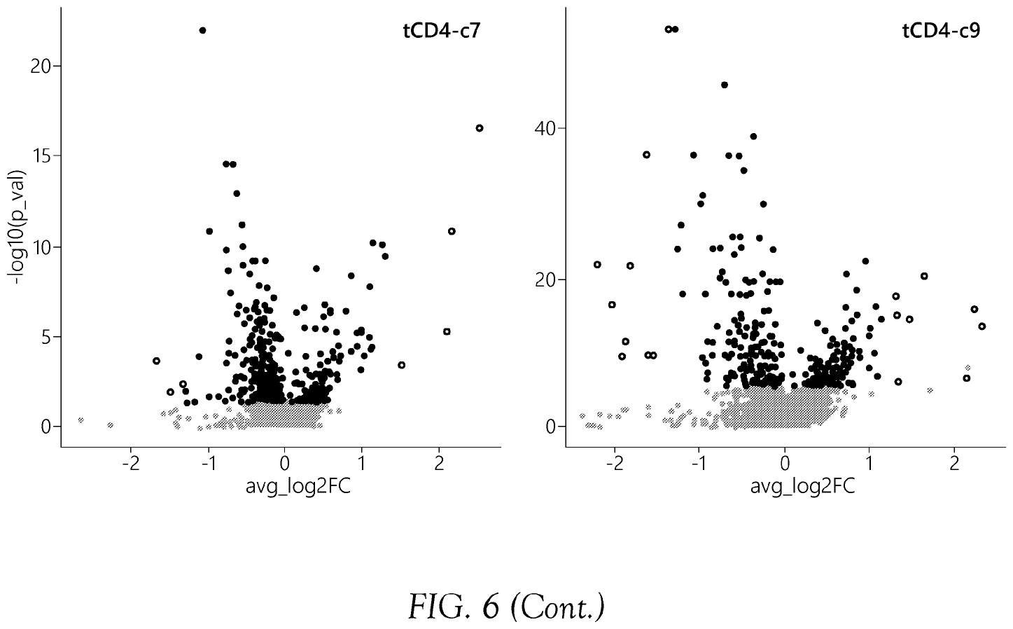

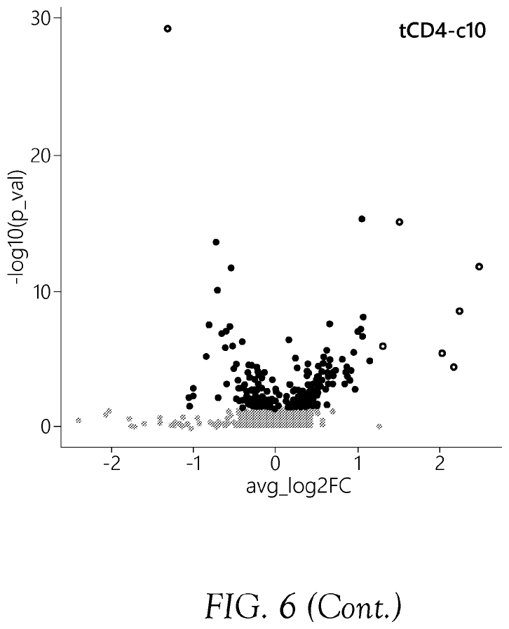

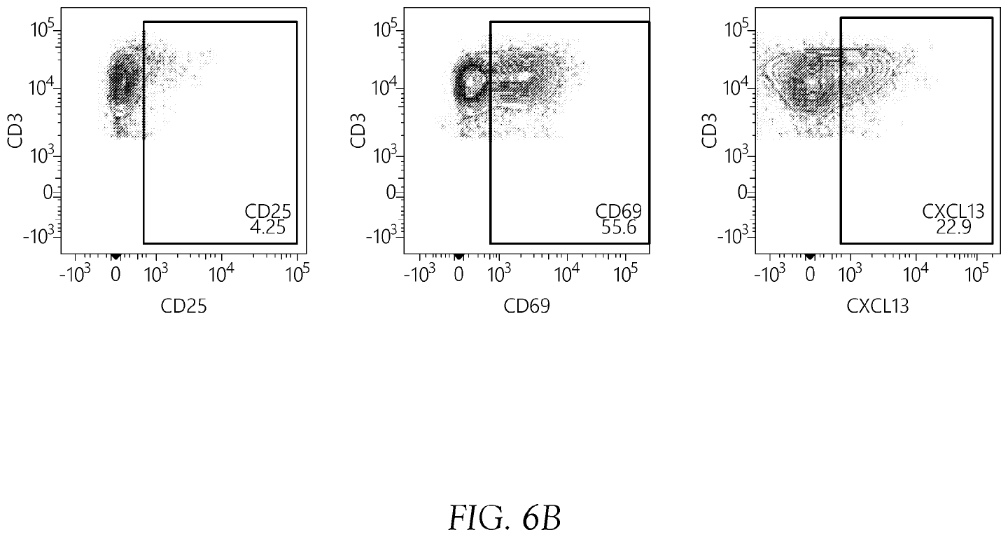

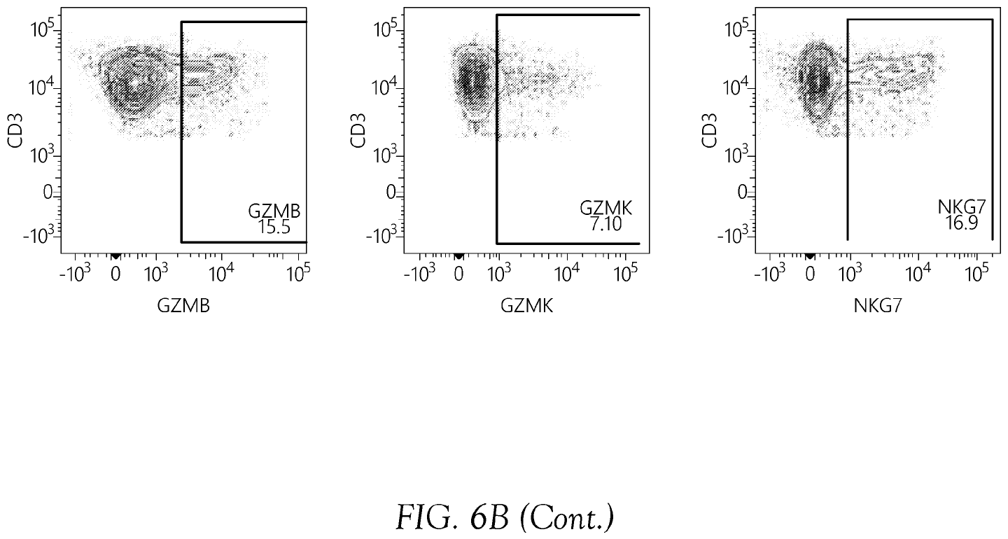

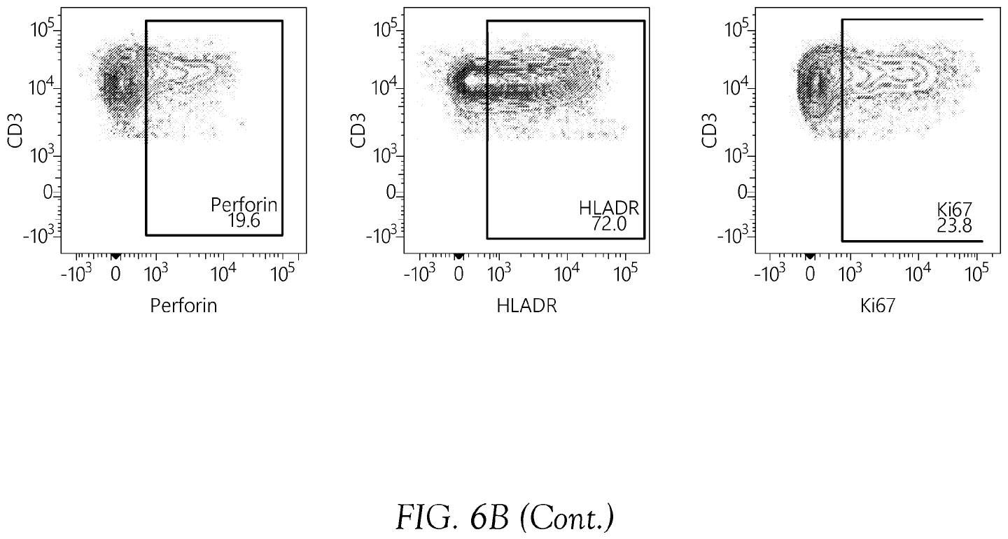

Given the lack of tumor enrichment of CD8⁺ populations and the higher frequency of CD4⁺ over CD8⁺ T cells in bladder tumors (FIG. 1B), subsequent experiments were performed to investigate CD4⁺ T cell heterogeneity in a similar fashion to determine their contribution to anti-tumor responses. In total, 18,979 tumor- and 3,263 non-malignant-infiltrating CD4⁺ T cells isolated from the same patients were sequenced and analyzed. Tumor- and non-malignant-derived CD4⁺ T cells form 14 clusters with representation from individual patients (FIG. 4A; FIG. 3). Because of their lack of expression of effector molecules (FIG. 5 gating strategy in FIG. 6), a CCR7′ central memory population was used as an internal control (tCD4-cl) and compared each of the remaining 13 clusters to tCD4-cl for differential expression. With this approach allowed for identification of 856 genes that were differentially expressed in at least one cluster (P_{adj}<0.05, llog2(FC)1>0.5, FIGS. 4B-4C). Several canonical populations of CD4⁺ T cells were identified, including an additional CCR7′ central memory population (tCD4-c2) and a population expressing high levels of CXCL13, LAG3 and IFNG (tCD4-c3: log2(FC)=2.8, 1.5, 0.92), whose presence has been associated with improved outcomes in breast and gastric cancer, and also is found in microsatellite-unstable colorectal carcinoma which is an immune-responsive tumor (Schmidt et al., 2018; Gu-Trantien et al., 2013; Gu-Trantien et al., 2017; Wei et al., 2018; Zhang et al., 2018). Other populations include an effector population expressing CD69 (tCD4-c8: log2(FC)=1.1) but not FOXP3 (log2(FC) <0.5); and a population of contaminating myeloid cells expressing CD 14, CD68, and CST3 (tCD4-c12) (FIG. 4C); as well as several important additional populations described in further detail below.

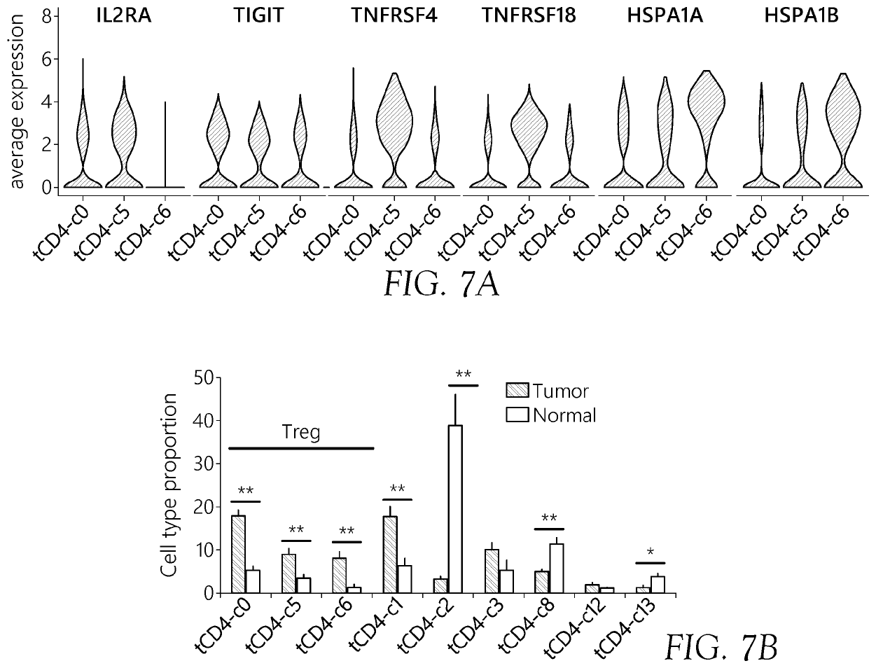

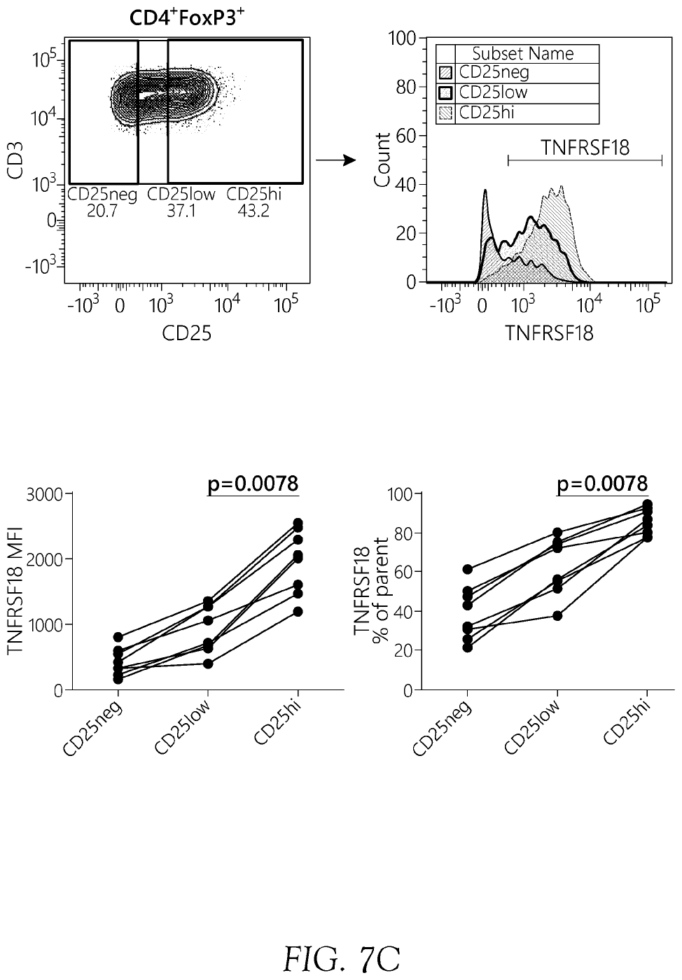

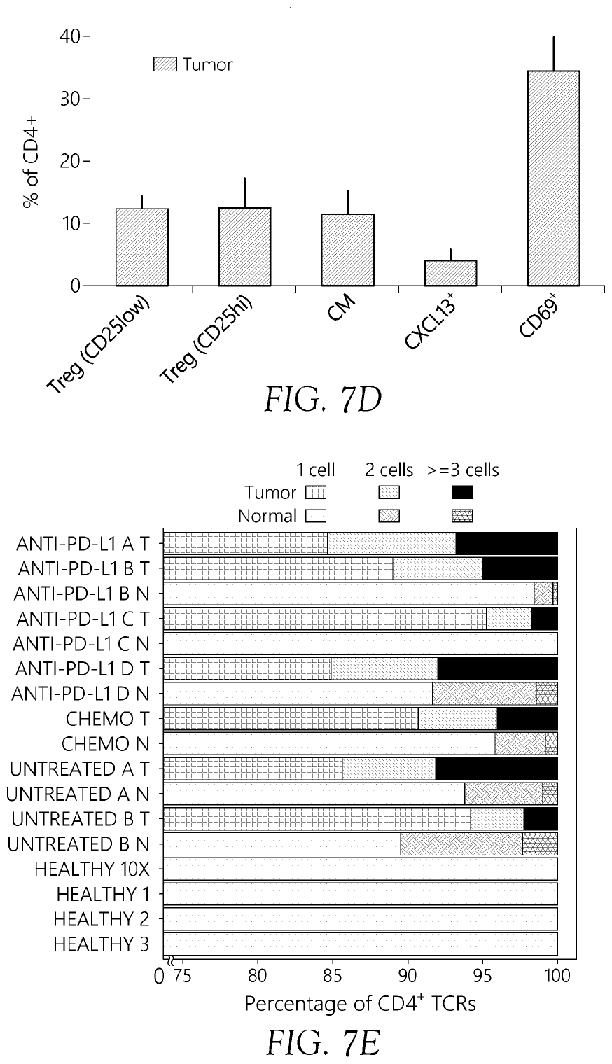

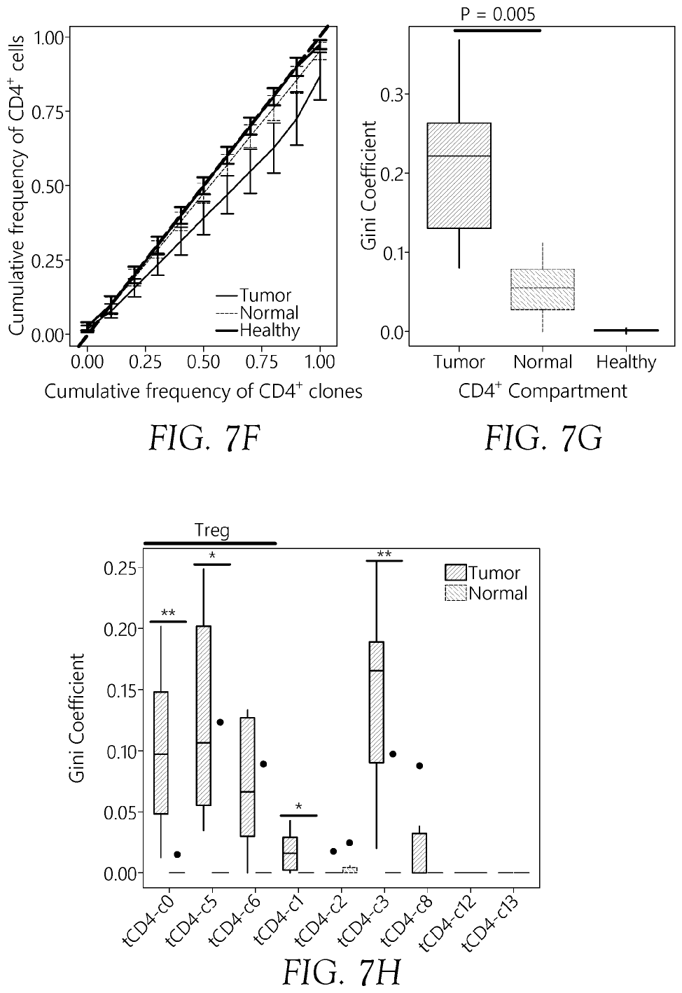

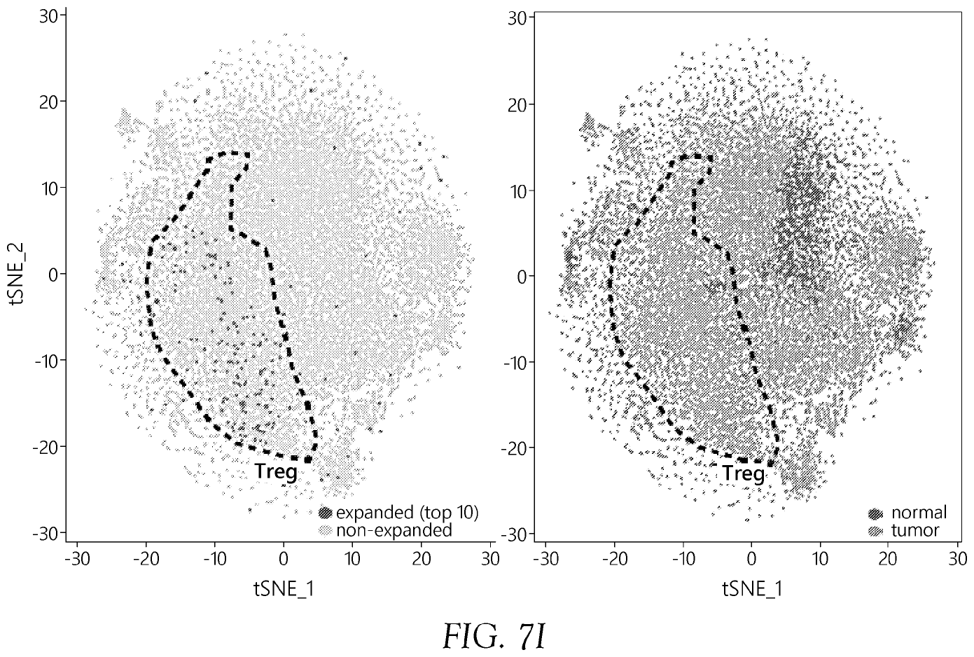

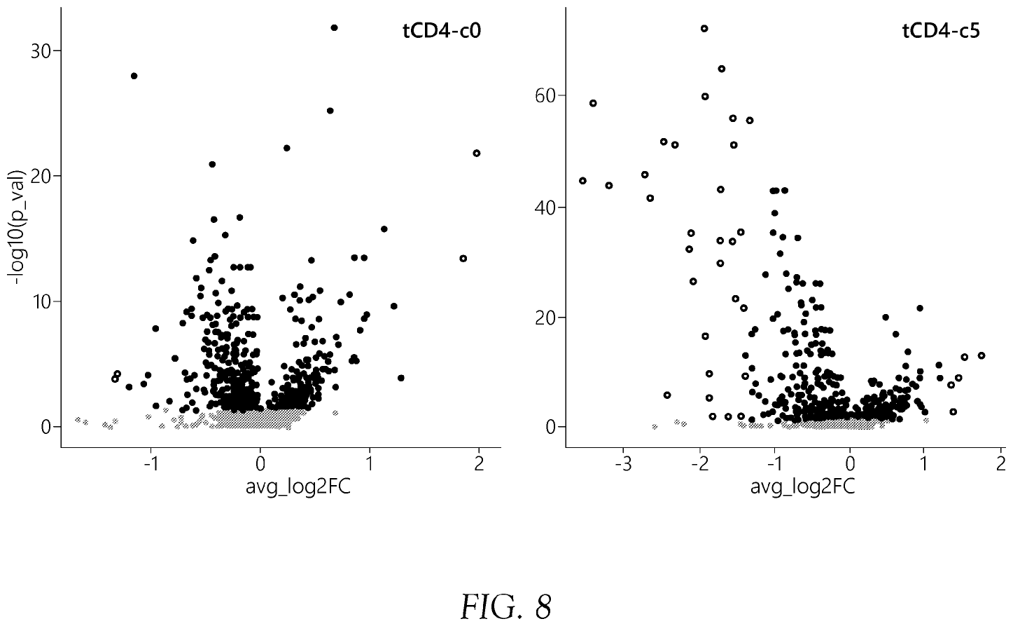

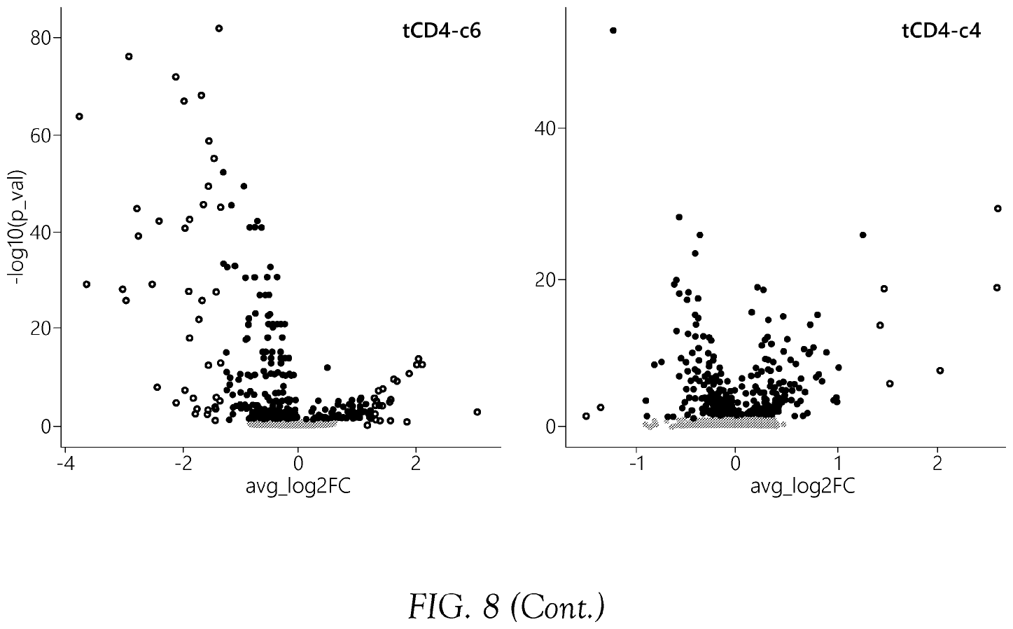

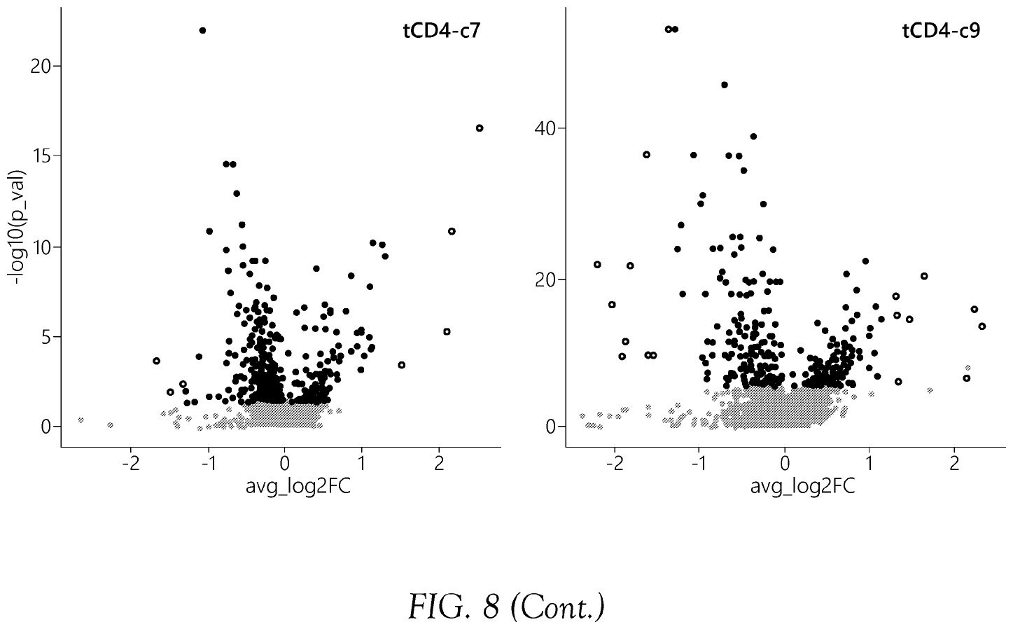

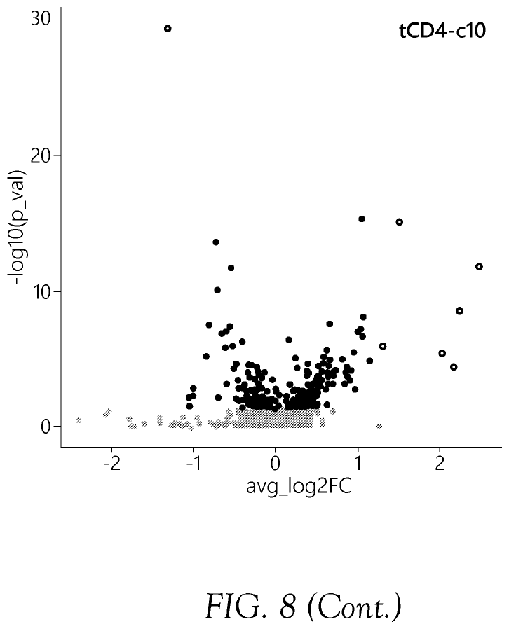

Regulatory CD4⁺ T cells are an abundant constituent of the bladder tumor microenvironment with demonstrated heterogeneity. In these experiments, it was observed that 3 states of regulatory T cells (tCD4-c0, tCD4-c5, tCD4-c6) together constituted 35 ±3.3% (mean ±s.e.m.) of tumor-infiltrating CD4⁺ cells, which expressed FOXP3 (tCD4-c0: log2(FC)=0.62; tCD4-c5: log2(FC)=0.73; tCD4-c6: log2(FC)=0.40) and known immune checkpoints (tCD4-c0 and tCD4-c5: log₂(FC) >0.82 for IL2RA, TIGIT, TNFRSF4/9/18, CD27; FIGS. 4B-4C, FIG. 7A). tCD4-c5 was distinguished from tCD4-c0 and tCD4-c6 based on higher expression of TNFRSF4/18 and LAGS, while tCD4-c6 is noted for higher expression of heat shock proteins (log2(FC) vs CCR7⁺ reference, P_{adj} <0.05, FIG. 7A). Notably, all 3 regulatory populations were significantly enriched in tumor compared to adjacent non-malignant tissue (tCD4-c0: 18.0 vs 5.3%, P=7.4 x 10⁻⁶; tCD4-c5: 9.1 vs 3.5%, P=0.0031; tCD4-c6: 8.2 vs 1.4%, P=0.0042; T-test, FDR <0.1, FIG. 7B). These experiments confirmed that multiple tumors contain distinct regulatory populations that expressed graded protein levels of IL2RA, and co-expressed significantly different levels of immune checkpoints such as TNFRSF 18, by flow cytometry from unrelated bladder tumors (n=7 tumors; P <0.05 for TNFRSF 18 expression in FOXP3⁺ CD25¹⁰′v versus CD25^{hi} populations by Wilcoxon signed-ranked test, FIGS. 7C-7D, gating strategy in FIG. 6). Regulatory T cells expressing higher levels of checkpoints such as TNFRSF9 have been shown to be correlated with poorer outcomes in non-small cell lung cancer (Guo et al., 2018). In these experiments, it was observed that all three regulatory populations showed tumor-specific expression of several heat shock proteins as compared with non-malignant tissue (FIG. 8). tCD4-c6 additionally demonstrated tumor-specific overexpression of immune-related transcripts such as 1L32, TNFRSF4, CD3D, and CCL5 (all genes with llog₂(FC) >0.5, P_{adj} <0.05, FIG. 8).

Differential expression analysis for CD8⁻' T cells between paired tumor/non-malignant compartments also revealed tumor-specific expression of heat shock genes, MHC class II alleles (e.g., HLA-DRA/-DRBI/-DPA1/-DPB1) and CD7 4 (Class II-associated invariant chain) which are likely reflective of activation by antigen (all genes with P_{A} <0.05, llog2(FC)1>0.5) (data not shown).

### Example 3

### Regulatory CD4⁺ T Cell Populations are Clonally Expanded in Bladder Tumors

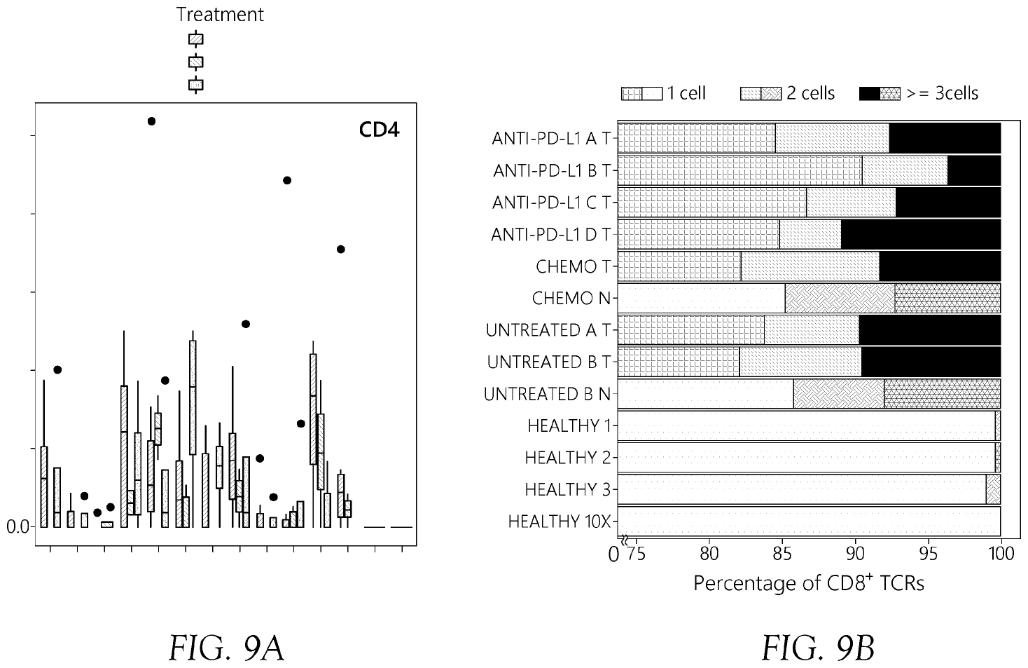

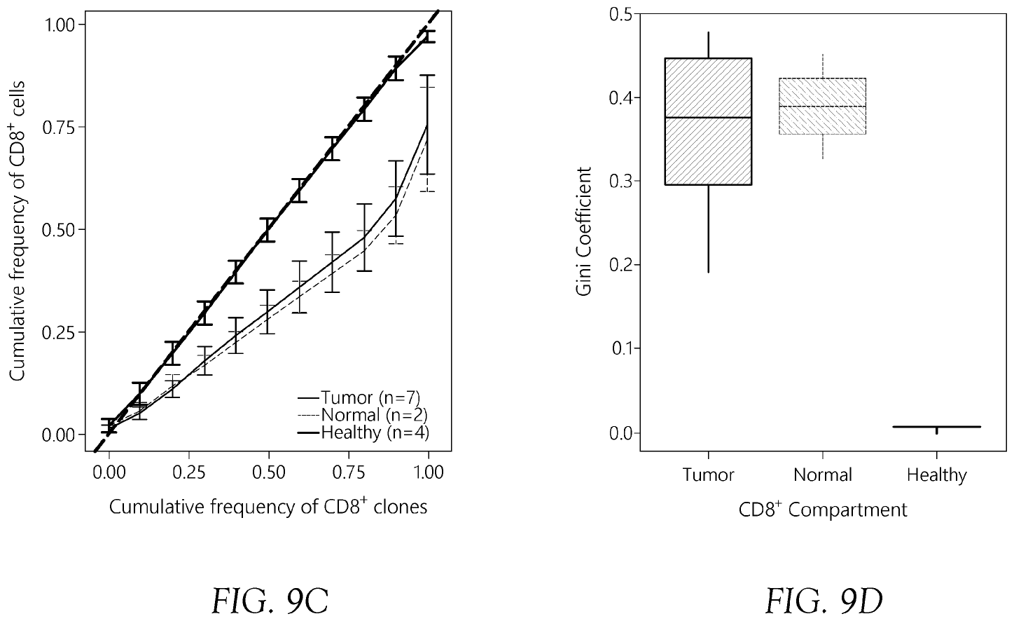

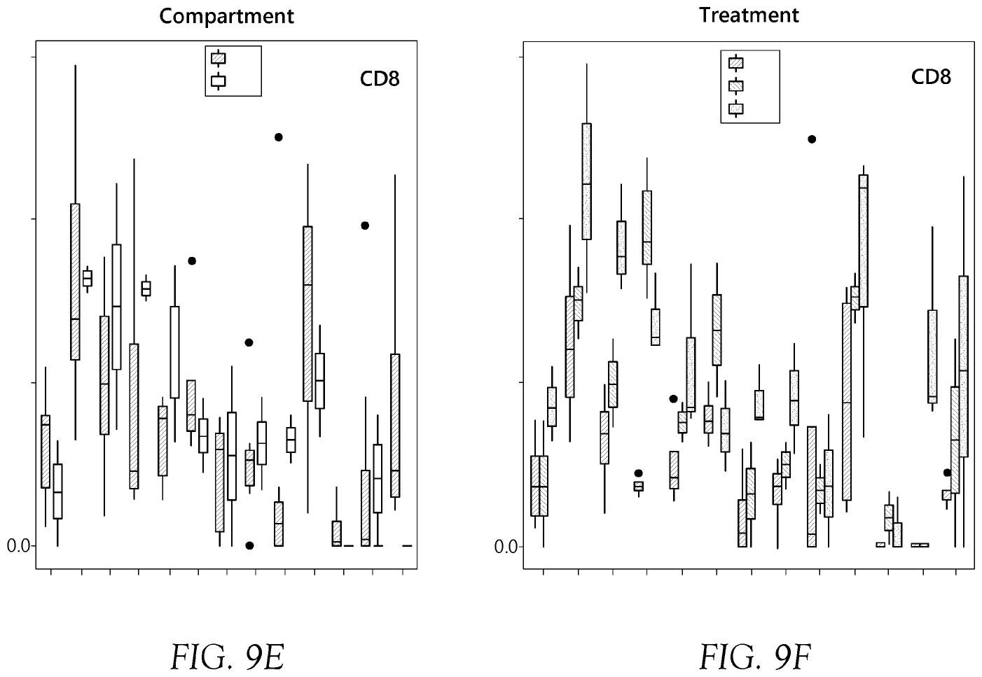

To query the TCR sequence in the same single cells for which whole-transcriptome data had been acquired previously, the complementarity-determining region 3 (CDR3) of the TCR alpha (TRA) and beta (TRB) loci from the barcoded full-length cDNA library were PCR-amplified and sequenced to saturation. After filtering, this approach yielded 11,081 CD4⁺ T cells (50% of cells with expression data) and 5,779 CD8⁺ T cells (46% of cells with expression data) with paired TRA and TRB CDR3 sequences. These results are consistent with expected frequencies based on the average recovery of individual TRA (CD4⁺ : 54%, CD8⁺ : 50%) and TRB (CD4⁺ : 68%, CD8⁺ : 67%) sequences across cells (data not shown). Overall, the TCR repertoire was found to be more restricted in the tumor microenvironment than adjacent non-malignant tissue based on two analyses. First, in intratumoral CD4⁺ T cells, 10.8 ±1.6% of unique clonotypes were shared by 2 or more cells; this degree of sharing was significantly greater than in the non-malignant compartment (5.1 ±1.6%; unpaired T-test, P=0.033), and was not seen in blood from healthy donors (0.12-0.16%) or from publicly available reference circulating CD4⁺ T cell data (0%) (FIG. 7E). Second, there was a skewing of the intratumoral CD4⁺ T cell repertoire towards an increased cumulative frequency of clonotypes over fewer cells (FIG. 7F) and a corresponding higher Gini coefficient (0.21 for tumor vs 0.05 for non-malignant, unpaired T-test, P=0.005, FIG. 7G) as compared to the non-malignant compartment and healthy controls. Assigning cells with expanded TCR sequences to their respective functional clusters revealed that regulatory CD4⁺ T cell clonal expansion contributes to intratumoral CD4⁺ T cell repertoire restriction. Compared to paired non-malignant tissue, two of the three regulatory populations exhibited increased Gini coefficients in tumor (tCD4-c0: Giniₜᵤₘₒᵣ 0.10 vs Giniₙₒᵣₘₐₗ 0.003, P <0.01, tCD4-c5: Giniₜᵤₘₒᵣ 0.13 vs Giniₙₒᵣₘₐₗ 0.02, P <0.05, Wilcoxon signed-rank test FDR <0.1, FIG. 7H). The most expanded clonotypes within the regulatory populations are private, being largely expressed only by regulatory cells and not other cell states (all single cells expressing top 10 expanded regulatory clonotypes shown in FIG. 71). The CXCL/3-expressing population tCD4-c3 (discussed in greater detail below) also was restricted in tumor (Giniₜᵤₘₒᵣ 0.14 vs Giniₙₒᵣₘₐₗ 0.02, P <0.01). Gini coefficients for CD4⁺ subpopulations did not differ significantly by anti-PD-L1 treatment (FIG. 9A). By contrast, although repertoire restriction was also seen in CD8⁺ T cells from the same samples, this was observed in both tumor (% unique clonotypes shared between cells: 15.1 ±1.1%; Giniₜᵤₘₒᵣ: 0.36 ±0.04) and non-malignant compartments (% unique clonotypes shared between cells: 14.6 ±0.2%; Giniₙₒᵣₘₐₗ: 0.39 ±0.06; FIGS. 9B-9D). Furthermore, no significant increase in Gini coefficient in tumor over non-malignant tissue was seen for any CD8⁺ subpopulation, including with anti-PD-L1 treatment (FIGS. 9E-9F). Hence, without being bound to any particular theory, an important contributor to increased repertoire restriction of tumor-infiltrating CD4⁺ over non-malignant tissue, which was not seen in the CD8⁺ compartment, appears to involve clonal expansion of several distinct regulatory T cell populations that differ in their levels of immune checkpoint expression, which may be driven by tumor-associated antigens and the tumor-specific microenvironment.

### Example 4

### Bladder Tumors Possess Multiple Cytotoxic CD4+ T Cell Populations

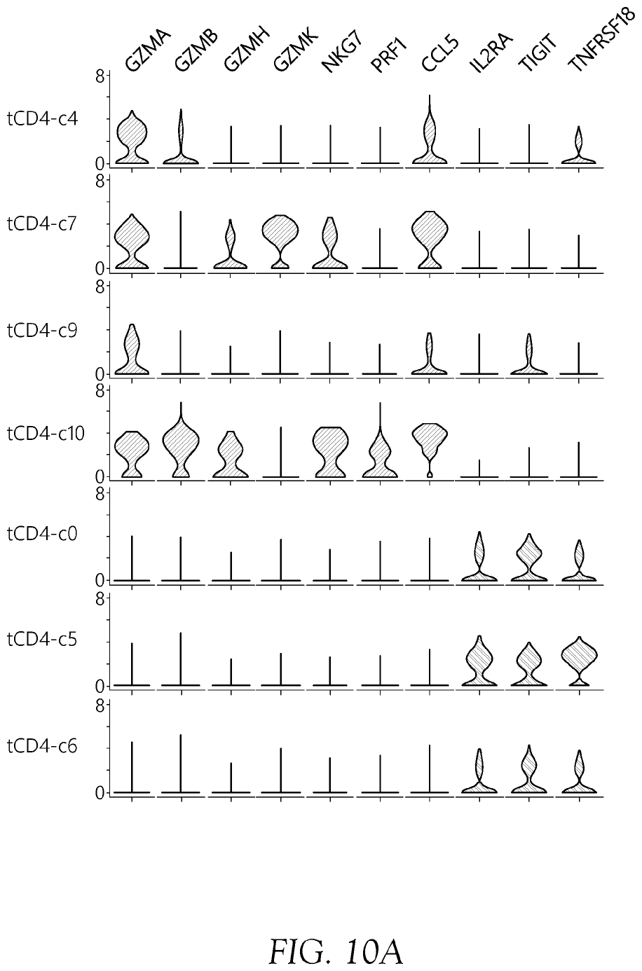

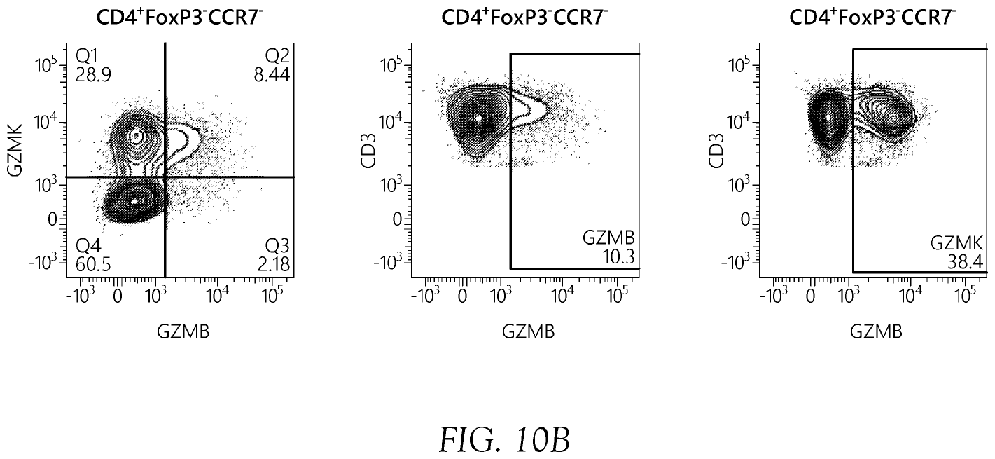

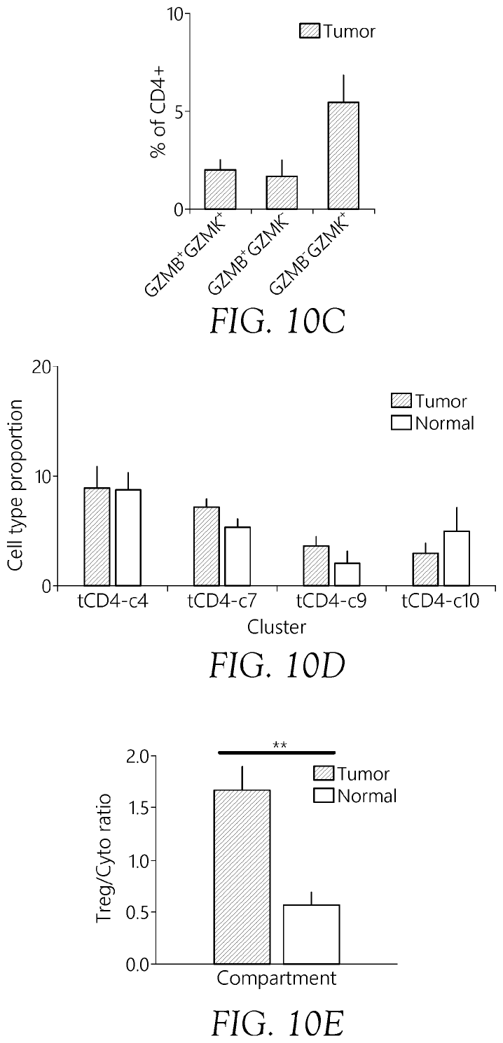

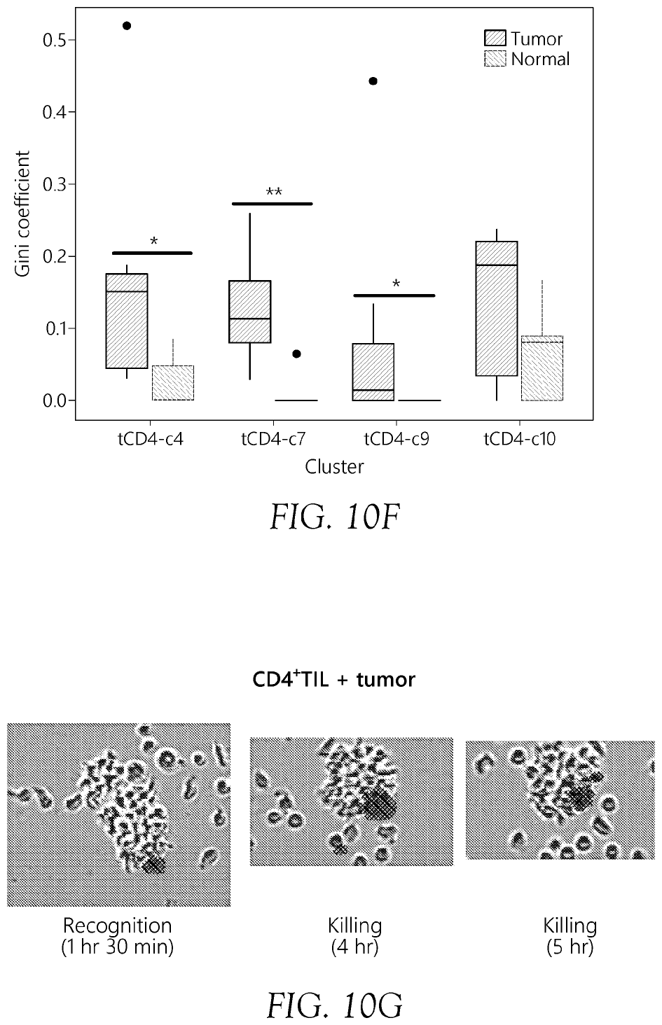

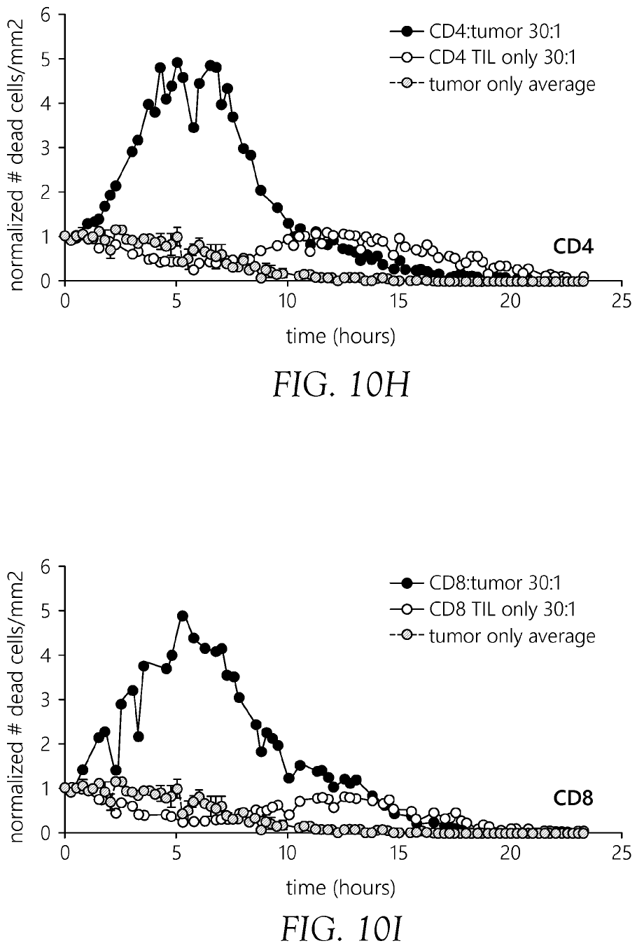

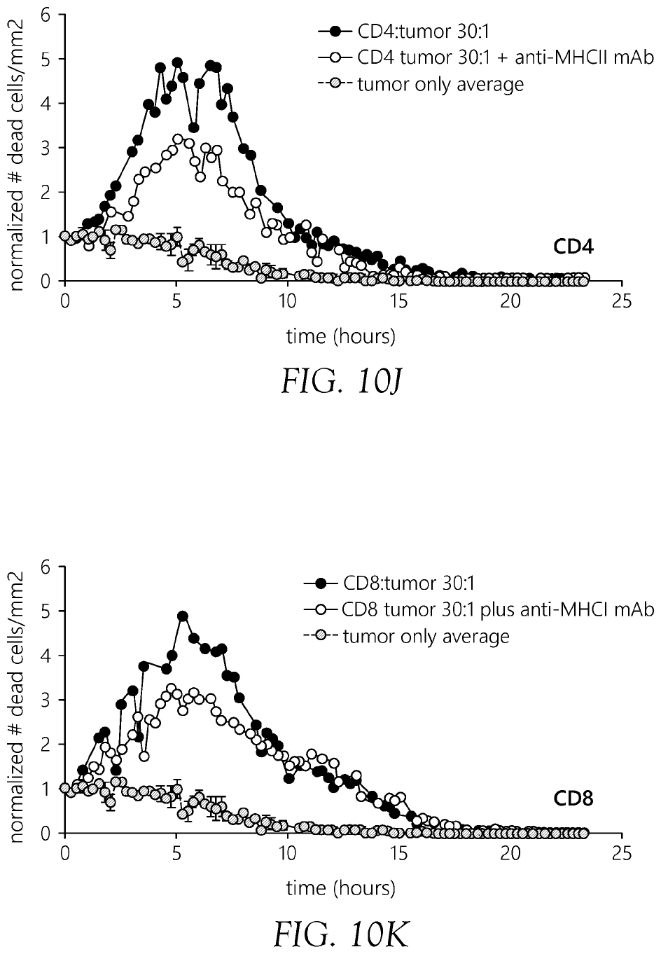

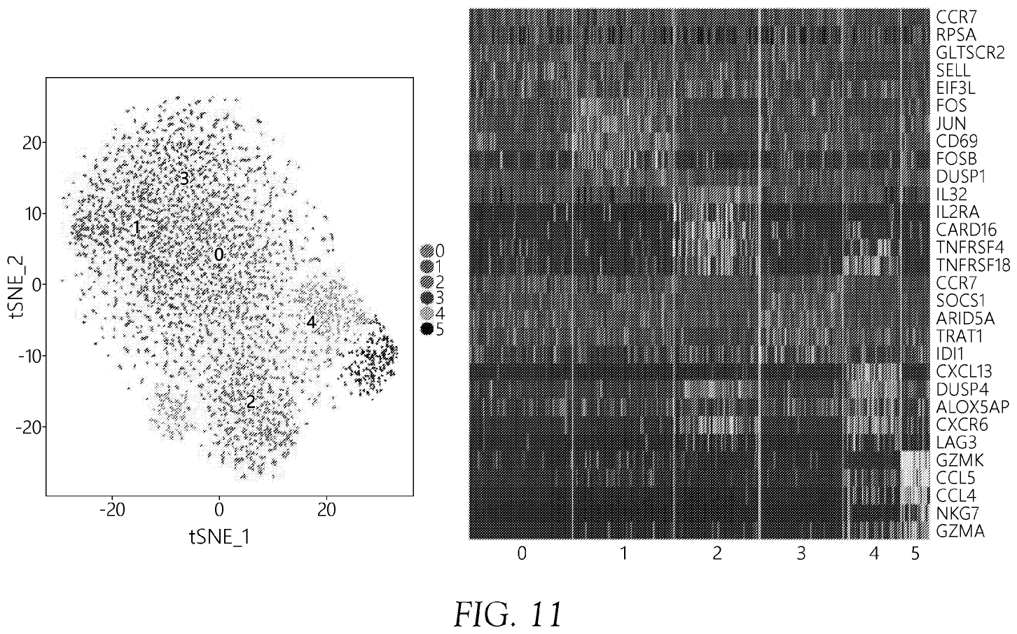

In addition to the regulatory populations described in Example 3 above, the results of additional experiments identified four (4) distinct populations of cytotoxic CD4⁺ T cells in all samples, which constituted 23 ±2.3% of tumor-infiltrating CD4⁺ T cells. Compared with the CCR7⁺ reference population, these populations all expressed (log₂(FC) >0.5, P_{adj} <0.05) a core set of cytolytic effector molecules: GZMA and GZMB and the granule-associated GNLY which is a pore-forming protein known to function in pathogen killing (Krensky and Clayberger, 2009) (FIGS. 4B-4C, FIG. 10A). Individual cytotoxic populations expressed higher levels of specific cytolytic molecules: GZMB (tCD4-c4: log2(FC)=1.7, tCD4-c10: log2(FC)=2.9), GZAIK (tCD4-c7: log₂(FC)=2.4), GNLY (tCD4-c4 and c9: log₂(FC)=1.5, tCD4-c10: log₂(FC)=3.3), and NKG7 (a granule protein that translocates to the surface of NK cells following target cell recognition suggesting a cytolytic role [Medley et al., 1996]) (tCD4-c7: log₂(FC)=2.2, tCD4-c10: log2(FC)=3.0). Specific cytotoxic populations also co-expressed additional genes which may further contribute to anti-tumor effector function: GZMH and PRF1 (perforin) in tCD4-c7 and tCD4-c10, IFNG in tCD4-c3/tCD4-c7/tCD4-c10 populations, and CXCR6 in tCD4-c4. The latter has been reported to be expressed in both regulatory and non-regulatory CD4 TILs from colorectal carcinoma, nasopharyngeal carcinoma, and renal cell carcinoma, and together with its ligand CXCL16, can mediate TIL chemotaxis (Löfroos et al., 2017; Parsonage et al., 2012; Oldham et al, 2012) (FIGS. 4B-4C, FIG. 10A). Overall the highest levels of GZMB, NKG7, and PRF1 were found in tCD4-c10, while tCD4-c7 demonstrated highest levels of GZMK with intermediate levels of GZMB/PRF1. Some cytotoxic CD4⁺ populations expressed the immune checkpoint LAG3 (tCD4-c7, -9, and -10: log2(FC)=0.95-1.1), as well as IL2RA (tCD4-c9: log₂FC=0.57) and HAVCR2 (TIM3) (tCD4-c7 and -c10: log₂(FC)=0.63-0,67) and TNFRSF18 (GITR) (tCD4-c4: log2(FC)=0.52), however other checkpoints associated with regulatory T cells were not expressed at high levels (FIG. 10A). Similar populations were found with unbiased clustering without CCA alignment for paired tumor- and non-malignant-derived CD4⁺ cells from individual patients (FIG. 11.

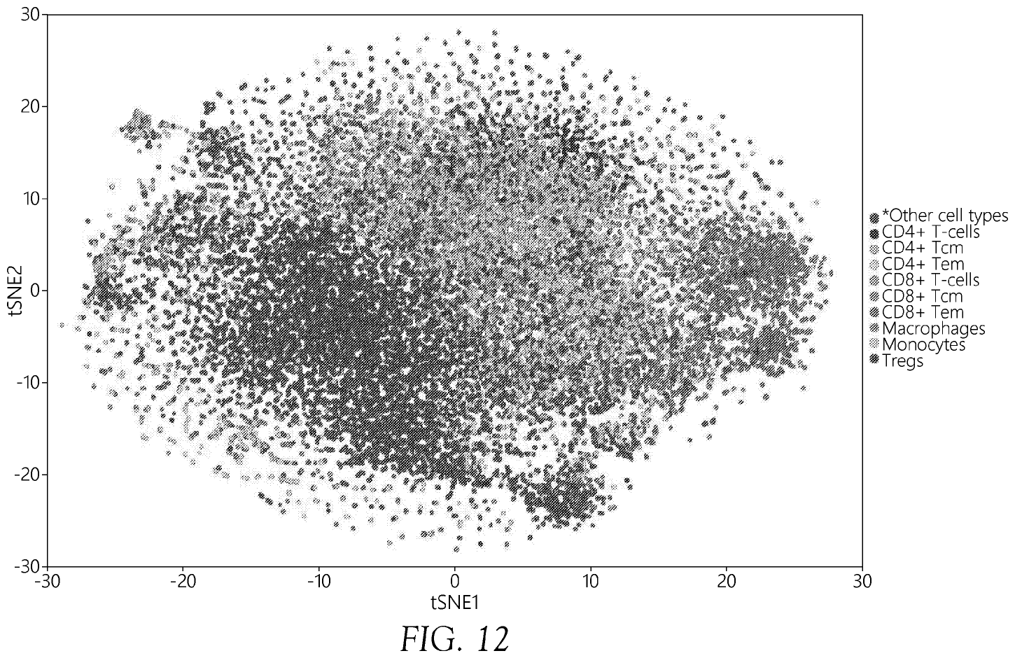

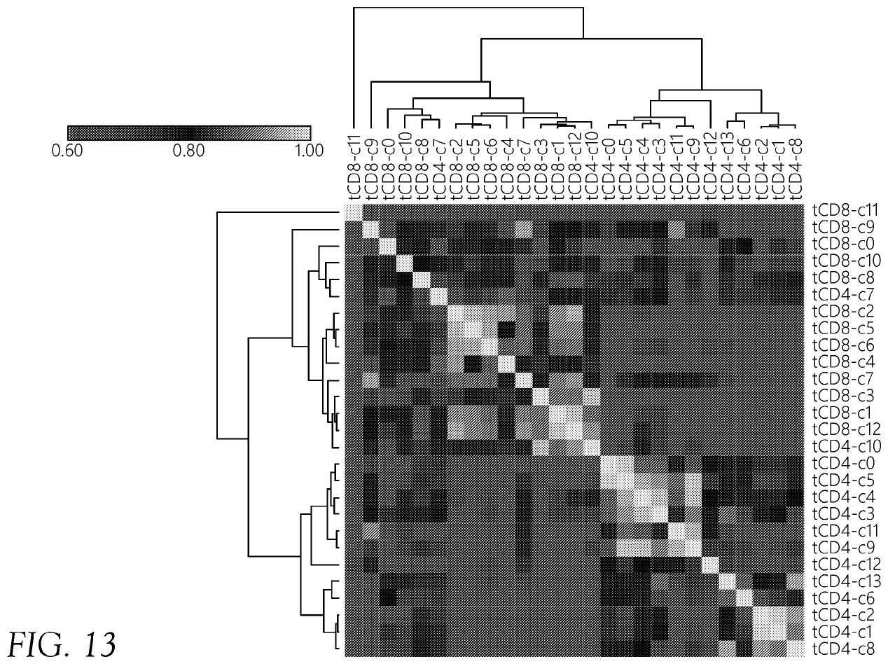

The presence and heterogeneity of cytotoxic CD4⁺ T cells were subsequently validated by flow cytometry and by comparisons to bulk and single-cell cytotoxic CD8+ expression profiles. First, the presence of cytotoxic CD4⁺ T cells with an effector memory (CCR7⁻ CD45RA⁻) or effector (CCR7⁻ CD45RA⁺ ) phenotype that express GZMB, GZMK, or both at the protein level was confirmed by flow cytometry in tumors from multiple patients separate from internal scRNAseq data set (n=7 tumors, FIGS. 10B-10C). Heterogeneity of cytotoxic CD4⁺ marked by variable expression of cytolytic genes in the scRNAseq data was also confirmed at the protein level by flow cytometry, as NKG7 expression was highest in GZMB+ cytotoxic CD4⁺ , PRF1 expression was most pronounced in GZMB⁺ GZMK⁻ CD4⁺ (as with tCD4-c10), and cytotoxic CD4⁺ expressed low levels of CD25 which was more strongly associated with regulatory T cells (FIG. 5). Importantly, it was found that CD45⁻ bladder tumor cells expressed multiple MHC II molecules (data not shown), which would allow for antigen recognition by TCRs expressing CD4 as a co-receptor. Second, overall annotation of clusters from the scRNAseq data is supported by an independent analysis that assigns each single cell to the best-known published immune subset profiled by bulk expression analysis after sorting (SingleR) (Aran et al., 2019). This corroborates the identification of regulatory T cells and demonstrates that multiple cytotoxic CD4⁺ populations are most similar to CD8⁺ central or effector memory T cells, reinforcing their cytotoxicity profile (FIG. 12). Finally, an internal comparison of all CD4⁺ and CD8⁺ TIL clusters from internal scRNAseq data indicates that while the correlation is generally higher amongst clusters in the CD4⁺ and CD8⁺ compartments, cytotoxic CD4⁺ T cells are an exception. The tCD4-c7 cytotoxic cells were most correlated with tCD8-c4 (R=0.86) and the tCD4-c10 cytotoxic cells were most correlated with tCD8-c3 (R=0.94) (FIG. 13) The tumor-specific gene expression program of these cytotoxic CD4⁺ populations were marked by heat shock protein expression, as well as overexpression of CXCL13 in tumor-infiltrating CD4⁺ from several populations (tCD4-c4, tCD4-c7, tCD4-c10, FIG. 8).

### Example 5

### Cytotoxic CD4+ T Cell Populations are Clonally Expanded in Bladder Tumors

It was observed that cytotoxic CD4⁺ populations were not significantly enriched in abundance in tumor (FIG. 10D). Overall the CD4⁺ compartment exhibited a bias towards regulatory over cytotoxic CD4⁺ T cells in tumor (regulatory CD4⁺ /cytotoxic CD4⁺ ratio=1.7 ±0.23), and towards cytotoxic CD4⁺ in non-malignant tissues (regulatory CDricytotoxic CD4⁺ ratio=0.6 ±0.13, P=0.002 by T-test, FIG. 10E) However, the cytotoxic CD4⁻ populations contribute to intratumoral CD4⁺ repertoire restriction. Several cytotoxic CD4⁺ populations have significantly increased Gini coefficients in tumor compared to non-malignant tissues, with tCD4-c4 representing the most restricted cytotoxic population in tumor (tCD4-c4: Gini ₜᵤₘₒᵣ 0.16 vs Giniₙₒᵣₘₐₗ 0.03; tCD4-c7: Giniₜᵤₘₒᵣ 0.13 vs Giniₜᵤₘₒᵣ0.01; tCD4-c9: Giniₜᵤₘₒᵣ 0.09 vs Giniₙₒᵣₘₐₗ 0; P <0.05, Wilcoxon test FDR <0.1, FIG. 10F). Hence, unbiased dscRNAseq revealed that heterogeneous cytotoxic CD4⁺ , a subset of which are closely related to conventional cytotoxic CD8⁺ based on their functional program, are an unexpected but frequent constituent of the bladder tumor microenvironment. The tumor-specific clonal expansion of several cytotoxic CD4⁺ populations suggests that although these populations may not be quantitatively enriched from recruitment into tumor, their restricted repertoire may result from recognition of cognate bladder tumor-associated antigens.

### Example 6

### Cytotoxic CD4+ T Cells can Lyse Autologous Tumor Cells

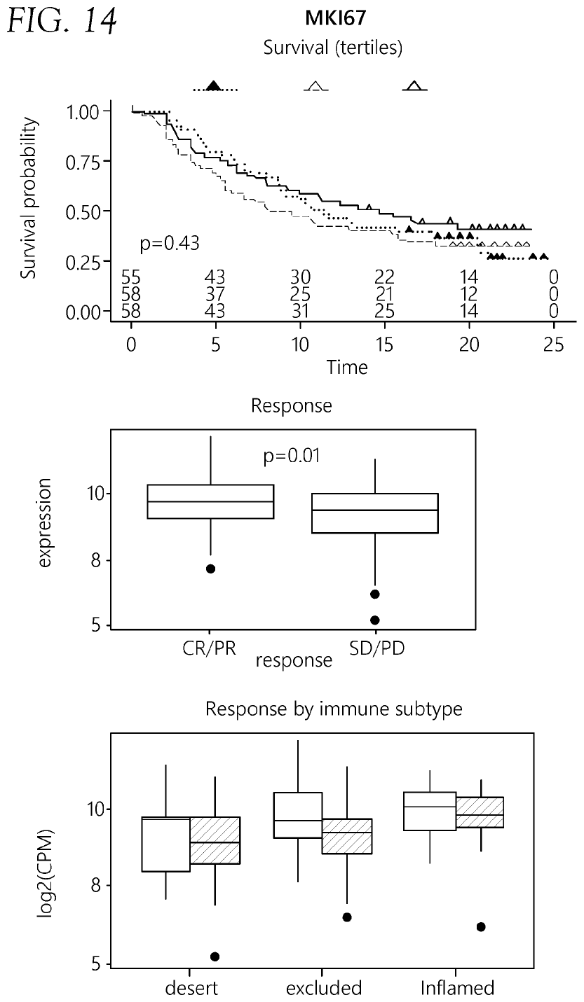

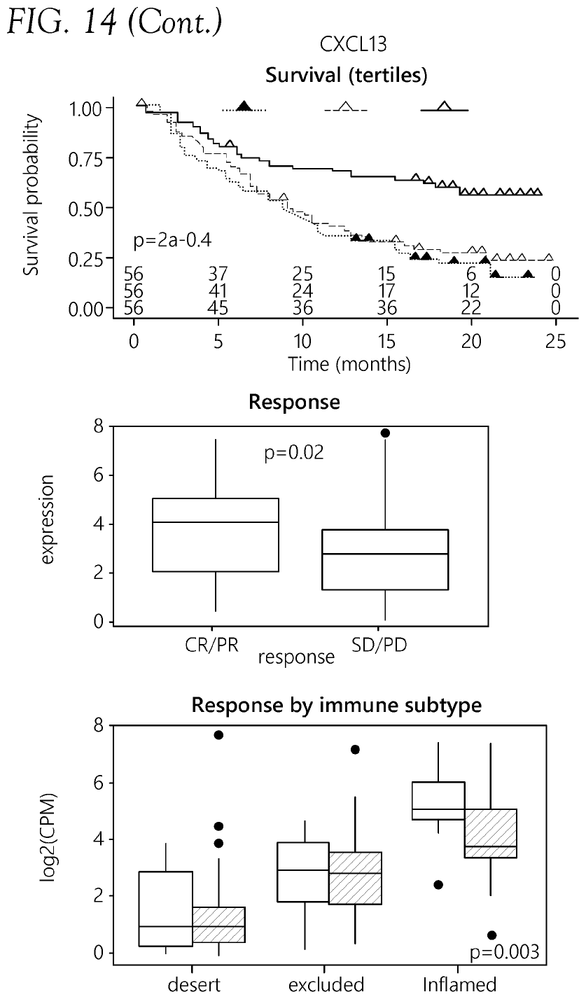

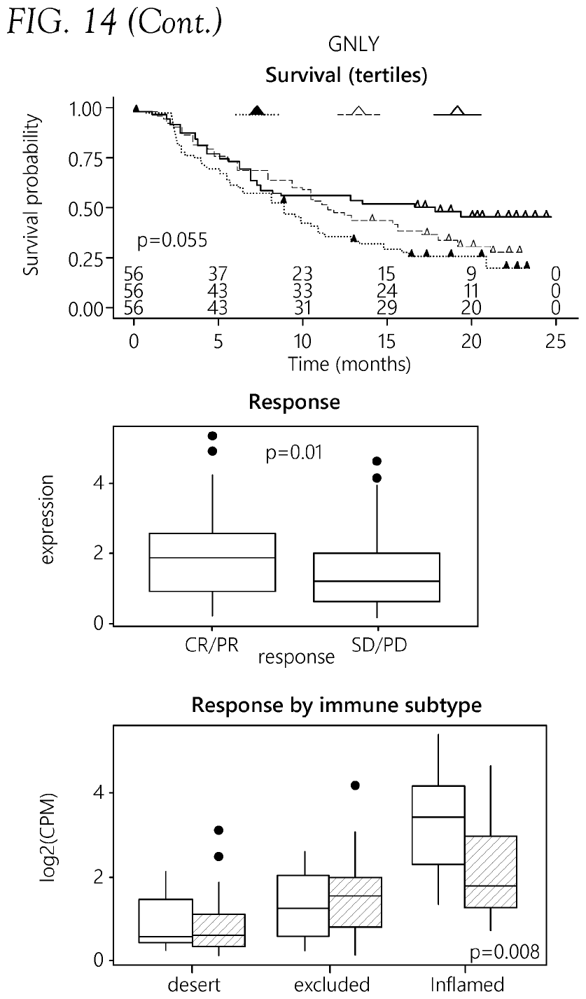

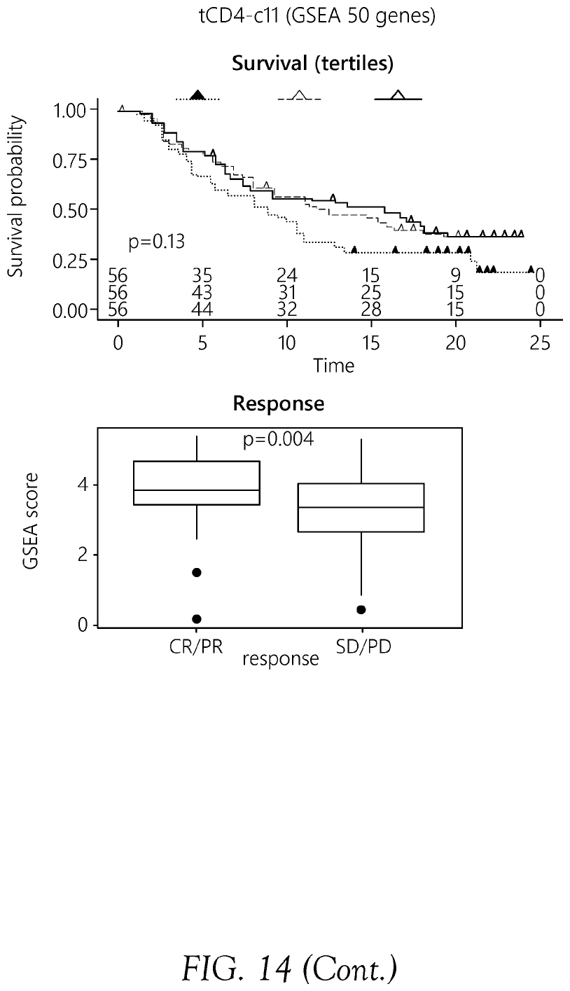

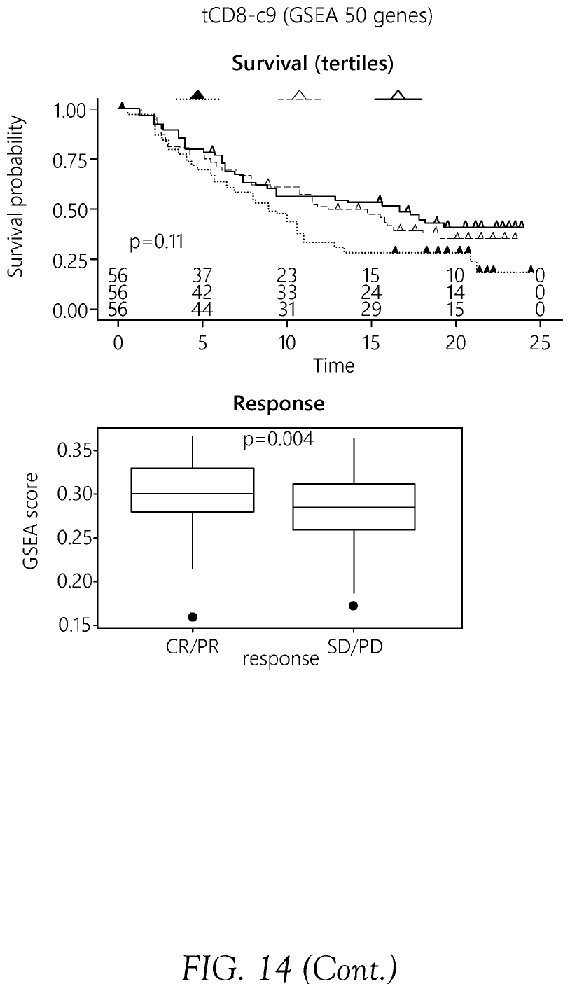

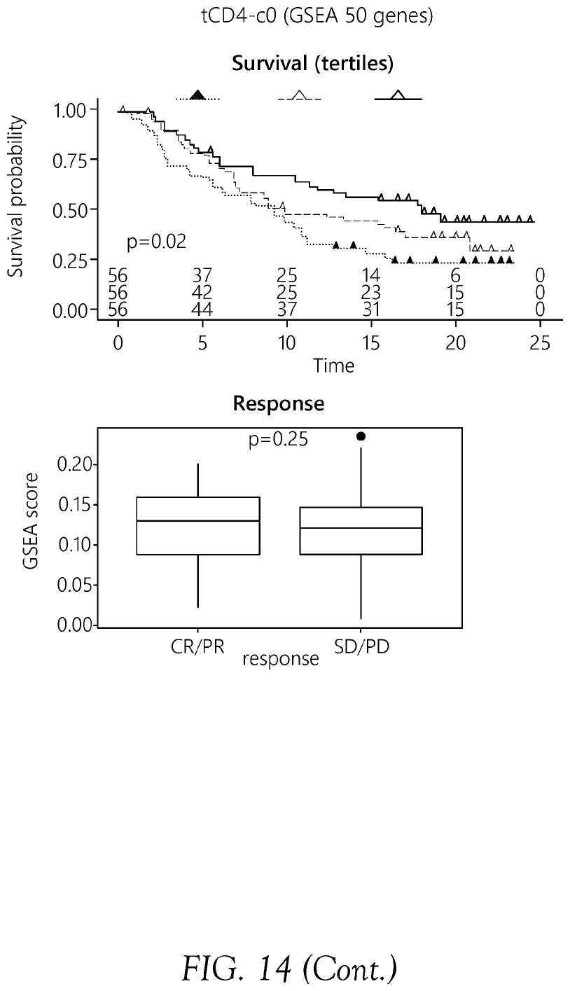

To validate the functional relevance of cytotoxic CD4⁺ in bladder tumors, CD4⁺ TILs depleted of regulatory T cells were isolated by FACS, and then cultured the remaining cells ex vivo with IL-2. These cells were then co-cultured with autologous tumor cells in an imaging-based time-lapse cytotoxicity assay. CD4⁺ TILs formed clusters around tumor cells within 1-2 hours of co-culture (indicative of tumor recognition) followed by killing of tumor cells (as measured by an increase in number of cells staining with a red fluorescent cell death indicator) within 4-5 hours (FIG. 10G). An increase in tumor cell death was seen at 5 hours indicative of rapid killing by CD4⁺ TIL (an 4.9x increase in death from baseline was observed for 30:1 ratio of TIL to tumor; FIG. 1011), which was not seen when analysis is restricted to surrounding TILs only from the same wells, or in tumor cells cultured alone in separate wells (TIL only at 30:1: 0.42x, tumor only: 0.99x). The kinetics and extent of autologous CD4⁺ T cell killing were similar to CD8⁺ T cell killing (CD8⁻ at 30:1 TIL to tumor ratio: 4.9x at 5.25 hrs; FIG. 31). It was observed that CD4⁺ killing was dose dependent across various effector:target ratios (30:1 ratio: 4.9x, 15:1 ratio: 3.9x at 5 hrs; FIG. 14) and was also partially blocked by pre-incubation of tumor cells with a pan-MHCII antibody (30:1 ratio: 3.2x at 5 hrs, 15:1 ratio: 1.95x at 6 hrs; FIG. 10J, FIG. 14). CD8⁺ autologous killing was also similarly blocked in part by MHCI blockade (30:1 ratio: 3.1x at 5 hrs; FIG. 10K). Hence, flow cytometry and functional analyses confirmed not only that cytotoxic CD4⁺ T cells express cytolytic proteins such as granzymes, but that these cells could recognize bladder tumor antigens in an MHC II-dependent fashion and were functionally competent to lyse tumor cells under conditions where co-existing regulatory T cells were excluded.

### Example 7

### Anti-PD-L1 Therapy Shifts T Cell Proliferation Towards Cytotoxic CD4+ T Cells

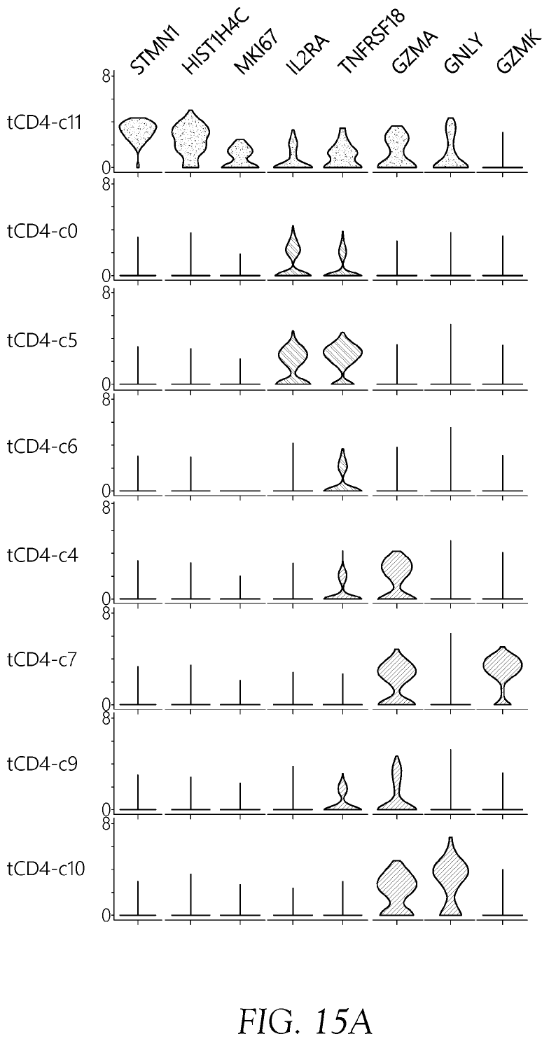

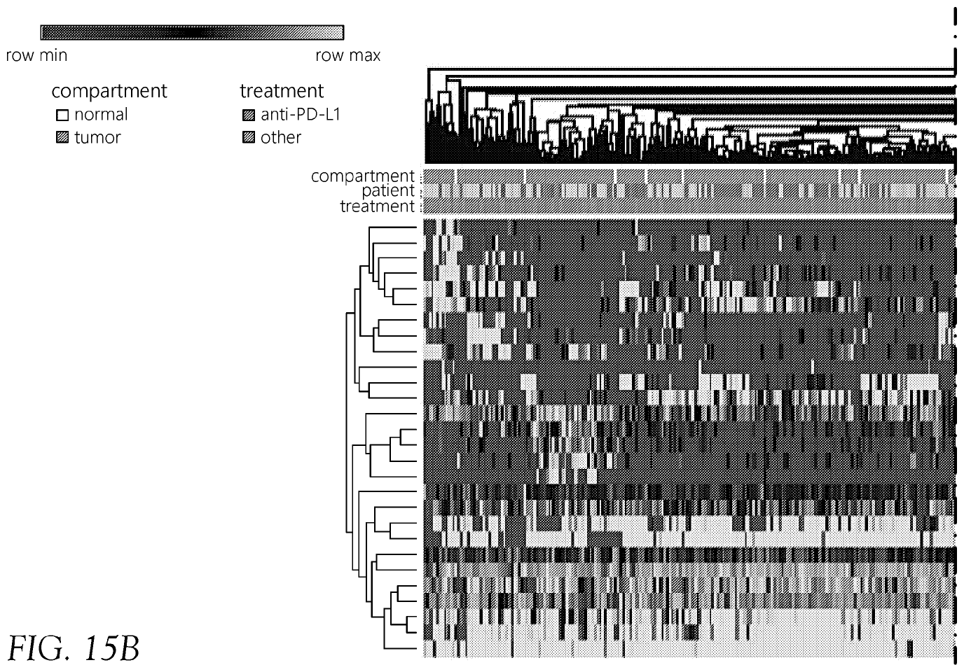

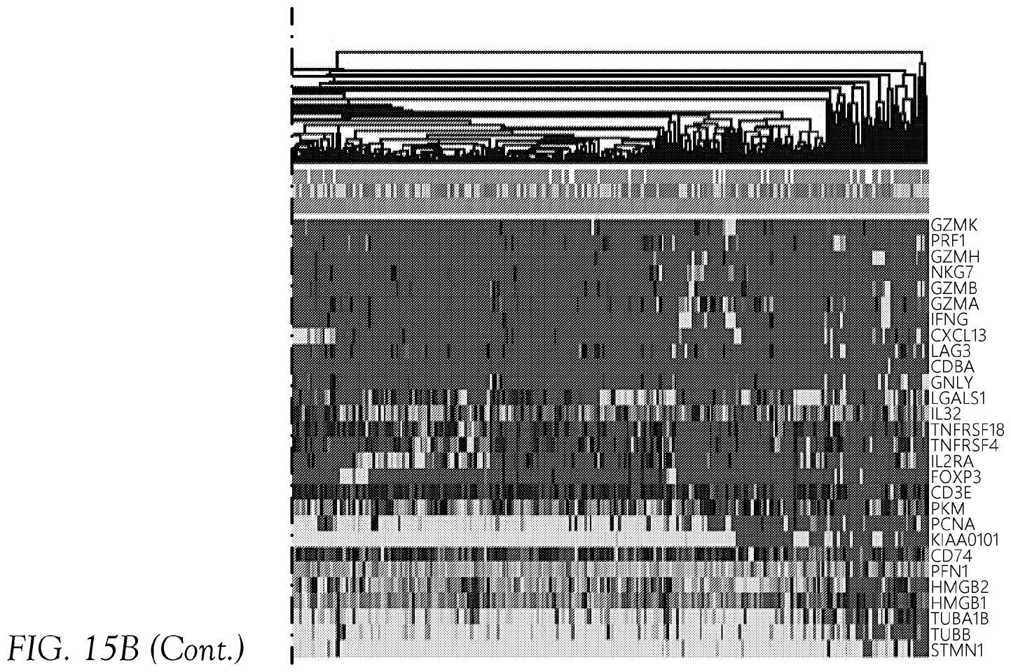

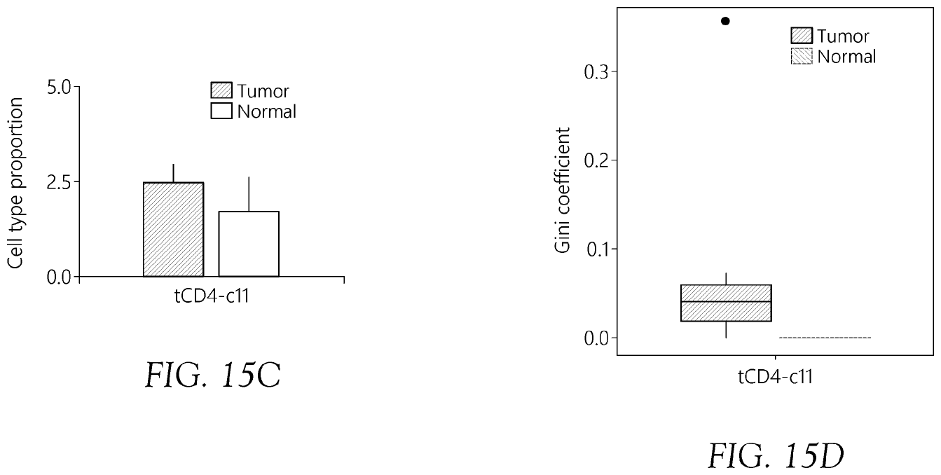

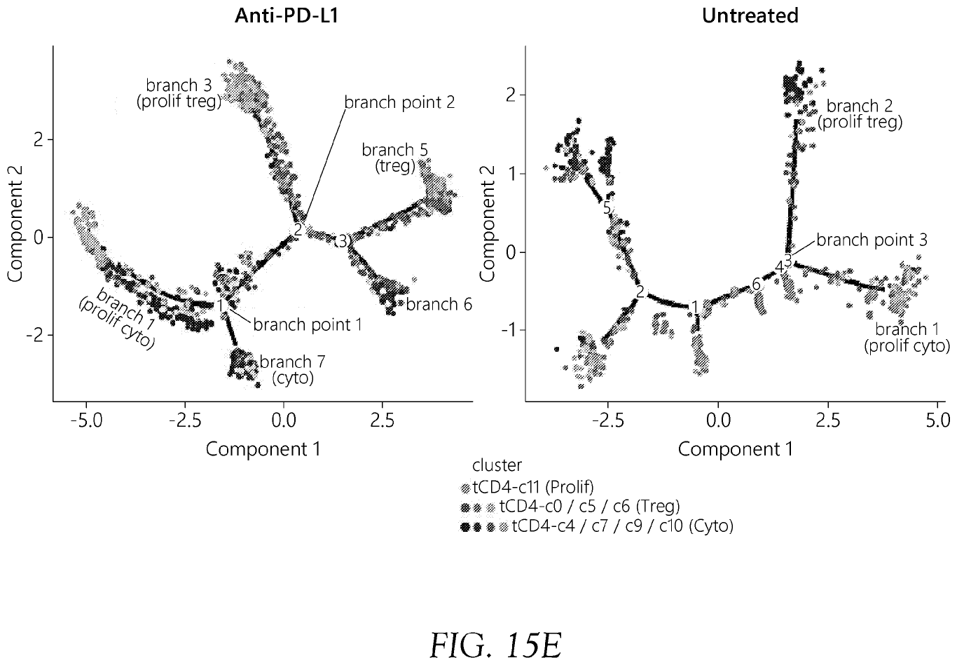

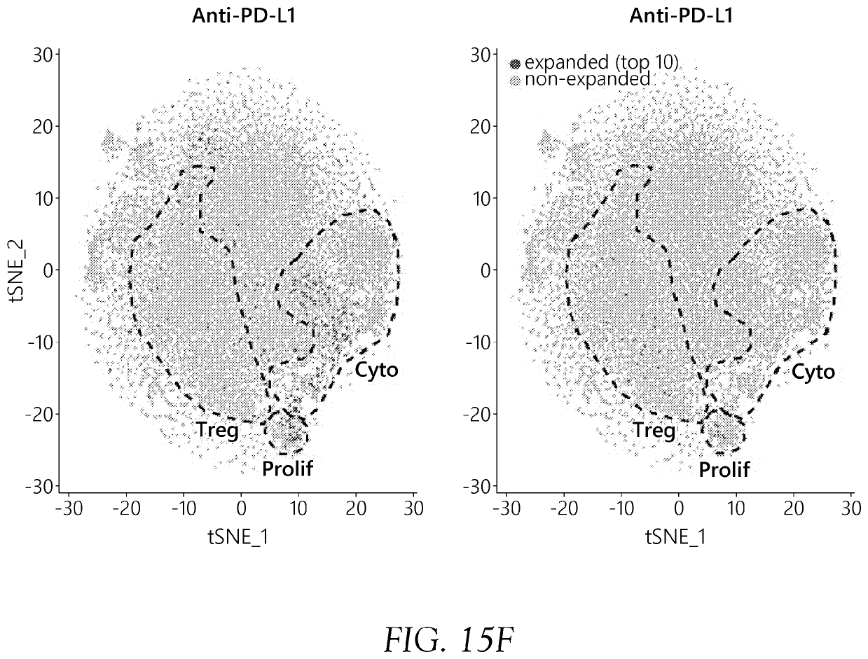

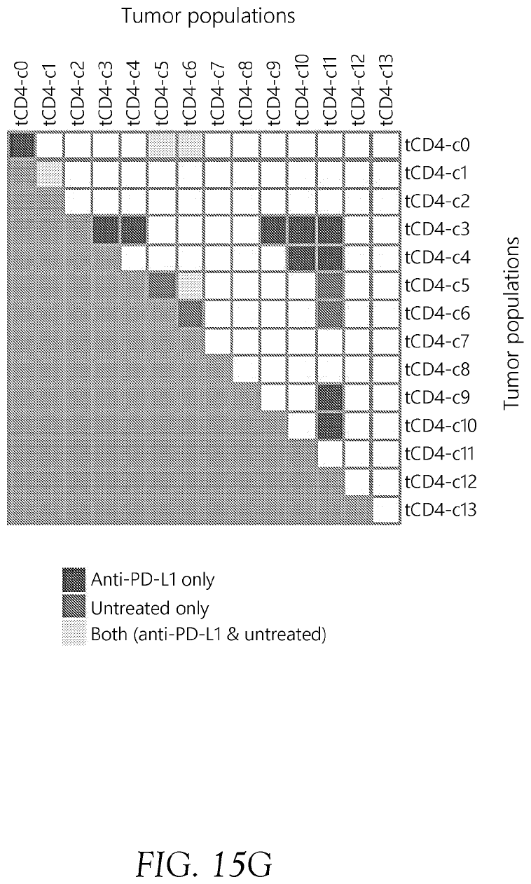

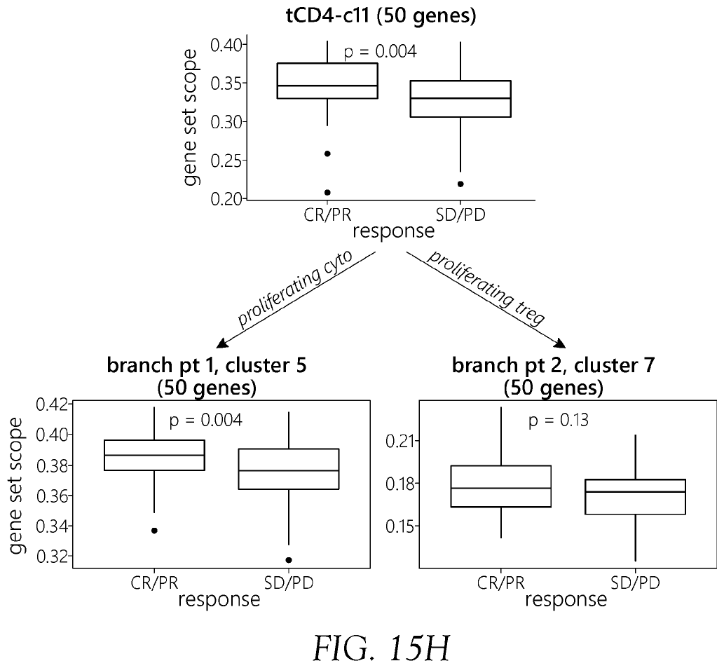

Within the tumor-infiltrating CD4⁺ T cell compartment, the experimental data presented herein also identified proliferating cells (tCD4-c11) expressing MKI67, microtubule-associated markers (e.g. STAIN1ITUBB1), the core histone HIST1H4C, and DNA-binding proteins associated with cell cycle progression such as PCNA, HMGB1, and HMGB2, which were expressed at lower levels in regulatory or cytotoxic CD4⁺ T cells (FIG. 14, FIG. 15A). A similar signature was also seen in the CD8+compartment (tCD8-c9, FIG. 2). A listing of the top 50 genes that were found upregulated in proliferating CD8⁺ cells (e.g., tCD8-c9) is presented in Table 2 below.

TABLE 2
Exemplary gene signatures of proliferating CD8⁺ cells. These are differentially
expressed marker genes for proliferating CD8⁺ population, identified using
Seurat for single cells in the tCD8-c9 cluster to the CCR7+ tCD8-c0 cluster.

| | Gene | | | | | |
|---|---|---|---|---|---|---|
| ID | Name | p_val | avg_logFC | pct.1 | pct.2 | p_val_adj |
| 1 | IGLL5 | 0.849428061 | 2.710763 | 0.002 | 0.002 | 1 |
| 2 | STMN1 | 4.61E−203 | 2.34059 | 0.814 | 0.119 | 5.84E−199 |
| 3 | TUBB | 5.97E−148 | 2.044267 | 0.692 | 0.112 | 7.56E−144 |
| 4 | CXCL13 | 1.60E−94 | 1.791903 | 0.395 | 0.035 | 2.02E−90 |
| 5 | GZMB | 1.35E−125 | 1.772245 | 0.831 | 0.247 | 1.71E−121 |
| 6 | TUBA1B | 9.51E−79 | 1.657271 | 0.728 | 0.33 | 1.20E−74 |
| 7 | KIAA0101 | 1.23E−185 | 1.652843 | 0.53 | 0.008 | 1.56E−181 |
| 8 | UBE2C | 6.75E−139 | 1.572464 | 0.4 | 0.005 | 8.55E−135 |
| 9 | HIST1H4C | 1.09E−66 | 1.514062 | 0.605 | 0.2 | 1.38E−62 |
| 10 | CCL3 | 4.46E−51 | 1.397739 | 0.337 | 0.064 | 5.65E−47 |
| 11 | MKI67 | 8.96E−137 | 1.378452 | 0.405 | 0.007 | 1.13E−132 |
| 12 | ACTB | 1.72E−151 | 1.364258 | 0.995 | 0.871 | 2.18E−147 |
| 13 | TOP2A | 2.16E−119 | 1.347275 | 0.371 | 0.01 | 2.74E−115 |
| 14 | HLA-DRA | 7.09E−103 | 1.344743 | 0.764 | 0.219 | 8.98E−99 |
| 15 | RRM2 | 2.69E−123 | 1.340961 | 0.342 | 0.001 | 3.41E−119 |
| 16 | CENPF | 5.48E−108 | 1.337551 | 0.402 | 0.025 | 6.94E−104 |
| 17 | GNLY | 2.33E−50 | 1.31914 | 0.388 | 0.092 | 2.95E−46 |
| 18 | HMGB2 | 3.56E−86 | 1.316955 | 0.843 | 0.418 | 4.51E−82 |
| 19 | TYMS | 5.89E−144 | 1.285154 | 0.4 | 0.003 | 7.46E−140 |
| 20 | CKS1B | 9.14E−134 | 1.246056 | 0.448 | 0.019 | 1.16E−129 |
| 21 | SMC4 | 1.49E−86 | 1.204545 | 0.545 | 0.099 | 1.89E−82 |
| 22 | NUSAP1 | 8.29E−106 | 1.201276 | 0.448 | 0.041 | 1.05E−101 |
| 23 | S100A4 | 5.45E−87 | 1.188979 | 0.841 | 0.338 | 6.90E−83 |
| 24 | GAPDH | 2.28E−115 | 1.173708 | 0.952 | 0.643 | 2.88E−111 |
| 25 | HMGB1 | 3.73E−90 | 1.153325 | 0.887 | 0.511 | 4.73E−86 |
| 26 | LGALS1 | 1.63E−57 | 1.14776 | 0.564 | 0.171 | 2.06E−53 |
| 27 | FKBP1A | 7.10E−96 | 1.125863 | 0.619 | 0.12 | 9.00E−92 |
| 28 | HAVCR2 | 1.69E−74 | 1.102883 | 0.352 | 0.038 | 2.14E−70 |
| 29 | HIST1H1D | 6.67E−60 | 1.09891 | 0.414 | 0.083 | 8.45E−56 |
| 30 | CORO1A | 1.31E−67 | 1.091264 | 0.684 | 0.264 | 1.66E−63 |
| 31 | HMGN2 | 9.92E−105 | 1.07916 | 0.501 | 0.058 | 1.26E−100 |
| 32 | NUCKS1 | 1.68E−84 | 1.067938 | 0.528 | 0.092 | 2.13E−80 |
| 33 | ACTG1 | 1.67E−80 | 1.064281 | 0.901 | 0.516 | 2.12E−76 |
| 34 | RPA3 | 2.21E−82 | 1.063796 | 0.494 | 0.079 | 2.80E−78 |
| 35 | BIRC5 | 1.52E−125 | 1.061272 | 0.345 | 0.001 | 1.92E−121 |
| 36 | ANXA5 | 1.42E−83 | 1.057944 | 0.614 | 0.134 | 1.80E−79 |
| 37 | TK1 | 2.05E−106 | 1.051159 | 0.308 | 0.003 | 2.60E−102 |
| 38 | PFN1 | 2.02E−108 | 1.048339 | 0.959 | 0.684 | 2.56E−104 |
| 39 | CALM3 | 9.53E−93 | 1.044721 | 0.665 | 0.152 | 1.21E−88 |
| 40 | NUDT1 | 2.64E−94 | 1.044572 | 0.414 | 0.038 | 3.34E−90 |
| 41 | MT2A | 1.49E−74 | 1.040166 | 0.776 | 0.283 | 1.89E−70 |
| 42 | RANBP1 | 4.06E−86 | 1.036226 | 0.598 | 0.129 | 5.15E−82 |
| 43 | UBE2T | 1.01E−84 | 1.032827 | 0.284 | 0.01 | 1.28E−80 |
| 44 | ANAPC11 | 1.25E−77 | 1.02124 | 0.554 | 0.114 | 1.59E−73 |
| 45 | HLA-DRB1 | 2.20E−70 | 1.014055 | 0.807 | 0.34 | 2.79E−66 |
| 46 | HOPX | 7.62E−50 | 1.007001 | 0.342 | 0.066 | 9.65E−46 |
| 47 | MAD2L1 | 4.78E−98 | 1.00404 | 0.366 | 0.022 | 6.05E−94 |
| 48 | DUT | 6.36E−72 | 1.001873 | 0.547 | 0.129 | 8.06E−68 |
| 49 | PKM | 3.81E−87 | 0.998756 | 0.622 | 0.138 | 4.83E−83 |
| 50 | PCNA | 5.62E−75 | 0.99384 | 0.424 | 0.065 | 7.11E−71 |

Flow cytometric analysis confirmed the presence of Ki67⁺ CD4⁺ T cells that also co-expressed HLA-DR in multiple tumors (FIG. 6A and data not shown). Of note, expression of markers for regulatory (e.g., IL2RA, TNFRSF18) and cytotoxic CD4⁺ T cells (e.g., GZMA and GNLY) was bimodal in this proliferating population (FIG. 15A). On closer examination, the bimodality can be explained by discrete groups of cells co-expressing either regulatory or cytotoxic genes, but not both simultaneously (FIG. 15B). The proliferating tCD4-c11 cells were not quantitatively enriched or clonally expanded in the tumor environment (FIGS. 15C-15D).

Given that the proliferating CD4⁺ T cells appeared to be composed of distinct groups of cells expressing modules of either regulatory or cytotoxic genes, further experiments were performed to investigate the developmental relationship between proliferating, cytotoxic, and regulatory CD4⁺ populations using pseudotime analysis (Qiu et al., 2017). This subdivided proliferative tCD4-c11 into two populations, each lying along a developmental trajectory specific for either cytotoxic and regulatory CD4+(FIG. 15E). Given the division of the proliferative tCD4-c11 population into distinct terminal branches, this observation suggests that this population is the end result of activation of distinct cytotoxic or regulatory cells. Interestingly, while proliferative cells in untreated bladder tumors were predominantly regulatory in nature, the proliferative population in anti-PD-Ll-treated bladder tumors was increasingly skewed towards cytotoxic CD4⁺ cells (FIG. 15E). An increase in relatedness between proliferative and cytotoxic CD4⁺ cells was also observed using orthogonal analysis of sharing of exact TRA/TRB sequences between populations. The top 10 expanded proliferative TCR clonotypes were predominantly shared with regulatory populations in untreated tumors, which shifted to primarily cytotoxic CD4⁺ populations in anti-PD-Ll-treated tumors (FIG. 15F). Quantitative analysis of clonotype sharing underscored this finding: all samples showed significant clonotype sharing between related regulatory populations (tCD4-c0 and either tCD4-c5 or -c6). However, only untreated tumors exhibited significant sharing between the proliferative tCD4c-11 and the regulatory tCD4-c5 and tCD4-c6 states, while anti-PD-Ll-treated tumors exclusively demonstrated sharing between the CXCL13⁺ tCD4-c3 and multiple cytotoxic populations, and importantly between proliferative tCD4-c11 and multiple cytotoxic populations (tCD4-c4, -c9, -c10) (permutation P value <0.05, FIG. 15G). Hence, distinct analyses using both transcriptional and clonotypic analysis indicated that anti-PD-L1 shifted the immune milieu in bladder tumors from activated regulatory cells in the untreated state towards activated cytotoxic CD4⁺ T cell effectors.

### Example 8

### A signature of Proliferative Cytotoxic CD4+ T Cells Predicts Clinical Response to Anti-PD-L1

This Example describes experiments performed to probe the biological importance of CD4⁺ T cell populations, where the top-ranked differentially expressed genes for each CD4⁺ population (by fold change) were used to perform single-sample gene set scoring (singscore, Foroutan et al., 2018), obtaining enrichment scores for each population's signature in bulk RNA sequencing data. This approach was applied to data from pre-treatment bladder tumors from a separate phase 2 trial of atezolizumab for metastatic bladder cancer (IMvigor 210 [Mariathasan et al., 2018]). In 168 metastatic bladder cancer patients with pre-treatment RNAseq data from bladder tumors as well as both response and survival data, a 50-gene signature from the proliferating tCD4-c11 was significantly correlated with clinical response to anti-PD-L1 therapy (P=0.004 by Wilcoxon signed-rank test, FIG. 1511). A listing of the top 50 genes that were found upregulated in proliferating CD4⁺ T cells (e.g., tCD4-c1 1) is presented in Table 3 below.

TABLE 3
Exemplary gene signatures of proliferating CD4⁺ cells. These are differentially
expressed marker genes for proliferating CD4⁺ population, identified using
Seurat for single cells in the tCD4-c11 cluster compared to the CCR7+ tCD4-c1 cluster.

| | Gene | | | | | |
|---|---|---|---|---|---|---|
| ID | Name | p_val | avg_logFC | pct.1 | pct.2 | p_val_adj |
| 1 | STMN1 | 0 | 2.75228234 | 0.966 | 0.076 | 0 |
| 2 | TUBB | 0 | 2.242899658 | 0.914 | 0.116 | 0 |
| 3 | HIST1H4C | 1.80E−264 | 2.188963892 | 0.826 | 0.155 | 2.36E−260 |
| 4 | TUBA1B | 3.56E−245 | 2.035998557 | 0.966 | 0.342 | 4.67E−241 |
| 5 | KIAA0101 | 0 | 1.976898094 | 0.792 | 0.005 | 0 |
| 6 | HLA-DRA | 1.54E−297 | 1.789003071 | 0.706 | 0.07 | 2.01E−293 |
| 7 | HMGB2 | 6.15E−231 | 1.73091924 | 0.951 | 0.354 | 8.07E−227 |
| 8 | GZMB | 1.93E−98 | 1.639274147 | 0.27 | 0.029 | 2.53E−94 |
| 9 | RRM2 | 0 | 1.616973754 | 0.624 | 0.001 | 0 |
| 10 | LGALS1 | 1.42E−141 | 1.608816201 | 0.605 | 0.13 | 1.87E−137 |
| 11 | TK1 | 0 | 1.607912826 | 0.695 | 0.003 | 0 |
| 12 | TYMS | 0 | 1.597239432 | 0.648 | 0.002 | 0 |
| 13 | GNLY | 1.14E−100 | 1.576485647 | 0.268 | 0.028 | 1.50E−96 |
| 14 | MT2A | 2.19E−215 | 1.573365468 | 0.856 | 0.192 | 2.88E−211 |
| 15 | UBE2C | 0 | 1.558500151 | 0.601 | 0.001 | 0 |
| 16 | PFN1 | 2.15E−230 | 1.553976004 | 0.987 | 0.663 | 2.82E−226 |
| 17 | GAPDH | 4.02E−227 | 1.553961741 | 0.987 | 0.623 | 5.27E−223 |
| 18 | ACTB | 4.71E−239 | 1.544357423 | 1 | 0.9 | 6.17E−235 |
| 19 | HLA-DRB1 | 6.27E−239 | 1.488693889 | 0.725 | 0.101 | 8.23E−235 |
| 20 | PKM | 2.24E−258 | 1.485994015 | 0.899 | 0.175 | 2.94E−254 |
| 21 | CKS1B | 0 | 1.4372702 | 0.7 | 0.018 | 0 |
| 22 | DUT | 3.54E−226 | 1.406099447 | 0.841 | 0.167 | 4.65E−222 |
| 23 | NUSAP1 | 0 | 1.395419003 | 0.657 | 0.021 | 0 |
| 24 | HMGB1 | 3.25E−210 | 1.390605871 | 0.974 | 0.465 | 4.27E−206 |
| 25 | PCNA | 0 | 1.344479513 | 0.708 | 0.06 | 0 |
| 26 | RANBP1 | 8.89E−252 | 1.338009593 | 0.861 | 0.15 | 1.17E−247 |
| 27 | CCL4 | 1.73E−31 | 1.326936464 | 0.155 | 0.033 | 2.27E−27 |
| 28 | TOP2A | 0 | 1.322868894 | 0.532 | 0.004 | 0 |
| 29 | MKI67 | 0 | 1.312204695 | 0.592 | 0.002 | 0 |
| 30 | CD74 | 7.40E−147 | 1.300117247 | 0.925 | 0.387 | 9.71E−143 |
| 31 | ZWINT | 0 | 1.298352884 | 0.579 | 0.004 | 0 |
| 32 | PTTG1 | 3.02E−262 | 1.283468625 | 0.794 | 0.105 | 3.96E−258 |
| 33 | TPI1 | 4.20E−191 | 1.275521275 | 0.912 | 0.276 | 5.51E−187 |
| 34 | CENPF | 0 | 1.264426295 | 0.543 | 0.015 | 0 |
| 35 | H2AFZ | 1.24E−174 | 1.242628175 | 0.961 | 0.475 | 1.62E−170 |
| 36 | S100A4 | 2.03E−131 | 1.216245039 | 0.942 | 0.524 | 2.67E−127 |
| 37 | ENO1 | 4.27E−152 | 1.212325709 | 0.884 | 0.327 | 5.60E−148 |
| 38 | ANXA5 | 1.02E−230 | 1.204440184 | 0.818 | 0.138 | 1.34E−226 |
| 39 | COTL1 | 7.89E−133 | 1.186710764 | 0.833 | 0.301 | 1.03E−128 |
| 40 | PPP1CA | 6.71E−247 | 1.18402312 | 0.858 | 0.142 | 8.81E−243 |
| 41 | BIRC5 | 0 | 1.178890068 | 0.577 | 0.001 | 0 |
| 42 | CORO1A | 1.67E−141 | 1.176789085 | 0.828 | 0.299 | 2.19E−137 |
| 43 | ACTG1 | 1.40E−149 | 1.171190716 | 0.964 | 0.581 | 1.83E−145 |
| 44 | MIR4435-1HG | 6.01E−254 | 1.167150594 | 0.659 | 0.065 | 7.89E−250 |
| 45 | CDK1 | 0 | 1.159528484 | 0.534 | 0.008 | 0 |
| 46 | NUDT1 | 0 | 1.151920255 | 0.7 | 0.035 | 0 |
| 47 | CALM3 | 5.45E−206 | 1.147193887 | 0.835 | 0.162 | 7.16E−202 |
| 48 | ARPC1B | 5.59E−134 | 1.125998591 | 0.91 | 0.372 | 7.34E−130 |
| 49 | HIST1H1D | 3.29E−195 | 1.115502622 | 0.526 | 0.056 | 4.31E−191 |
| 50 | HLA-DPA1 | 7.46E−173 | 1.110797371 | 0.708 | 0.133 | 9.79E−169 |

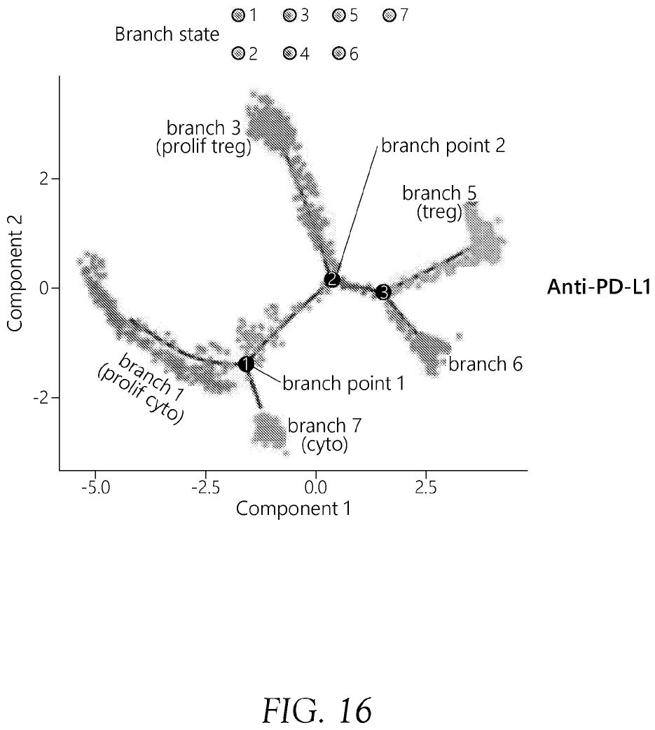

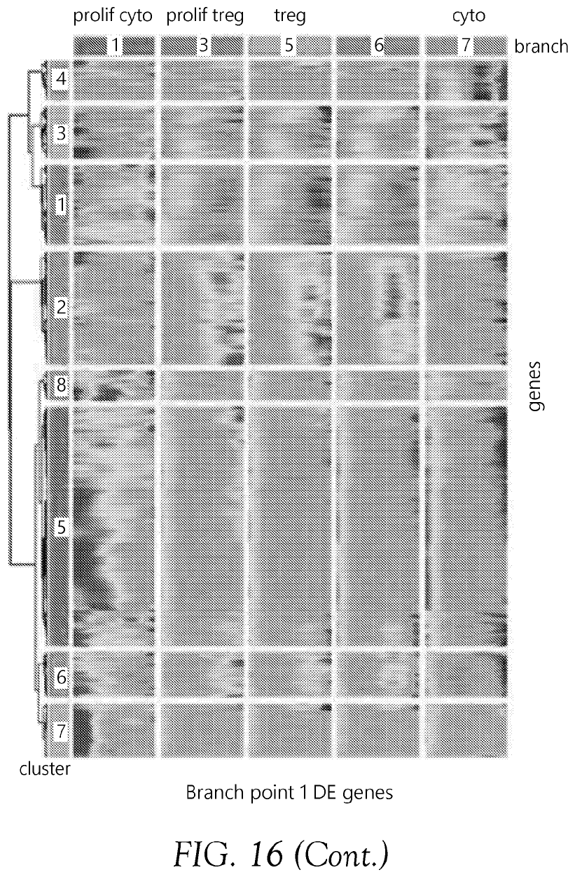

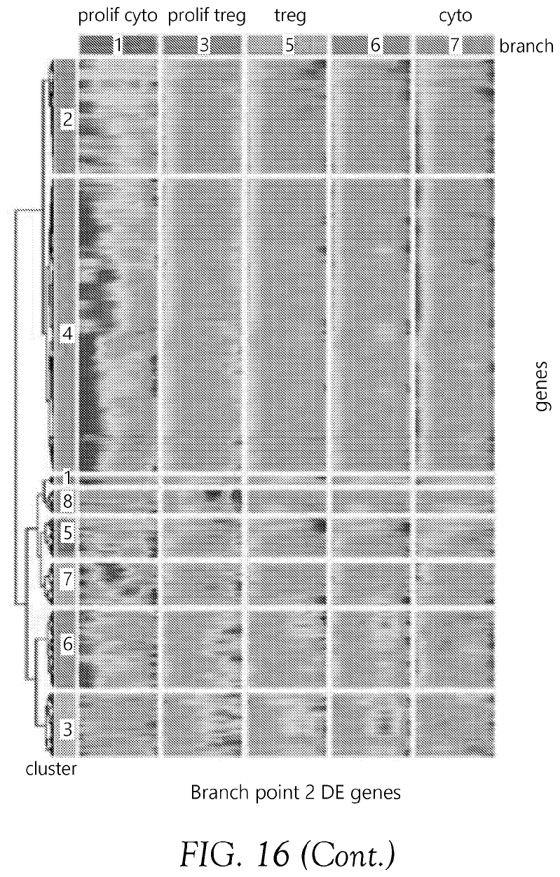

The level of significance was similar across a range of 30-100 genes tested from the signature. To obtain a specific signature for proliferative cytotoxic or regulatory CD4⁺ T cells, branched expression analysis modeling (BEAM) was performed to identify all genes with branch-dependent differential expression at branch point 1 (splitting proliferative and non-proliferative cytotoxic CD4⁺ T cells) and branch point 2 (splitting proliferative and non-proliferative regulatory CD4⁺ T cells). Clustering of these genes based on their shared up- or down-regulation in specific branches identified specific gene signatures that were coordinately upregulated in proliferative cytotoxic or regulatory populations, but not in their non-proliferative counterparts (clusters 5-8 for cytotoxic cells at branch point 1, clusters 3 and 5-8 for regulatory cells at branch point 2, all genes with q <0.05, branch-specific signatures, heatmap of cluster-specific gene expression in FIG. 16). A listing of the top 50 genes that were found upregulated in regulatory CD4⁺ T cells (e.g., tCD4-c0) is presented in Table 4.

TABLE 4
Exemplary gene signatures of regulatory CD4⁺ cells.

| | Gene | | | | | |
|---|---|---|---|---|---|---|
| ID | Name | p_val | avg_logFC | pct.1 | pct.2 | p_val_adj |
| 1 | IL2RA | 9.67E−292 | 1.522759678 | 0.432 | 0.065 | 1.27E−287 |
| 2 | IL32 | 0 | 1.468906705 | 0.95 | 0.611 | 0 |
| 3 | MIR4435-1HG | 0 | 1.405264483 | 0.466 | 0.065 | 0 |
| 4 | TIGIT | 0 | 1.40223346 | 0.572 | 0.105 | 0 |
| 5 | CARD16 | 0 | 1.370199507 | 0.571 | 0.125 | 0 |
| 6 | MAGEH1 | 9.82E−189 | 1.264234455 | 0.312 | 0.048 | 1.29E−184 |
| 7 | PMAIP1 | 0 | 1.25554208 | 0.646 | 0.194 | 0 |
| 8 | HLA-DRB1 | 2.60E−210 | 1.199723026 | 0.415 | 0.101 | 3.41E−206 |
| 9 | LINC00152 | 1.97E−292 | 1.171404329 | 0.577 | 0.172 | 2.58E−288 |
| 10 | CD74 | 0 | 1.141415387 | 0.792 | 0.387 | 0 |
| 11 | CD27 | 2.10E−250 | 1.134869392 | 0.507 | 0.142 | 2.76E−246 |
| 12 | HLA-DRA | 1.16E−138 | 1.127378494 | 0.295 | 0.07 | 1.53E−134 |
| 13 | SAT1 | 0 | 1.058018153 | 0.856 | 0.539 | 0 |
| 14 | TNFRSF9 | 1.23E−173 | 1.057912451 | 0.277 | 0.036 | 1.62E−169 |
| 15 | CTSC | 6.10E−188 | 1.042118394 | 0.397 | 0.102 | 8.00E−184 |
| 16 | DUSP4 | 8.10E−245 | 1.032101173 | 0.584 | 0.209 | 1.06E−240 |
| 17 | AC002331.1 | 7.45E−138 | 0.988736898 | 0.201 | 0.017 | 9.78E−134 |
| 18 | TNFRSF18 | 1.97E−156 | 0.975276653 | 0.33 | 0.08 | 2.59E−152 |
| 19 | BATF | 2.47E−187 | 0.938287058 | 0.54 | 0.215 | 3.24E−183 |
| 20 | HLA-DPB1 | 1.02E−162 | 0.935983573 | 0.413 | 0.13 | 1.33E−158 |
| 21 | TNFRSF4 | 2.72E−112 | 0.928642028 | 0.34 | 0.121 | 3.57E−108 |
| 22 | CXCR6 | 4.75E−148 | 0.927934804 | 0.359 | 0.104 | 6.23E−144 |
| 23 | AC017002.1 | 1.21E−146 | 0.922959693 | 0.199 | 0.012 | 1.58E−142 |
| 24 | LAYN | 5.03E−137 | 0.907759172 | 0.188 | 0.012 | 6.59E−133 |
| 25 | HPGD | 2.28E−101 | 0.877303409 | 0.195 | 0.035 | 2.99E−97 |
| 26 | RTKN2 | 3.99E−131 | 0.877022674 | 0.235 | 0.037 | 5.23E−127 |
| 27 | ICA1 | 1.01E−114 | 0.863385623 | 0.213 | 0.036 | 1.33E−110 |
| 28 | LAIR2 | 7.36E−103 | 0.833739835 | 0.169 | 0.021 | 9.66E−99 |
| 29 | HTATIP2 | 3.69E−120 | 0.830722523 | 0.243 | 0.049 | 4.84E−116 |
| 30 | IL1R2 | 2.79E−110 | 0.824573126 | 0.154 | 0.01 | 3.66E−106 |
| 31 | HLA-DPA1 | 1.18E−131 | 0.815448839 | 0.384 | 0.133 | 1.55E−127 |
| 32 | CTLA4 | 2.39E−123 | 0.810739084 | 0.278 | 0.068 | 3.14E−119 |
| 33 | GBP2 | 1.83E−148 | 0.810140338 | 0.433 | 0.161 | 2.40E−144 |
| 34 | GLRX | 9.20E−115 | 0.809759456 | 0.337 | 0.114 | 1.21E−110 |
| 35 | CST7 | 2.42E−135 | 0.796254618 | 0.411 | 0.15 | 3.17E−131 |
| 36 | S100A4 | 8.16E−231 | 0.793009892 | 0.824 | 0.524 | 1.07E−226 |
| 37 | DNPH1 | 2.69E−110 | 0.774594689 | 0.332 | 0.116 | 3.53E−106 |
| 38 | ACP5 | 2.28E−94 | 0.770412583 | 0.168 | 0.025 | 2.99E−90 |
| 39 | SOX4 | 1.85E−65 | 0.769828984 | 0.111 | 0.014 | 2.43E−61 |
| 40 | ENTPD1 | 1.91E−106 | 0.755928559 | 0.209 | 0.039 | 2.51E−102 |
| 41 | HLA-DQA1 | 1.18E−78 | 0.723195987 | 0.163 | 0.032 | 1.55E−74 |
| 42 | LTB | 2.10E−116 | 0.691321203 | 0.731 | 0.528 | 2.75E−112 |
| 43 | HLA-DMA | 1.52E−85 | 0.671339696 | 0.176 | 0.034 | 2.00E−81 |
| 44 | BTG3 | 2.56E−91 | 0.668386066 | 0.356 | 0.15 | 3.36E−87 |
| 45 | HLA-DRB5 | 1.31E−61 | 0.653017305 | 0.167 | 0.047 | 1.72E−57 |
| 46 | TBC1D4 | 4.22E−87 | 0.646044853 | 0.21 | 0.052 | 5.53E−83 |
| 47 | PARK7 | 1.44E−109 | 0.635886988 | 0.55 | 0.302 | 1.90E−105 |
| 48 | USP15 | 3.87E−83 | 0.63433343 | 0.352 | 0.155 | 5.08E−79 |
| 49 | UCP2 | 1.96E−92 | 0.63115351 | 0.412 | 0.194 | 2.58E−88 |
| 50 | GBPS | 2.22E−82 | 0.630288033 | 0.194 | 0.046 | 2.91E−78 |

Testing these signatures against the bulk RNAseq data from IMvigor210, it was observed that a signature of proliferative cytotoxic CD4⁺ T cells (branch point 1, cluster 5) was associated with response to anti-PD-L1 at a similar level of significance as the proliferating tCD4-c11 signature (P=0.004 by Wilcoxon for 50 genes) and remained significant with the addition of up to 100 genes. A listing of the top 50 genes that were found upregulated in proliferative cytotoxic CD4⁺ T cells (e.g., branch 1 cluster 5) is presented in Table 5 below.

TABLE 5
Exemplary gene signatures of proliferative cytolytic CD4⁺ cells.

| ID | Gene Name | p_val |
|---|---|---|
| 1 | TMSB10 | 1.04E−26 |
| 2 | ACTB | 1.06E−16 |
| 3 | MYL6 | 1.48E−14 |
| 4 | ATP5E | 1.64E−12 |
| 5 | KIF15 | 5.64E−12 |
| 6 | MYBL2 | 3.31E−10 |
| 7 | ACTG1 | 3.34E−09 |
| 8 | ARPC1B | 2.43E−08 |
| 9 | ENO1 | 4.69E−08 |
| 10 | UQCRB | 1.14E−07 |
| 11 | DNA2 | 1.14E−07 |
| 12 | UQCR11.1 | 2.11E−07 |
| 13 | TPI1 | 5.25E−07 |
| 14 | YWHAB | 8.26E−07 |
| 15 | STMN1 | 1.72E−06 |
| 16 | PKM | 1.85E−06 |
| 17 | CDT1 | 2.39E−06 |
| 18 | DMC1 | 2.56E−06 |
| 19 | COX7C | 4.62E−06 |
| 20 | KIAA0101 | 4.99E−06 |
| 21 | LDHB | 8.78E−06 |
| 22 | C9orf16 | 9.15E−06 |
| 23 | NDUFA13 | 1.02E−05 |
| 24 | ZNF724P | 1.62E−05 |
| 25 | TMEM258 | 1.65E−05 |
| 26 | EIF3H | 1.82E−05 |
| 27 | NDUFA4 | 2.15E−05 |
| 28 | COX5B | 2.36E−05 |
| 29 | TRAPPC1 | 2.53E−05 |
| 30 | PARK7 | 2.58E−05 |
| 31 | ECH1 | 3.07E−05 |
| 32 | CALM3 | 3.43E−05 |
| 33 | CHAF1B | 3.73E−05 |
| 34 | UCK2 | 3.85E−05 |
| 35 | CDC6 | 4.37E−05 |
| 36 | GAPDH | 4.56E−05 |
| 37 | PRDX5 | 4.81E−05 |
| 38 | FAM72B | 6.77E−05 |
| 39 | ATP5A1 | 7.12E−05 |
| 40 | MKI67 | 9.26E−05 |
| 41 | HNRNPA1 | 0.000112 |
| 42 | ATP5J2 | 0.000119 |
| 43 | FKBP1A | 0.000123 |
| 44 | PPP1R7 | 0.000126 |
| 45 | RPL23 | 0.000126 |
| 46 | SHMT1 | 0.00013 |
| 47 | PPM1G | 0.00014 |
| 48 | DBNL | 0.00014 |
| 49 | DPP7 | 0.000164 |
| 50 | NOP10 | 0.000168 |

Of note, this 50-gene proliferative cytotoxic CD4⁺ signature did share a limited number of genes with the 50-gene proliferating tCD4-c11 signature (6 genes: STMN1, KIAA0101, PKAT MKI67, TPI1, EN01) or with the 115-gene list pooled from 50-gene signatures of all cytotoxic CD4⁺ populations (3 genes: FKBP1A,TMSB10, MYL6). However, 36 of 50 genes in the signature were specific to proliferative cytotoxic CD4⁺ T cells, including top-ranked genes by q value such as ATP5E, KIF15, MYBL2, UQCRB, and DNA2, and this reduced 36-gene signature (with overlapping genes removed) was similarly significant to the 50-gene proliferative cytotoxic CD4⁺ signature (P=0.005 by Wilcoxon). In contrast, the association with the most significant proliferative regulatory signature (branch point 2, cluster 7) was much weaker (P=0.13 by Wilcoxon for 50 genes) (see, e.g., FIG. 1511). Hence, a signature of activated cytotoxic CD4⁺ T cell effectors is specifically associated with response to PD-1 blockade in a large orthogonal data set. The presence of this signature in pre-treatment bladder tumors prior to anti-PD-L1 in responding patients suggests that anti-PD-L1 therapy may enhance pre-existing cytotoxic CD4⁺ T cell activation with further activation of these cells upon treatment.

### Example 9

### Canonical CD8+ T Cell States Were not Enriched in the Bladder Tumor Microenvironment

With reference to Example 1 above, this Example describes additional experiments performed to assess the T cell composition of the tumor environment. T cells from dissociated bladder tumors and adjacent uninvolved bladder tissues were profiled using single-cell RNA and TCR sequencing.

The 10× Genomics Chromium platform (Zheng et al., 2017b) was used to sequence 8,833 tumor-derived and 1,929 non-malignant tissue-derived CD8+ T cells from 7 patients (Table 6). All samples were muscle-invasive bladder cancer (MIBC) from 2 standard-of-care-untreated patients (“untreated”), 1 chemotherapy-treated patient (gemcitabine +carboplatin, “chemo”), and 4 anti-PD-Ll-treated patients (“anti-PD-L1”) with detailed clinical annotations (Table 6).

To assess the shared heterogeneity of T cells across samples, the analysis was restricted to highly variable genes and used an empirical Bayes approach (ComBat; Johnson et aL, 2007; Butner et al., 2019) to account for preparation batch among individual samples. Leiden clustering (Traag et al., 2019) was subsequently used to define clusters that were visualized using uniform manifold approximation and projection (UMAP) (McInnes and Healy, 2018). It was observed that tumor- and non-malignant-derived CD8+ T cells formed 11 clusters, each populated by cells from all samples suggestive of shared states in TCC regardless of the treatment regimen (see, e.g., FIG. 17A). Differential expression analyses comparing each cluster with all other cells combined identified 1,067 differentially expressed genes in at least one cluster (adjusted P value (Padj) <0.05, llog2(fold change, FC)1>0.5) (see, e.g., Table 7).

TABLE 6
Characteristics of samples analyzed by scRNAseq for this study.

| | | | MIBC or | | Tumor at | Path | Path | # tumor | # normal | # tumor | # normal |
|---|---|---|---|---|---|---|---|---|---|---|---|
| Pt ID | Age | M/F | NMIBC | Neoadj tx | surgery | T stage | N stage | CD4 | CD4 | CD8 | CD8 |
| Anti-PD-L1 A | 74 | M | MIBC | Atezo x 1 | Y (2.0 cm) | ypTa | ypN0 | 1984 | NA | 1101 | NA |
| Anti-PD-L1 B | 64 | M | MIBC | Atezo x 2 | Y (6.5 cm) | ypT4b | ypN2 | 2459 | 425 | 627 | NA |
| Anti-PD-L1 C | 68 | F | MIBC | Atezo x 2 | Y (6.8 cm) | ypT1 | ypN0 | 3592 | 709 | 1383 | NA |
| Anti-PD-L1 D | 71 | M | MIBC | Atezo x 2 | Y (1.5 cm) | ypT2b | ypN0 | 3552 | 326 | 993 | NA |
| Chemo | 67 | F | MIBC | Chemo | Y (<0.1 cm) | ypTis | ypN0 | 1350 | 967 | 1859 | 417 |
| Untreated A | 82 | M | MIBC | None | Y (3 cm) | pT3b | pN0 | 3031 | 130 | 1513 | NA |
| Untreated B | 76 | M | MIBC | None | Y (4 cm) | pT3b | pN0 | 1027 | 290 | 1357 | 1512 |
| Healthy | NA | NA | NA | NA | NA | NA | NA | NA | 13634 | NA | 7034 |
| | | | | | | | | | (blood) | | (blood) |

To assess the shared heterogeneity of T cells across samples, the analysis was restricted to highly variable genes and used an empirical Bayes approach (ComBat; Johnson et al., 2007; Butner et al., 2019) to account for preparation batch among individual samples. Leiden clustering (Traag et al., 2019) was subsequently used to define clusters that were visualized using uniform manifold approximation and projection (UMAP) (McInnes and Healy, 2018). It was observed that tumor- and non-malignant-derived CD8+ T cells formed 11 clusters, each populated by cells from all samples suggestive of shared states in TCC regardless of the treatment regimen (see, e.g., FIG. 17A). Differential expression analyses comparing each cluster with all other cells combined identified 1,067 differentially expressed genes in at least one cluster (adjusted P value (Padj) <0.05, llog2(fold change, FC)1>0.5) (see, e.g., Table 7).

TABLE 7
Exemplary gene signature of proliferating CD8⁺ cells. These
are differentially expressed (e.g., upregulated) marker genes
for each CD8+ population (versus all other CD8+ populations),
identified using scanpy on single cells. A listing of the top biomarker
genes that were found differentially expressed in proliferating
CD8⁺ cells is presented in Table 7 below.

| ID | Gene Name | log2FC | Adjusted P value |
|---|---|---|---|
| 1 | UBE2C | 9.485194 | 1.57E−24 |
| 2 | SPC25 | 9.450514 | 0.000190039 |
| 3 | AURKB | 9.302567 | 0.001249987 |
| 4 | DLGAP5 | 8.786396 | 0.001267253 |
| 5 | BIRC5 | 8.238404 | 1.37E−11 |
| 6 | RRM2 | 7.9543743 | 2.28E−09 |
| 7 | CCNB2 | 7.6935663 | 5.55E−06 |
| 8 | APOBEC3B | 7.5169587 | 0.031054653 |
| 9 | CDCA8 | 7.2748227 | 0.031678597 |
| 10 | GTSE1 | 7.0310216 | 0.020059406 |
| 11 | ZWINT | 6.83033 | 1.15E−07 |
| 12 | TK1 | 6.7371426 | 1.25E−09 |
| 13 | RAD51AP1 | 6.595976 | 0.004578171 |
| 14 | KIAA0101 | 6.5304985 | 3.01E−19 |
| 15 | MKI67 | 6.5043926 | 7.74E−16 |
| 16 | STMN1 | 6.445233 | 2.68E−64 |
| 17 | TYMS | 6.4317255 | 9.57E−09 |
| 18 | CDC20 | 6.425998 | 0.000451602 |
| 19 | KIFC1 | 6.408081 | 0.020957358 |
| 20 | CCNA2 | 6.379993 | 6.50E−05 |
| 21 | TOP2A | 6.368994 | 1.08E−13 |
| 22 | NUF2 | 6.367468 | 0.000447898 |
| 23 | ASPM | 6.190957 | 6.27E−08 |
| 24 | ORC6 | 6.1272855 | 0.004855995 |
| 25 | CENPW | 6.0449123 | 3.68E−06 |
| 26 | SGOL1 | 5.9409366 | 0.013872343 |
| 27 | NCAPG | 5.729861 | 0.003051741 |
| 28 | TPX2 | 5.7204523 | 3.92E−05 |
| 29 | CKAP2L | 5.679569 | 0.0009829 |
| 30 | ASF1B | 5.675704 | 0.008849401 |
| 31 | CKS1B | 5.504723 | 3.19E−20 |
| 32 | CDKN3 | 5.496895 | 1.70E−08 |
| 33 | HIST1H2AJ | 5.391901 | 0.003288949 |
| 34 | CDK1 | 5.3743987 | 1.91E−10 |
| 35 | UBE2T | 5.3647037 | 5.70E−07 |
| 36 | HIST1H1B | 5.327435 | 0.024237378 |
| 37 | CENPU | 5.3136015 | 4.64E−05 |
| 38 | NUSAP1 | 5.3110094 | 1.28E−26 |
| 39 | CCNB1 | 5.241927 | 0.039570622 |
| 40 | GGH | 5.073245 | 0.000687127 |
| 41 | TUBB | 5.031186 | 3.54E−45 |
| 42 | CENPF | 5.000442 | 3.99E−14 |
| 43 | MAD2L1 | 4.887172 | 9.41E−14 |
| 44 | SMC2 | 4.6155853 | 3.51E−09 |
| 45 | PRC1 | 4.6070633 | 0.001576133 |
| 46 | CLSPN | 4.4965763 | 0.000287779 |
| 47 | RNASEH2A | 4.399392 | 0.013410552 |
| 48 | CENPE | 4.309716 | 0.003070148 |
| 49 | MCM7 | 4.286918 | 0.000341896 |
| 50 | FBXO5 | 4.254769 | 0.034728437 |

The identified states include known CD8+subtypes (FIG. 17B and FIG. 17C): cells expressing HAVCR2 (TIM-3), LAG3, ENTPDJ, as well as the chemokine CXCL13 (CD8ENTpm: log2(FC)=1.4-3.7), described previously as tumor-reactive CD8+ T cells (Duhen et al., 2018); effector cells expressing FGFBP2 and GNLY, a granule-associated pore-forming protein known to function in pathogen killing (Krensky and Clayberger, 2009) (CD8Fon3p2: 1og2(FC)=3.6-5.3); naive cells expressing CCR7 and GZMK (CD8NAIvE: log2(FC)=0.9-2.8); central memory cells expressing CCR7 and SELL (L-selectin) (CD8cm: log2(FC)=1.5-1.7); and mucosal-associated invariant T (MAIT) cells expressing KLRB1 (CD8MAIT: log2(FC)=2.7) that preferentially use the known semi-invariant TCR a chains TRAM-2 and/or TRAJ33 (Kurioka et al., 2016; FIG. 17D). In particular, MKI67+proliferating cells (CD8pRouF: log2(FC)=6.5) as well as cells expressing the chemokines ATCL//2 (CD8x_{d}: log2(FC)=5.2-5.6) were also found in this experiment. Similar states were also identified in the tumor environment of hepatocellular carcinoma based on scRNA-seq (Zheng et al., 2017a). Surprisingly, although the frequency of CD8_{ENT}p_{Di} cells was higher in tumors, none of the CD8+states displayed statistically significant differences in frequency between the tumor and non-malignant bladder (exact permutation test; FIG. 17E; density plots in FIG. 17F).

### Example 10

### Regulatory T Cells Included Heterogeneous States that are Enriched in Bladder Tumors

With referenced to Example 2 above, this Example describes the results of additional experiments performed to investigate CD4⁺ T cell heterogeneity in a similar fashion to determine their contribution to anti-tumor responses. In total, 16,995 tumor- and 2,847 non-malignant tissue-infiltrating CD4+ T cells isolated from the same patients were sequenced and analyzed. Tumor-derived and non-malignant tissue-derived CD4+ T cells formed 11 clusters each with representation from all individual patients (FIG. 18A). A total of 1,511 differentially expressed genes were identified in at least one cluster (Padj <0.05, llog2(FC)1>0.5; see also Table 8; FIG. 18B and FIG. 218) defining several canonical CD4+ T cell states. These include CCR7+cells, which demonstrated a central memory phenotype (CD4cm) based on parallel flow cytometry data showing that these were CD45RA⁻ (see below) as well as cells expressing high levels of CXCL13 and IFNG (CD4cxcLi3: log2(FC)=5.9 and 1.4), which were also likely to be exhausted based on overexpression of TOX (log2(FC)=1.9) and whose presence has been associated with improved outcomes in breast, gastric, and microsatellite-unstable colorectal carcinoma, which is an immune-responsive tumor. Other states included Th17 cells expressing IL17A (CD4_{TH17}: log2(FC)=4.7), which represented important anti-tumor effectors, activated cells expressing CD69 (CD4_{A}cTivATED:log2(FC)=2.2) but not FOXP3 (log2(FC) <0.5) (FIG. 18B and FIG. 18C); as well as several important additional states described in further detail below. Notably, some of these states were selectively enriched in specific compartments. CD4cxcLi3 demonstrated significant enrichment in tumor compared with non-malignant tissue (tumor versus non-malignant: 6.5% versus 3.0%, p=0.015, exact permutation test), whereas states enriched in non-malignant tissue included CD4cm (tumor versus non-malignant: 30% versus 42%, p=0.008) and CD4ACTIVATED (tumor versus non-malignant: 7.5% versus 10%, p=0.02) (density plots in FIG. 18D; tumor and non-malignant frequencies in FIG. 18E). A listing of the top biomarker genes that were found differentially expressed in 2 populations of regulatory CD4+ T cells (CD4m2RAxi, CD4m2RAL0) is presented in Table 8 below.

TABLE 8
Exemplary gene signature of regulatory CD4⁺ cells.
These are differentially expressed (e.g., upregulated)
marker genes for each CD4+ population (versus all
other CD4+ populations), identified using scanpy on single cells.

| | | | |
|---|---|---|---|
| ID | Gene Name | log2FC | Adjusted P value |

Upregulated genes in CD4_{IL2RAHI}

| | | | |
|---|---|---|---|
| 1 | IL1R2 | 4.1890783 | 6.99E−124 |
| 2 | IL2RA | 3.6013145 | 0 |
| 3 | EBI3 | 3.524415 | 1.39E−05 |
| 4 | AC145110.1 | 3.3836255 | 5.46E−14 |
| 5 | TNFRSF4 | 3.3037114 | 0 |
| 6 | C14orf182 | 3.1750631 | 4.99E−14 |
| 7 | CADM1 | 2.9656603 | 3.92E−29 |
| 8 | LAIR2 | 2.9488478 | 5.98E−95 |
| 9 | TNFRSF18 | 2.8933306 | 3.00E−306 |
| 10 | FANK1 | 2.8928053 | 7.94E−22 |
| 11 | AC017002.1 | 2.8106666 | 7.52E−97 |
| 12 | LAYN | 2.8076181 | 1.85E−67 |
| 13 | CUL9 | 2.664441 | 1.92E−12 |
| 14 | MZB1 | 2.6608176 | 5.36E−18 |
| 15 | FOXP3 | 2.6518457 | 1.39E−37 |
| 16 | SOX4 | 2.637437 | 6.77E−38 |
| 17 | ZBTB32 | 2.6331844 | 2.42E−11 |
| 18 | LAPTM4B | 2.6117795 | 8.46E−09 |
| 19 | AC002331.1 | 2.5963533 | 6.69E−63 |
| 20 | TNFRSF9 | 2.4803348 | 8.77E−105 |
| 21 | NGFRAP1 | 2.4254808 | 1.69E−32 |
| 22 | IL32 | 2.4124053 | 0 |
| 23 | CRADD | 2.389674 | 5.75E−25 |
| 24 | PTPLA | 2.3780098 | 3.40E−07 |
| 25 | CARD16 | 2.3760853 | 2.94E−271 |
| 26 | MAGEH1 | 2.3666537 | 1.36E−107 |
| 27 | GCNT1 | 2.3362417 | 1.02E−07 |
| 28 | CD79B | 2.330269 | 3.97E−30 |
| 29 | CD27 | 2.2939112 | 5.48E−232 |
| 30 | EPHX2 | 2.2846296 | 3.35E−07 |
| 31 | SYNGR2 | 2.2718766 | 1.25E−48 |
| 32 | HLF | 2.2631228 | 0.000500769 |
| 33 | LTA | 2.2349174 | 7.37E−05 |
| 34 | ACP5 | 2.233677 | 2.50E−46 |
| 35 | PTP4A3 | 2.2089796 | 1.96E−05 |
| 36 | TIGIT | 2.1930175 | 6.29E−224 |
| 37 | DNPH1 | 2.179427 | 2.10E−169 |
| 38 | CTSC | 2.1694787 | 1.07E−179 |
| 39 | HTATIP2 | 2.116239 | 4.69E−68 |
| 40 | PKM | 2.0981705 | 4.48E−189 |
| 41 | SAT1 | 2.062402 | 0 |
| 42 | BATF | 2.0623808 | 1.92E−222 |
| 43 | OTUD5 | 2.04023 | 7.86E−08 |
| 44 | ADAT2 | 2.0244803 | 0.00010732 |
| 45 | OAS1 | 2.0076995 | 1.86E−13 |
| 46 | CTLA4 | 2.0070717 | 4.71E−111 |
| 47 | GLRX | 2.0028965 | 7.69E−107 |
| 48 | MIR4435-1HG | 2.0020456 | 9.51E−135 |
| 49 | LTB | 1.9985057 | 2.94E−271 |
| 50 | TBC1D4 | 1.9641753 | 3.05E−49 |

Upregulated genes in CD4_{IL2RALO}

| | | | |
|---|---|---|---|
| 1 | FANK1 | 1.675839 | 0.001288122 |
| 2 | IL2RA | 1.6441387 | 8.91E−47 |
| 3 | AC002331.1 | 1.642346 | 4.13E−13 |
| 4 | RTKN2 | 1.6299212 | 3.71E−18 |
| 5 | TNFRSF9 | 1.5882788 | 3.51E−25 |
| 6 | RP11-1399P15.1 | 1.4550278 | 4.68E−12 |
| 7 | SAT1 | 1.4457726 | 7.71E−127 |
| 8 | PMAIP1 | 1.3851801 | 5.56E−70 |
| 9 | IL32 | 1.2847056 | 2.23E−125 |
| 10 | LAYN | 1.2706443 | 3.43E−06 |
| 11 | HPGD | 1.2191882 | 1.04E−05 |
| 12 | MAGEH1 | 1.2093228 | 1.04E−14 |
| 13 | TIGIT | 1.2051893 | 1.92E−44 |
| 14 | MIR4435-1HG | 1.1903769 | 2.81E−29 |
| 15 | FOXP3 | 1.1896392 | 0.005114286 |
| 16 | CARD16 | 1.1778805 | 1.33E−41 |
| 17 | HTATIP2 | 1.1391697 | 8.08E−10 |
| 18 | TBC1D4 | 1.1227332 | 2.41E−08 |
| 19 | LTB | 1.1142904 | 3.06E−63 |
| 20 | LINC00152 | 1.0199449 | 3.65E−38 |

Tregs were abundant constituents of the bladder tumor microenvironment with demonstrated heterogeneity. Two states of Tregs were found: CD4m2RAHI and CD4m2RALo, together constituting 26% +1.9% (mean +SEM) of tumor-infiltrating CD4+cells, which co-expressed FO)a) 3 (CD4_{m2RAHI}: log2(FC)=2.7; CD4_{IL2RALO}: log2(FC)=1.2) and known immune checkpoints, including IL2RA, TIGIT, TNFRSF4/9/18, and CD27 (CD4m2RAFH and CD4IL2RALO: log2(FC) >0.65; FIGS. 18B, 18C, and 19A). With the exception of TIGIT, these immune checkpoints are minimally expressed by other CD4+states, such as CD4cm (FIG. 19A). The two Treg states were distinguished by higher expression of IL2RA, TNFRSF4, TNFRSF9, and TNFRSF18 in CD4m2RAHI cells (CD4m2RAHI: log2(FC)=2.5-3.6; CD4_{IL2RALO}: log2(FC)=0.4-1.6; FIG. 19A; Table 8). Of note, both Treg states were significantly enriched in tumor compared with adjacent non-malignant tissue (CD4m2RAHI: 14.3% versus 4.6%, p=0.002; CD4m2RALO: 11.1% versus 6.7%, p=0.002; exact permutation test; FIG. 18E). It was confirmed, by flow cytometry from 7 additional bladder tumors, that multiple tumors contained distinct regulatory states that expressed graded protein levels of IL2RA and co-expressed significantly different levels of immune checkpoints, such as TNFRSF 18 (p <0.05 for TNFRSF18 expression in FOXP3+CD25^{b0}w versus CD25^{hi} populations by Wilcoxon signed-ranked test; FIG. 19B). This heterogeneity may be consequential because Tregs expressing higher levels of immune checkpoints have been shown to be correlated with poorer outcomes in non-small cell lung cancer. Both regulatory states also demonstrated a common tumor-specific gene expression program that included several heat shock proteins compared with non-malignant tissue.

### Example 11

### Regulatory T cells are Clonally Expanded in Bladder Tumors

With reference to Example 3 above, this Example describes the results of additional experiments performed to investigate the TCR sequence in the same single cells for which the whole-transcriptome data had been acquired previously. In these experiments, the complementarity-determining region 3 (CDR3) of the TCR alpha (TRA) and beta (TRB) loci from the barcoded full-length cDNA library were PCR-amplified and sequenced to saturation. After filtering or matching whitelisted cell barcodes (Cell Range), this approach yielded 11,081 CD4+ T cells and 5,779 CD8+ T cells with paired TRA and TRB CDR3 sequences (e.g., 49% and 47% recovery, respectively). These results are consistent with expected frequencies based on the average recovery of individual TRA (CD4+, 54%; CD8+, 50%) and TRB (CD4+, 68%; CD8+, 67%) sequences across whitelisted cells. Overall, the TCR repertoire was more restricted in the tumor microenvironment than in adjacent non-malignant tissue based on two analyses. First, in intratumoral CD4+ T cells, 10.8% +1.6% of unique clonotypes are shared by 2 or more cells; this degree of sharing was significantly greater than in the non-malignant compartment (5.1% +1.6%, unpaired t test, p=0.033) and was not seen in blood from healthy donors (0.12%-0.16%) or from publicly available reference circulating CD4+ T cell data (0%) (data not shown). Second, there was a skewing of the intratumoral CD4+ T cell repertoire toward an increased cumulative frequency of clonotypes over fewer cells and a corresponding higher Gini coefficient (0.21 for tumor versus 0.05 for non-malignant tissue, Wilcoxon signed-rank test with Benj amini-Hochberg correction, p=0.009) compared with the non-malignant compartment and healthy controls. Assigning TCR sequences to cells with cluster identities (9,770 CD4+and 5,151 CD8+ T cells with a paired TRA/TRB had an assigned phenotypic cluster or 49% and 48% of all T cells with assigned clusters, respectively; merged TCR sequences and phenotypic clusters for CD4+and CD8+ T cells) revealed that clonal expansion of Tregs contributes to intratumoral CD4⁺ T cell repertoire restriction. Compared with paired non-malignant tissue, both regulatory states exhibited increased Gini coefficients in tumors (CD4m2RAF11: Gini ₜᵤmor 0.17 versus Gininormai 0, p=0.003; CD4_{IL2RALO}: Giniₜᵤₘₒᵣ 0.06 versus Gini ₙₒᵣₘₐₗ 0.003, p=0.009; exact permutation test; FIG. 19C). The most expanded clonotypes within the Tregs were specific to regulatory cells but not other cell states (all single cells expressing the top expanded regulatory clonotypes are shown in FIG. 19D). The CXCL13-expressing state CD4_{c}x_{c}ii3 (discussed in greater detail below) was also restricted in tumors (Giniₜᵤₘₒᵣ 0.07 versus Giniₙₒᵣₘₐₗ 0, p=0.02, exact permutation test; FIG. 19C). Gini coefficients for CD4+states did not differ significantly by anti-PDL1 treatment. In contrast, although repertoire restriction was also seen in CD8+ T cells from the same samples, this was observed in both tumor (percent unique clonotypes shared between cells: 15.1% ±1.1%; Giniₜₕₘₒᵣ: 0.36% +0.04%) and non-malignant compartments (percent unique clonotypes shared between cells: 14.6% +0.2%; Giniₙₒᵣₘₐₗ: 0.39% +0.06). Furthermore, no significant increase in Gini coefficient in tumor over non-malignant tissue was seen for any CD8+state, including with anti-PD-L1 treatment. Hence, an important contributor to increased repertoire restriction of tumor-infiltrating CD4+over non-malignant tissue, which was not seen in the CD8+ compartment, involved clonal expansion of several distinct regulatory T cell states that differed in their levels of immune checkpoint expression, which may be driven by tumor-associated antigens and the tumor-specific microenvironment.

### Example 12

### Bladder Tumors Possess Multiple Cytotoxic CD4+ T Cell States

This Example describes the results of additional experiments illustrating that bladder tumors possess multiple cytotoxic CD4+cell states. In addition to the regulatory populations described in Example 11 above, the results from additional experiments identified two (2) distinct populations of cytotoxic CD4⁺ T cells in all samples, which constituted 15 ±0.9% of tumor-infiltrating CD4⁺ T cells. CD4_{GZMB} and CD4_{GZMK} cytotoxic cells expressed a core set of cytolytic effector molecules (1og2(-FC) >0.5, Padj <0.05): GZMA (granzyme A), GZMB (granzyme B), and NKG7 (a granule protein that translocates to the surface of natural killer (NK) cells following target cell recognition; Medley et al., 1996) (FIGS. 18B, 18C, and 20A; Table 9). Each cytotoxic CD4+state was distinguished by the expression of specific effector molecules. CD4_{GZMB} cells co-expressed high levels of GZMB, the pore-forming protein PRF1 (perforin), and the granule-associated proteins GNLY and NKG7 (CD4_{GZMB}: log2(-FC)=5.7, 3.4, 5.1, and 4.4, respectively), whereas CD4_{GZMK} cells co-expressed high levels of the distinct GZMK (granzyme K) and lower levels of NKG7 (CD4_{GZMK}:log2(FC)=6.3 and 3.9) (see, e.g., FIG. 20A and Table 9). These shared cytolytic molecules were not expressed by other CD4+ states, including regulatory and central memory T cells (see, e.g., FIG. 20A). Cytotoxic CD4+ cells co-expressed additional molecules, which may further contribute to anti-tumor effector function. Notably, IFNG was expressed by both cytotoxic states, which may contribute to tumor cell death, including ferroptosis (CD4_{GZMB} and CD4_{GZMK}: log2(-FC)=2.1). Notably, the minority of CD4_{GZMB} cells that expressed IFNG appeared to also express TNF as well as specific immune checkpoints, such as PDCD1, LAGS, and HAVCR2 (TIM3) (FIG. 20A). A larger proportion of CD4_{GZMB} cells expressed CXCR6 (CD4_{G}zmB: log2(FC)=1.3; FIG. 20A). It was reported previously that this chemokine is expressed in regulatory and non-regulatory CD4+ TILs from colorectal carcinoma, nasopharyngeal carcinoma, and renal cell carcinoma and, together with its ligand CXCL16, can mediate TIL chemotaxis. Finally, CD4_{G}zivifi and CD4_{G}zmK cells did not express high levels of other checkpoints associated with regulatory T cells, such as IL2RA, TIGIT, or TNFRSF4/9/18 (log2(FC) <0.5; FIG. 20A), nor did they express the exhaustion marker TOX (Table 9). Similar states were found with unbiased clustering without batch correction for paired tumor- and non-malignant-derived CD4+cells from individual patients (data not shown). A listing of the top biomarker genes that were found differentially expressed in 2 populations of cytotoxic CD4+cells overexpressing GZMB (CD4_{GZMB}) or GZMK (CD 4 _{GZMK}) is presented in Table 9 below.

TABLE 9
Exemplary gene signature of cytotoxic CD4⁺ cells.
These are differentially expressed (e.g., upregulated)
marker genes for each CD4+ population (versus all
other CD4+ populations), identified using scanpy on single cells.

| | | | |
|---|---|---|---|
| ID | Gene Name | log2FC | Adjusted P value |

Upregulated genes in cytotoxic CD4+ cells
overexpressing GZMB (CD4_{GZMB})

| | | | |
|---|---|---|---|
| 1 | FGFBP2 | 7.0650206 | 5.35E−16 |
| 2 | GZMB | 5.7132688 | 0 |
| 3 | KLRD1 | 5.677851 | 0.000613253 |
| 4 | GNLY | 5.137486 | 6.78E−179 |
| 5 | GZMH | 4.7940173 | 5.37E−106 |
| 6 | CCL5 | 4.6393914 | 0 |
| 7 | NKG7 | 4.3652754 | 1.67E−129 |
| 8 | CCL4 | 4.1093554 | 4.42E−196 |
| 9 | GZMA | 3.4416566 | 2.48E−247 |
| 10 | CCL3 | 3.421906 | 6.98E−08 |
| 11 | PRF1 | 3.4135134 | 5.78E−84 |
| 12 | HOPX | 3.3846326 | 6.55E−38 |
| 13 | CSF2 | 2.7414842 | 0.00450887 |
| 14 | ITGA1 | 2.6843643 | 0.007120228 |
| 15 | CTSW | 2.647971 | 3.76E−22 |
| 16 | ZEB2 | 2.3496747 | 5.13E−08 |
| 17 | HAVCR2 | 2.2172813 | 4.05E−17 |
| 18 | CLIC3 | 2.1982718 | 0.018107221 |
| 19 | AC092580.4 | 2.18053 | 6.62E−43 |
| 20 | LAG3 | 2.1443207 | 1.11E−28 |
| 21 | IFNG | 2.1095948 | 1.36E−14 |
| 22 | NBL1 | 2.0776591 | 0.037218552 |
| 23 | CXCL13 | 2.0132163 | 8.23E−26 |
| 24 | DAPK2 | 1.9157312 | 0.014739453 |
| 25 | C12orf75 | 1.8908454 | 1.80E−25 |
| 26 | F2R | 1.7739519 | 0.004375431 |
| 27 | APOBEC3G | 1.7378328 | 6.97E−23 |
| 28 | ID2 | 1.7112797 | 8.98E−85 |
| 29 | PTMS | 1.7090127 | 0.000355587 |
| 30 | SYTL2 | 1.6670508 | 0.020850646 |
| 31 | CEBPD | 1.6360918 | 0.000514797 |
| 32 | PDCD1 | 1.5955342 | 2.64E−10 |
| 33 | ANKRD32 | 1.5700119 | 0.00225073 |
| 34 | CD63 | 1.5541701 | 4.19E−35 |
| 35 | SLA2 | 1.5468614 | 0.00054131 |
| 36 | GDE1 | 1.5249468 | 3.12E−05 |
| 37 | CTSD | 1.4980711 | 1.57E−15 |
| 38 | ALOX5AP | 1.4735985 | 4.91E−50 |
| 39 | CHST12 | 1.4732744 | 7.89E−06 |
| 40 | BHLHE40 | 1.4659352 | 3.87E−11 |
| 41 | CST7 | 1.4341874 | 8.69E−51 |
| 42 | LMO4 | 1.400079 | 0.010790392 |
| 43 | CD52 | 1.3964664 | 1.74E−109 |
| 44 | OASL | 1.3411924 | 0.001011162 |
| 45 | CD99 | 1.3325849 | 2.93E−53 |
| 46 | SH3BGRL3 | 1.3077241 | 3.07E−111 |
| 47 | LGALS1 | 1.307164 | 4.98E−24 |
| 48 | MYO1F | 1.3060452 | 0.016486095 |
| 49 | CXCR6 | 1.30304 | 1.49E−30 |
| 50 | MAP3K8 | 1.2687898 | 2.99E−05 |

Upregulated genes in cytotoxic CD4+ cells
overexpressing GZMK (CD4_{GZMK})

| | | | |
|---|---|---|---|
| 1 | GZMK | 6.299831 | 0 |
| 2 | CRTAM | 5.3761077 | 1.55E−20 |
| 3 | CCL5 | 4.397413 | 0 |
| 4 | CCL4 | 4.3362465 | 5.51E−195 |
| 5 | NKG7 | 3.935101 | 1.29E−100 |
| 6 | GZMA | 3.4660788 | 1.08E−241 |
| 7 | IL10 | 3.274969 | 0.001347646 |
| 8 | GZMH | 2.8829727 | 1.13E−24 |
| 9 | CHI3L2 | 2.4820707 | 0.013633846 |
| 10 | ITM2C | 2.3212488 | 3.35E−10 |
| 11 | CST7 | 2.199797 | 1.39E−118 |
| 12 | SRRT | 2.1822314 | 8.74E−05 |
| 13 | IFNG | 2.1427507 | 2.90E−12 |
| 14 | CTSW | 2.0981808 | 1.41E−10 |
| 15 | LYAR | 2.0675004 | 3.16E−20 |
| 16 | CMC1 | 1.7968861 | 1.37E−06 |
| 17 | TUBA4A | 1.6023986 | 6.41E−26 |
| 18 | ANKRD32 | 1.5524027 | 0.047775583 |
| 19 | APOBEC3G | 1.5023665 | 4.39E−12 |
| 20 | DUSP2 | 1.4793888 | 2.20E−61 |
| 21 | NUCB2 | 1.452009 | 0.00035042 |
| 22 | OASL | 1.4459441 | 0.00437414 |
| 23 | TRAT1 | 1.4313006 | 2.05E−36 |
| 24 | CLDND1 | 1.3736662 | 2.27E−16 |
| 25 | TC2N | 1.3587799 | 5.67E−05 |
| 26 | LITAF | 1.2814052 | 1.28E−18 |
| 27 | ANXA1 | 1.2292562 | 7.74E−35 |
| 28 | GIMAP4 | 1.211042 | 6.22E−12 |
| 29 | IFNGR1 | 1.1924714 | 7.68E−06 |
| 30 | CXCR4 | 1.1355659 | 5.30E−101 |
| 31 | GZMB | 1.0293607 | 1.24E−05 |
| 32 | PRF1 | 1.0262915 | 0.010170617 |

Further experiments were perform to validate the presence and functional heterogeneity of cytotoxic CD4+ T cells using several orthogonal and complementary methods. Using flow cytometry, the presence of cytotoxic CD4+ T cells with an effector memory (CCRT CD45RA⁻) or effector (CCRT CD45RA+) phenotype that express GZMB, GZMK, and perforin protein was confirmed by flow cytometry in tumors from multiple independent replicate samples (N=7 tumors; FIG. 20B). Across this sample set, 9% +2.9% (mean +SEM) of CD4+FOXP3⁻ CCR7″ cells expressed GZMB, whereas 16% +4.5% expressed GZMK and 5.3% +2.6% expressed perforin (FIG. 20C, left panel), at lower frequencies than CCRT CD8+cytotoxic cells from the same patients. Importantly, 25.9% ±8.7% of GZMB+CD4+FOXP3″ CCRT and 8.6% +3.5% of GZMK+CD4+FOXP3⁻ CCM⁻ cytotoxic T cells showed co-expression of perforin with granzymes, in agreement with the scRNA-seq data (FIG. 20C, right panel); these frequencies of granzyme and perforin co-expression were lower than those of CCM' CD8+ cytotoxic cells from the same patients. Importantly, CD45⁻ bladder tumor cells express multiple major histocompatibility complex (MHC) class II molecules (data not shown), which would allow antigen recognition by TCRs expressing CD4 as a co-receptor. Flow cytometry of a separate set of 11 muscle-invasive bladder tumors confirms the functional capacity of cytotoxic CD4+ T cells to produce multiple cytokines. In agreement with the scRNA-seq data, 56%+4.8% (mean +SEM) of CD4+CCR7⁻ cells were polyfunctional and could produce both IFNγand tumor necrosis factor alpha (TNF-α), whereas a minority of these cells only secrete IFNγ alone or TNF-αalone after stimulation and, therefore, may demonstrate signs of exhaustion (IFNγ+TNF-α⁻: 2.0% ±0.76%; IFNγ⁻TNF-α+: 19% ±3.3%) (FIGS. 20C and 20F). The frequency of polyfunctional cytotoxic CD4+ T cells was similar to stimulated CD8+CCR7⁻ T cells from the same patients (IFNγ+TNF-α+: 55% +6.3%), although CD8+CCR7⁻ T cells that were monofunctional demonstrated an increased trend toward preferential IFNγproduction alone over TNF-αproduction compared with cytotoxic CD4+ T cells (IFNγ+TNF-α⁻: 14% +4.7%; IFNγ⁻TNF-α+: 7.2% +2.1%).

As further validation of the cytotoxic CD4+ T cell phenotype in tissue, multiplex immunofluorescence tissue staining of bladder tumor tissue from a patient in the scRNA-seq dataset demonstrated CD4+ T cells that also expressed GZMB or GZMK (FIG. 20F, top row) at levels not seen with negative control staining (FIG. 20F, bottom row).

Overall annotation of clusters from the scRNA-seq data was supported by an independent analysis that assigns each single cell to the best-known published immune subset profiled by bulk expression analysis after sorting (SingleR). This corroborated the identification of Tregs (90% and 78% of CD4IL2RAHI and CD4Th2RALO cells are assigned to Treg annotations, respectively) and further demonstrated that both cytotoxic CD4+states are most similar to CD8+ effector memory T cells (37% and 45% of CD4_{GZMB} and CD4_{GZMK} cells, respectively, are assigned to effector memory CD8+cell annotations), reinforcing their cytotoxicity profile. Finally, an internal comparison of the transcriptional profiles from CD4+and CD8+ TIL clusters from our scRNA-seq data indicated that, although most CD4+clusters are most similar to other CD4+clusters, cytotoxic CD4+ T cells are an exception. CD4_{GZMB} cytotoxic cells were most correlated with tumor-specific CD8ENTPD1 cells (Pearson correlation coefficient=0.92), whereas CD4_{GZMK} cytotoxic cells were most correlated with CD8o4 and CD8NAivE cells (Pearson correlation coefficient=0.98 for both). The tumor-specific gene expression program of these cytotoxic CD4+cells was marked by heat shock protein expression in both states as well as tumor overexpression of CXCL13 and numerous immune checkpoints (TNERSF181LAG3ITIGITI HAVCR2) as well as ENTPD1 within CD4_{G}zivfl3 cells (see, e.g., Table 7).

### Example 13

### Cytotoxic CD4+ T Cells were Enriched and Clonally Expanded in Bladder Tumors

Of the 2 cytotoxic CD4+states, CD4_{GZMK} cells were significantly enriched in abundance in tumors (CD4_{GZMK} in tumor versus nonmalignant tissues: 7.2% +0.5% versus 5.0% +0.5%, exact permutation test, p=0.01; FIG. 18E). Overall, the CD4+compartment exhibited a bias toward regulatory over cytotoxic CD4+ T cells in tumors (regulatory CD4+/cytotoxic CD4+ ratio=1.8 +0.2) compared with non-malignant tissues, where proportions of regulatory and cytotoxic CD4+ T cells were more balanced (regulatory CD4+/cytotoxic CD4+ratio=1.1 +0.2, t test, p=0.04; FIG. 20G). Cytotoxic CD4+ T cell states contributed to intratumoral CD4+ repertoire restriction. Both cytotoxic CD4+ T cell states have significantly increased Gini coefficients in tumor compared with non-malignant tissues, with CD4_{GZMS}representing the more restricted cytotoxic state in tumors (CD4_{GZMB}: Ginitᵢᵢ.0.21 versus Gini..₁0.06; CD4G_{ZMK}: Ginitumor 0.12 versus Gininormal 0; exact permutation test, p=0.04 for CD4_{G}zmi3 and p=0.002 for CD4_{G}ZMK; FIG. 2011). Hence, unbiased dscRNA-seq revealed that heterogeneous cytotoxic CD4+ T cells, a subset of which are closely related to conventional cytotoxic CD8+ T cells based on their functional program, are unexpected but frequent constituents of the bladder tumor microenvironment, some of which are quantitatively enriched in tumors. The tumor-specific clonal expansion of both cytotoxic CD4+states suggests that their restricted repertoire may result from recognition of MHC class II cognate antigens that may include bladder tumor antigens.

### Example 14

### Cytotoxic CD4+ T Cells Possessed Lytic Capacity Against Autologous Tumor Cells that was Restricted by Autologous Tregs

Additional experiments were performed to validate the functional relevance of cytotoxic CD4+in bladder tumors. In these experiments, CD4+ TILs were isolated by fluorescence-activated cell sorting (FACS) and then cultured the cells ex vivo with interleukin-2 (IL-2). These cells were then co-cultured with autologous tumor cells in an imaging-based time-lapse cytotoxicity assay, assessing for cell death with Annexin V. It was observed that expanded CD4+ TILs were cytotoxic and could trigger increased tumor apoptosis (“CD4ₜₒₜₐutumor,” FIG. 201, left panel). However, when the same co-cultures was performed but with CD4+ TILs from the same patient that were depleted of Tregs, it was observed that killing was increased (“CD4_{eff}:tumor,” FIG. 201, left panel), indicating that autologous Tregs can inhibit the activity of cytotoxic CD4+ T cells. Significant tumor death was seen in co-cultures with CD4eff TILs compared with tumors alone (FIG. 201, left panel; representative of 3 independent experiments from different patients). Furthermore, the cytotoxic activity of CD4_{eff}was at least partially dependent on MHC class II recognition because tumor apoptosis was inhibited with pre-incubation with a pan-anti-MHC class II antibody that was not seen with an isotype control antibody (FIG. 201, right panel; representative of 2 independent patients). Independent experiments with an alternative death indicator (Cytotox Red) confirmed increased autologous tumor killing with tumor/CD4_{eff}co-cultures, MHC class II dependence of CD4_{eff} killing as well as similar MHC class I-dependent autologous tumor killing with expanded CD8+ T cells. Hence, flow cytometry and functional analyses from multiple independent patients confirmed not only that cytotoxic CD4+ T cells expressed cytolytic proteins, such as granzymes and perforin, in tumor tissue but that these cells can recognize bladder tumor antigens in an MHC class II-dependent fashion and were functionally competent to lyse autologous tumor cells in a manner that can be suppressed by autologous Tregs.

### Example 15

### Proliferating CD4+ T Cells Contained Regulatory and Cytotoxic Cells

As discussed above, induction of proliferating T cells can be beneficial for anti-tumor immune responses. Proliferating CD4+ T cells are rapidly induced in the periphery within weeks of initiating checkpoint blockade in prostate cancer patients and in separate cohorts of thymic epithelial tumors and non-small cell lung cancer treated with anti-PD-1; a higher fold change in Ki67+cells among PD-1+CD8+ T cells in the periphery after a week was predictive of durable clinical benefit, progression- free survival, and (in the non-small cell lung cancer cohorts) overall survival. Within the tumor-infiltrating CD4+ T cell compartment in transitional cell carcinoma (TCC), the results of these experiments also identified proliferating cells (CD4PROLIF) expressing MK/67, microtubule-associated markers (e.g., STMN1ITUBB), and DNA-binding proteins associated with cell cycle progression, such as PCNA, HMGBJ, and HMGB2, which were expressed at lower levels in regulatory and cytotoxic CD4+ T cells (CD4pRouF:log2(FC) >2.1; FIG. 18C; Table 10). A similar signature was also seen in the CD8+ compartment (CD8p_{R}ouF; FIG. 17; Table 7). Higher-resolution clustering revealed that this proliferating state is comprised of discrete groups of cells co-expressing regulatory or cytotoxic genes but not both simultaneously (see, e.g., FIG. 21A). Flow cytometry analysis of separate TCC samples confirmed the presence of discrete regulatory or cytotoxic populations of Ki67+ CD4+ T cells that co-expressed CD25, GZMB, or GZMK (see, e.g., FIG. 21B). Across multiple independent samples, 4.7% +1.0% (mean +SEM) of CD4⁺ FOXP3⁺ cells co-expressed Ki67 and CD25, whereas 1.2% 0.5% of CD4+FOXP3⁻ CCRT cells co-expressed Ki67 and GZMB, and 1.0% +0.1% of CD4+FOXP3⁻ CCM' cells co-expressed Ki67 and GZMK (N=7 tumors). Proliferating Ki67⁺ GZMB⁺ cells are also seen, using flow cytometry, within the CD8+ compartment of TCC patients. Examination of exact TCR clonotype sharing of the most expanded CD4_{PROLIF} clones identified sharing with regulatory and cytotoxic CD4⁺ T cells, further underscoring the contribution of each state to CD4PROLIF cells (see, e.g., FIG. 21C). A listing of the top biomarker genes that were found differentially expressed in proliferating CD4⁺ cells is presented in Table 10 below.

TABLE 10
Exemplary gene signature of proliferating CD4⁺ cells.
These are differentially expressed (e.g., upregulated)
marker genes for each CD4+ population (versus all
other CD4+ populations), identified using scanpy on single cells.

| ID | Gene Name | log2FC | Adjusted P value |
|---|---|---|---|
| 1 | RRM2 | 8.237012 | 7.28E−06 |
| 2 | KIAA0101 | 7.893176 | 8.75E−36 |
| 3 | UBE2C | 7.8063097 | 2.18E−07 |
| 4 | TK1 | 7.7976174 | 1.85E−14 |
| 5 | TYMS | 7.6552815 | 1.49E−13 |
| 6 | BIRC5 | 7.598636 | 0.000930033 |
| 7 | CCNB2 | 6.3249803 | 0.018405642 |
| 8 | MKI67 | 6.241481 | 1.02E−05 |
| 9 | GGH | 6.2379036 | 3.39E−05 |
| 10 | RAD51AP1 | 6.210985 | 0.018203133 |
| 11 | CCNA2 | 6.1506104 | 0.041789775 |
| 12 | ZWINT | 6.080827 | 0.000380669 |
| 13 | ASF1B | 5.7857056 | 0.019733783 |
| 14 | TOP2A | 5.677682 | 2.36E−05 |
| 15 | CENPU | 5.54058 | 0.000440188 |
| 16 | CENPW | 5.5232306 | 0.005637506 |
| 17 | STMN1 | 5.454512 | 4.47E−49 |
| 18 | CLSPN | 4.960939 | 6.28E−05 |
| 19 | FBXO5 | 4.5409217 | 0.01850428 |
| 20 | CKS1B | 4.5127125 | 3.02E−11 |
| 21 | MCM7 | 4.4613986 | 2.60E−05 |
| 22 | CDK1 | 4.297339 | 0.031570099 |
| 23 | CENPF | 4.281929 | 1.89E−06 |
| 24 | UBE2T | 4.2393255 | 0.001466211 |
| 25 | NUSAP1 | 4.1594667 | 6.89E−12 |
| 26 | DTYMK | 3.917083 | 0.000443845 |
| 27 | SMC2 | 3.8965776 | 0.000162269 |
| 28 | CDKN3 | 3.8838048 | 0.003118731 |
| 29 | TMEM106C | 3.6883872 | 5.65E−08 |
| 30 | FEN1 | 3.6828945 | 0.040516826 |
| 31 | TUBB | 3.6041403 | 9.19E−22 |
| 32 | MAD2L1 | 3.5300026 | 0.000255325 |
| 33 | CENPK | 3.512722 | 0.04635914 |
| 34 | NUDT1 | 3.3271449 | 1.92E−09 |
| 35 | MCM3 | 3.3189616 | 0.004198779 |
| 36 | MCM5 | 3.2263293 | 0.001661108 |
| 37 | RFC2 | 3.134066 | 0.045366373 |
| 38 | PCNA | 3.0682387 | 1.56E−08 |
| 39 | TUBA1B | 3.0375028 | 3.44E−29 |
| 40 | DUT | 2.74817 | 2.32E−18 |
| 41 | EZH2 | 2.7216113 | 0.035343693 |
| 42 | HIST1H4C | 2.613 | 3.17E−10 |
| 43 | DEK | 2.5249553 | 5.33E−18 |
| 44 | SAE1 | 2.4889328 | 0.004709804 |
| 45 | HMGB2 | 2.4746351 | 1.05E−26 |
| 46 | STRA13 | 2.4414928 | 3.08E−05 |
| 47 | NME1 | 2.440946 | 0.007066155 |
| 48 | HLA-DRA | 2.4323235 | 1.14E−14 |
| 49 | DNAJC9 | 2.4208682 | 4.04E−05 |
| 50 | CBX5 | 2.3869922 | 0.043464544 |

Given that regulatory and cytotoxic CD4+ T cells were heterogenous and composed of cells that were proliferating to a different extent, existing clusters may fail to resolve the separate contribution of specific expression programs from subsets with different proliferative capacity. Hence, pseudotime analysis was used to separate regulatory and cytotoxic cells into proliferating and non-proliferating components. This analysis divided CD4pRouF cells into two groups, each lying along a branch specific for proliferating regulatory or cytotoxic CD4+ T cells, with separate branches for non-proliferating regulatory and cytotoxic cells (FIG. 21D). This underscored that regulatory and cytotoxic CD4⁺ T cells consist of distinct proliferating and non-proliferating states in TCC, based on transcriptomic and clonotypic analyses.

### Example 16

### A Signature of Cytotoxic CD4+ T Cells Predicts Clinical Response to Anti-PD-L1

The Example describes the results of experiments performed to assess the importance of the specific proliferating and non-proliferating cytotoxic CD4⁺ T cell states for patient outcomes. In these experiments, branched expression analysis modeling (BEAM) was performed to identify all genes that were differentially expressed between branches at branchpoint 1 of the pseudotime trajectory. This branchpoint divided proliferating cytotoxic CD4⁺ T cells, non-proliferating cytotoxic CD4⁺ T cells, and all other regulatory cells (FIG. 21D, right panel). Hierarchical clustering identified genes upregulated preferentially in the proliferating cytotoxic branch (cluster 7) or the non-proliferating cytotoxic branch (cluster 4) but not in regulatory branches within this analysis (all genes with q <0.05; heatmap of clusters and branches in FIG. 21E). From this analysis, a gene signature was developed and consisted of genes that were upregulated specifically in proliferating or non-proliferating cytotoxic CD4⁺ T cells (from cluster 7: ABCB1; from cluster 4: APBA2, SLAMF7, GPR18, and PEG10; FIG. 21E) but were not upregulated in any of the CD8⁺ T cell states from our scRNA-seq analysis (Table 7). Additional analyses were performed to test this gene signature's ability to predict treatment response using bulk RNA-seq data from pre-treatment tumors from a separate phase 2 trial of atezolizumab for metastatic bladder cancer (IMvigor210). In 244 metastatic bladder cancer patients with pre-treatment RNA-seq data, immunohistochemistry (IHC) information regarding immune phenotype (immune desert, immune excluded, or inflamed), and information regarding clinical response, this gene signature was significantly correlated with clinical response to anti-PD-L1 therapy in inflamed samples (p=0.037, two-tailed t test, N=62 inflamed samples; FIG. 21F), which was not seen in samples with an immune-excluded or immune desert phenotype. Hence, in these analyses, a composite signature containing genes that discriminated proliferating and non-proliferating cytotoxic CD4+ T cells was used to assess the specific contributions of these discrete states and found that this signature is associated with response to PD-1 blockade in a large cohort of TCC patients. This result highlights the potential clinical importance of possessing intratumoral cytotoxic CD4+ T cell activity in response to anti-PD-L1 treatment.

### Example 17

### Circulating Cytotoxic CD4⁺ T Cells Share Exact Antigenic Specificity with Intratumoral Cytotoxic CD4⁺ T Cells in Bladder Cancer Patients

To assess the degree of shared immune responses in the periphery of bladder cancer patients, sorted CD3⁺ CD4⁺ or CD3⁺ CD8⁺ peripheral blood mononuclear cells (PBMCs) were also sequenced from the same individuals from whom tumor and adjacent normal tissue sequenced had been obtained, at the time of surgical resection. For the 4 individuals treated with atezolizumab prior to surgery, an additional timepoint was also sequenced prior to starting atezolizumab. Droplet-based scRNAseq (10× Genomics) with custom amplification of the TCR alpha and beta locus was obtained as with tumor. Expression data was obtained from a total of 157,052 single cells (CD3⁺ CD4⁺ and CD3⁺ CD8⁺ , blood and tissue), with recovery of paired TCR alpha and beta chain information from 63,572 of these cells (40% paired TCR recovery). Unbiased clustering was conducted using the scanpy pipeline (Wolf et al. Genome Biol. 2018) with ComBat batch correction (Johnson et al., Biostatistics, 2007). Adjusting batch effects in microarray expression data using empirical Bayes methods. Biostatistics. 8, 118-27). This revealed the presence of canonical T cell populations, notably including GZMB+and GZMK+T cells as well as a population of proliferating MKI67+GZMK+T cells (FIGS. 22A-22B), which included contributions from both sorted CD4 and CD8+ T cells and were found in the circulation as well as the tumor of these patients (FIG. 22C).

TCR repertoire analysis from combined blood and tumor clustering revealed that cytotoxic CD4+circulate in the periphery of bladder cancer patients where they share a restricted repertoire with the tumor. GZMB+and GZMK+CD4+ T cells in the blood are clonally expanded, with >50% of GZMB+and >25% of GZMK+CD4+unique TCR clonotypes being shared by 3 or more cells (FIG. 23A). Moreover, GZMB+and GZMK+CD4+ T cells comprise a substantial fraction of the circulating T cells that share clonotypes with tumor in both pre- and post-treatment blood samples from atezolizumab-treated patients (FIG. 23B), and cytotoxic CD4+in the circulation that share clonotypes with tumor demonstrate a trend towards higher Gini coefficient (indicative of a more restricted TCR repertoire) compared with circulating T cells that do not share specificity with tumor-resident T cells (FIG. 23C). In the tumor, GZA18+ and GZMK+CD4+ T cells are one of several clonally expanded populations (FIGS. 23D and FIG. 23F), and comprise a large proportion of the CD4+ T cell populations that share specificity with blood (FIG. 23E).

By comparison, a similar analysis of GZMB+and GZMK+CD8+ T cells from the same patients demonstrated that in the blood, these cells, like their CD4+counterparts, are clonally expanded (FIG. 24A) and compromise a large proportion of the CD8+ T cells sharing specificity with tumor (FIG. 24B), with a trend towards increased Gini coefficient (repertoire restriction) in circulating CD8+ T cells with TCR clonotypes shared with tumor compared with circulating CD8+ T cells without shared tumor specificity (FIG. 24C). Similar trends are seen in tumor, with GZMB+and GZMK+CD8+ T cells demonstrating clonal expansion in particular in those T cells sharing clonotypes with blood (FIG. 24D and FIG. 24F), and GZMB+but in particular GZMK+CD8+ T cells representing a dominant fraction of all intratumoral CD8+ T cell sharing specificity with blood (FIG. 24E).

### Example 18

### Activated and Exhausted Cytotoxic CD4+ and CD8+ T Cell Phenotypes in Blood are Associated with Bladder Cancer, Atezolizumab Treatment, and Response to Atezolizumab

To validate surface protein expression of cytotoxic T cell populations in the blood, flow cytometry was conducted on paired pre- and post-treatment PBMCs from the blood of 14 bladder cancer patients treated with atezolizumab on this clinical trial, including 4 patients who had responses (pathologic downstaging of their tumor at the time of surgical cystectomy compared to initial diagnostic biopsy), and 10 patients who did not have responses. These included the 4 patients for whom scRNAseq/TCRseq data were obtained. Staining was also performed on PBMCs from 8 healthy individuals for comparison. This confirmed that GZMB+and GZMK+CD45+CD3+CD4+ T cells were found both in the blood (PBMC) as well as tumor and normal adjacent tissue (NAT) of bladder cancer patients (FIG. 25A). Additionally, significant correlations were found with specific cytotoxic CD4+ T cell subsets in the blood and the bladder cancer state, immunotherapy treatment, or clinical response to immunotherapy. CD4+FOXP3- CCR7- GZMB+HLA-DR+cytotoxic T cells that were not Tregs or naïve T cells and were activated were significantly increased in abundance with atezolizumab treatment (FIG. 25B), as are CD4+FOXP3- CCR7- GZMK+T cells that are exhausted and express PD-1 (PDCD1) and Tim3 (FIG. 25C). Notably, a specific activated subset of GZMK+CD4+ T cells that are HLA-DR+are significantly higher in abundance in the blood after atezolizumab treatment (“post”) of responders (“R”) compared to non-responders (“N”; *, p<0.05) indicating a specific correlation of cytotoxic CD4+ T cells with response to immunotherapy (FIG. 25D). Several CD4+CXCL13+states are significantly increased in pre-treatment blood of bladder cancer patients compared to healthy controls (CD4+FOXP3- CCR7- CXCL13+that are proliferating KI67+, activated HLA-DR+KI67+, or exhausted PD-1+; FIGS. 25E-25G).

As GZMB+and GZMK+CD8+ T cells are also found in the blood and tumor/adjacent normal tissue of these same patients (FIG. 25H), several cytotoxic CD8+subsets are also associated with atezolizumab treatment. Specifically, exhausted CD8+CCR7- GZMB+and GZMK+T cells that are Tim3+or PD-1+Tim3+(FIGS. 25I-25L), and activated CD8+ CCR7- GZMK+T cells that express HLA-DR, Ki67, or both (FIGS. 25M-250), are all significantly increased in abundance in the peripheral of atezolizumab treated patients on this trial.

### Example 19

### KLRG1+Cytotoxic T Cells have Enhanced Cytolytic Potential Against Autologous Bladder Tumor

Flow cytometry analysis also identifies KLRG1 as a marker of cytolytic activity in cytotoxic CD4+and CD8+ T cells. KLRG1+cells identify a substantial fraction of GZMB+and GZMK+CD4+and CD8+ T cells, particularly in blood where KLRG1+cells identify a significantly higher proportion of GZMB+and GZMK+cells than in tumor or in non-cytotoxic GZMB- GZMK- subsets in blood (FIGS. 26A, 26B). KLRG1 also identifies the subset of cytotoxic CD4+and CD8+ T cells with enhanced killing potential. Cultures of CD4+and CD8+ T cells from digests of bladder tumors were expanded with IL-2, sorted for KLRG1+or KLRG1-subsets, and then co-cultured with autologous bladder tumors from the same patient in time-lapse image-based cytotoxicity assays (Incucyte) using Annexin V as an apoptotic indicator. This demonstrated that KLRG1+CD4+and CD8+ TIL had enhanced cytotoxic activity which was blocked by an MHC-blocking antibody (FIGS. 26C, 26E), compared to KLRG1- TIL from the same patient which had reduced killing activity (FIGS. 26D, 26F). Furthermore, killing of IL-2-expanded CD4 from blood or tumor (after sorting prior to expansion to remove regulatory T cells, yielding “CD4_{eff}” effector T cells) in an experiment from a separate patient was not only higher in KLRG1+fractions, but also was enhanced by incubation with an antibody directed against the adhesion molecule E-cadherin which represents another means by which the cytotoxic activity of GZM+CD4+can be enhanced (blood killing in FIGS. 26G and 26H, TIL killing in FIGS. 26I and 26J, KLRG1+killing in FIGS. 26G and 26I, KLRG1- killing in FIGS. 26H and 26J).

### Example 20

### Experimental Procedures

### Tissue Processing

Tissues were obtained from patients with localized bladder transitional cell carcinoma (TCC) who either received 1-2 doses of neoadjuvant atezolizumab as part of an ongoing clinical trial (UCSF IRB# 14-15423), or standard of care treatments including chemotherapy (gemcitabine/carboplatin), or no systemic therapy prior to planned cystectomy. Cystectomy surgical specimens were obtained fresh from the operating field, and dissected in surgical pathology where grossly apparent tumor or adjacent bladder not grossly affected by tumor (“non-malignant”) were isolated, minced, and transported at room temperature immersed in L15 media with 15 mM HEPES and 600 mg% glucose. Once received, these were digested using Liberase TL as well as mechanical dissociation with heat (gentleMACS') using standard protocols. Single cell suspensions were obtained and counted for viability before staining for FACS. Healthy donor blood was separately collected, processed by gradient centrifugation to peripheral blood mononuclear cells (PBMCs), and cryopreserved to be thawed later for control experiments. Flow cytometry/FACS

Freshly dissociated TILs and previously frozen healthy donor PBMCs were used for sorting. Samples were stained with designated panels for 30 minutes at 4° C. and washed twice with FACS buffer (PBS, 2% FBS, 1 mM EDTA). Cells were incubated with DRAQ7™ (Biolegend, Cat# 424001) for 5 minutes at room temperature to stain dead cells. Samples were sorted on a FACSAria ^{TM} Fusion (Becton Dickinson) using FACSDiva™ software with single channel compensation controls acquired on the same day. For RNA sequencing flow validation, previously frozen TILs were thawed into FACS buffer and washed twice with FACS buffer. Live/dead fixable Near-IR dead cell stain (Invitrogen, Cat# L34975) was incubated with cells for 30 minutes at room temperature and washed once with FACS buffer. Samples were stained with designated panels for 30 minutes at 4° C. and washed twice with FACS buffer. Cells requiring intracellular staining were fixed and permeabilized with eBioscience FoxP3/ Transcription factor staining buffer set (Cat# 00-5523-00) according to the manufacturer's protocol. Intracellular staining with antibodies was carried out for 30 minutes at room temperature and washed twice with FACS wash. Cells were fixed with FluoroFix^{Tm} buffer (Biolegend, Cat# 422101) and washed once with FACS buffer. Cells were acquired the next day on a FACSymphony™ (Becton Dickinson) using FACSDiva™ with single channel compensation controls acquired on the same day. Data was analyzed off-line using FlowJo analysis software (FlowJo, LLC). Absolute counts (per ml of blood) for each immune subset was calculated by multiplying the percentage of each subset with the preceding parent subset and with the absolute lymphocyte count quantitated on the day of blood drawn. Single cell RNA sequencing

Droplet-based single-cell RNA sequencing (dscRNAseq) was performed using the 10× Genomics Chromium Single Cell 3′ platform, version 1, according to manufacturer's instructions. CD3⁺ CD4⁺ and CD3⁺ CD8⁺ T cells were sorted from digested tumor and non-malignant tissues, or Ficoll-purified and previously cryopreserved healthy control PBMCs, into 500 pi of PSA/0.04% BSA for loading onto 10X. Following library preparation, sequencing was performed on an Illumina HiSeq 2500 (Rapid Run mode). Paired samples from the same experiment and patient were processed in parallel during library preparation, and sequenced on the same flowcell to minimize batch effects. TCR sequencing

In brief, approximately 10% of the barcoded cDNA from the 10× Genomics workflow was utilized for TCR analysis. A pool of forward Vcc and V13 primers containing the Illumina read 1 primer sequence were used in conjunction with a reverse P7 primer to amplify CDR3 sequences from the TCR alpha and beta loci. An additional amplification step using forward primers containing the read 1 primer sequence in addition to the Illumina P5 and i5 sequences was used with a reverse P7 primer to create final TCR libraries for sequencing. Deep sequencing was performed on an Illumina NovaSeq Si with separate lanes for the TCR alpha and TCR beta sequencing. Read 1 contained 280 bp of the TCR alpha or beta CDR3 sequence, and the i7 read contained the 14 bp 10× Genomics barcode.

### Expression Analysis

After 10× sequencing data was processed through the Cell Ranger pipeline (version 1.1) with default settings, filtered gene-barcode matrices for single tumors were processed in Seurat (version 2.2.1, Rahul Satij a lab, New York Genome Center; Butler et al., 2018). Broadly, data processing was performed according to the Guided Clustering Tutorial at //satijalab.org/seurat/pbmc3k_tutorial.html and the CCA-Alignment Tutorial found at //satijalab.org/seurat/immune_alignment.html. Cells that expressed fewer than 150 genes were filtered out and genes that were expressed in fewer than 5 cells were also filtered out. Next, the gene expression measurements for each cell was non-malignantized by the total expression, which was multiplied by a scale factor of 10,000, and log-transformed the result. Further, the non-malignantized dataset was then scaled to remove confounding sources of variation by regressing out the signals driven by percent of mitochondrial gene expression and number of UMIs.

Multiple Canonical Correlation Analysis (MCCA) was then used to dimensionally reduce the dataset to 30 dimensions and align the dataset before further analysis. As the inputs to this algorithm, the dataset was first filtered down to 1168 genes for CD4⁺ tissue or 1171 genes for CD8⁺ tissue, which were found in the following way: for each “population”, which was defined as subset of the dataset consisting of a patient and tissue type, the 250 top variable genes were identified and the union of all of these genes was taken to create the input gene list. After examining the Metagene Bicorrelation Plot, a drop off in signal was observed after around CC20, and therefore CC 1-20 were chosen for the alignment, for which tissue was chosen as the grouping variable.

To discover subtle differences among the cells, KNN graph-based Louvain clustering was next performed. For CD4⁺ ′ and CD8⁺ TIL, a resolution of 1.2 in Seurat's “FindClusters” command was used. The lower bound for resolution chosen for clustering was based on whether the minimum number of known phenotypic categories for CD4⁺ and CD8⁺ TIL were represented and also based on iterative comparison with parallel FACS staining which validated expression of markers within specific clusters, while the upper bound was informed by the presence of clusters with minimal numbers of cells which would indicate overclustering. t-Stochastic Neighbor Embedding (tSNE) plots were used for visualization purposes.

Seurat's “FindConservedMarkers” command was next used to run differential expression analysis between each cluster and a CCR7^{high} central memory cluster and identify expression markers that define a given cluster regardless of tissue type. Significance was determined by non-parametric Wilcoxon rank sum test, with adjusted p value determined by Bonferroni correction. Heatmaps displaying conserved marker genes for each cluster were corrected across patients by fitting a linear model to remove sample-specific means. The gene lists were compared to known literature to label the clusters, SingleR (Aran et al., 2019) was used to map the expression signature for each cluster to the best correlated candidate immune reference signature, using the Blueprint, and Encode microarray and RNAseq references described within (Aran et al., 2019).

Differential expression testing between tumor and non-malignant compartments was performed with single cell expression data in a similar fashion, where testing between tumor and non-malignant compartments was restricted to samples that had paired cells available from both compartments. Differential expression testing between anti-PD-Ll-treated and untreated samples (excluding the chemotherapy sample) were performed using pseudobulk representations for each sample and DESeq2 (Love et al., 2014) after filtering out genes with fewer than 100 reads.

Correlation analysis between gene expressions from distinct clusters was performed by restricting to genes expressed across all clusters being tested, and then correlating the scaled expression of the multidimensional vector of shared genes between pairs of clusters.

Various unique features have been developed to derive the gene signatures described in Tables 2-4. For example, to determine the list of variable genes used for downstream clustering and differential expression testing (between clusters), the top 250 variable genes for each individual sample (n=7 tumors) were selected, and the union of these lists across all patients were analyzed to determine the final gene list for further analysis. Following unbiased clustering, each of the clusters (e.g., tCD4-c11, tCD8-c9, tCD4-c0) were compared to a single cluster expressing high levels of CCR7 (central memory cells). Prior to the present application, this approach has not been used by others, and was intended to provide an internal control within the data set described herein that is more sensitive to detect differentially expressed genes. This approach is justified as these CCR7+cells are devoid of expression of genes associated with other effector T cell types. In contrast, most existing differential expression testing compares each cluster versus the sum of all other clusters (one versus all). TCR analysis

TRA and TRB CDR3 nucleotide reads were demultiplexed by matching reads to 10× barcodes from cells with existing expression data that passed filtering in the Cell Ranger pipeline, excluding cell barcodes that overlapped between multiple samples. Following demultiplexing of the TRA and TRB CDR3 s, reads were aligned against known TRA/TRB CDR3 sequences then assembled into clonotype families using miXCR (Bolotin et al., 2015) with similar methodologies to a previous study (Zemmour et al., 2018). For any given 10X barcode, the most abundant TRA or TRB clonotype was accepted for further analysis; if 2 TRA or TRB clonotypes were equally abundant for a given 10× barcode, the clonotype with the highest sequence alignment score was used for further analysis. Detailed sequencing statistics and saturation analysis were also performed (data not shown). Only cells with paired TRA and TRB were used for further downstream analysis. Analysis utilizing TCR data only (number of unique cells sharing a specific TRA/TRB clonotype sequence, Gini coefficient) utilized cells both with and without a specific functional population that had been assigned by clustering. Analysis involving both TCR clonotype and function was restricted to cells with both a mapped TRA/TRB and a functional population from clustering.

To determine the enrichment of shared clonotypes between clusters, permutation tests were performed by randomly shuffling the cluster identities from all aggregated cells with paired TRA and TRB a total of 1,000 times with replacement, followed by generating a null hypothesis by counting the number of shared TCR clonotypes between clusters. Empirical p-values were calculated by comparing the observed number of shared TCR clones and those by the null hypothesis to determine significance. Specifically, the probability of obtaining the observed number (or greater) of shared TCR clones by chance was calculated as 1— the cumulative distribution function for that pair of populations, based on the mean and standard deviation of the randomly shuffled distribution. The level of significance for this analysis was set at 0.05.

### Isolation and Culturing of Tumor-Infiltrating Lymphocytes (TILs)

Single-cell suspensions from processed and digested bladder tumors were viably frozen at -80 C and stored prior to culture setup. To sort the TILs, frozen cancer cell aliquots were thawed, washed once with PBS, and counted by Vicell. Cells were subsequent stained and sorted by FACS. CD4 TIL (Draq7⁻CD45⁺ CD3⁺ CD4⁺ that were not CD25⁺ CD127¹⁰) and CD8 TIL (Drag?⁻CD45⁺ CD3⁺ CD8⁺ ) were sorted into ImmunoCult^{im} XF complete medium (Medium +10% FCS +1% penicillin/streptomycin; STEMCELL Technologies # 10981). T cells were pooled together for culturing. After centrifugation, T cells were suspended in ImmunoCult^{im} XF complete medium, and Dynabeads (Gibco # 11162D) were added to the culture per manufacturer's protocol. T cells were cultured in 96 well U-bottom plates, and briefly centrifuged to ensure cell contact with Dynabeads. T cell expansion was managed in two phases.

For the first week of T cell expansion, TILs were maintained with ImmunoCult' XF complete medium +200 IU/ml of human recombinant IL-2 (Peprotech # 200-02). From the second week onward, IL-2 concentration was gradually increased from 200 IU/ml to 2000 IU/ml based on cell growth kinetics (which varied by patient sample). T cells were harvested between 5-8 weeks for functional killing assays.

### Cytotoxic T Lymphocyte (CTL) Killing Assay

After expansion, TILs were again sorted for either CD4 or CD8 as distinct effector populations. Primary cancer cells from frozen aliquots were freshly thawed and sorted on CD45⁻ Draq7⁻ as target cells. To achieve various effector-to-target (E:T) ratios, 3000 cancer cell targets were suspended in ImmunoCult™ XF complete medium and seeded into each well. Different ratios of TILs were serially diluted and added to the corresponding wells. Each well contained 200 μl of medium supplemented with 0.25 μl of IncuCyte Red Cytotoxicity Reagent (Essen Bioscience # 4632). For WWI and MHCII blockade, 10 μg of blocking antibody was added into wells containing cancer cells and cultured at 37 C for 1 hour prior to co-culture with TILs. Cell culture was monitored by the IncuCyte Zoom system (EssenBioscience) at 15-30 minute intervals for a total of 12-24 hours. 2 independent experiments were performed using distinct aliquots from the same patient with co-culture of CD4+and CD8⁺ effectors with autologous tumor; representative results from 1 experiment are shown. Analysis was performed using the IncuCyte Zoom software. Red fluorescent images were background subtracted using a tophat filter with radius of 10 μm, and objects with a subtracted intensity of greater than 15 units were considered for further analysis. Tumor cells were larger than TIL based on inspection of wells with tumor cells alone or free TILs in wells containing TILs; based on this observation, the number of dying tumor cells per mm² was determined using a minimum area threshold of 75 μm², and in separate analyses the number of dying single TILs in wells containing TILs was determined using a minimum area threshold of 10 μm² and maximum area threshold of 65 μm². All numbers were normalized to the number at the start of the experiment. Out of focus frames were discarded, as were any wells where the first timeframe was out of focus precluding accurate normalization.

### Pseudotime Analysis

Pseudotime analysis was performed using standard methods for all input genes from these cells, to determine which cells are most developmentally related to other cells, and to arrange cells in trees with distinct branches based on relatedness, and specific branch points that separate these branches.

In particular, pseudotime analysis, including branched expression analysis modeling (BEAM) to identify all genes with branch-dependent differential expression followed by unbiased clustering of genes based on patterns of co-expression in specific branches, was performed using Monocle v2.10.1 as described previously (Qiu et al., 2017), for the combination of proliferating (tCD4-c11), regulatory (tCD4-c0, tCD4-c5, tCD4-c6) and cytotoxic (tCD4-c4, tCD4-c7, tCD4-c9, tCD4-c10) CD4⁺ populations from scRNAseq unbiased clustering described above.

Various unique features have been developed to derive the gene signatures described in Table 5 (proliferating cytotoxic CD4⁺ ; branch 1 cluster 5). The cells selected for this analysis were those that fell into proliferating CD4⁺ (tCD4-c11), regulatory CD4⁺ (tCD4-c0, tCD4-c5, tCD4-c6) and cytotoxic CD4⁺ (tCD4-c4, tCD4-c7, tCD4-c9, tCD4-c10) populations from scRNAseq clustering as described above.

Pseudotime analysis was performed using standard methods for all input genes from these cells, to determine which cells are most developmentally related to other cells, and to arrange cells in trees with distinct branches based on relatedness, and specific branch points that separate these branches. Individual branch points that separated proliferating cytotoxic CD4+ from their non-proliferating cytotoxic CD4+counterparts were then identified.

For these branch points, branched expression analysis modeling (BEAM) as performed as described in the referenced population to identify all genes with branch-dependent differential expression. Subsequently, unbiased clustering of these genes was then performed to divide them into groups of genes that had similar patterns of up- or down-regulation in specific branches. The clusters of genes (that were differentially expressed between branches) were then inspected to look for clusters that were upregulated in proliferating cytotoxic CD4+cells, while either showing no upregulation or downregulation in non-proliferating cytotoxic CD4+cells. (e.g., selecting for modules/clusters of genes that were coordinately upregulated in proliferating cytotoxic CD4+, and NOT upregulated in non-proliferating cytotoxic CD4).

For clusters of differentially expressed genes meeting this criteria (specific upregulation in proliferating cytotoxic CD4⁺ , e.g., the “branch 1 cluster 5” signature), gene set scoring was performed as described above to look for correlations with response to anti-PD-Ll or overall survival.

### Single Sample Gene Set Scoring

This was performed as described previously (Foroutan et al. 2018) for all CD4⁺ tissue-infiltrating populations using top-ranked differentially expressed genes (ranked by fold change) from the scRNAseq data set or from branch-specific signatures from BEAM analysis based on pseudotime analysis, applied to bulk RNAseq data from the IMvigor 210 trial of atezolizumab for metastatic bladder cancer using pre-treatment samples from bladder tumors where response information (by RECIST) and overall survival were both available (n=168 samples).

### Statistics

Specific statistical tests used for comparisons are described in the text. The chemotherapy sample was included in unbiased clustering, testing for conserved marker genes and tumor vs non-malignant testing, but was excluded from analyses of treatment effect (anti-PD-L1 vs untreated). For multiple testing correction, the Benjamini-Hochberg method was used with a false discovery rate <0.1 as implemented in the p.adjust function within the stats package within R.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### REFERENCES

Aran, D., Looney, A. P., Liu, L., Wu, E., Fong, V., Hsu, A., Chak, S., Naikawadi, R. P., Wolters, P. J., Abate, A. R., et al. (2019). Reference-based analysis of lung single-cell sequencing reveals a transitional profibrotic macrophage. Nat. Immunol. 20, 163-172.
Ayers, M., Lunceford, J., Nebozhyn, M., Murphy, E., Loboda, A., Kaufman, D. R., Albright, A., Cheng, J. D., Kang, S. P., Shankaran, V., et al. (2017). IFN-γ-related mRNA profile predicts clinical response to PD-1 blockade. J. Clin. Invest. 127, 2930-2940.
Baras, A. S., Drake, C., Liu, J. J., Gandhi, N., Kates, M., Hogue, M. O., Meeker, A., Hahn, N., Taube, J. M., Schoenberg, M. P., et al. (2016). The ratio of CD8 to Treg tumor-infiltrating lymphocytes is associated with response to cisplatin-based neoadjuvant chemotherapy in patients with muscle invasive urothelial carcinoma of the bladder. Oncoimmunology 5, el134412.
Blondel, V. D., Guillaume, J.-L., Lambiotte, R., and Lefebvre, E. (2008). Fast unfolding of communities in large networks. J. Stat. Mech. P10008.
Bolotin, D. A., Poslaysky, S., Mitrophanov, I., Shugay, M., Mamedov, I. Z., Putintseva, E. V., and Chudakov, D. M. (2015). MiXCR: software for comprehensive adaptive immunity profiling. Nat. Methods. 12, 380-1.
Butler, A., Hoffman, P., Smibert, P., Papalexi, E., and Satija, R. (2018). Integrating single-cell transcriptomic data across different conditions, technologies, and species. Nat. Biotechnol. 36, 411-420.
Cancer Genome Atlas Research Network. (2008). Comprehensive genomic characterization defines human glioblastoma genes and core pathways. Nature 455, 1061-8.
De Simone, M., Arrigoni, A., Rossetti, G., Gruarin, P., Ranzani, V., Politano, C., Bonnal, R. J. P., Provasi, E., Sarnicola, M.L., Panzeri, I., et al. (2016). Transcriptional Landscape of Human Tissue Lymphocytes Unveils Uniqueness of Tumor-Infiltrating T Regulatory Cells. Immunity 45, 1135-1147.
Duhen, T., Duhen, R., Montler, R., Moses, J., Moudgil, T., de Miranda, N. F., Goodall, C. P., Blair, T. C., Fox, B. A., McDermott, J. E., et al. (2018). Co-expression of CD39 and CD103 identifies tumor-reactive CD8 T cells in human solid tumors. Nat. Commun. 9, 2724.
Foroutan, M., Bhuva, D. D., Lyu, R., Horan, K., Cursons, J., and Davis, M. J. (2018). Single sample scoring of molecular phenotypes. BMC Bioinformatics 19, 404.
Gu-Trantien, C., Loi, S., Garaud, S., Equeter, C., Libin, M., de Wind, A., Ravoet, M., Le Buanec, H., Sibille, C., Manfouo-Foutsop, G., et al. (2013). CD4⁺ follicular helper T cell infiltration predicts breast cancer survival. J. Clin. Invest. 123, 2873-92.
Gu-Trantien, C., Migliori, E., Buisseret, L., de Wind, A., Brohee, S., Garaud, S., Noel, G., Dang Chi, V. L., Lodewyckx, J. N., Naveaux, C., et al. (2017). CXCL13-producing TFH cells link immune suppression and adaptive memory in human breast cancer. J. Clin. Invest. Insight 2, pii: 91487.
Guo, X., Zhang, Y., Zheng, L., Zheng, C., Song, J., Zhang, Q., Kang, B., Liu, Z., Jin, L., Xing, R., et al. (2018). Global characterization of T cells in non-small-cell lung cancer by single-cell sequencing. Nat. Med. 24, 978-985.
Hargadon, K. M., Johnson, C. E., and Williams, C. J. (2018). Immune checkpoint blockade therapy for cancer: An overview of FDA-approved immune checkpoint inhibitors. Int. Immunopharmacol. 62, 29-39.
Herbst, R. S., Soria, J. C., Kowanetz, M., Fine, G. D., Hamid, O., Gordon, M. S., Sosman, J. A., McDermott, D. F., Powderly, J. D., Gettinger, S. N., et al. (2014). Predictive correlates of response to the anti-PD-L1 antibody MPDL3280A in cancer patients. Nature 515, 563-7.
Kitano, S., Tsuji, T., Liu, C., Hirschhorn-Cymerman, D., Kyi, C., Mu, Z., Allison, J. P., Gnjatic, S., Yuan, J. D., and Wolchok, J. D. (2013). Enhancement of tumor-reactive cytotoxic CD4⁺ T cell responses after ipilimumab treatment in four advanced melanoma patients. Cancer Immunol Res. 1, 235-44.
Johnson, W. E., Li, C., and Rabinovic, A. (2007). Adjusting batch effects in microarray expression data using empirical Bayes methods. Biostatistics. 8, 118-27
Koshkin, V. S., and Grivas, P. (2018). Emerging Role of Immunotherapy in Advanced Urothelial Carcinoma. Curr. Oncol. Rep. 20, 48.
Krensky, A. M., and Clayberger, C. (2009). Biology and clinical relevance of granulysin. Tissue Antigens 73, 193-8.
Kurioka, A., Walker, L. J., Klenerman, P., Willberg, C. B. (2016). MAIT cells: new guardians of the liver. Clin. Transl. Immunology 5, e98.
Löfroos, A. B., Kadivar, M., Resic Lindehammer, S., and Marsal, J. (2017). Colorectal cancer-infiltrating T lymphocytes display a distinct chemokine receptor expression profile. Eur. J. Med. Res. 22, 40.
Love, M. I., Huber, W., and Anders, S. (2014). Moderated estimation of fold change and dispersion for RNA-seq data with DESeq2. Genome Biol, 15, 550.
Mariathasan, S., Turley, S. J., Nickles, D., Castiglioni, A., Yuen, K., Wang, Y., Kadel, E. E. III, Koeppen, H., Astarita, J. L., Cubas, R., et al. (2018). TGFI3 attenuates tumour response to PD-L1 blockade by contributing to exclusion of T cells. Nature 554, 544-548.
Martincorena, I., and Campbell, P. J. (2015). Somatic mutation in cancer and normal cells. Science 349, 1483-9.
Medley, Q. G., Kedersha, N., O'Brien, S., Tian, Q., Schlossman, S. F., Streuli, M., and Anderson, P. (1996). Characterization of GMP-17, a granule membrane protein that moves to the plasma membrane of natural killer cells following target cell recognition. Proc. Natl. Acad. Sci. U.S.A. 93, 685-9.
Oldham, K. A., Parsonage, G., Bhatt, R. I., Wallace, D. M., Deshmukh, N., Chaudhri, S., Adams, D. H., and Lee, S. P. (2012). T lymphocyte recruitment into renal cell carcinoma tissue: a role for chemokine receptors CXCR3, CXCR6, CCR5, and CCR6. Eur. Urol. 61, 385-94.
Ott, P. A., Hu, Z., Keskin, D. B., Shukla, S. A., Sun, J., Bozym, D. J., Zhang, W., Luoma, A., Giobbie-Hurder, A., Peter, L., et al. (2017). An immunogenic personal neoantigen vaccine for patients with melanoma. Nature 547, 217-221.
Parsonage, G., Machado, L. R., Hui, J. W., McLarnon, A., Schmaler, T., Balasothy, M., To, K. F., Vlantis, A. C., van Hasselt, C. A., Lo, K. W., et al. (2012). CXCR6 and CCR5 localize T lymphocyte subsets in nasopharyngeal carcinoma. Am. J. Pathol. 180, 1215-22.
Patil, V. S., Madrigal, A., Schmiedel, B. J., Clarke, J., O'Rourke, P., de Silva, A. D., Harris, E., Peters, B., Seumois, G., Weiskopf, D., et al. (2018). Precursors of human CD4⁺ cytotoxic T lymphocytes identified by single-cell transcriptome analysis. Sci. Immunol. 3, pii: eaan8664.
Philip, M., Fairchild, L., Sun, L., Horste, E. L., Camara, S., Shakiba, M., Scott, A. C., Viale, A., Lauer, P., Merghoub, T., et al. (2017). Chromatin states define tumour-specific T cell dysfunction and reprogramming. Nature 545, 452-456.
Plitas, G., Konopacki, C., Wu, K., Bos, P. D., Morrow, M., Putintseva, E. V., Chudakov, D. M., and Rudensky, A. Y. (2016). Regulatory T Cells Exhibit Distinct Features in Human Breast Cancer. Immunity 45, 1122-1134.
Preston, C. C., Maurer, M. J., Oberg, A. L., Visscher, D. W., Kalli, K. R., Hartmann, L. C., Goode, E. L., and Knutson, K. L. (2013). The ratios of CD8⁺ T cells to CD4⁺ CD25⁺ FOXP3⁺ and FOXP3⁻ T cells correlate with poor clinical outcome in human serous ovarian cancer. PLoS One 8, e80063
Qiu, X., Mao, Q., Tang, Y., Wang, L., Chawla, R., Pliner, H. A., and Trapnell, C. (2017). Reversed graph embedding resolves complex single-cell trajectories. Nat. Methods 14, 979-982.
Sahin, U., Derhovanessian, E., Miller, M., Kloke, B. P., Simon, P., Lower, M., Bukur, V., Tadmor, A. D., Luxemburger, U., Schrors, B., et al. (2017). Personalized RNA mutanome vaccines mobilize poly-specific therapeutic immunity against cancer. Nature 547, 222-226.
Sato, E., Olson, S. H., Ahn, J., Bundy, B., Nishikawa, H., Qian, F., Jungbluth, A. A., Frosina, D., Gnjatic, S., Ambrosone, C., et al. (2005). Intraepithelial CD8⁺ tumor-infiltrating lymphocytes and a high CD8^{H}iregulatory T cell ratio are associated with favorable prognosis in ovarian cancer. Proc. Natl. Acad. Sci. U.S.A. 102, 18538-43.
Schmidt, M., Weyer-Elberich, V., Hengstler, J. G., Heimes, A. S., Almstedt, K., Gerhold-Ay, A., Lebrecht, A., Battista, M. J., Hasenburg, A., Sahin, U., et al. (2018). Prognostic impact of CD4-positive T cell subsets in early breast cancer: a study based on the FinHer trial patient population. Breast Cancer Res. 20, 15.
Takada, K., Kashiwagi, S., Goto, W., Asano, Y., Takahashi, K., Takashima, T., Tomita, S., Ohsawa, M., Hirakawa, K., and Ohira, M. (2018). Use of the tumor-infiltrating CD8 to FOXP3 lymphocyte ratio in predicting treatment responses to combination therapy with pertuzumab, trastuzumab, and docetaxel for advanced HER2-positive breast cancer. J. Transl. Med. 16, 86.
Tirosh, I., Izar, B., Prakadan, S. M., Wadsworth, M. H. 2nd, Treacy, D., Trombetta, J. J., Rotem, A., Rodman, C., Lian, C., Murphy, G., et al. (2016). Dissecting the multicellular ecosystem of metastatic melanoma by single-cell RNA-seq. Science 352, 189-96.
Tumeh, P. C., Harview, C. L., Yearley, J. H., Shintaku, I. P., Taylor, E. J., Robert, L., Chmielowski, B., Spasic, M., Henry, G., Ciobanu, V., et al. (2014). PD-1 blockade induces responses by inhibiting adaptive immune resistance. Nature 515, 568-71.
van der Maaten, L. J. P., and Hinton, G. E. (2008). Visualizing data using t-SNE. J. Machine Learning Res. 9, 2579-2605.
Wei, Y., Lin, C., Li, H., Xu, Z., Wang, J., Li, R., Liu, H., Zhang, H., He, H., and Xu, J. (2018). CXCL13 expression is prognostic and predictive for postoperative adjuvant chemotherapy benefit in patients with gastric cancer. Cancer Immunol. Immunother. 67, 261-269.
Wolf, F., Angerer, P., and Theis, F. (2018). SCANPY: large-scale single-cell gene expression data analysis. Genome Biol. 19, 15.
Zemmour, D., Zilionis, R., Kiner, E., Klein, A. M., Mathis, D., and Benoist, C. (2018). Single-cell gene expression reveals a landscape of regulatory T cell phenotypes shaped by the TCR. Nat. Immunol. 19, 291-301.
Zhang, L., Yu, X., Zheng, L., Zhang, Y., Li, Y., Fang, Q., Gao, R., Kang, B., Zhang, Q., Huang, J. Y., et al (2018). Lineage tracking reveals dynamic relationships of T cells in colorectal cancer. Nature 564, 268-272.
Zheng, C., Zheng, L., Yoo, J. K., Guo, H., Zhang, Y., Guo, X., Kang, B., Hu, R., Huang, J. Y., Zhang, Q., et al. (2017a). Landscape of Infiltrating T Cells in Liver Cancer Revealed by Single-Cell Sequencing. Cell /69, 1342-1356.e16.
Zheng, G. X., Terry, J. M., Belgrader, P., Ryvkin, P., Bent, Z. W., Wilson, R., Ziraldo, S. B., Wheeler, T. D., McDermott, G. P., Zhu, J., et al. (2017b). Massively parallel digital transcriptional profiling of single cells. Nat. Commun. 8, 14049.

What is claimed is:

1. A method for predicting responsiveness of an individual having or suspected of having bladder cancer to a therapy comprising a Programmed Death Ligand 1 (PD-L1) antagonist, the method comprising:
a) profiling expression levels of a panel of genes associated with T-cell specialization and/or T-cell exhaustion expressed in a T cell population from a biological sample obtained from said individual to generate a cell composition profile of the T cell population;
b) determining the presence of a gene signature biomarker in the T cell population based at least in part upon the measured expression levels and the generated cell composition profile, wherein said gene signature biomarker comprises one or more genes whose expression is specifically upregulated in proliferating and/or non-proliferating cytotoxic CD4+ T cells while remains unchanged in CD8+ T cells;
c) identifying the individual as predicted to have an increased responsiveness to the anti-PD-L1 therapy if the gene signature is present in the biological sample.

2. A method for selecting an individual having bladder cancer to be subjected to a therapy comprising a PD-L1 antagonist, the method comprising:
a) profiling expression levels of a panel of genes associated with T-cell specialization and/or T-cell exhaustion expressed in a T cell population from a biological sample obtained from said individual to generate a cell composition profile of the T cell population;
b) determining the presence of a gene signature biomarker in the T cell population based at least in part upon the measured expression levels and the generated cell composition profile, wherein said gene signature biomarker comprises one or more genes whose expression is specifically upregulated in proliferating and/or non-proliferating cytotoxic CD4+ T cells while remains unchanged in CD8+ T cells;
c) selecting the individual who is determined to have the gene signature present in the biological sample as an individual to be subjected to a therapy comprising a PD-L1 antagonist.

3. A method for treating an individual having bladder cancer, the method comprising:
a) profiling expression levels of a panel of genes associated with T-cell specialization and/or T-cell exhaustion expressed in a T cell population from a biological sample obtained from said individual to generate a cell composition profile of the T cell population;
b) determining the presence of a gene signature biomarker in the T cell population based at least in part upon the measured expression levels and the generated cell composition profile, wherein said gene signature biomarker comprises one or more genes whose expression is specifically upregulated in proliferating and/or non-proliferating cytotoxic CD4+ T cells while remains unchanged in CD8+ T cells;
c) selecting a therapy comprising a PD-L1 antagonist; and
d) administering a therapeutically effective amount of the selected therapy to said individual.

4. The method of any one of claims 1 to 3, wherein the cell composition profile comprises relative proportions of the following T cell subpopulations: tumor-reactive ENTPD1+CD8+ T cells, naïve CD8+ T cells, HSP+CD8+ T cells, mucosal-associated invariant CD8+ T cells, FGFBP2+CD8+ T cells, XCL1+XCL2+CD8+ T cells, central memory CD8+ T cells, effector memory CD8+ T cells, exhausted CD8+ T cells, proliferating CD8+ T cells, regulatory CD4+ T cells, central memory CD4+ T cells, exhausted CD4+ T cells, proliferating cytotoxic CD4+ T cells, and non-proliferating cytotoxic CD4+ T cells.

5. The method of any one of claims 1 to 4, wherein the gene signature biomarker comprises one or more of the following parameters:
i. one or more genes identified in Table 2 or Table 7 as upregulated in proliferating CD8+ T cells;
ii. one or more genes identified in Table 3 or Table 10 as upregulated in proliferating CD4⁺ T cells;
iii. one or more genes identified in Table 4 or Table 8 as upregulated in regulatory CD4⁺ T cells;
iv. one or more genes identified in Table 9 as upregulated in cytotoxic CD4+ T cells; and
v. one of more genes identified in Table 5 as upregulated in proliferative cytotoxic CD4⁺ T cells.

6. The method of any one of claims 1 to 5, wherein the gene signature biomarker comprises at least 2, at least 3, at least 5, at least 10, at least 20, at least 30, at least 40, at least 50 genes.

7. The method of any one of claims 1 to 6, wherein the gene signature biomarker comprises one or more of ABCB1, ACTB, APBA2, ATP5E, CARD16, CXCL13, GPR18, GZMB, HIST1H4C, IGLL5, IL2RA, IL32, KIAA0101, KIF15, MIR4435-1HG, MYL6, PEG10, SLAMF7, STMN1, TIGIT, TMSB10, TUBA1B, TUBB, GZMK, HLA-DR, PDCD1, TIM3, KLRG, and combinations of any thereof

8. The method of claim 7, wherein the gene signature biomarker comprises one or more of ABCB1, ACTB, APBA2, GPR18, HIST1H4C, IGLL5, IL2RA, IL32, MIR4435-1HG, MYL6, SLAMF7, STMN1, TMSB10, TUBB, and combinations of any thereof.

9. The method of claim 7, wherein the gene signature biomarker comprises one or more of GZMK, HLA-DR, PDCD1, TIM3, KLRG1, and combinations of any thereof.

10. The method of any one of claims 1 to 9, wherein the biological sample comprises bladder cancer cell.

11. The method of any one of claims 1 to 9, wherein the biological sample comprises peripheral blood.

12. The method of any one of claims 1 to 11, wherein the bladder cancer is selected from the group consisting of squamous cell carcinoma, non-squamous cell carcinoma, adenocarcinoma, and small cell carcinoma.

13. The method of any one of claims 1 to 12, wherein the bladder cancer is selected from the group consisting of metastatic bladder cancer, non-metastatic bladder cancer, early-stage bladder cancer, non-invasive bladder cancer, muscle-invasive bladder cancer (MIBC), non-muscle-invasive bladder cancer (NMIBC), primary bladder cancer, advanced bladder cancer, locally advanced bladder cancer, bladder cancer in remission, progressive bladder cancer, and recurrent bladder cancer.

14. The method of claim 13, wherein the bladder cancer is metastatic bladder cancer.

15. The method of any one of claims 1 to 14, wherein the PD-L1 antagonist comprises an anti-PD-L1 antibody.

16. The method of claim 15, wherein the anti-PD-L1 antibody comprises one or more of atezolizumab (MPDL3280A), BMS-936559 (MDX-1105), durvalumab (MEDI4736), avelumab (MSB0010718C), YW243.55.570, and combinations of any thereof.

17. The method of claim 16, wherein the anti-PD-L1 antibody comprises atezolizumab.

18. The method of any one of claims 1 to 14, wherein the PD-L1 antagonist comprises an anti-PD1 antibody.

19. The method of claim 18, wherein the anti-PD1 antibody comprises one or more of pembrolizumab, nivolumab, cemiplimab, pidilizumab, lambrolizumab, MEDI-0680, PDR001, REGN2810, and combinations of any thereof.

20. The method of claim 19, wherein the anti-PD1 antibody comprises pembrolizumab.

21. The method of claim 17, wherein the gene signature biomarker comprises one or more genes whose expression is upregulated in proliferating CD4+ T cells and/or upregulated in non-proliferating CD4+ T cells while remains substantially unchanged in CD8+ T cells.

22. The method of claim 21, wherein the gene signature biomarker comprises one or more genes selected from the group consisting of ABCB1, APBA2, SLAMF7, GPR18, PEG10, and combinations of any thereof.

23. The method of claim 21, wherein the gene signature biomarker comprises one or more genes whose expression is upregulated in cytotoxic CD4+ T cells.

24. The method of claim 23, wherein the gene signature biomarker comprises one or more genes selected from the group consisting of GZMK, GZMB, HLA-DR, PDCD1, TIM3, and combinations of any thereof.

25. The method of claim 24, wherein the gene signature biomarker comprises a gene combination selected from the group consisting of:
(a) expression of CD4, GZMB, and HLA-DR;
(b) expression of CD4, GZMK, and HLA-DR; and
(c) expression of CD4, GZMK, PDCD1, and TIM3.

26. The method of claim 25, wherein the gene signature biomarker further comprises undetectable expression of FOXP3 and CCR7.

27. The method of claim 17, wherein the gene signature biomarker comprises one or more genes whose expression is upregulated in cytotoxic CD8+ T cells.

28. The method of claim 27, wherein the gene signature biomarker comprises one or more genes selected from the group consisting of GZMB, GZMK, HLA-DR, PDCD1, Ki67, TIM3, and combinations of any thereof.

29. The method of claim 28, wherein the gene signature biomarker comprises a gene combination selected from the group consisting of:
(a) expression of CD8, GZMB and TIM3;
(b) expression of CD8, GZMB, PDCD1, and TIM3;
(c) expression of CD8, GZMK and TIM3;
(d) expression of CD8, GZMK, PDCD1, and TIM3;
(e) expression of CD8, GZMK and HLA-DR;
(f) expression of CD8, GZMK and Ki67; and
(g) expression of CD8, GZMK, HLA-DR, and Ki67.

30. The method of claim 29, wherein the gene signature biomarker futher comprises undetectable expression of CCR7.

31. The method of any one of claims 1 to 29, wherein said profiling expression levels of a panel of genes associated with T-cell specialization and/or T-cell exhaustion comprises a nucleic-acid-based analytical assay selected from the group consisting of single-cell RNA sequencing, T-cell receptor (TCR) sequencing, single sample gene set enrichment analysis, northern blotting, fluorescent in-situ hybridization (FISH), polymerase chain reaction (PCR), real-time PCR, reverse transcription polymerase chain reaction (RT-PCR), quantitative reverse transcription PCR (qRT-PCR), serial analysis of gene expression (SAGE), microarray, tiling arrays.

32. The method of claim 30, wherein the nucleic acid-based analytical assay comprises single-cell RNA sequencing.

33. The method of any one of claims 1 to 29, wherein said profiling expression levels of a panel of genes associated with T-cell specialization and/or T-cell exhaustion comprises a protein expression-based analytical assay selected from the group consisting of ELISA, immunohistochemistry, western blotting, mass spectrometry, flow cytometry, protein-microarray, immunofluorescence, multiplex detection assay, and combinations of any thereof.

34. The method of claim 33, wherein the protein expression-based analytical assay comprises flow cytometry.

35. The method of any one of claims 1 to 34, further comprising treating the bladder cancer by administering to the individual a first therapy comprising a therapeutically effective amount of the PD-L1 antagonist.

36. The method of any one of claims 1 to 34, further comprising:
a) selecting a PD-L1 antagonist appropriate for the therapy of the bladder cancer in the individual based on whether the gene signature biomarker is present in the individual; and
b) administering a first therapy comprising a therapeutically effective amount of the selected PD-L1 antagonist to the individual.

37. The method of any one of claims 35 to 36, the gene signature biomarker is associated with longer survival of the individual following the therapy with the PD-L1 antagonist.

38. The method of any one of claims 35 to 37, wherein the first therapy is administered to the individual in combination with a second therapy.

39. The method of claim 38, wherein the second therapy is selected from the group consisting of chemotherapy, radiation therapy, immunotherapy, immunoradiotherapy, hormonal therapy, toxin therapy, and surgery.

40. The method of any one of claims 38 to 39, wherein the second therapy is an anti- PD-1 therapy.

41. The method of any one of claims 38 to 39, wherein the second therapy is an anti-transforming growth factor p (TGF-(3) therapy.

42. The method of any one of claims 38 to 41, wherein the first therapy and the second therapy are administered concomitantly.

43. The method of any one of claims 38 to 41, wherein the first therapy and the second therapy are administered sequentially.

44. The method of claim 43, wherein the first therapy is administered before the second therapy.

45. The method of claim 43, wherein the first therapy is administered after the second therapy.

46. The method of any one of claims 38 to 39, wherein the first therapy is administered before and/or after the second therapy.

47. The method of any one of claims 38 to 39, wherein the first therapy and the second therapy are administered in rotation.

48. The method of any one of claims 38 to 47, wherein the first therapy and the second therapy are administered together in the same composition or in separate compositions.

49. The method of claim 48, wherein the first therapy and the second therapy are administered together in a single formulation.

50. A kit comprising:
a) one or more detection reagents for profiling expression levels of a panel of genes associated with T-cell specialization and/or T-cell exhaustion expressed in a T cell population from a biological sample obtained from an individual; and
b) instructions for use in predicting responsiveness of a bladder cancer to an anti-PD-Ll therapy and/or in treating a bladder cancer in an individual.

51. The kit of claim 50, further comprising an antagonist of PD-L1 and optionally an antagonist of PD-1 or a combination thereof

52. A system comprising:
a) at least one processor; and
b) at least one memory including program code which when executed by the one memory provides operations for performing a method according to any one of claims 1 to

49.

53. The system of claim 52, wherein the operations comprise:
a) acquiring knowledge of the presence of a gene signature biomarker in a biological sample from an individual; and
b) providing, via a user interface, a prognosis for the subject based at least in part on the acquired knowledge.
